(19) United States
(12) Patent Application Publication
(10) Pub. No.: US 2026/0308208 A1
(43) Pub. Date: Oct. 8, 2026

(54) RHIZAVIDIN VARIANTS

(71) Applicant: AFFINIVAX, INC., Wilmington, DE (US)

(72) Inventors: Heidi BURKE, Saugus, MA (US); Brian CIESLEWICZ, Brookline, MA (US); Teresa BROERING, Brookline, MA (US)

(73) Assignee: AFFINIVAX, INC., Wilmington, DE (US)

(21) Appl. No.: 19/480,476

(22) PCT Filed: May 2, 2024
(86) PCT No.: PCT/US2024/027331

§ 371 Date: Oct. 31, 2025

Related U.S. Application Data
(60) Provisional application No. 63/464,129, filed on May 4, 2023.

(51) Int. Cl. C07K 14/195; A61K 39/00; A61K 39/02; A61K 39/09; A61K 39/215; A61P 37/04; C12R 1/41

(52) U.S. Cl. CPC C07K 14/195; A61K 39/001156; A61K 39/0208; A61K 39/092; A61K 39/215; A61P 37/04; A61K 2039/6068; A61K 2039/645; C07K 2319/00; C12R 2001/41

(57) ABSTRACT

The present disclosure provides technologies (e.g., compositions, methods, and kits, etc.) for rhizavidin variants. In particular, the disclosure provides a variant rhizavidin polypeptide, comprising a mutation at one or more N-linked glycosylation sites of a wild-type rhizavidin polypeptide, fusion proteins comprising the variant rhizavidin polypeptide, compositions comprising the variant rhizavidin polypeptide or fusion protein and uses in immunization or methods of immunization.

### BACKGROUND

Rhizavidin, a naturally occurring dimeric protein in the avidin protein family, was first discovered in Rhizobium etli, a symbiotic bacterium of the common bean. Rhizavidin is a biotin-binding protein, which forms a noncovalent homodimer, rather than a homotetramer as formed by many avidins such as avidin, streptavidin, or bradavidin. Rhizavidin binds non-covalently to biotin with high affinity (dissociation constant [KD]≈10⁻¹⁵ M).

### SUMMARY

The present disclosure, among other things, provides insights and technologies for engineered rhizavidin variants. In many embodiments, engineered rhizavidin variants described herein are useful as biotin-binding proteins for various applications, e.g., in some embodiments, to couple two molecular entities (e.g., small molecules, proteins, lipids, polysaccharides, etc.) via non-covalent biotin-rhizavidin interaction.

In one aspect, the present disclosure recognizes that, while rhizavidin is a bacterial protein, which has been commonly recombinantly expressed in bacteria, such as E. coli, it may be desirable to express rhizavidin using a eukaryotic (e.g., mammalian, yeast, or insect) expression system in certain circumstances or applications. For example, such certain circumstances or applications include, e.g., production of a fusion protein comprising a rhizavidin polypeptide and a polypeptide of interest present in a eukaryotic cell (e.g., a mammalian cell), virus, fungus, parasite, or in some instances present in a bacterial cell, where post-translation modification and/or processing of the polypeptide of interest in eukaryotic cells are useful. In some embodiments, polypeptides of interest that may benefit from post-translational modification and/or processing in eukaryotic cells may include polypeptides that contain multiple disulfide bonds, polypeptides that are hydrophobic, and/or polypeptides that require additional chaperones present in eukaryotic cells to facilitate their proper expression and folding. In some embodiments, such a polypeptide of interest is or comprises a polypeptide antigen present in a eukaryotic cell (e.g., a mammalian cell). In some embodiments, such a polypeptide antigen present in a eukaryotic cell (e.g., a mammalian cell) may be or comprise a tumor antigen. In some embodiments, such fusion proteins may be useful in a variety of applications, e.g., in some embodiments, therapeutic applications including, e.g., but not limited to immunogenic compositions based on MAPS platform as described in International Patent Application Nos. PCT/US2012/037412, PCT/US2019/050907, PCT/US2022/042964, and PCT/US2022/043156.

In one aspect, the present disclosure identifies the source of problems with expressing wild-type rhizavidin or a fragment thereof in eukaryotic cells (e.g., mammalian, yeast, or insect cells). For example, the present disclosure recognizes that rhizavidin has 4-5 N-linked glycosylation sites as bioinformatically predicted, which are likely not glycosylated in bacteria from which it is derived or when recombinantly expressed in E. coli, but are variably glycosylated when expressed in eukaryotic (e.g., mammalian, yeast, or insect) expression systems. Without wishing to be bound by any particular theory, various patterns of N-linked glycosylation present in rhizavidin expressed in eukaryotic cells can lead to poor expression in eukaryotic cells and/or generation of glycosylated rhizavidin with various molecular weights that make purification of rhizavidin challenging.

In some embodiments, the present disclosure provides technologies (including compositions, methods, kits, etc.) that solve such problems, among other things, by engineering rhizavidin variants to remove glycosylation activity, while retaining desirable characteristics of a wild-type rhizavidin, including, e.g., but not limited to biotin-binding activity, dimerization activity, etc. In some embodiments, the present disclosure provides insights that certain amino acid positions and/or mutations within glycosylation motifs can adversely impact dimerization activity and/or expression of rhizavidin. For example, it is demonstrated that a mutation at a position corresponding to the amino acid residue N106 (e.g., in some embodiments, N106A) within a glycosylation motif of rhizavidin (as set forth in SEQ ID NO: 1) reduced expression of rhizavidin in eukaryotic cells (e.g., mammalian, yeast, or insect cells), as compared to a mutation at a position corresponding to the amino acid residue T108 within the same glycosylation motif (e.g., in some embodiments, T108A). It is also demonstrated that a mutation at a position corresponding to the amino acid residue S119 (e.g., in some embodiments, S119A) within a secondary glycosylation motif of rhizavidin (as set forth in SEQ ID NO: 1) reduced glycosylation and promoted expression of rhizavidin in eukaryotic cells (e.g., mammalian, yeast, or insect cells). Additionally, it is unexpectedly found that a less conservative amino acid substitution (e.g., N to A substitution) present in a glycosylation motif of rhizavidin allowed rhizavidin to form dimers as observed in wild-type rhizavidin, while a more conservative amino acid substitution (e.g., N to Q substitution) produced monomeric rhizavidin. Accordingly, in some embodiments, the present disclosure provides engineered rhizavidin variants comprising certain combinations of amino acid mutations (e.g., as described herein) that are particularly useful for expression in eukaryotic cells (e.g., mammalian, yeast, or insect cells).

One aspect of the present disclosure provides a variant rhizavidin polypeptide. In some embodiments, the variant rhizavidin polypeptide comprises a mutation at one or more N-linked glycosylation sites of a wild-type rhizavidin polypeptide. In some embodiments, the variant rhizavidin polypeptide comprises a mutation at each of four or more N-linked glycosylation sites of a wild-type rhizavidin polypeptide. In some embodiments, the variant rhizavidin polypeptide comprises a mutation at each of five or more N-linked glycosylation sites of a wild-type rhizavidin polypeptide. In some embodiments, at least one of the N-linked glycosylation sites comprises a consensus sequence of NXaaS or NXaaT, wherein Xaa is any amino acid residue except proline (P).

In some embodiments, each of the N-linked glycosylation sites comprises a consensus sequence of NXaaS or NXaaT, wherein Xaa is any amino acid residue except proline (P). In some embodiments, the wild-type rhizavidin polypeptide comprises the amino acid sequence as set forth below:
FDASNFKDFSSIASASSSWQNQSGSTMIIQVDSFGNVSGQYVNRAQGTGCQNSPYPLTGR VNGTFIAFSVGWNNSTENCNSATGWTGYAQVNGNNTEIVTSWNLAYEGGSGPAIEQGQ DTFQYVPTTENKSLL (SEQ ID NO: 2), wherein the amino acid sequence of SEQ ID NO: 2 corresponds to amino acid residues 45-179 of the wild-type rhizavidin polypeptide.

In some embodiments, the wild-type rhizavidin polypeptide comprises the amino acid sequence as set forth below:
MIITSLYATFGTIADGRRTSGGKTMIRTNAVAALVFAVATSALAFDASNFKDFSSIASASS SWQNQSGSTMIIQVDSFGNVSGQYVNRAQGTGCQNSPYPLTGRVNGTFIAFSVGWNNS TENCNSATGWTGYAQVNGNNTEIVTSWNLAYEGGSGPAIEQGQDTFQYVPTTENKSLL KD (SEQ ID NO: 1), wherein the amino acid sequence of SEQ ID NO: 1 corresponds to amino acid residues 1-179 of the wild-type rhizavidin polypeptide.

In some embodiments, at least one of the N-linked glycosylation sites does not comprise amino acid residues 65-67 or 173-175 of a wild-type rhizavidin polypeptide.

In some embodiments, at least one of the N-linked glycosylation sites comprises amino acid residues selected from the group consisting of amino acid residues 80-82, 106-108, 117-119, 118-120, and 138-140 of the wild-type rhizavidin polypeptide.

In some embodiments, each of the N-linked glycosylation sites comprises amino acid residues selected from the group consisting of amino acid residues 80-82, 106-108, 117-119, 118-120, and 138-140 of the wild-type rhizavidin polypeptide. In some embodiments, the one or more N-linked glycosylation sites are each of amino acid residues 80-82, 106-108, 117-119, 118-120, and 138-140 of the wild-type rhizavidin polypeptide.

In some embodiments, each of the N-linked glycosylation sites comprises amino acid residues selected from the group consisting of amino acid residues 80-82, 106-108, 118-120, and 138-140 of the wild-type rhizavidin polypeptide. In some embodiments, the one or more N-linked glycosylation sites are each of amino acid residues 80-82, 106-108, 118-120, and 138-140 of the wild-type rhizavidin polypeptide.

In some embodiments, at least one of the mutations is at a position selected from the group consisting of amino acid residues 80, 106, 108, 118, 119, and 138 of the wild-type rhizavidin polypeptide. In some embodiments, each of the mutations is at a position independently selected from the group consisting of amino acid residues 80, 106, 108, 118, 119, and 138 of the wild-type rhizavidin polypeptide.

In some embodiments, each of the mutations is at a position independently selected from the group consisting of amino acid residues 80, 108, 118, 119, and 138 of the wild-type rhizavidin polypeptide. In some embodiments, a variant rhizavidin polypeptide comprises mutations at each of amino acid residues 80, 108, 118, 119, and 138 of the wild-type rhizavidin polypeptide.

In some embodiments, each of the mutations is at a position independently selected from the group consisting of amino acid residues 80, 106, 118, 119, and 138 of the wild-type rhizavidin polypeptide. In some embodiments, a variant rhizavidin polypeptide comprises mutations at each of amino acid residues 80, 106, 118, 119, and 138 of the wild-type rhizavidin polypeptide.

In some embodiments, each of the mutations is at a position independently selected from the group consisting of amino acid residues 80, 106, 118, and 138 of the wild-type rhizavidin polypeptide. In some embodiments, a variant rhizavidin polypeptide comprises mutations at each of amino acid residues 80, 106, 118, and 138 of the wild-type rhizavidin polypeptide.

In some embodiments, at least one of the mutations is selected from the group consisting of: (i) residue 80 N to A/Q, (ii) residue 106 N to A/Q, (iii) residue 108 T to A/Q, (iv) residue 118 N to A/Q, (v) residue 119 S to A/Q, and (vi) residue 138 N to A/Q. In some embodiments, each of the mutations is independently selected from the group consisting of: (i) residue 80 N to A/Q, (ii) residue 106 N to A/Q, (iii) residue 108 T to A/Q, (iv) residue 118 N to A/Q, (v) residue 119 S to A/Q, and (vi) residue 138 N to A/Q.

In some embodiments, each of the mutations is independently selected from the group consisting of: (i) residue 80 N to A, (ii) residue 108 T to A, (iii) residue 118 N to A, (iv) residue 119 S to A, and (v) residue 138 N to A. In some embodiments, a variant rhizavidin polypeptide comprises mutations at each of: (i) residue 80 N to A, (ii) residue 108 T to A, (iii) residue 118 N to A, (iv) residue 119 S to A, and (v) residue 138 N to A.

In some embodiments, each of the mutations is independently selected from the group consisting of: (i) residue 80 N to A, (ii) residue 106 N to A, (iii) residue 118 N to A, (iv) residue 119 S to A, and (v) residue 138 N to A. In some embodiments, a variant rhizavidin polypeptide comprises mutations at each of: (i) residue 80 N to A, (ii) residue 106 N to A, (iii) residue 118 N to A, (iv) residue 119 S to A, and (v) residue 138 N to A.

In some embodiments, each of the mutations is independently selected from the group consisting of: (i) residue 80 N to A, (ii) residue 106 N to A, (iii) residue 118 N to A, and (iv) residue 138 N to A. In some embodiments, a variant rhizavidin polypeptide comprises mutations at each of: (i) residue 80 N to A, (ii) residue 106 N to A, (iii) residue 118 N to A, and (iv) residue 138 N to A.

In some embodiments, each of the mutations is independently selected from the group consisting of: (i) residue 80 N to Q, (ii) residue 106 N to Q, (iii) residue 118 N to Q, and (iv) residue 138 N to Q. In some embodiments, a variant rhizavidin polypeptide comprises mutations at each of: (i) residue 80 N to Q, (ii) residue 106 N to Q, (iii) residue 118 N to Q, and (iv) residue 138 N to Q.

In some embodiments, a variant rhizavidin polypeptide described herein does not comprise amino acid residues 1-44 of wild-type rhizavidin.

In some embodiments, a variant rhizavidin polypeptide described herein is or comprises an amino acid sequence having at least 80%, at least 85%, at least 90%, at least 95%, at least 96%, at least 97%, at least 98%, or at least 99% identity to the amino acid sequence of any one of SEQ ID NOs: 4-15. In some embodiments, the variant rhizavidin polypeptide is or comprises an amino acid sequence that is identical to the amino acid sequence of any one of SEQ ID NOs: 4-15.

In some embodiments, a variant rhizavidin polypeptide described herein is characterized in that it binds to biotin or a derivative or mimic molecule thereof, at an affinity that is at least 50%, at least 60%, at least 70%, at least 80%, or at least 90% of a wild-type rhizavidin polypeptide. In some embodiments, the variant rhizavidin polypeptide is characterized in that it binds to a biotin derivative, lipoic acid, HABA (hydroxyazobenzene-benzoic acid), dimethyl-HABA, or an amine-PEG3-biotin ((+)-biotinylation-3-6,9-trixaundecanediamine), at an affinity that is at least 50%, at least 60%, at least 70%, at least 80%, or at least 90% of a wild-type rhizavidin polypeptide.

In some embodiments, a variant rhizavidin polypeptide described herein is characterized in that it is substantially non-glycosylated when expressed in a mammalian cell. In some embodiments, the variant rhizavidin polypeptide is characterized in that its degree of glycosylation when expressed in a mammalian cell is reduced by at least 70%, at least 80%, or at least 90% as compared to a wild-type rhizavidin polypeptide.

In some embodiments, the variant rhizavidin polypeptide is characterized in that when a population of the variant rhizavidin polypeptide is expressed in mammalian cells the population of variant rhizavidin polypeptides have an average monomeric molecular weight between 17 kDa and 19 kDa. In some embodiments, the variant rhizavidin polypeptide is characterized in that when a population of the variant rhizavidin polypeptide is expressed in mammalian cells the population of variant rhizavidin polypeptides have an average dimeric molecular weight between 26 kDa and 28 kDa.

Another aspect of the present disclosure provides a fusion protein comprising a variant rhizavidin polypeptide disclosed herein and at least one additional polypeptide. In some embodiments, the at least one additional polypeptide is or comprises one or more polypeptide antigens. In some embodiments, at least one of the polypeptide antigens is or comprises an antigen selected from the group consisting of: bacterial polypeptide antigens, fungal polypeptide antigens, parasitic polypeptide antigens, viral polypeptide antigens, and mammalian polypeptide antigens (e.g., tumor antigens). In some embodiments, at least one of the polypeptide antigens is or comprises an antigen selected from the group consisting of: streptococcal antigens (e.g., S. pneumoniae, group A, group B, and viridans antigens), tuberculosis antigens, tetanus antigens, anthrax antigens, pertussis antigens, staphylococcal antigens (e.g., S. aureus), Haemophilus antigens, Enterobacter, antigens, Acinetobacter antigens, Citrobacter antigens, Serratia antigens, Clostridia antigens, Campylobacter antigens, Vibriocholera antigens, Pseudomonas antigens, meningococcal antigens, Neisseria gonorrhoeae antigens, Chlamydia trachomatis antigens, Klebsiella antigens, Shigella antigens, Salmonella antigens, E. coli antigens, malaria antigens, HIV antigens, HPV antigens, influenza (e.g., seasonal or epidemic) antigens, coronavirus antigens (e.g., SARS-CoV-2 antigens), herpes (e.g., HSV) antigens, tumor antigens, and combinations thereof.

In some embodiments, at least one of the polypeptide antigens is or comprises a pneumococcal polypeptide antigen. In some embodiments, at least one polypeptide antigen is or comprises a polypeptide antigen selected from the group consisting of: a pneumolysin polypeptide antigen, a SP1500 polypeptide antigen, a SP0785 polypeptide antigen, and a SP0435 polypeptide antigen. In some embodiments, the fusion protein comprises: (i) an SP1500 polypeptide antigen or fragment thereof, and (ii) an SP0785 polypeptide antigen or fragment thereof. In some embodiments, the fusion protein comprises: (i) a pneumolysin polypeptide antigen or fragment thereof, and (ii) an SP0435 polypeptide antigen or fragment thereof.

In some embodiments, at least one of the polypeptide antigens has an amino acid sequence that is or comprises the amino acid sequence of any one of SEQ ID NOs: 619-628.

In some embodiments, a fusion protein described herein further comprises a peptide linker positioned between the variant rhizavidin polypeptide and the at least one additional polypeptide. In some embodiments, a fusion protein described herein further comprises a bacterial signal sequence at the N-terminus. In some embodiments, a fusion protein described herein further comprises a purification tag at the C-terminus. In some embodiments, the purification tag is a histidine tag, a c-myc tag, a Halo tag, a FLAG tag, or a combination thereof.

In some embodiments, a fusion protein described herein has an amino acid sequence that is or comprises the amino acid sequence of any one of SEQ ID NOs: 801-868.

Another aspect of the present disclosure provides a nucleic acid comprising a nucleotide sequence encoding a variant rhizavidin polypeptide described herein or a fusion protein described herein. In some embodiments, a nucleic acid described herein further comprises one or more regulatory sequences that directs expression of the variant rhizavidin polypeptide or the fusion protein. In some embodiments, the one or more regulatory sequences comprise one or more of the following: a promoter, an intron, an enhancer, a polyadenylation signal, a terminator, a silencer, a TATA box, or a Woodchuck Hepatitis Virus (WHP) Posttranscriptional Regulatory Element (WPRE).

Another aspect of the present disclosure provides a vector comprising a nucleic acid described herein.

Another aspect of the present disclosure provides a method of producing the variant rhizavidin polypeptide described herein or a fusion protein described herein. In some embodiments, such a method comprises using a nucleic acid described herein or a vector described herein to express the variant rhizavidin polypeptide or fusion protein in a host cell. In some embodiments, the host cell is a bacterial cell, a yeast cell, an insect cell, or a mammalian cell.

Another aspect of the present disclosure provides a composition comprising a variant rhizavidin polypeptide described herein or a fusion protein described herein. In some embodiments, such a composition further comprises a biotinylated polymer comprising biotin and a polymer, wherein the biotinylated polymer is non-covalently associated with the variant rhizavidin polypeptide. In some embodiments, the polymer is or comprises a polysaccharide antigen. In some embodiments, the polysaccharide antigen is or comprises a polysaccharide selected from the group consisting of: a bacterial polysaccharide antigen, a fungal polysaccharide antigen, a parasitic polysaccharide antigen, a viral polysaccharide antigen, a mammalian polysaccharide antigen (e.g., a tumor polysaccharide antigen). In some embodiments, the polysaccharide antigen is or comprises a polysaccharide selected from the group consisting of: Salmonella polysaccharide, pneumococcal polysaccharides, Haemophili polysaccharides, meningococcal polysaccharides, staphylococcal polysaccharides, Bacillus anthracis polysaccharide, Streptococcus polysaccharide, Pseudomonas polysaccharide, Klebisella polysaccharide, Cryptococcus polysaccharide, other bacterial capsular or cell wall polysaccharides, viral polysaccharides (e.g., viral glycoproteins), or combinations thereof.

Another aspect of the present disclosure provides a pharmaceutical composition comprising a composition described herein, and a pharmaceutically acceptable carrier. In some embodiments, such a pharmaceutical composition further comprises one or more adjuvants. In some embodiments, such the one or more adjuvants are or comprise a co-stimulation factor. In some embodiments, such the one or more adjuvants are selected from the group consisting of: aluminum phosphate, aluminum hydroxide, and phosphate aluminum hydroxide. In some embodiments, such the one or more adjuvants are or comprise aluminum phosphate. In some embodiments, such the pharmaceutical composition is formulated for injection.

In some embodiments, upon administration to a subject, a pharmaceutical composition described herein induces an immune response.

In some embodiments, a composition described herein or a pharmaceutical composition described herein is for use in administration to a subject to immunize the subject.

Another aspect of the present disclosure provides a use of a composition described herein or a pharmaceutical composition described herein, in the manufacture of a medicament for administration to a subject to immunize the subject.

Another aspect of the present disclosure provides a method of immunizing a subject, comprising administering to the subject a composition described herein or the pharmaceutical composition described herein.

In some embodiments, a subject is a human. In some embodiments, a subject is an agricultural or non-domestic animal. In some embodiments, a subject is a domestic animal.

In some embodiments, administration of a composition described herein or a pharmaceutical composition described herein is by subcutaneous, intranasal, intradermal, or intramuscular injection.

In some embodiments, a composition described herein or a pharmaceutical composition described herein induces an immune response. In some embodiments, the immune response comprises an antibody or B cell response. In some embodiments, the immune response comprises a CD4+ T cell response (e.g., T_{H}1, T_{H}2, or T_{H}17 response), a CD8+ T cell response, a CD4+ and CD8+ T cell response, or a CD4−/CD8− T cell response. In some embodiments, the immune response comprises (i) an antibody or B cell response, and (ii) a T cell response. In some embodiments, the immune response comprises (i) an antibody or B cell response, and (ii) a CD4+ T cell response (e.g., T_{H}1, T_{H}2, or T_{H}17 response), a CD8+ T cell response, a CD4+ and CD8+ T cell response, or a CD4−/CD8− T cell response.

### BRIEF DESCRIPTION OF THE DRAWINGS

FIG. 1 shows the predicted N-linked glycosylation positions of wild-type rhizavidin [amino acids 45-179]. FIG. 1A: Graphic display of N-linked glycosylation potential of glycosylation motifs present within wild-type rhizavidin mapped against sequence position.

FIG. 1B: Glycosylation motifs with glycosylation potential values and N-Glyc predicted glycosylation status.

FIG. 2 shows sequence alignment of rhizavidin [amino acids 45-179] and engineered rhizavidin A4, Q4, A5, and A5T variants. Protein sequence alignment of rhizavidin (Rhizobium etli CFN 42) with and without substitutions was generated and analyzed using ClustalW2 [Larkin et al, 2007] and ESPript 3.x. FIG. 2A: Sequence alignment of rhizavidin [amino acids 45-179] and rhizavidin A5T variant. The secondary structure of rhizavidin is displayed above the alignment (PDB: 3EW2). Beta-sheets are rendered as arrows and strict beta-turns as TT letters. Predicted glycosylation positions are shown in black boxes, glycosylation sites having NXT/S motifs are outlined in black, and residues involved in biotin binding are shown in dark gray boxes. A5T substitutions are in underlined black lettering. FIG. 2B: Sequence alignment of rhizavidin [amino acids 45-179] against rhizavidin A4, Q4, A5, and A5T variants. Consensus amino acids are highlighted in gray.

FIG. 3 shows a model of rhizavidin A5T variant dimer and monomer. The model is based on native rhizavidin structure (PBD: 3EW2) [Meir et al., 2009] and created in PyMol (Schrödinger, LLC, New York, NY, USA). Beta-sheets are rendered as flat arrows. Bound biotin molecules are shown as a stick structure, residues involved in biotin binding as black boxes, and (A5T) substitutions in spheres (dark gray for N substitutions, light gray for S/T). The N and C termini are labeled with black N and C, respectively. FIG. 3A: Rhizavidin A5T variant dimer. FIG. 3B: Rhizavidin A5T variant monomer. FIG. 3C: Rotated and expanded rhizavidin A5T variant monomer, displaying amino acid residues 116-119 and 106-108.

FIG. 4 compares biotin-binding activity and presence of dimers in mammalian-expressed rhizavidin A4, Q4, A5, and A5T variants. FIG. 4A: Samples of purified mammalian-expressed rhizavidin A4, Q4, A5, and A5T variants and reference bacterially-expressed rhizavidin were combined with biotin-4-fluorescein (B4F) and separated by SDS-PAGE. Fluorescein imaging produced a single ~27 kDa band in reference rhizavidin, and in A5 and A5T variant samples, consistent with elimination of N-linked glycosylation sites in the A5 and A5T variant constructs and presence of rhizavidin dimers able to bind biotin. FIG. 4B: Coomassie staining showed equivalent amounts of protein in each of the rhizavidin A4, Q4, A5, and A5T variant lanes. FIG. 4C: Western blot with an anti-histidine monoclonal antibody detected a band at ~27 kDa in reference rhizavidin, and A5 and A5T variant samples, consistent with the presence of rhizavidin dimers. In FIG. 4C, the lower molecular weight band in reference rhizavidin, and A5 and A5T variant samples is likely monomeric rhizavidin at ~18 kDa. The smeared bands in A4 and Q4 variant samples may represent glycosylated dimers and monomers.

FIG. 5 compares glycosylation, biotin-binding activity, and presence of dimers in bacterially-expressed (B) and mammalian-expressed (M) reference rhizavidin to bacterially- and mammalian-expressed rhizavidin A5T variant. PNGase F, an amidase, which cleaves the innermost GlcNAc and asparagine residues in oligosaccharides, was used to cleave all N-linked glycans from purified reference rhizavidin and rhizavidin A5T variant under heat denaturing conditions. The cleavage products were run on SDS-PAGE with biotin-4-fluorescein (B4F); gels were imaged on fluorescein and then stained with Coomassie. FIGS. 5A and 5B: Samples of purified bacterially-expressed (B) and mammalian-expressed (M) reference rhizavidin and rhizavidin A5T variant were incubated in the presence or absence of PNGase F, as indicated, then combined with B4F and separated by SDS-PAGE. In the absence of PNGase F cleavage, bacterially-expressed reference rhizavidin, bacterially-expressed rhizavidin A5T variant, and mammalian-expressed rhizavidin A5T variant produced a band at ~27 kDa by Coomassie staining (FIG. 5A, left) and fluorescein imaging (FIG. 5B, left). FIG. 5C: Samples of purified bacterially-expressed (B) and mammalian-expressed (M) reference rhizavidin and rhizavidin A5T variant were heated at 100° C. for 10 minutes with a reducing agent, separated by SDS-PAGE, and Coomassie stained. Bacterially-expressed reference rhizavidin, bacterially-expressed rhizavidin A5T variant, and mammalian-expressed rhizavidin A5T variant collapsed completely into a band at ~18 kDa, the expected size of rhizavidin monomers. As in FIGS. 5A and 5B, the smeared higher molecular weight bands seen with mammalian-expressed reference rhizavidin are consistent with glycosylation.

FIG. 6 illustrates protein purity based on densitometry results for select samples of FIGS. 5A-5C. FIG. 6A summarizes densitometry results and purity of PNGase F-cleaved proteins of FIG. 5A. FIG. 6B summarizes densitometry results and purity of heated rhizavidin proteins of FIG. 5C.

FIG. 7 shows microscale thermophoresis (MST) biotin-binding kinetics of rhizavidin A5T variant compared to reference rhizavidin, obtained by maintaining constant Biotin Flamma concentration while titrating protein. FIG. 7A: Binding curves with dose response. Bacterially-expressed reference rhizavidin is shown as dots, bacterially-expressed rhizavidin A5T variant as squares, and mammalian-expressed rhizavidin A5T variant as triangles. FIG. 7B: Normalized Kd and confidence intervals calculated in MO.Control software were normalized by dividing them by Kd for bacterially-expressed reference rhizavidin. FIG. 7C: Calculated equilibrium constants of rhizavidin proteins to biotin-Flamma 648 with confidence intervals.

FIG. 8 is a schematic diagram showing MAPS technology according to certain embodiments described herein.

FIG. 9 shows normalized mass fraction distribution determined by HPLC-SEC-MALS of mammalian-expressed ancestral and variant S-RBD-rhizavidin A5T fusion proteins, MAPS complexes comprising ancestral and variant S-RBD-rhizavidin A5T fusion proteins, and pneumococcal capsular polysaccharide, together with correlation by SDS-PAGE. FIG. 9A: Peaks for fusion proteins (Proteins), polysaccharide, and MAPS complexes (MAPS) are labeled. The molecular weight distribution curve for all three fusion proteins displayed a single peak corresponding to their calculated molecular weight (39.5 kDa). The polysaccharide profile shows a highly polydisperse molecular weight distribution, similarly observed for the three MAPS complexes. The profile of MAPS complexes is shifted to the right due to the association of polysaccharide and fusion protein; different MAPS complexes showed increased average molecular weight. FIG. 9B: Molecular weights and polydispersity of each sample determined by HPLC-SEC-MALS in tabular form. FIG. 9C: Coomassie stained SDS-PAGE loaded with 3 μg of pneumococcal capsular polysaccharide (PS), 1.5 μg of the indicated mammalian-expressed S-RBD-rhizavidin A5T fusion proteins, 1.5 μg of the indicated MAPS complexes (by protein) combined with 1× reducing Laemmli buffer. Samples to the left of each set were not heated; samples to the right of each set were heated for 10 minutes at 100° C. Bands at ~80 kDa corresponding to S-RBD-rhizavidin A5T fusion protein dimers are noted. Polysaccharide samples and unheated MAPS complexes were retained in the well due to their size; however, after heating, ~40 kDa bands corresponding to S-RBD-rhizavidin A5T fusion protein monomers were observed as well.

FIG. 10 illustrates purity based on densitometry results for fusion proteins and MAPS complexes of FIG. 9B. FIG. 10A: Raw Coomassie stained SDS-PAGE. FIG. 10B: Summary of densitometry results and purity.

FIG. 11 shows immunogenicity results for exemplary S-RBD monovariant and multivariant MAPS vaccines. Anti-S-RBD IgG titers of mice that were immunized with any of the MAPS vaccines increased significantly compared to the saline control group. Immunization with any of the three S-RBD MAPS vaccines produced similar anti-S-RBD IgG titers. There was no significant difference between IgG titers against any of the strains of SARS-CoV2 S-RBD when animals were immunized with the multivariant MAPS vaccine compared to monovariant S-RBD MAPS vaccines. FIG. 11A: Schematic of the study, FIG. 11B: IgG levels directed to ancestral S-RBD. FIG. 11C: IgG levels directed to delta S-RBD. FIG. 11D: IgG levels directed to gamma S-RBD.

FIG. 12 shows the ability of rhizavidin A5T variant to form a fusion protein with a further protein TRP2 and retain biotin binding activity. Samples of 3 μg Nickel purified mammalian expressed His-tagged proteins were combined with 50 ng of biotin-4-fluorescein (B4F) and SDS sample buffer and were loaded unheated and separated on a 4-12% Bis-Tris SDS-PAGE. Panel A shows the results of fluorescein imaging and Panel B shows the Coomassie stained gel.

### CERTAIN DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastrical, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. For example, in some embodiments, affinity of binding of a rhizavidin variant polypeptide described herein and to biotin can be quantitatively determined using a microscale thermophoresis (MST) assay; see e.g., [Jerabek-Willemsen et al., 2014]. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological-setting). In some embodiments, affinity is assessed relative to a reference (e.g., that has a known affinity above a particular threshold [a “positive control” reference] or that has a known affinity below a particular threshold [a “negative control” reference”]. In some embodiments, affinity may be assessed relative to a contemporaneous reference; in some embodiments, affinity may be assessed relative to a historical reference. Typically, when affinity is assessed relative to a reference, it is assessed under comparable conditions.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively, or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amino acid: In its broadest sense, the term “amino acid”, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Non-standard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments, the term “amino acid” may be used to refer to an amino acid residue of a polypeptide.

Antigen: The term “antigen”, as used herein, refers to (i) an agent that induces an immune response; and/or (ii) an agent that binds to a T cell receptor (e.g., when presented by an MHC molecule) or to an antibody. In some embodiments, an antigen induces a humoral response (e.g., including production of antigen-specific antibodies); in some embodiments, an antigen induces a cellular response (e.g., involving T cells whose receptors specifically interact with the antigen). In some embodiments, an antigen induces a humoral response and a cellular response. In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments, other than a biologic polymer (e.g., other than a nucleic acid or amino acid polymer)), etc. In some embodiments, an antigen is or comprises a polypeptide. In some embodiments, an antigen is or comprises a polysaccharide. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, an antigen is provided in a crude form. In some embodiments, an antigen is a recombinant antigen. In some embodiments, an antigen is a polypeptide or a polysaccharide that, upon administration to a subject, induces a specific and/or clinically relevant immune response to such polypeptide or polysaccharide. In some embodiments, an antigen is selected to induce a specific and/or clinically relevant immune response to such an antigen.

Associated with: Two entities are “associated” with one another, as that term is used herein, if the presence, level and/or form of one is correlated with that of the other. In some embodiments, two or more entities are physically “associated” with one another if they interact, directly or indirectly, so that they are and/or remain in physical proximity with one another. In some embodiments, two or more entities that are physically associated with one another are covalently linked to one another. In some embodiments, two or more entities that are physically associated with one another are not covalently linked to one another but are non-covalently associated, for example by means of affinity interactions, electrostatic interactions, hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; “indirect” binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

Biotin-binding moiety: The term “biotin-binding moiety”, as used herein, refers to a biotin-binding protein, a biotin-binding fragment thereof, or a biotin-binding domain thereof.

Carrier protein: As used herein, the term “carrier protein” refers to a protein or peptide that is coupled, or complexed, or otherwise associated with a hapten (e.g., a small peptide or lipid) or less immunogenic antigen (e.g., a polysaccharide) and that induces or improves an immune response to such a coupled, or complexed, or otherwise associated hapten (e.g., a small peptide or lipid) or less immunogenic antigen (e.g., a polysaccharide). In some embodiments, such an immune response is or comprises a response to a hapten or less immunogenic antigen that is coupled, or complexed, or otherwise associated with such a carrier protein. In some embodiments, such an immune response is or comprises a response to both a carrier protein and a hapten or less immunogenic antigen that is coupled, or complexed, or otherwise associated with such a carrier protein. In some embodiments, no significant immune response to a carrier protein itself occurs. In some such embodiments, a carrier protein may act as an adjuvant. In some embodiments, immune response to a carrier protein may be detected; in some embodiments, immune response to such a carrier protein is strong. In some embodiments, a carrier protein is coupled, or complexed, or otherwise associated with one or more other molecules.

Colonization: As used herein, the term “colonization” generally refers to the ability of a microbe to grow at a target site or surface. For example, the terms “colonization” refers to the ability of a microbe (e.g., a bacterium) to grow at an anatomical site (e.g., a mucosal membrane, gastrointestinal tract, injury site, organ, etc.) of a host.

Combination therapy: As used herein, the term “combination therapy” refers to those situations in which a subject is exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Derivative: As used herein, the term “derivative”, or grammatical equivalents thereof, refers to a structural analogue of a reference substance. That is, a “derivative” is a substance that shows significant structural similarity with the reference substance, for example sharing a core or consensus structure, but also differs in certain discrete ways. Such a substance would be said to be “derived from” said reference substance. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, α-helix character, β-sheet character, coiled-coil character, random coil character, etc.), and/or by a particular functional feature (e.g., binding activity, enzymatic activity, folding activity, signaling activity, etc.).

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature. For example, in some embodiments described and/or utilized herein, an engineered polynucleotide comprises a regulatory sequence that is found in nature in operative association with a first coding sequence but not in operative association with a second coding sequence, is linked by the hand of man so that it is operatively associated with the second coding sequence. Comparably, a polypeptide may be considered to be “engineered” if encoded by or expressed from an engineered polynucleotide, and/or if produced other than natural expression in a cell. For example, a polypeptide is considered to be “engineered” if one or more amino acid mutations are selectively introduced into the polypeptide for a particular purpose, characteristic, or property. Analogously, a cell or organism is considered to be “engineered” if it has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment includes a discrete portion of the whole which discrete portion shares one or more functional characteristics found in the whole. In some embodiments, a fragment consists of such a discrete portion. In some embodiments, a fragment consists of or comprises a characteristic structural element or moiety found in the whole. In some embodiments, a fragment of a polymer, e.g., a polypeptide or a polysaccharide, comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more contiguous monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. In some embodiments, such a polymer fragment may comprise or consist of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more contiguous monomeric units (e.g., residues) as found in the whole polymer. The whole material or entity may, in some embodiments, be referred to as the “parent” of the whole.

Glycosylation Site: As used herein, the term “glycosylation site” refers to an amino acid sequence within a polypeptide sequence that matches an amino acid sequence motif determined to be recognized by a glycosylation-associated enzyme. As will be understood by those skill in the art, while the presence of a glycosylation site may be necessary, it alone is not a sufficient criterion for glycosylation. In some instance, a glycosylation site may not found to be glycosylated. Various types and/or mechanisms of protein glycosylation are known in the art, including e.g., but not limited to, N-linked glycosylation, O-linked glycosylation, C-linked glycosylation, glypiation, and phosphoglycosylation. Those of skill in the art will appreciate that different types of glycosylation are associated with distinct amino acid sequence motifs. See, e.g., Reily et al., Nat. Rev. Nephrol. 15(6):346-366 (2019) and Eichler, Curr. Biol. 29 (7): R229-R231 (2019). In some embodiments, glycosylation site(s) that are particularly useful in accordance with the present disclosure are or comprise N-linked glycosylation site(s). For example, N-linked glycosylation is typically associated with the amino acid sequence motifs: NXaaT and NXaaS, and in some cases NXaaC, where Xaa represents any amino acid residue except proline (P). Accordingly, in some embodiments, an N-linked glycosylation site refers to an amino acid sequence within a polypeptide sequence that matches one of the amino acid sequence motifs: NXaaT, NXaaS, and NXaaC, where Xaa represents any amino acid residue except proline (P). In some embodiments, an N-linked glycosylation site refers to an amino acid sequence within a polypeptide sequence that matches one of the amino acid sequence motifs: NXaaT and NXaaS, where Xaa represents any amino acid residue except proline (P).

In some embodiments, an amino acid residue within a glycosylation site that is predicted to be glycosylated is referred to as a glycosylation position. For example, a glycosylation position within an N-linked glycosylation site comprising the amino acid sequence of NXaaT or NXaaS is the amino acid residue N. In some embodiments, a glycosylation position within a glycosylation site is determined to be glycosylated. In some embodiments, a glycosylation position within a glycosylation site is determined to be not glycosylated. Various experimental methods to determine whether a glycosylation site or particularly a glycosylation position is glycosylated are known in the art, including, e.g., nuclear magnetic resonance, x-ray crystallography, mass spectrometry; see e.g., An et al., Curr. Opin. Chem. Biol. 13(4):421-426.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% identical. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Improve, increase, inhibit or reduce: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single subject) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Immunologically effective amount or immunologically effective dose: As used herein, “immunologically effective amount” or “immunologically effective dose” refers to an amount of an antigenic or immunogenic substance, e.g., an antigen, immunogen, immunogenic complex, immunogenic composition, vaccine, or pharmaceutical composition, which when administered to a subject, either in a single dose or as part of a series of doses, that is sufficient to enhance a subject's own immune response against a subsequent exposure to an antigen. Non-limiting examples of antigens include antigens from tumors, pathogens, etc. An immunologically effective amount may vary based on the subject to be treated, the species of the subject, the degree of immune response desired to induce, etc. In some embodiments, an immunologically effective amount is sufficient for treatment or protection of a subject having or at risk of having disease. In some embodiments, an immunologically effective amount refers to a non-toxic but sufficient amount that can be an amount to treat, attenuate, or prevent infection and/or disease (e.g., infection, cancer, a symptom or complication associated with cancer or infection) in any subject. In some embodiments, an immunologically effective amount is sufficient to induce an immunoprotective response upon administration to a subject.

Immunoprotective response or protective response: As used herein, “immunoprotective response” or “protective response” refers to an immune response that mediates antigen or immunogen-induced immunological memory. In some embodiments, an immunoprotective response is induced by the administration of a substance, e.g., an antigen, immunogen, immunogenic complex, immunogenic composition, vaccine, or pharmaceutical composition to a subject. In some embodiments, immunoprotection involves one or more of active immune surveillance, a more rapid and effective response upon immune activation as compared to a response observed in a naïve subject, efficient clearance of the activating agent or pathogen, followed by rapid resolution of inflammation. In some embodiments, an immunoprotective response is an adaptive immune response. In some embodiments, an immunoprotective response is sufficient to protect an immunized subject from productive infection by a particular pathogen or pathogens to which a vaccine is directed. In some embodiments, an immunoprotective response is sufficient to protect an immunized subject from cancer associated with a particular tumor antigen to which a vaccine is directed.

Immunization: As used herein, “immunization”, or grammatical equivalents thereof, refers to a process of inducing an immune response to an antigen (e.g., an antigen from tumor or an infectious organism or agent) in a subject (“active immunization”), or alternatively, providing immune system components against an antigen (e.g., an antigen from a tumor or an infectious organism or agent) to a subject (“passive immunization”). In some embodiments, immunization involves the administration of one or more antigens, immunogens, immunogenic complexes, vaccines, immune molecules such as antibodies, immune sera, immune cells such as T cells or B cells, or pharmaceutical compositions to a subject. In some embodiments, immunization is performed by administering an immunologically effective amount of a substance, e.g., an antigen, immunogen, immunogenic complex, immunogenic composition, vaccine, immune molecule such as an antibody, immune serum, immune cell such as a T cell or B cell, or pharmaceutical composition to a subject. In some embodiments, immunization results in an immunoprotective response in the subject. In some embodiments, active immunization is performed by administering to a subject an antigenic or immunogenic substance, e.g., an antigen, immunogen, immunogenic complex, vaccine, or pharmaceutical composition. In some embodiments, passive immunization is performed by administering to a subject an immune system component, e.g., an immune molecule such as an antibody, immune serum, or immune cell such as a T cell or B cell.

Isolated: As used herein, the term “isolated”, or grammatical equivalents thereof, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polysaccharide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide or polysaccharide that is chemically synthesized or is synthesized in a cellular system different from that which produces it in nature is considered to be an “isolated” polypeptide or polysaccharide. Alternatively or additionally, in some embodiments, a polypeptide or polysaccharide that has been subjected to one or more purification techniques may be considered to be an “isolated” polypeptide or polysaccharide to the extent that it has been separated from other components a) with which it is associated in nature; and/or b) with which it was associated when initially produced.

Linker: As used herein, the term “linker” is used to refer to an entity that connects two or more elements to form a multi-element agent. For example, those of ordinary skill in the art appreciate that, in some embodiments, a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker (L). In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) are known in the art (Holliger et al, 1993; Poljak, 1994).

Mutation: As used herein, the term “mutation” is used to refer to a difference in the sequence of a nucleic acid or polypeptide as compared to a reference sequence. Exemplary types of mutations include, but are not limited to, insertions, deletions, and substitutions. For the avoidance of doubt, the term “mutation” is understood by those of skill in the art to include a difference as compared to a reference, and does not necessarily refer to or imply a change having occurred within any particular sequence of interest.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Polysaccharide: The term “polysaccharide” as used herein refers to a polymeric carbohydrate molecule composed of long chains of monosaccharide units bound together by glycosidic, phosphodiester, or other linkages and on hydrolysis give the constituent monosaccharides or oligosaccharides. Polysaccharides range in structure from linear to highly branched. Examples include storage polysaccharides such as starch and glycogen, structural polysaccharides such as cellulose and chitin and microbial polysaccharides, and antigenic polysaccharides found in microorganisms including, but not limited to, capsular polysaccharides (CPS), O polysaccharides (OPS), core O polysaccharides (COPS), and lipopolysaccharides (LPS).

Polypeptide: The term “polypeptide”, as used herein, generally has its art-recognized meaning of a polymer of at least three amino acids, e.g., linked to each other by peptide bonds. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof.

Prevention: The term “prevent” or “prevention”, as used herein in connection with a disease, disorder, and/or medical condition, refers to reducing the risk of developing the disease, disorder and/or condition, and/or a delay of onset, and/or reduction in frequency and/or severity of one or more characteristics or symptoms of a particular disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. In some embodiments, prevention may be considered complete when onset of a disease, disorder or condition has been delayed for a pre-defined period of time.

Protein: As used herein, the term “protein” encompasses a polypeptide. Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain 1-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Recombinant: As used herein, the term “recombinant” is intended to refer to polypeptides that are designed, engineered, prepared, expressed, created, manufactured, and/or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Reference: As used herein, the term “reference” describes a standard or control relative to which a comparison is performed. For example, in some embodiments, an agent, animal, subject, population, sample, sequence or value of interest is compared with a reference or control agent, animal, subject, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, a “response” to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. A subject's response may be measured according to a wide variety of criteria, including clinical criteria and objective criteria. Techniques for assessing response include, but are not limited to, clinical examination, positron emission tomography, chest X-ray CT scan, MRI, ultrasound, endoscopy, laparoscopy, presence or level of biomarkers in a sample obtained from a subject, cytology, and/or histology. The exact response criteria can be selected in any appropriate manner, provided that when comparing groups of subjects, the groups to be compared are assessed based on the same or comparable criteria for determining response rate. One of ordinary skill in the art will be able to select appropriate criteria.

Risk: As will be understood from context, “risk” of a disease, disorder, and/or condition refers to a likelihood that a particular subject will develop the disease, disorder, and/or condition. In some embodiments, risk is expressed as a percentage. In some embodiments, risk is from 0%, 1%, 2%, 3%, 4%, 5%, 6%, 7%, 8%, 9%, 10%, 20%, 30%, 40%, 50%, 60%, 70%, 80%, 90% up to 100%. In some embodiments, risk is expressed as a risk relative to a risk associated with a reference sample or group of reference samples. In some embodiments, a reference sample or group of reference samples have a known risk of a disease, disorder, condition and/or event. In some embodiments, a reference sample or group of reference samples are from subjects comparable to a particular subject. In some embodiments, relative risk is 0, 1, 2, 3, 4, 5, 6, 7, 8, 9, 10, or more.

Serotype: As used herein, the term “serotype”, also referred to as a serovar, refers to a distinct variation within a species of bacteria or virus or among immune cells of different subjects. These microorganisms, viruses, or cells are classified together based on their cell surface antigens, allowing the epidemiologic classification of organisms to the sub-species level. A group of serovars with common antigens may be referred to as a serogroup or sometimes serocomplex.

Species: As used herein, the term “species” refers to a distinct immunogenic complex comprising (i) a biotinylated polymer (e.g., polysaccharide antigen) and (ii) a fusion protein comprising a biotin-binding moiety and one or more polypeptides (e.g., polypeptide antigens). In some embodiments, a distinct species can differ in one or more of (i) a biotinylated polymer (e.g., polysaccharide antigen) and (ii) a fusion protein comprising a biotin-binding moiety and one or more polypeptides (e.g., polypeptide antigens).

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is a subject to whom diagnosis and/or therapy is and/or has been administered.

Susceptible to: A subject who is “susceptible to” a disease, disorder, or condition is at risk for developing the disease, disorder, or condition. In some embodiments, a subject who is susceptible to a disease, disorder, or condition does not display any symptoms of the disease, disorder, or condition. In some embodiments, a subject who is susceptible to a disease, disorder, or condition has not been diagnosed with the disease, disorder, and/or condition. In some embodiments, a subject who is susceptible to a disease, disorder, or condition is a subject who has been exposed to conditions associated with development of the disease, disorder, or condition. In some embodiments, a risk of developing a disease, disorder, and/or condition is a population-based risk (e.g., family members of subjects suffering from the disease, disorder, or condition).

Symptoms are reduced: As used herein, “symptoms are reduced” when one or more symptoms of a particular disease, disorder or condition is reduced in magnitude (e.g., intensity, severity, etc.) and/or frequency, e.g., to a statistically and/or clinically significant or relevant level. For purposes of clarity, a delay in the onset of a particular symptom is considered one form of reducing the frequency of that symptom.

Treatment: As used herein, the term “treatment” (also “treat” or “treating”) refers to any administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Vaccination: As used herein, the term “vaccination” refers to the administration of a composition intended to generate an immune response, for example to a disease-causing agent. For the purposes of the present disclosure, vaccination can be administered before, during, and/or after exposure to a disease-causing agent, and in certain embodiments, before, during, and/or shortly after exposure to the agent. In some embodiments, vaccination includes multiple administrations, appropriately spaced in time, of a vaccinating composition. In some embodiments, vaccination initiates immunization.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 modifications (e.g., substitutions, additions or deletions) at the N-terminus portion, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 modifications (e.g., substitutions, additions or deletions) at the C-terminus portion, as compared to the reference. In some embodiments, a reference polypeptide or nucleic acid is one found in nature.

### DETAILED DESCRIPTION OF CERTAIN EMBODIMENTS

The present disclosure relates, generally to compositions, systems, kits, and methods that include rhizavidin variants. Engineered rhizavidin variants described herein are useful as biotin-binding proteins for various applications, e.g., in some embodiments, to couple two molecular entities (e.g., small molecules, proteins, lipids, polysaccharides, etc.) via non-covalent biotin-rhizavidin interaction. Rhizavidin variants can be used e.g., for production of fusion proteins containing the rhizavidin variant in eukaryotic (e.g., mammalian, yeast, or insect) expression systems.

In one aspect, the present disclosure recognizes that, while rhizavidin is a bacterial protein, which has been commonly recombinantly expressed in bacteria, such as E. coli, it may be desirable to express rhizavidin using a eukaryotic (e.g., mammalian, yeast, or insect) expression system in certain circumstances or applications. For example, such certain circumstances or applications include, e.g., production of a fusion protein comprising a rhizavidin polypeptide and a polypeptide of interest present in a eukaryotic cell (e.g., a mammalian cell), virus, fungus, parasite, or in some instances present in a bacterial cell, where post-translation modification and/or processing of the polypeptide of interest in eukaryotic cells are useful. In some embodiments, polypeptides of interest that may benefit from post-translational modification and/or processing in eukaryotic cells may include polypeptides that contain multiple disulfide bonds, polypeptides that are hydrophobic, and/or polypeptides that require additional chaperones present in eukaryotic cells to facilitate their proper expression and folding. In some embodiments, such a polypeptide of interest is or comprises a polypeptide antigen present in a eukaryotic cell (e.g., a mammalian cell). In some embodiments, such a polypeptide antigen present in a eukaryotic cell (e.g., a mammalian cell) may be or comprise a tumor antigen. In some embodiments, such fusion proteins may be useful in a variety of applications, e.g., in some embodiments, therapeutic applications including, e.g., but not limited to immunogenic compositions based on MAPS platform as described in International Patent Application Nos. PCT/US2012/037412, PCT/US2019/050907, PCT/US2022/042964, and PCT/US2022/043156.

In one aspect, the present disclosure identifies the source of problems with expressing wild-type rhizavidin or a fragment thereof in eukaryotic cells (e.g., mammalian, yeast, or insect cells). For example, the present disclosure recognizes that rhizavidin has 4-5 N-linked glycosylation sites as bioinformatically predicted, which are likely not glycosylated in bacteria from which it is derived or when recombinantly expressed in E. coli, but are variably glycosylated when expressed in eukaryotic (e.g., mammalian, yeast or insect) expression systems. Without wishing to be bound by any particular theory, various patterns of N-linked glycosylation present in rhizavidin expressed in eukaryotic cells can lead to poor expression in eukaryotic cells and/or generation of glycosylated rhizavidin with various molecular weights that make purification of rhizavidin challenging. In some embodiments, the present disclosure provides technologies (including compositions, methods, kits, etc.) that solve such problems, among other things, by engineering rhizavidin variants to remove glycosylation activity, while retaining desirable characteristics of a wild-type rhizavidin, including, e.g., but not limited to biotin-binding activity, dimerization activity, etc. In some embodiments, the present disclosure provides insights that certain amino acid positions and/or mutations within glycosylation motifs can adversely impact dimerization activity and/or expression of rhizavidin. For example, it is demonstrated that a mutation at a position corresponding to the amino acid residue N106 (e.g., in some embodiments, N106A) within a glycosylation motif of rhizavidin (as set forth in SEQ ID NO: 1) reduced expression of rhizavidin in eukaryotic cells (e.g., mammalian, yeast, or insect cells), as compared to a mutation at a position corresponding to the amino acid residue T108 within the same glycosylation motif (e.g., in some embodiments, T108A). It is also demonstrated that a mutation at a position corresponding to the amino acid residue S119 (e.g., in some embodiments, S119A) within a secondary glycosylation motif of rhizavidin (as set forth in SEQ ID NO: 1) reduced glycosylation and promoted expression of rhizavidin in eukaryotic cells (e.g., mammalian, yeast, or insect cells). Additionally, it is unexpectedly found that a less conservative amino acid substitution (e.g., N to A substitution) present in a glycosylation motif of rhizavidin allowed rhizavidin to form dimers as observed in wild-type rhizavidin, while a more conservative amino acid substitution (e.g., N to Q substitution) produced monomeric rhizavidin. Accordingly, in some embodiments, the present disclosure provides engineered rhizavidin variants comprising certain combinations of amino acid mutations (e.g., as described herein) that are particularly useful for expression in eukaryotic cells (e.g., mammalian, yeast, or insect cells).

### Rhizavidin

Rhizavidin, a naturally occurring dimeric protein in the avidin protein family, was first discovered in Rhizobium etli, a symbiotic bacterium of the common bean. Rhizavidin binds non-covalently to biotin with high affinity (dissociation constant [KD]≈10⁻¹⁵ M). Rhizavidin has only a 22% amino acid identity with chicken avidin, a protein commonly found in eggs, but with high conservation of amino acid residues involved in biotin binding. Rhizavidin has no significant homology with human proteins. No cross-reactivity to rhizavidin is observed in human serum samples obtained from subjects exposed to avidin [Helppolainen et al, 2007], suggesting that rhizavidin antibodies may not cross-react with chicken avidin.

In some embodiments, a rhizavidin polypeptide is a wild-type rhizavidin polypeptide. In some embodiments, a wild-type rhizavidin polypeptide can be used as a reference for comparison to another rhizavidin polypeptide (e.g., a variant rhizavidin polypeptide described herein). In some embodiments, a wild-type rhizavidin polypeptide comprises a wild-type rhizavidin or a biotin-binding domain or biotin-binding fragment thereof, e.g., as further described in WO 2012/155053, the contents of which are herein incorporated by reference in their entirety for the purposes described herein. The term “wild-type rhizavidin” is accorded the usual definition associated with such phrase, e.g., in some embodiments, a naturally occurring rhizavidin (e.g., a rhizavidin that is produced by a capable bacterial source). In some embodiments, a wild-type rhizavidin polypeptide comprises a polypeptide of SEQ ID NO: 1 or a biotin-binding domain or biotin-binding fragment thereof. In some embodiments, a rhizavidin polypeptide that is useful in accordance with the present disclosure comprises a polypeptide of SEQ ID NO: 2 or SEQ ID NO: 3, or a biotin-binding domain or biotin-binding fragment thereof.

In some embodiments, a wild-type rhizavidin polypeptide includes a portion of a wild-type rhizavidin (e.g., a portion of a rhizavidin polypeptide of SEQ ID NO: 1), which portion includes at least 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170 or more contiguous amino acids of the wild-type rhizavidin. In some embodiments, a wild-type rhizavidin polypeptide corresponds to a polypeptide having amino acids 45-179 of the amino acid sequence set forth in SEQ ID NO: 1.

### Rhizavidin Variants

As discussed above, rhizavidin is a bacterial protein, which can present challenges for production of rhizavidin or a biotin-binding domain or biotin-binding fragment thereof, or a polypeptide comprising the same (e.g., fusion constructs), using a eukaryotic expression system. The present disclosure recognizes that rhizavidin has 4-5 N-linked glycosylation sites as bioinformatically predicted, which are likely not glycosylated in bacteria from which it is derived or when recombinantly expressed in E. coli, but are variably glycosylated when expressed in eukaryotic (e.g., mammalian, yeast, or insect) expression systems. Without wishing to be bound by any particular theory, various patterns of N-linked glycosylation present in rhizavidin expressed in eukaryotic cells can lead to poor expression in eukaryotic cells and/or generation of glycosylated rhizavidin with various molecular weights that make purification of rhizavidin challenging.

Accordingly, one aspect of the present disclosure encompasses rhizavidin polypeptides that are engineered to improve their expression in eukaryotic systems (e.g., mammalian cells, yeast cells, insect cells, etc.) while retaining certain characteristics (including, e.g., but not limited to biotin-binding activity) of a wild-type rhizavidin polypeptide, as well as technologies involving the same (e.g., fusion constructs, compositions, and methods). In some embodiments, such a variant rhizavidin polypeptide is substantially non-glycosylated (e.g., when expressed in eukaryotic cells (e.g., insect, yeast, or mammalian cells)). As used herein, the phrase “substantially non-glycosylated” means that the degree of glycosylation in a provided variant rhizavidin polypeptide is reduced by at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or up to 100%, as compared to that a reference rhizavidin polypeptide (e.g., a wild-type rhizavidin polypeptide). In some embodiments, an engineered variant rhizavidin polypeptide as described herein is characterized in that it is not glycosylated and/or has no detectable glycosylation (e.g., when expressed in eukaryotic cells (e.g., insect, yeast, or mammalian cells)). Methods of measuring glycosylation of polypeptides are known in the art, and non-limiting examples include, staining (e.g., staining based on the periodic acid-Schiff (PAS) reaction, chromogenic staining with acid fuchsin, fluorescent staining using periodate oxidation to attach a fluorescent hydride, and staining with alcian blue or Stains-All), affinity-based methods (e.g., using saccharide-binding proteins (such as lectins), using enzyme-based methods (e.g., using β-1,4-galactosyltransferase enzyme to add to GlcNAc an azidogalactose which can be conjugated to a detectable label using Click chemistry), and using antibodies (e.g., CTD 110.1 and RL2)), and/or detection based on apparent molecular weight of the polypeptide (e.g., when assessed by gel electrophoresis); see, e.g., Roth et al., Identification and Quantification of Protein Glycosylation, Intl. J. Carb. Chem. 2012:640923 (2012).

In some embodiments, an engineered variant rhizavidin polypeptide as described herein is characterized in that when a population of the variant rhizavidin is expressed in eukaryotic cells (e.g., insect, yeast, or mammalian cells), the population of variant rhizavidin polypeptides has less variability in molecular weights compared expression of a reference rhizavidin polypeptide (e.g., a wild-type rhizavidin polypeptide). In some embodiments, an engineered variant rhizavidin polypeptide as described herein is characterized in that when a population of the variant rhizavidin is expressed in eukaryotic cells (e.g., insect, yeast, or mammalian cells), the population of variant rhizavidin polypeptides has an average dimeric molecular weight between 26 kDa to 28 kDa. In some embodiments, an engineered variant rhizavidin polypeptide as described herein is characterized in that when a population of the variant rhizavidin is expressed in eukaryotic cells (e.g., insect, yeast, or mammalian cells), the population of variant rhizavidin polypeptides has an average monomeric molecular weight between 17 kDa to 19 kDa.

In some embodiments, a variant rhizavidin polypeptide is characterized in that when expressed in a eukaryotic expression system, its expression is increased relative to a reference rhizavidin polypeptide (e.g., a wild-type rhizavidin as set forth in SEQ ID NO: 1). In some embodiments, such an increase in the expression of a provided variant rhizavidin polypeptide is at least 5%, at least 10%, at least 15%, at least 20%, at least 20%, at least 30%, at least 30%, at least 35%, least 40%, at least 45%, at least 50%, at least 60%, at least 70%, at least 80%, at least 90%, at least 95%, at least 98%, at least 99%, or more relative to a reference rhizavidin polypeptide (e.g., a wild-type rhizavidin as set forth in SEQ ID NO: 1). In some embodiments, such an increase in the expression of a provided variant rhizavidin polypeptide is at least 1.1-fold, at least 1.2-fold, at least 1.3-fold, at least 1.4-fold, at least 1.5-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold, or more relative to a reference rhizavidin polypeptide (e.g., a wild-type rhizavidin as set forth in SEQ ID NO: 1)

In some embodiments, a variant rhizavidin polypeptide is characterized in that it retains the ability of forming dimers. In some embodiments, a variant rhizavidin polypeptide is characterized in that it does not form dimers.

In some embodiments, a variant rhizavidin polypeptide comprises one or more amino acid alterations (e.g., deletion, substitution, and/or insertion) from a naturally-occurring wild-type rhizavidin polypeptide sequence. For example, a variant rhizavidin polypeptide may contain an amino acid sequence that is at least 60% or more (e.g., at least 65%, 70%, 75%, 80%, 85%, 90%, 95%, or 98%) identical to SEQ ID NO: 1 or a portion thereof (e.g., at least 7, 8, 9, 10, 11, 12, 13, 14, 15, 16, 17, 18, 19, 20, 25, 30, 35, 45, 50, 55, 60, 65, 70, 75, 80, 85, 90, 95, 100, 110, 120, 130, 140, 150, 160, 170 or more consecutive amino acids of the sequence shown in SEQ ID NO: 1). Alternatively, a variant rhizavidin polypeptide may contain a portion (e.g., at least 7, 8, 9, 10, 11, 12, 13, 14, 15, 16, 17, 18, 19, 20, 25, 30, 35, 45, 50, 55, 60, 65, 70, 75, 80, 85, 90, 95, 100, 110, 120, 130, 140, 150, 160, 170 or more consecutive amino acids) of a sequence that is at least 60% or more (e.g., at least 65%, 70%, 75%, 80%, 85%, 90%, 95%, or 98%) identical to SEQ ID NO: 1.

In some embodiments, a variant rhizavidin polypeptide is or comprises a polypeptide having at least 80%, at least 85%, at least 90%, at least 95%, at least 96%, at least 97%, at least 98%, at least 99%, or 100% identity to wild-type rhizavidin, or a biotin-binding domain or biotin-binding fragment thereof. In some embodiments, a variant rhizavidin polypeptide is or comprises a polypeptide having at least 80%, at least 85%, at least 90%, at least 95%, at least 96%, at least 97%, at least 98%, at least 99% to the sequence of any of SEQ ID NOs: 1, 2, or 3, or a biotin-binding domain or biotin-binding fragment thereof.

In some embodiments, a variant rhizavidin polypeptide comprises a reference rhizavidin polypeptide (e.g., as described herein), with at least one mutation at one or more N-linked glycosylation sites present in the reference rhizavidin polypeptide. Without wishing to be bound by any particular theory, in some embodiments, glycosylation occurs at the amino acid residue N within N-linked glycosylation sites, which can be predicted based on the amino acid sequence motifs: NXaaT and NXaaS, where Xaa represents any amino acid residue except proline (P). In some embodiments, the amino acid position corresponding to the amino acid residue N within an N-linked glycosylation motifs (e.g., NXaaT and NXaaS) can be referred to as a glycosylation position. In some embodiments, an N-linked glycosylation site can be predicted using a bioinformatic prediction tool (e.g., NetNGlyc [Gupta et al., 2002]). In some embodiments, a reference rhizavidin polypeptide comprises N-linked glycosylation sites at amino acid positions: 65-67, 80-82, 106-108, 117-119, 118-120, 138-140, and 173-175 of wild-type rhizavidin (e.g., a wild-type rhizavidin as set forth in SEQ ID NO: 1). Without wishing to be bound by any particular theory, in some embodiments, mutation of any one or more of the residues of an N-linked glycosylation site in a rhizavidin polypeptide, such that the resulting amino acid sequence at the site does not comprise the motif NXaaT or NXaaS, where Xaa represents any amino acid residue except proline, can prevent glycosylation at the glycosylation position of the mutated N-linked glycosylation site when the variant rhizavidin polypeptide is expressed in eukaryotic cells (e.g., a mammalian, yeast, or insect cells). In some embodiments, a reference rhizavidin polypeptide is a polypeptide comprising an amino acid sequence that is at least 80%, at least 85%, at least 90%, at least 95%, at least 96%, at least 97%, at least 98%, at least 99%, or more identical to the amino acid sequence of any of SEQ ID NOs: 1-3, or a biotin-binding domain or biotin-binding fragment thereof.

In some embodiments, a variant rhizavidin polypeptide comprises a reference rhizavidin polypeptide (e.g., as described herein), with at least one mutation at 1, 2, 3, 4, 5, 6, or 7 N-linked glycosylation sites present in the reference polypeptide. In some embodiments, an N-linked glycosylation site that is mutated in a variant rhizavidin polypeptide described herein does not have an amino acid sequence motif of NXaaT or NXaaS, where Xaa represents any amino acid residue except proline (P). In some embodiments, such a mutated N-linked glycosylation site is not substantially glycosylated (e.g., at its glycosylation position) when expressed in a eukaryotic expression system. In some embodiments, a variant rhizavidin polypeptide comprising at least one mutation at one or more N-linked glycosylation sites is not substantially glycosylated (e.g., at its glycosylation position) when expressed in a eukaryotic expression system.

In some embodiments where a variant rhizavidin polypeptide comprises an N-linked glycosylation site having a motif of NXaaT, Nis mutated to any amino acid that is not N. In some embodiments where a variant rhizavidin polypeptide comprises an N-linked glycosylation site having a motif of NXaaT, N is mutated to A. In some embodiments where a variant rhizavidin polypeptide comprises an N-linked glycosylation site having a motif of NXaaT, N is mutated to an amino acid that is a conservative substitution of A. An exemplary conservative substitution of A is G. In some embodiments, a conservative substitution of A is not C or S. In some embodiments where a variant rhizavidin polypeptide comprises an N-linked glycosylation site having a motif of NXaaT, N is mutated to Q. In some embodiments where a variant rhizavidin polypeptide comprises an N-linked glycosylation site having a motif of NXaaT, Tis mutated to any amino acid that is not S or T. In some embodiments where a variant rhizavidin polypeptide comprises an N-linked glycosylation site having a motif of NXaaT, Tis mutated to A. In some embodiments where a variant rhizavidin polypeptide comprises an N-linked glycosylation site having a motif of NXaaT, T is mutated to an amino acid that is a conservative substitution of A. An exemplary conservative substitution of A is G. In some embodiments, a conservative substitution of A is not C or S. In some embodiments where a variant rhizavidin polypeptide comprises an N-linked glycosylation site having a motif of NXaaT, T is mutated to Q. In some embodiments where a variant rhizavidin polypeptide comprises an N-linked glycosylation site having a motif of NXaaT, Xaa is mutated to P.

In some embodiments where a variant rhizavidin polypeptide comprises an N-linked glycosylation site having a motif of NXaaS, N is mutated to any amino acid that is not N. In some embodiments where a variant rhizavidin polypeptide comprises an N-linked glycosylation site having a motif of NXaaS, N is mutated to A. In some embodiments where a variant rhizavidin polypeptide comprises an N-linked glycosylation site having a motif of NXaaS, N is mutated to an amino acid that is a conservative substitution of A. An exemplary conservative substitution of A is G. In some embodiments, a conservative substitution of A is not C or S. In some embodiments where a variant rhizavidin polypeptide comprises an N-linked glycosylation site having a motif of NXaaS, Nis mutated to Q. In some embodiments where a variant rhizavidin polypeptide comprises an N-linked glycosylation site having a motif of NXaaS, S is mutated to any amino acid that is not S or T. In some embodiments where a variant rhizavidin polypeptide comprises an N-linked glycosylation site having a motif of NXaaS, S is mutated to A. In some embodiments where a variant rhizavidin polypeptide comprises an N-linked glycosylation site having a motif of NXaaS, S is mutated to an amino acid that is a conservative substitution of A. An exemplary conservative substitution of A is G. In some embodiments, a conservative substitution of A is not C or S. In some embodiments where a variant rhizavidin polypeptide comprises an N-linked glycosylation site having a motif of NXaaS, S is mutated to Q. In some embodiments where a variant rhizavidin polypeptide comprises an N-linked glycosylation site having a motif of NXaaS, Xaa is mutated to P.

In some embodiments, where a variant rhizavidin polypeptide comprises an N-linked glycosylation site having a motif of NXaaT, Nis mutated to any amino acid that is not N or Q. In some embodiments, where a variant rhizavidin polypeptide comprises an N-linked glycosylation site having a motif of NXaaT, T is mutated to any amino acid that is not Q, S, or T. In some embodiments, such a variant rhizavidin polypeptide retains its ability to form a dimer.

In some embodiments, where a variant rhizavidin polypeptide comprises an N-linked glycosylation site having a motif of NXaaS, N is mutated to any amino acid that is not N or Q. In some embodiments, where a variant rhizavidin polypeptide comprises an N-linked glycosylation site having a motif of NXaaS, S is mutated to any amino acid that is not Q, S, or T. In some embodiments, such a variant rhizavidin polypeptide retains its ability to form a dimer.

In some embodiments, a variant rhizavidin polypeptide comprises a mutation at one or more glycosylation sites corresponding to amino acid residues: 65-67, 80-82, 106-108, 117-119, 118-120, 138-140, and 173-175 of a wild-type rhizavidin (e.g., a wild-type rhizavidin as set forth in SEQ ID NO: 1). In some embodiments, a variant rhizavidin polypeptide comprises a mutation at 1, 2, 3, 4, 5, 6, or 7 glycosylation sites corresponding to amino acid residues: 65-67, 80-82, 106-108, 117-119, 118-120, 138-140, and 173-175 of a wild-type rhizavidin (e.g., a wild-type rhizavidin as set forth in SEQ ID NO: 1). In some embodiments, a variant rhizavidin polypeptide comprises a mutation at a glycosylation site corresponding to amino acid residues 65-67 of a wild-type rhizavidin. In some embodiments, a variant rhizavidin polypeptide comprises a mutation at a glycosylation site corresponding to amino acid residues 80-82 of a wild-type rhizavidin. In some embodiments, a variant rhizavidin polypeptide comprises a mutation at a glycosylation site corresponding to amino acid residues 106-108 of a wild-type rhizavidin. In some embodiments, a variant rhizavidin polypeptide comprises a mutation at a glycosylation site corresponding to amino acid residues 117-119 of a wild-type rhizavidin. In some embodiments, a variant rhizavidin polypeptide comprises a mutation at a glycosylation site corresponding to amino acid residues 118-120 of a wild-type rhizavidin. In some embodiments, a variant rhizavidin polypeptide comprises a mutation at a glycosylation site corresponding to amino acid residues 138-140 of a wild-type rhizavidin. In some embodiments, a variant rhizavidin polypeptide comprises a mutation at a glycosylation site corresponding to amino acid residues 173-175 of a wild-type rhizavidin.

In some embodiments, a variant rhizavidin polypeptide comprises a mutation at one or more glycosylation sites corresponding to amino acid residues: 80-82, 106-108, 117-119, 118-120, and 138-140 of a wild-type rhizavidin (e.g., a wild-type rhizavidin as set forth in SEQ ID NO: 1). In some embodiments, a variant rhizavidin polypeptide comprises a mutation at 1, 2, 3, 4, or 5 glycosylation sites corresponding to amino acid residues: 80-82, 106-108, 117-119, 118-120, and 138-140 of a wild-type rhizavidin (e.g., a wild-type rhizavidin as set forth in SEQ ID NO: 1). In some embodiments, a variant rhizavidin polypeptide comprises a mutation at 3 or more glycosylation sites corresponding to amino acid residues: 80-82, 106-108, 117-119, 118-120, and 138-140 of a wild-type rhizavidin (e.g., a wild-type rhizavidin as set forth in SEQ ID NO: 1). In some embodiments, a variant rhizavidin polypeptide comprises a mutation at 4 or more of glycosylation sites corresponding to amino acid residues: 80-82, 106-108, 117-119, 118-120, and 138-140 of a wild-type rhizavidin (e.g., a wild-type rhizavidin as set forth in SEQ ID NO: 1). In some embodiments, a variant rhizavidin polypeptide comprises a mutation at all of glycosylation sites corresponding to amino acid residues: 80-82, 106-108, 117-119, 118-120, and 138-140 of a wild-type rhizavidin (e.g., a wild-type rhizavidin as set forth in SEQ ID NO: 1). In some such embodiments, a variant rhizavidin polypeptide does not comprise a mutation at a glycosylation site corresponding to amino acid residues 65-67 of a wild-type rhizavidin. In some such embodiments, a variant rhizavidin polypeptide does not comprises a mutation at a glycosylation site corresponding to amino acid residues 173-175 of a wild-type rhizavidin.

In some embodiments, a variant rhizavidin polypeptide comprises a mutation at position(s) corresponding to one or more of amino acid residues: 80, 106, 108, 118, 119, and 138 of a wild-type rhizavidin (e.g., a wild-type rhizavidin as set forth in SEQ ID NO: 1). In some embodiments, a variant rhizavidin polypeptide comprises a mutation at position(s) corresponding to 1, 2, 3, 4, 5, or 6 of amino acid residues: 80, 106, 108, 118, 119, and 138 of a wild-type rhizavidin (e.g., a wild-type rhizavidin as set forth in SEQ ID NO: 1). In some embodiments, a variant rhizavidin polypeptide comprises a mutation at position(s) corresponding to 4 or more of amino acid residues: 80, 106, 108, 118, 119, and 138 of a wild-type rhizavidin (e.g., a wild-type rhizavidin as set forth in SEQ ID NO: 1). In some embodiments, a variant rhizavidin polypeptide comprises a mutation at position(s) corresponding to 5 of amino acid residues: 80, 106, 108, 118, 119, and 138 of a wild-type rhizavidin (e.g., a wild-type rhizavidin as set forth in SEQ ID NO: 1). In some embodiments, a variant rhizavidin polypeptide comprises a mutation at position(s) corresponding to all of amino acid residues: 80, 108, 118, 119, and 138 of a wild-type rhizavidin (e.g., a wild-type rhizavidin as set forth in SEQ ID NO: 1). In some embodiments, a variant rhizavidin polypeptide comprises a mutation at a position corresponding to amino acid residue 80 of a wild-type rhizavidin. In some embodiments, a variant rhizavidin polypeptide comprises a mutation at a position corresponding to amino acid residue 106 of a wild-type rhizavidin. In some embodiments, a variant rhizavidin polypeptide comprises a mutation at a position corresponding to amino acid residue 108 of a wild-type rhizavidin. In some embodiments, a variant rhizavidin polypeptide comprises a mutation at a position corresponding to amino acid residue 118 of a wild-type rhizavidin. In some embodiments, a variant rhizavidin polypeptide comprises a mutation at a position corresponding to amino acid residue 119 of a wild-type rhizavidin. In some embodiments, a variant rhizavidin polypeptide comprises a mutation at a position corresponding to amino acid residue 138 of a wild-type rhizavidin. In some embodiments, a mutation at the aforementioned amino acid residues is an amino acid substitution by A. In some embodiments, a mutation at the aforementioned amino acid residues is an amino acid substitution by an amino acid that is a conservative substitution of A. An exemplary conservative substitution of A is G. In some embodiments, a conservative substitution of A is not C or S. In some embodiments, a mutation at the aforementioned amino acid residues is an amino acid substitution by Q.

In some embodiments, a variant rhizavidin polypeptide comprises one or more mutations at position(s) corresponding to one or more of the following mutations present in a wild-type rhizavidin (e.g., a wild-type rhizavidin as set forth in SEQ ID NO: 1): N80A/Q, N106A/Q, T108A, N118A/Q, S119A, N138A/Q. In some embodiments, a variant rhizavidin polypeptide comprises 1, 2, 3, 4, 5, or 6 of the following mutations in a wild-type rhizavidin (e.g., a wild-type rhizavidin as set forth in SEQ ID NO: 1): N80A/Q, N106A/Q, T108A, N118A/Q, S119A, and N138A/Q. In some embodiments, a variant rhizavidin polypeptide comprises 4 or more mutations at positions corresponding to four or more of the following mutations present in a wild-type rhizavidin (e.g., a wild-type rhizavidin as set forth in SEQ ID NO: 1): N80A/Q, N106A/Q, T108A, N118A/Q, S119A, N138A/Q. In some embodiments, a variant rhizavidin polypeptide comprises 5 or more mutations at positions corresponding to five or more of the following mutations present in a wild-type rhizavidin (e.g., a wild-type rhizavidin as set forth in SEQ ID NO: 1): N80A/Q, N106A/Q, T108A, N118A/Q, S119A, N138A/Q. In some embodiments, a variant rhizavidin polypeptide comprises mutations at positions corresponding to all of the following mutations present in a wild-type rhizavidin (e.g., a wild-type rhizavidin as set forth in SEQ ID NO: 1): N80A/Q, T108A, N118A/Q, S119A, N138A/Q. In some embodiments, a variant rhizavidin polypeptide comprises a mutation at a position corresponding to an N80A mutation in a wild-type rhizavidin. In some embodiments, a variant rhizavidin polypeptide comprises a mutation at a position corresponding to an N80Q mutation in a wild-type rhizavidin. In some embodiments, a variant rhizavidin polypeptide comprises a mutation at a position corresponding to an N106A mutation in a wild-type rhizavidin. In some embodiments, a variant rhizavidin polypeptide comprises a mutation at a position corresponding to an N106Q mutation in a wild-type rhizavidin. In some embodiments, a variant rhizavidin polypeptide comprises a mutation at a position corresponding to a T108A mutation in a wild-type rhizavidin. In some embodiments, a variant rhizavidin polypeptide comprises a mutation at a position corresponding to an N118A mutation in a wild-type rhizavidin. In some embodiments, a variant rhizavidin polypeptide comprises a mutation at a position corresponding to an N118Q mutation in a wild-type rhizavidin. In some embodiments, a variant rhizavidin polypeptide comprises a mutation at a position corresponding to a S119A mutation in a wild-type rhizavidin. In some embodiments, a variant rhizavidin polypeptide comprises a mutation at a position corresponding to an N138A mutation in a wild-type rhizavidin. In some embodiments, a variant rhizavidin polypeptide comprises a mutation at a position corresponding to an N138Q mutation in a wild-type rhizavidin.

In some embodiments, a variant rhizavidin polypeptide comprises a mutation at positions corresponding to each of amino acid residues: 80-82, 106-108, 118-120, and 138-140 of wild-type rhizavidin (e.g., a wild-type rhizavidin as set forth in SEQ ID NO: 1). In some embodiments, a variant rhizavidin polypeptide comprises a mutation at positions corresponding to each of amino acid residues: 80, 106, 118, and 138 of wild-type rhizavidin (e.g., a wild-type rhizavidin as set forth in SEQ ID NO: 1). In some embodiments, a variant rhizavidin polypeptide comprises mutations at positions corresponding to each of the following mutations in a wild-type rhizavidin (e.g., a wild-type rhizavidin as set forth in SEQ ID NO: 1): N80A, N106A, N118A, and N138A (referred to as “A4 variant”). In some embodiments, a variant rhizavidin polypeptide is or comprises the amino acid sequence of SEQ ID NO: 4. In some embodiments, a variant rhizavidin polypeptide is or comprises the amino acid sequence of SEQ ID NO: 5 or SEQ ID NO: 6.

In some embodiments, a variant rhizavidin polypeptide comprises a mutation at positions corresponding to each of amino acid residues: 80-82, 106-108, 118-120, and 138-140 of wild-type rhizavidin (e.g., a wild-type rhizavidin as set forth in SEQ ID NO: 1). In some embodiments, a variant rhizavidin polypeptide comprises a mutation at positions corresponding to each of amino acid residues: 80, 106, 118, and 138 of wild-type rhizavidin (e.g., a wild-type rhizavidin as set forth in SEQ ID NO: 1). In some embodiments, a variant rhizavidin polypeptide comprises mutations at positions corresponding to each of the following mutations in a wild-type rhizavidin (e.g., a wild-type rhizavidin as set forth in SEQ ID NO: 1): N80Q, N106Q, N118Q, and N138Q (referred to as “Q4 variant”). In some embodiments, a variant rhizavidin polypeptide is or comprises the amino acid sequence of SEQ ID NO: 7. In some embodiments, a variant rhizavidin polypeptide is or comprises the amino acid sequence of SEQ ID NO: 8 or SEQ ID NO: 9. In some embodiments, rhizavidin Q4 variant does not form dimers.

In some embodiments, a variant rhizavidin polypeptide comprises a mutation at positions corresponding to each of amino acid residues: 80-82, 106-108, 117-119, 118-120, and 138-140 of wild-type rhizavidin (e.g., a wild-type rhizavidin as set forth in SEQ ID NO: 1). In some embodiments, a variant rhizavidin polypeptide comprises a mutation at positions corresponding to each of amino acid residues: 80, 106, 118, 119, and 138 of wild-type rhizavidin (e.g., a wild-type rhizavidin as set forth in SEQ ID NO: 1). In some embodiments, a variant rhizavidin polypeptide comprises mutations at positions corresponding to each of the following mutations in a wild-type rhizavidin (e.g., a wild-type rhizavidin as set forth in SEQ ID NO: 1): N80A, N106A, N118A, S119A, and N138A (referred to as “A5 variant”). In some embodiments, a variant rhizavidin polypeptide is or comprises the amino acid sequence of SEQ ID NO: 10. In some embodiments, a variant rhizavidin polypeptide is or comprises the amino acid sequence of SEQ ID NO: 11 or SEQ ID NO: 12.

In some embodiments, a variant rhizavidin polypeptide comprises a mutation at positions corresponding to each of amino acid residues: 80-82, 106-108, 117-119, 118-120, and 138-140 of wild-type rhizavidin (e.g., a wild-type rhizavidin as set forth in SEQ ID NO: 1). In some embodiments, a variant rhizavidin polypeptide comprises a mutation at positions corresponding to each of amino acid residues: 80, 108, 118, 119, and 138 of wild-type rhizavidin (e.g., a wild-type rhizavidin as set forth in SEQ ID NO: 1). In some embodiments, a variant rhizavidin polypeptide comprises mutations at positions corresponding to each of the following mutations in a wild-type rhizavidin (e.g., a wild-type rhizavidin as set forth in SEQ ID NO: 1): N80A, T108A, N118A, S119A, and N138A (referred to as “AST variant”). In some embodiments, a variant rhizavidin polypeptide is or comprises the amino acid sequence of SEQ ID NO: 13. In some embodiments, a variant rhizavidin polypeptide is or comprises the amino acid sequence of SEQ ID NO: 14 or SEQ ID NO: 15.

In some embodiments, a variant rhizavidin polypeptide is a portion of a variant rhizavidin polypeptide (e.g., a portion of any of the amino acid sequences set forth in any one of SEQ ID NOs: 4-15), which portion includes at least 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170 or more contiguous amino acids of any one of SEQ ID NOs: 4-15. In some embodiments, a variant rhizavidin polypeptide contains one or more amino acid alterations (e.g., deletions, substitution, and/or insertion) from a variant rhizavidin polypeptide comprising the amino acid sequence of any one of SEQ ID NOs: 4-15.

For example, a variant rhizavidin polypeptide may comprise an amino acid sequence that is at least 60% or more (e.g., at least 65%, at least 70%, at least 75%, at least 80%, at least 85%, at least 90%, at least 95%, at least 96%, at least 97%, at least 98%, at least 99%, or more) identical to any one of SEQ ID NOs: 4-15, wherein the variant rhizavidin polypeptide comprises a mutation corresponding to one of the following mutations relative to SEQ ID NO: 1: N80A/Q, N106A/Q, T108A, N118A/Q, S119A, and/or N138A/Q. In some embodiments, a variant rhizavidin polypeptide may comprise a portion of the amino acid sequence as set forth in any one of SEQ ID NOs: 4-15 (e.g., at least 7, 8, 9, 10, 11, 12, 13, 14, 15, 16, 17, 18, 19, 20, 25, 30, 35, 45, 50, 55, 60, 65, 70, 75, 80, 85, 90, 95, 100, 1100, 110, 120, 130, 140, 150, 160, 170 or more consecutive amino acids of the sequence shown in any one of SEQ ID NOs: 4-15), wherein the variant rhizavidin polypeptide comprises a mutation corresponding to one of the following mutations relative to SEQ ID NO: 1: N80A/Q, N106A/Q, T108A, N118A/Q, S119A, and/or N138A/Q. In some embodiments, a variant rhizavidin polypeptide may contain a portion (e.g., at least 7, 8, 9, 10, 11, 12, 13, 14, 15, 16, 17, 18, 19, 20, 25, 30, 35, 45, 50, 55, 60, 65, 70, 75, 80, 85, 90, 95, 100, 100, 110, 120, 130, 140, 150, 160, 170 or more consecutive amino acids) of a sequence that is at least 60% or more (e.g., at least 65%, at least 70%, at least 75%, at least 80%, at least 85%, at least 90%, at least 95%, at least 96%, at least 97%, at least 98%, at least 99%, or more) identical to any one of SEQ ID NOs: 4-15, wherein the variant rhizavidin polypeptide comprises a mutation corresponding to one of the following mutations relative to SEQ ID NO: 1: N80A/Q, N106A/Q, T108A, N118A/Q, S119A, and/or N138A/Q. In some embodiments, a variant rhizavidin described herein, e.g., a portion of the amino acid sequence as set forth in SEQ ID NOs: 4-15, contains at least a portion corresponding to residues 60-167 of a wild-type rhizavidin polypeptide (e.g., as set forth in SEQ ID NO: 1). In some embodiments, a variant rhizavidin described herein, e.g., a portion of the amino acid sequence as set forth in SEQ ID NOs: 4-15, includes the amino acid residue C at a position corresponding to residue 94 and/or residue 123 of a wild-type rhizavidin polypeptide (e.g., as set forth in SEQ ID NO: 1). In some embodiments, a variant rhizavidin polypeptide may comprises no more than 25 (including, e.g., no more than 20, no more than 15, no more than 10, no more than 9, no more than 8, no more than 7, no more than 6, no more than 5, no more than 4, no more than 3, no more than 2) amino acid modifications (e.g., deletion, substitution, and/or insertion) within the sequence of any one of SEQ ID NOs: 4-15 or a portion thereof as described herein, wherein the variant rhizavidin polypeptide comprises a mutation corresponding to one of the following mutations relative to SEQ ID NO: 1: N80A/Q, N106A/Q, T108A, N118A/Q, S119A, and/or N138A/Q. In some embodiments, such amino acid modifications may be present in the N-terminal portion and/or C-terminal portion.

In some embodiments, a variant rhizavidin polypeptide is characterized in that is binds to biotin or a derivative or mimic molecule thereof. In some embodiments, a variant rhizavidin polypeptide is characterized in that is binds to biotin or a derivative or mimic molecule thereof, at an affinity that is at least 50%, at least 60%, at least 70%, at least 80%, at least 85%, at least 90%, at least 95%, at least 96%, at least 97%, at least 98%, at least 99%, or more of a reference rhizavidin polypeptide (e.g., a wild-type rhizavidin polypeptide).

In some embodiments, a variant rhizavidin polypeptide is characterized in that is binds to a biotin derivative, lipoic acid, HABA (hydroxyazobenzene-benzoic acid), dimethyl-HABA, or an amine-PEG3-biotin ((+)-biotinylation-3-6,9-trixaundecanediamine). In some embodiments, a variant rhizavidin polypeptide is characterized in that is binds to a biotin derivative, lipoic acid, HABA (hydroxyazobenzene-benzoic acid), dimethyl-HABA, or an amine-PEG3-biotin ((+)-biotinylation-3-6,9-trixaundecanediamine), at an affinity that is at least 50%, at least 60%, at least 70%, at least 80%, at least 85%, at least 90%, at least 95%, at least 96%, at least 97%, at least 98%, at least 99%, or more of a reference rhizavidin polypeptide (e.g., a wild-type rhizavidin polypeptide).

A nucleic acid comprising a nucleotide sequence encoding a variant rhizavidin polypeptide described herein is also within the scope of the present disclosure. In some embodiments, such a nucleic acid comprises one or more regulatory sequences that directs expression of the variant rhizavidin polypeptide. Exemplary regulatory sequences include, but are not limited to a promoter, an intron, an enhancer, a polyadenylation signal, a terminator, a silencer, a TATA box, or a Woodchuck Hepatitis Virus (WHP) Posttranscriptional Regulatory Element (WPRE).

### Compositions Comprising Rhizavidin Variants Described Herein

Variant rhizavidin polypeptides described herein can be useful as biotin-binding proteins for various applications, e.g., in some embodiments, to couple two molecular entities (e.g., small molecules, proteins, lipids, polysaccharides, etc.) via non-covalent biotin-rhizavidin interaction. For example, in some embodiments, variant rhizavidin polypeptides described herein can be useful in compositions, systems, or methodologies involving an avidin-biotin system. As one of skill in the art is well aware, an avidin-biotin system can be used for numerous laboratory methods, such as bioconjugation; target molecule detection; target molecule isolation, purification, or enrichment from a sample; protein detection; nucleic acid detection; protein isolation, purification, or enrichment; nucleic acid isolation; purification, or enrichment; ELISA, flow cytometry; and the like. Accordingly, compositions comprising variant rhizavidin polypeptides described herein, can be used for, but are not limited to, bioconjugation; target molecule detection; target molecule isolation, purification, or enrichment from a sample; protein detection; nucleic acid detection; protein isolation, purification, or enrichment; nucleic acid isolation; purification, or enrichment; ELISA, flow cytometry; and the like.

In some embodiments, a composition comprises a variant rhizavidin polypeptide and a molecule of interest. In some embodiments, such a molecule of interest is a polypeptide, polysaccharide, nucleic acid, lipid, or small molecule. In some embodiments, such a molecule of interest is a polypeptide (e.g., in some embodiments an antigenic polypeptide described herein). In some embodiments, such a molecule of interest is an antigen.

In some embodiments, a variant rhizavidin polypeptide in a composition is associated with a molecule of interest. For example, in some embodiments, a variant rhizavidin polypeptide is non-covalently associated with a molecule of interest. In some embodiments, a variant rhizavidin polypeptide is covalently linked with a molecule of interest. In some embodiments, a composition comprising a variant rhizavidin polypeptide and a molecular interest is a fusion protein comprising rhizavidin polypeptide and a molecule of interest. In some such embodiments, the molecule of interest is a polypeptide.

### Antigenic Polypeptides

In some embodiments, a composition described herein comprises one or more polypeptide antigens. In some embodiments, a composition described herein comprises a rhizavidin polypeptide (e.g., a variant rhizavidin polypeptide disclosed herein) and one or more polypeptide antigens. In some embodiments, a polypeptide antigen is or comprises a bacterial polypeptide antigen, a fungal polypeptide antigen, a parasitic polypeptide antigen, a viral polypeptide antigen, and/or a mammalian polypeptide antigen. An exemplary mammalian polypeptide antigen is or comprises a tumor antigen.

In some embodiments, one or more polypeptide antigens included in a composition described herein comprise two or more polypeptide antigens from the same source, e.g., in some embodiments, associated with the same pathogen (including, e.g., a bacterium, a fungus, a parasite, a virus) or associated with the same cancer or tumor. In some embodiments, one or more polypeptide antigens included in a composition described herein comprise polypeptide antigens associated with different pathogens (including, e.g., bacteria, fungi, parasites, viruses) or associated with a different cancer or tumors. For example, one or more polypeptide antigens included in a composition described herein comprises (i) a polypeptide antigen associated with a first bacterium, fungus, parasite, virus, or cancer or tumor, and (ii) a polypeptide antigen associated with a second bacterium, fungus, parasite, virus, or cancer or tumor.

In some embodiments, a polypeptide antigen is or comprises a bacterial polypeptide antigen. In some embodiments, a bacterial polypeptide antigen is a polypeptide antigen of, or derived from, a Gram-negative or a Gram-positive bacterium. In some embodiments, a bacterial polypeptide antigen is or comprises a pneumococcal (e.g., group A, group B, and viridans) antigen, a tuberculosis antigen, an anthrax antigen, a pertussis antigen, a staphylococcal (e.g., S. aureus) antigen, a meningococcal antigen, a Haemophilus antigen, a Shigella antigen, a Salmonella antigen, a Pseudomonas antigen, a Klebsiella antigen, an E. coli antigen, or combinations thereof. In some embodiments, a bacterial polypeptide antigen is a polypeptide antigen of, or derived from, S. pneumoniae.

In some embodiments, a polypeptide antigen is or comprises a viral polypeptide antigen. In some embodiments, a viral polypeptide antigen is or comprises a coronavirus antigen, an HIV antigen, an HSV (e.g., HSV-1 or HSV-2) antigen, an HPV antigen, an influenza (e.g., seasonal or epidemic) antigen, or combinations thereof. In some embodiments, a viral polypeptide antigen is a polypeptide antigen of, or derived from, a coronavirus. In some embodiments, a viral polypeptide antigen is a polypeptide antigen of, or derived from, SARS-CoV-2.

In some embodiments, a polypeptide antigen is or comprises a fungal polypeptide antigen. In some embodiments, a fungal polypeptide antigen is or comprises an apergillosis antigen, a blastomycosis antigen, a candidiasis antigen, a chromoblastomycosis antigen, a coccidioidomycosis antigen, a emergomycosis antigen, a fungal eye infection antigen, a fungal nail infection antigen, a fusariosis antigen, a histoplasmosis antigen, a mucormycosis antigen, a mycetoma antigen, a paracoccidioidomycosis antigen, a Pneumocystis pneumonia antigen, a ringworm antigen, a scedosporiosis antigen, a sporotrichosis antigen, a talaromycosis antigen, or combinations thereof. In some embodiments, a polypeptide antigen is, or is derived from, an Aspergillus antigen, a Blastomyces antigen, a Candida (e.g., C. auris) antigen, a Coccidioides antigen, a Cryptococcus antigen (e.g., C. neoformans and C. gattii), an Epidermophyton antigen, a Fusarium antigen, a Histoplasma antigen, a Lomentospora antigen, a Madurella antigen, Mucormycetes antigen, a Microsporum antigen, a Paracoccidioidomycosis antigen, a Pneumocystis (e.g., P. jirovecii) antigen, a Scedosporium antigen, a Sporothrix antigen, a Talaromyces antigen, a Trichophyton antigen, or combinations thereof.

In some embodiments, a polypeptide antigen is or comprises a parasitic protozoan polypeptide antigen. In some embodiments, a parasitic protozoan polypeptide antigen is or comprises an amoebiasis antigen, a baesiosis antigen, a Blastocystis antigen, a Chagas disease antigen, cryptosporidiosis, a giardiasis antigen, a lambliasis antigen, a malaria antigen, a toxoplasmosis antigen, trichomoniasis antigen, or combinations thereof. In some embodiments, a polypeptide antigen is, or is derived from, a Babesia (e.g., B. microti) antigen, a Balantidium antigen, a Cryptosporidium antigen, a Entamoeba antigen, a Giardia (e.g., G. lamblia) antigen, a Leishmania antigen, a Plasmodium (e.g., P. falciparum) antigen, a Trichomoniasis (e.g., T. vaginalis) antigen, a Trypanosoma (e.g., T. cruzi and T. brucei) antigen, or combinations thereof.

In some embodiments, a polypeptide antigen is or comprises a tumor antigen. In some embodiments, a tumor antigen is, or is derived from, carcinoembryonic antigen (CEA), cancer/testis antigens (e.g., New York esophageal squamous cell carcinoma 1 (NY-ESO1), mucin-1 (MUC1), and Sialyl Tn (STn)), gangliosides (e.g., GM3 and GD2), p53, HER2/neu, EGFR (e.g., EGFRvIII), melanocyte/melanoma differentiation antigens (e.g., tyrosinase, MART1, gp100, melanoma antigen (MAGE) family proteins, prostate-specific antigen (PSA), leukemia-associated antigens (LAAs) (e.g., BCR-ABL, Wilms' tumor protein and proteinase 3, idiotype (Id) antibodies, or combinations thereof. See e.g., Mitchell, Curr. Opinion. Investig. Drugs 150 (2002), Dao & Scheinberg, Best Pract. Res. Clin. Haematol. 391 (2008). In some embodiments, a tumor antigen is or comprises a tumor antigen from a solid tumor. In some embodiments, a tumor antigen is or comprises a tumor antigen from a hematologic tumor. In some embodiments, a tumor antigen is or comprises a tumor antigen associated with a cancer. Examples of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, ovarian and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

In some embodiments, a composition described herein includes one or more of the following antigenic polypeptides, or portions thereof.

### Exemplary Coronavirus Polypeptide Antigens

In some embodiments, an antigenic polypeptide described herein is or comprises a coronavirus polypeptide antigen. Coronavirus genomes encode non-structural polyprotein and structural proteins, including the Spike(S), Envelope (E), Membrane (M) and Nucleocapsid (N) proteins. In some embodiments, a polypeptide antigen is a Spike(S) protein or antigenic fragment thereof, an Envelope (E) protein or antigenic fragment thereof, a Membrane (M) protein or antigenic fragment thereof; and/or a Nucleocapsid (N) protein or antigenic fragment thereof.

As seen notably with SARS-CoV, neutralizing antibodies and/or T-cell immune responses can be raised against several proteins but mostly target the S glycoprotein, suggesting that S glycoprotein-induced specific immune responses play important parts in the natural response to coronavirus infection (Saif L J, Vet Microbiol. 1993 November; 37(3-4):285-97). The S glycoprotein has key roles in the viral cycle, as it is involved in receptor recognition, virus attachment and entry, and is thus a crucial determinant of host tropism and transmission capacity. Expressed as precursor glycoprotein, S is cleaved in two subunits (S1, which contains the receptor binding domain (RBD), and S2) by proteases.

SEQ ID NO: 16 is the amino acid sequence of the Spike(S) glycoprotein of the 2019 novel coronavirus initially named 2019-nCov and renamed SARS-CoV-2 (Severe acute respiratory syndrome coronavirus 2). The S glycoprotein comprises a signal peptide (SP) from position 1 to 18 which is cleaved in the mature S glycoprotein. The S glycoprotein is cleaved by proteases into two subunits, S1, which contains the receptor binding domain (RBD), and S2. S1 is from positions 19 to 661 of SEQ ID NO: 16 and S2 is from positions 662 to 1270 of SEQ ID NO: 16. The receptor binding domain (RBD) is from positions 331 to 524 in SEQ ID NO: 16. By simple sequence alignment with SEQ ID NO: 16, one skilled in the art can easily determine the positions of the RBD in the sequence of an S glycoprotein antigen variant or fragment thereof.

S-RBD is believed to mediate entry of the lineage B SARS coronavirus to respiratory epithelial cells by binding to the cell surface receptor angiotensin-converting enzyme 2 (ACE2). In particular, a receptor binding motif (RBM) in the virus S-RBD is believed to interact with ACE2. The amino acid sequence of SARS-CoV-2 Wuhan-Hu-1 S-RBD is provided in SEQ ID NO: 17. The amino acid sequence of SARS-CoV-2 Wuhan-Hu-1 RBM is provided in SEQ ID NO: 18.

In some embodiments, a coronavirus polypeptide antigen has or comprises an amino acid sequence that is at least 90%, at least 91%, at least 92%, at least 93%, at least 94%, at least 95%, at least 96%, at least 97%, at least 98%, at least 99%, or at least 99.5%, or 100% identical to the amino acid sequence of SEQ ID NOs: 16-18, or a portion thereof (e.g., lacking 1, 2, 3, 4, 5, or more amino acids of SEQ ID NOs: 16-18).

There have been a number of emerging SARS-CoV-2 variants. Some SARS-CoV-2 variants contain an N439K mutation, which has enhanced binding affinity to the human ACE2 receptor (Thomson, E. C., et al., The circulating SARS-CoV-2 Spike variant N439K maintains fitness while evading antibody-mediated immunity. bioRxiv, 2020). Some SARS-CoV-2 variants contain an N501Y mutation, which is associated with increased transmissibility, including the lineages B.1.1.7 (also known as201/501Y.V1 and VOC 202012/01; (del69-70, del144, N501Y, A570D, D614G, P681H, T716I, S982A, and D1118H mutations)) and B.1.351 (also known as 20H/501Y.V2; L18F, D80A, D215G, R246I, K417N, E484K, N501Y, D614G, and A701V mutations), which were discovered in the United Kingdom and South Africa, respectively (Tegally, H., et al., Emergence and rapid spread of a new severe acute respiratory syndrome-related coronavirus 2 (SARS-CoV-2) lineage with multiple Spike mutations in South Africa. medRxiv, 2020: p. 2020.12.21.20248640; Leung, K., et al., Early empirical assessment of the N501Y mutant strains of SARS-CoV-2 in the United Kingdom, October to November 2020. medRxiv, 2020: p. 2020.12.20.20248581). B.1.351 also include two other mutations in the RBD domain of SARS-CoV-2 Spike protein, K417N and E484K (Tegally, H., et al., Emergence and rapid spread of a new severe acute respiratory syndrome-related coronavirus 2 (SARS-CoV-2) lineage with multiple Spike mutations in South Africa. medRxiv, 2020: p. 2020.12.21.20248640). Other SARS-CoV-2 variants include the Lineage B.1.1.28, which was first reported in Brazil; the Variant P.1, lineage B.1.1.28 (also known as20J/501Y.V3), which was first reported in Japan; Variant L452R, which was first reported in California in the United States (Pan American Health Organization, Epidemiological update: Occurrence of variants of SARS-CoV-2 in the Americas, Jan. 20, 2021, available at iris.paho.org/bitstream/handle/10665.2/53219/EpiUpdate20January2021_eng.pdf). Other SARS-CoV-2 variants include a SARS-CoV-2 of clade 19A; SARS-CoV-2 of clade 19B; a SARS-CoV-2 of clade 20A; a SARS-CoV-2 of clade 20B; a SARS-CoV-2 of clade 20C; a SARS-CoV-2 of clade 20D; a SARS-CoV-2 of clade 20E (EU1); a SARS-CoV-2 of clade 20F; a SARS-CoV-2 of clade 20G; and SARS-CoV-2 B.1.1.207; and other SARS-CoV-2 lineages described in Rambaut, A., et al., A dynamic nomenclature proposal for SARS-CoV-2 lineages to assist genomic epidemiology. Nat Microbiol 5,1403-1407 (2020). The foregoing SARS-CoV-2 variants, and the amino acid and nucleotide sequences thereof, are incorporated herein by reference. Those skilled in the art are aware of various SARS-CoV-2 variants and their mutations, for example, relative to a Wuhan strain sequence. In some embodiments, such variants of SARS-CoV-2 may be identified based on publicly available data (e.g., data provided in the GISAID Initiative database: https://www.gisaid.org, and/or data provided by the World Health Organization WHO (e.g., as provided at https://www.who.int/activities/tracking-SARS-CoV-2-variants).

In some embodiments, a coronavirus polypeptide antigen is or comprises a SARS-CoV-2 glycoprotein. In some embodiments, a coronavirus polypeptide antigen is or comprises a SARS-CoV-2 glycoprotein S RBD listed in Table 1 (or an antigenic fragment thereof), or comprises a SARS-CoV-2 glycoprotein S RBD (or an antigenic fragment thereof) that includes one or more mutations listed in Table 1:

TABLE 1
Representative Mutations of SARS-CoV-2 Glycoprotein S RBD [aa 331-524]

| Mutations | Strain Designation | SEQ ID NO: |
|---|---|---|
| Reference | Wuhan D614G | SEQ ID NO: 19 |
| N501Y | Alpha, B.1.1.7, UK | SEQ ID NO: 20 |
| K417N, E484K, N501Y | Beta, B.1.351, South Africa | SEQ ID NO: 21 |
| L452R, T478K | Delta, B.1.617.2, India | SEQ ID NO: 22 |
| K417N, L452, T478K | Delta plus, AY.1, India | SEQ ID NO: 23 |
| K417T, E484K, N501Y | Gamma, P.1, Japan/Brazil | SEQ ID NO: 24 |
| L452R | Epsilon, B.1.427, B.1.429, CA | SEQ ID NO: 25 |
| E484K | Eta, B.1.525, UK/Nigeria | SEQ ID NO: 26 |
| L452R, E484K | Iota, B.1.526, US/NY | SEQ ID NO: 27 |
| L452R, E484Q | Kappa, B.1.617.1, India | SEQ ID NO: 28 |
| K417N, S477N, E484A, Q493K, | Omicron, B.1.1.529, Multiple | SEQ ID NO: 29 |
| G496S, N501Y, Y505H | Countries | |
| K417N, L452R, E484Q, N501Y | Potential VOC #1 | SEQ ID NO: 30 |
| K417N, L452R, E484K, N501Y | Potential VOC #2 | SEQ ID NO: 31 |
| L452R, E484Q, N501Y | Potential VOC #3 | SEQ ID NO: 32 |
| L452R, E484K, N501Y | Potential VOC #4 | SEQ ID NO: 33 |
| E484Q | TBD | |
| K417N, E484Q | TBD | |
| K417N, E484K | TBD | |
| K417N, L452R, E484K | TBD | |
| N501Y | TBD | |
| K417N, N501Y | TBD | |
| K417N, E484Q, N501Y | TBD | |
| K417N, E484K, N501Y | TBD | |
| K417N, L452R, N501Y | TBD | |

### Exemplary Pneumococcal Polypeptide Antigens: Pneumolysin Polypeptides

In some embodiments, an antigenic polypeptide described herein is or comprises a pneumococcal polypeptide antigen. In some embodiments, a pneumococcal polypeptide antigen is or comprises pneumolysin polypeptide antigen. Pneumolysin (Ply) is a S. pneumoniae protein toxin. Ply is a cholesterol-dependent toxin of the thiol-activated cytolysin family. In some embodiments, a Ply polypeptide antigen is or comprises a full-length Ply polypeptide. For example, in some embodiments, a full-length Ply polypeptide has 470 amino acids (53 kDa) and is represented by the amino acid sequence as set forth in SEQ ID NO: 34. In some embodiments, a Ply polypeptide antigen includes a portion of a Ply polypeptide (e.g., a portion of a Ply polypeptide of SEQ ID NO: 34, which portion includes at least 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450 or more contiguous amino acids of SEQ ID NO: 34). In some embodiments, a portion of a Ply polypeptide corresponds to a protein having amino acids 2-470 of the amino acid sequence set forth in SEQ ID NO: 34. In some embodiments, a Ply polypeptide antigen contains one or more amino acid alterations (e.g., deletion, substitution, and/or insertion) from a naturally-occurring wild-type Ply polypeptide sequence. For example, a Ply polypeptide antigen may contain an amino acid sequence that is at least 60% or more (e.g., at least 65%, 70%, 75%, 80%, 85%, 90%, 95%, or 98%) identical to SEQ ID NO: 34 or a portion thereof (e.g., at least 7, 8, 9, 10, 11, 12, 13, 14, 15, 16, 17, 18, 19, 20, 25, 30, 35, 45, 50, 55, 60, 65, 70, 75, 80, 85, 90, 95, 100, 150, 200, 250, 300, 350, 400, 450 or more consecutive amino acids of the sequence shown in SEQ ID NO: 34). Alternatively, a Ply polypeptide antigen may contain a portion (e.g., at least 7, 8, 9, 10, 11, 12, 13, 14, 15, 16, 17, 18, 19, 20, 25, 30, 35, 45, 50, 55, 60, 65, 70, 75, 80, 85, 90, 95, 100, 150, 200, 250, 300, 350, 400, 450 or more consecutive amino acids) of a sequence that is at least 60% or more (e.g., at least 65%, 70%, 75%, 80%, 85%, 90%, 95%, or 98%) identical to SEQ ID NO: 34. In some embodiments, a nucleotide sequence encoding a Ply polypeptide is provided herein as SEQ ID NO: 44.

Pneumolysins are exotoxins produced by bacteria that can cause hemolytic activity and complement activation. While highly immunogenic, their use in vaccines can be limited because they cause lysis of red blood cells. Accordingly, in some embodiments, a Ply polypeptide antigen is or comprises a Ply variant (also designated herein as a mutant Ply or “mPly”) that is substantially non-hemolytic. As used herein, the phrase “substantially non-hemolytic” means the ability of lysing red blood cells being reduced by at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or up to 100%, as compared to that of an equivalent concentration of a reference Ply (e.g., a wild-type Ply). In some embodiments, hemolytic activity of substantially non-hemolytic Ply is at least 5%, at least 10%, at least 15%, at least 20%, at least 20%, at least 30%, at least 30%, at least 35%, least 40%, at least 45%, at least 50%, at least 55%, at least 60%, at least 65%, at least 70%, at least 75%, at least 80%, at least 85%, at least 90%, at least 95% lower than an equivalent concentration of a reference Ply (e.g., a wild-type Ply). In some embodiments, the substantially non-hemolytic Ply has no detectable hemolytic activity. The term “wild-type Ply” is accorded the usual definition associated with such phrase, e.g., in some embodiments, a naturally occurring Ply (e.g., a Ply that is naturally secreted by a capable bacterial source). In some embodiments, a wild-type Ply protein is represented by the amino acid sequence as set forth in SEQ ID NO: 34.

In some embodiments, a mutant Ply (e.g., non-hemolytic Ply) comprises a wild-type Ply amino acid sequence (e.g., an amino acid sequence as set forth in SEQ ID NO: 34) or an antigenic fragment thereof, with one or more amino acid substitutions. In some embodiments, a mutant Ply (e.g., non-hemolytic Ply) comprises a wild-type Ply amino acid sequence (e.g., an amino acid sequence as set forth in SEQ ID NO: 34) or an antigenic fragment thereof, with one or more of the following amino acid substitutions: residue D385 substituted with N; residue C428 substituted with G, and residue W433 substituted with F. See, for example, Berry et al., “Effect of defined point mutations in the pneumolysin gene on the virulence of Streptococcus pneumoniae”. Infect Immune 1995 63(5):1969-1974). In some embodiments, a mutant Ply (e.g., non-hemolytic Ply) carrying the amino acid substitutions D385N, C428G, and W433F is referred to as PdT. In some embodiments, a PdT is or comprises the amino acid sequence as set forth in SEQ ID NO: 35.

In some embodiments, a mutant Ply (e.g., non-hemolytic Ply) comprises a wild-type Ply amino acid sequence (e.g., an amino acid sequence as set forth in SEQ ID NO: 34) or an antigenic fragment thereof, with G294 substituted with P (See, e.g., Oloo et al., “Structure-guided antigen engineering yields pneumolysin mutants suitable for vaccination against pneumococcal disease”. J Biol Chem. 2011 286(14):12133-12140). In some embodiments, a mutant Ply (e.g., non-hemolytic Ply) comprises a wild-type Ply amino acid sequence (e.g., an amino acid sequence as set forth in SEQ ID NO: 34) or an antigenic fragment thereof, with all of the following amino acid substitutions: residue D385 substituted with N; residue C428 substituted with G; residue W433 substituted with F; and G294 substituted with P. In some embodiments, a mutant Ply (e.g., non-hemolytic Ply) carrying the amino acid substitutions G294P, D385N, C428G, and W433F is referred to as PdT (G294P). In some embodiments, a PdT (G294P) is or comprises the amino acid sequence as set forth in SEQ ID NO: 36.

In some embodiments, a mutant Ply (e.g., non-hemolytic Ply) is a portion of a PdT (G294P) polypeptide (e.g., a portion of the PdT (G294P) polypeptide of SEQ ID NO: 36, which portion includes at least 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450 or more contiguous amino acids of SEQ ID NO: 36. In some embodiments, such a portion of PdT (G294P) polypeptide include the four amino acid substitutions described herein. In some embodiments, a portion of a PdT (G294P) polypeptide corresponds to a protein having amino acids 2-470 of the amino acid sequence set forth in SEQ ID NO: 36. In some embodiments, a mutant Ply (e.g., non-hemolytic Ply) contains one or more amino acid alterations (e.g., deletion, substitution, and/or insertion) from the PdT (G294P) polypeptide sequence of SEQ ID NO: 36. For example, a mutant Ply (e.g., non-hemolytic Ply) may contain an amino acid sequence that is at least 60% or more (e.g., at least 65%, 70%, 75%, 80%, 85%, 90%, 95%, or 98%) identical to SEQ ID NO: 36 or a portion thereof (e.g., at least 7, 8, 9, 10, 11, 12, 13, 14, 15, 16, 17, 18, 19, 20, 25, 30, 35, 45, 50, 55, 60, 65, 70, 75, 80, 85, 90, 95, 100, 150, 200, 250, 300, 350, 400, 450 or more consecutive amino acids of the sequence shown in SEQ ID NO: 36). Alternatively, a mutant Ply (e.g., non-hemolytic Ply) may contain a portion (e.g., at least 7, 8, 9, 10, 11, 12, 13, 14, 15, 16, 17, 18, 19, 20, 25, 30, 35, 45, 50, 55, 60, 65, 70, 75, 80, 85, 90, 95, 100, 150, 200, 250, 300, 350, 400, 450 or more consecutive amino acids) of a sequence that is at least 60% or more (e.g., at least 65%, 70%, 75%, 80%, 85%, 90%, 95%, or 98%) identical to SEQ ID NO: 36. In some embodiments, a mutant Ply (e.g., non-hemolytic Ply) may comprises no more than 25 (including, e.g., no more than 20, no more than 15, no more than 10, no more than 9, no more than 8, no more than 7, no more than 6, no more than 5, no more than 4, no more than 3, no more than 2) amino acid modifications (e.g., deletion, substitution, and/or insertion) within the sequence of SEQ ID NO: 36 or a portion thereof as described herein. In some embodiments, such amino acid modifications may be present in the N-terminal portion and/or C-terminal portion.

### Exemplary Pneumococcal Polypeptide Antigens: SP0435 Polypeptides

In some embodiments, an antigenic polypeptide described herein is or comprises a pneumococcal polypeptide antigen. In some embodiments, a pneumococcal polypeptide antigen is or comprises a SP0435 polypeptide. SP0435 is a S. pneumoniae protein. In some embodiments, an SP0435 polypeptide is an elongation factor P. In some embodiments, an SP0435 polypeptide is or comprises a full-length SP0435 polypeptide. For example, in some embodiments, a full-length SP0435 polypeptide has 186 amino acids (20 kDa) and is represented by the amino acid sequence as set forth in SEQ ID NO: 38. Without wishing to be bound by a particular theory, amino acids 1-61 of SEQ ID NO: 38 are predicted to be a dimerization domain of an SP0435 polypeptide (amino acids 1-61 of the full-length protein). Accordingly, in some embodiments, a SP0435 polypeptide may exclude such a dimerization domain, for example in some embodiments, to minimize or avoid the possibility of crosslinking or interference with a rhizavidin moiety. In some embodiments, an SP0435 polypeptide includes a portion of an SP0435 polypeptide (e.g., a portion of the SP0435 polypeptide of SEQ ID NO: 38, which portion includes at least 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, or more contiguous amino acids of SEQ ID NO: 38). In some embodiments, a portion of an SP0435 polypeptide corresponds to a protein having amino acids 62-185 of the amino acid sequence set forth in SEQ ID NO: 38. In some embodiments, an SP0435 polypeptide contains one or more amino acid alterations (e.g., deletion, substitution, and/or insertion) from a naturally-occurring wild-type SP0435 polypeptide sequence. For example, an SP0435 polypeptide may contain an amino acid sequence that is at least 60% or more (e.g., at least 65%, 70%, 75%, 80%, 85%, 90%, 95%, or 98%) identical to SEQ ID NO: 38 or a portion thereof (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or more consecutive amino acids of the sequence shown in SEQ ID NO: 38). Alternatively, an SP0435 polypeptide may contain a portion (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or more consecutive amino acids) of a sequence that is at least 60% or more (e.g., at least 65%, 70%, 75%, 80%, 85%, 90%, 95%, or 98%) identical to SEQ ID NO: 38. In some embodiments, a nucleotide sequence encoding an SP0435 polypeptide is provided herein as SEQ ID NO: 45. In some embodiments, an SP0434 polypeptide may comprises no more than 25 (including, e.g., no more than 20, no more than 15, no more than 10, no more than 9, no more than 8, no more than 7, no more than 6, no more than 5, no more than 4, no more than 3, no more than 2) amino acid modifications (e.g., deletion, substitution, and/or insertion) within the sequence of SEQ ID NO: 38 or a portion thereof as described herein. In some embodiments, such amino acid modifications may be present in the N-terminal portion and/or C-terminal portion. In some embodiments, an SPN0435 polypeptide is one described in WO 2011/112906, the entire content of which is incorporated herein by reference for the purposes described herein.

### Exemplary Pneumococcal Polypeptide Antigens: SP0785 Polypeptides

In some embodiments, an antigenic polypeptide described herein is or comprises a pneumococcal polypeptide antigen. In some embodiments, a pneumococcal polypeptide antigen is or comprises a SP0785 polypeptide. SP0785 is a conserved hypothetical S. pneumoniae protein, for example, in some embodiments as described in WO 2014/124228, the entire content of which is incorporated herein by reference for the purposes described herein. In some embodiments, an SP0785 polypeptide is an efflux transporter protein conserved across S. pneumoniae strains. In some embodiments, an SP0785 polypeptide is or comprises a full-length SP0785 polypeptide. For example, in some embodiments, a full-length SP0785 polypeptide has 399 amino acids (38 kDa) and is represented by the amino acid sequence as set forth in SEQ ID NO: 40. Without wishing to be bound by a particular theory, amino acids 1-32 of SEQ ID NO: 40 are predicted to be a signal sequence and transmembrane domain of an SP0785 polypeptide (amino acids 1-32 of the full-length protein). Accordingly, in some embodiments, an SP0785 polypeptide may exclude such a signal sequence and transmembrane domain. In some embodiments, an SP0785 polypeptide includes a portion of an SP0785 polypeptide (e.g., a portion of the SP0785 polypeptide of SEQ ID NO: 40, which portion includes at least 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, or more contiguous amino acids of SEQ ID NO: 40). In some embodiments, a portion of an SP0785 polypeptide corresponds to a protein having amino acids 33-399 of the amino acid sequence set forth in SEQ ID NO: 40. In some embodiments, an SP0785 polypeptide contains one or more amino acid alterations (e.g., deletion, substitution, and/or insertion) from a naturally-occurring wild-type SP0785 polypeptide sequence. For example, an SP0785 polypeptide may contain an amino acid sequence that is at least 60% or more (e.g., at least 65%, 70%, 75%, 80%, 85%, 90%, 95%, or 98%) identical to SEQ ID NO: 40 or a portion thereof (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or more consecutive amino acids of the sequence shown in SEQ ID NO: 40). Alternatively, an SP0785 polypeptide may contain a portion (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids) of a sequence that is at least 60% or more (e.g., at least 65%, 70%, 75%, 80%, 85%, 90%, 95%, or 98%) identical to SEQ ID NO: 40. In some embodiments, a nucleotide sequence encoding an SP0785 polypeptide is provided herein as SEQ ID NO: 46. In some embodiments, an SP0785 polypeptide may comprises no more than 25 (including, e.g., no more than 20, no more than 15, no more than 10, no more than 9, no more than 8, no more than 7, no more than 6, no more than 5, no more than 4, no more than 3, no more than 2) amino acid modifications (e.g., deletion, substitution, and/or insertion) within the sequence of SEQ ID NO: 40 or a portion thereof as described herein. In some embodiments, such amino acid modifications may be present in the N-terminal portion and/or C-terminal portion.

### Exemplary Pneumococcal Polypeptide Antigens: SP 1500 Polypeptides

In some embodiments, an antigenic polypeptide described herein is or comprises a pneumococcal polypeptide antigen. In some embodiments, a pneumococcal polypeptide antigen is or comprises a SP1500 polypeptide. SP1500 is a S. pneumoniae protein, for example, in some embodiments as described in WO 2014/124228, the entire content of which is incorporated herein by reference for the purposes described herein. In some embodiments, an SP1500 polypeptide is an Amino Acid ABC Transporter, amino acid-binding polypeptide conserved across S. pneumoniae strains. In some embodiments, an SP1500 polypeptide is or comprises a full-length SP1500 polypeptide. For example, in some embodiments, a full-length SP1500 polypeptide has 278 amino acids (28 kDa) and is represented by the amino acid sequence as set forth in SEQ ID NO: 42. Without wishing to be bound by a particular theory, amino acids 1-26 of SEQ ID NO: 42 are predicted to be a signal sequence of an SP1500 polypeptide (amino acids 1-26 of the full-length protein). Accordingly, in some embodiments, a SP1500 polypeptide may exclude such a signal sequence. In some embodiments, an SP1500 polypeptide includes a portion of an SP1500 polypeptide (e.g., a portion of the SP1500 polypeptide of SEQ ID NO: 42, which portion includes at least 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, or more contiguous amino acids of SEQ ID NO: 42). In some embodiments, a portion of an SP1500 polypeptide corresponds to a protein having amino acids 27-278 of the amino acid sequence set forth in SEQ ID NO: 42. In some embodiments, an SP1500 polypeptide contains one or more amino acid alterations (e.g., deletion, substitution, and/or insertion) from a naturally-occurring wild-type SP1500 polypeptide sequence. For example, an SP1500 polypeptide may contain an amino acid sequence that is at least 60% or more (e.g., at least 65%, at least 70%, at least 75%, at least 80%, at least 85%, at least 90%, at least 95%, or at least 98%) identical to SEQ ID NO: 42 or a portion thereof (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, or more consecutive amino acids of the sequence shown in SEQ ID NO: 42). Alternatively, an SP1500 polypeptide may contain a portion (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, or more consecutive amino acids) of a sequence that is at least 60% or more (e.g., at least 65%, at least 70%, at least 75%, at least 80%, at least 85%, at least 90%, at least 95%, or at least 98%) identical to SEQ ID NO: 42. In some embodiments, a nucleotide sequence encoding an SP1500 polypeptide is provided herein as SEQ ID NO: 47. In some embodiments, an SP1500 polypeptide may comprises no more than 25 (including, e.g., no more than 20, no more than 15, no more than 10, no more than 9, no more than 8, no more than 7, no more than 6, no more than 5, no more than 4, no more than 3, no more than 2) amino acid modifications (e.g., deletion, substitution, and/or insertion) within the sequence of SEQ ID NO: 42 or a portion thereof as described herein. In some embodiments, such amino acid modifications may be present in the N-terminal portion and/or C-terminal portion.

In some embodiments, nucleic acid sequences encoding a pneumolysin polypeptide (SEQ ID NO: 34), an SP0435 polypeptide (SEQ ID NO: 38), SP0785 polypeptide (SEQ ID NO: 40), and an SP1500 polypeptide (SEQ ID NO: 42) are provided as SEQ ID NO: 44, SEQ ID NO: 45, SEQ ID NO: 46, and SEQ ID NO: 47, respectively. Due to degeneracy in the genetic code, those of ordinary skill in the art would understand that other DNA sequences (including codon-optimized sequences) could encode these polypeptides, as well as the others disclosed herein.

In some embodiments, the present disclosure includes nucleic acid sequences encoding any of the amino acids described herein. Due to degeneracy in the genetic code, those of ordinary skill in the art would understand that other DNA sequences (including codon-optimized sequences) could encode these polypeptides, as well as the others disclosed herein.

### Linkers or Spacers

In some embodiments, a rhizavidin polypeptide described herein (including, e.g., a variant rhizavidin polypeptide described herein) is coupled to one or more entities of interest (e.g., small molecules, polypeptides, polynucleotides, lipids, polysaccharides, etc.) with one or more linkers. For example, in some embodiments, a composition comprising a rhizavidin polypeptide comprises one or more linkers. In some embodiments, a fusion protein comprising a rhizavidin polypeptide and a polypeptide of interest (e.g., as described herein) comprises one or more linkers between the rhizavidin polypeptide and the polypeptide of interest. In some embodiments, a linker is or comprises one or more amino acids.

In some embodiments, a linker interposes a structure between two protein moieties. In some embodiments, the structure is or comprises an α-helix. In some embodiments, the structure is or comprises a β-strand. In some embodiments, the structure is or comprises a coil/bend. In some embodiments, the structure is or comprises a turn. In some embodiments, a linker decreases steric hindrance between two protein moieties joined by the linker. In some embodiments, a linker decreases unfavorable interactions between two protein moieties joined by the linker. In some embodiments, a linker comprises a mixture of glycine and serine residues. In some embodiments, the linker may additionally comprise threonine, proline, and/or alanine residues. In some embodiment, a linker is hydrophilic. In some embodiments, a linker is hydrophobic. In some embodiments, a linker increases the stability of a fusion protein containing the linker.

In some embodiments, a linker does not interfere with the folding or activity of a polypeptide of interest (e.g., in some embodiments, an antigenic polypeptide) to which it is joined. In some embodiments, a linker does not interfere with the antigenicity of an antigenic polypeptide to which it is joined. In some embodiments, a linker does not reduce the antigenicity of an antigenic polypeptide to which it is joined. In some embodiments, a linker does not eliminate the antigenicity of an antigenic polypeptide to which it is joined. In some embodiments, the effect of the linker is determined by comparing the polypeptide with the polypeptide joined to the linker.

In some embodiments, a linker does not interfere with the folding of a biotin-binding moiety (including, e.g., a rhizavidin polypeptide (including, e.g., a variant rhizavidin polypeptide)) to which it is joined. In some embodiments, a linker does not interfere with the biotin-binding ability of a biotin-binding moiety to which it is joined. In some embodiments, a linker does not reduce the biotin-binding ability of a biotin-binding moiety to which it is joined. In some embodiments, a linker does not eliminate the biotin-binding ability of a biotin-binding moiety to which it is joined. In some embodiments, the effect of the linker is determined by comparing the biotin-binding moiety with the biotin-binding moiety joined to the linker.

In some embodiments, a linker is not antigenic. In some embodiments, a linker does not elicit a T cell response. In some embodiments, a linker does not elicit a B cell response. In some embodiments, a linker does not induce a T cell or a B cell response.

In some embodiments, a linker comprises two or more amino acids. In some embodiments, a linker may be 3-100, 5-100, 10-100, 20-100 30-100, 40-100, 50-100, 60-100, 70-100, 80-100, 90-100, 5-55, 10-50, 10-45, 10-40, 10-35, 10-30, 10-25, 10-20, 10-15, 3-10, 3-9, 3-8, 3-7, 3-6, 3-5, 3-4, or 2-3 amino acids in length. In some embodiments, a linker comprises between 10-100, 10-90, 10-80, 10-70, 10-60, 10-50, 10-40, 10-30, 10-20, or 10-15 amino acids. In some embodiments, the linker comprises at least 10, 15, 20, 25, 30, 35, 40, 45, 50, 55, 60, 65, 70, 75, 80, 85, 90, or 95 amino acids. In some embodiments, a linker is or comprises a peptidyl linker comprising 2, 3, 4, 5, 6, 7, 8, 9, 10, 15, 20, 25, 30, 35, 40, 45, 50, 55, 60, 65, 70, 75, 80, 85, 90, 95, or 100 amino acids.

In some embodiments, a linker is a flexible linker. Flexible linkers may be useful for joining domains that require a certain degree of movement or interaction and may include small, non-polar (e.g., Gly) or polar (e.g., Ser or Thr) amino acids. Incorporation of Ser or Thr can also maintain the stability of the linker in aqueous solutions by forming hydrogen bonds with water molecules, and therefore reduce unfavorable interactions between the linker and the protein moieties. In some embodiments, a linker comprises small non-polar (e.g. Gly) or polar (e.g. Ser or Thr) amino acids. In some embodiments, a linker is a Gly-Ser linker.

In some embodiments, a linker is or comprises an amino acid sequence of GGGGSSS (SEQ ID NO: 48). In some embodiments, a linker is or comprises a sequence of (GGGGS)ₙ (SEQ ID NO: 49), where n represents the number of repeating GGGGS (SEQ ID NO: 50) units and is 1, 2, 3, 4, 5, 6, 7, 8, 9, 10, 11, 12, 13, 14, 15, 16, 17, 18, 19, 20, 25, 30 or more. In some embodiments, a polypeptide linker may have an amino acid sequence that is or comprises GGGGSGGGGSGGGGS (SEQ ID NO: 51) (i.e., (GGGGS)₃) or GGGGSGGGGSGGGGSGGGGSGGGGSGGGGS (SEQ ID NO: 52) (i.e., (GGGGS)₆). In some embodiments, a linker comprises one or more of Gly, Ser, Thr, Ala, Lys, and Glu. In some embodiments, a linker is or comprises KESGSVSSEQLAQFRSLD (SEQ ID NO: 53). In some embodiments, a linker is or comprises EGKSSGSGSESKST (SEQ ID NO: 54). In some embodiments, a linker is or comprises (Gly)ₙ (SEQ ID NO: 55) where n represents the number of repeating Gly residues and is 1, 2, 3, 4, 5, 6, 7, 8, 9, 10, 11, 12, 13, 14, 15, 16, 17, 18, 19, 20, 25, 30 or more. In some embodiments, a linker is or comprises GGG. In some embodiments, a linker is or comprises (Gly)₆ (SEQ ID NO: 56). In some embodiments, a linker is or comprises (Gly)₈ (SEQ ID NO: 57). In some embodiments, a linker is or comprises GSAGSAAGSGEF (SEQ ID NO: 58). In some embodiments, a linker is or comprises an amino acid sequence AAA. In some embodiments, such a linker may be synthesized, or derived from amino acid residues from a restriction site (e.g., a Not I restriction site).

In some embodiments, a linker is a rigid linker. Rigid linkers are useful to keep a fixed distance between domains and to maintain their independent functions. Rigid linkers may also be useful when a spatial separation of the domains is critical to preserve the stability or bioactivity of one or more components in the fusion. In some embodiments, a linker is or comprises (EAAAK)ₙ (SEQ ID NO: 59) where n represents the number of repeating EAAAK (SEQ ID NO: 60) units and is 1, 2, 3, 4, 5, 6, 7, 8, 9, 10, 11, 12, 13, 14, 15, 16, 17, 18, 19, 20, 25, 30 or more. In some embodiments, a linker is or comprises A(EAAAK)ₙA, (SEQ ID NO: 61) where n represents the number of repeating EAAAK (SEQ ID NO: 60) units and is 1, 2, 3, 4, 5, 6, 7, 8, 9, 10, 11, 12, 13, 14, 15, 16, 17, 18, 19, 20, 25, 30 or more. In some embodiments, a linker is or comprises A(EAAAK)ₙA (SEQ ID NO: 62), where n represents the number of repeating EAAAK (SEQ ID NO: 60) units and is 2, 3, 4, or 5. In some embodiments, a linker is or comprises A(EAAAK)₄ALEA(EAAAK)₄A (SEQ ID NO: 63). In some embodiments, a linker is or comprises [A(EAAAK)ₙA]ₘ, (SEQ ID NO: 64) wherein n is 2, 3, or 4 and m is 1 or 2. In some embodiments, a linker is or comprises AEAAAKEAAAKA (SEQ ID NO: 65).

In some embodiments, a linker is or comprises (X-Pro)ₙ (SEQ ID NO:66), with X designating any amino acid, where n represents the number of repeating X-Pro units and is 1, 2, 3, 4, 5, 6, 7, 8, 9, 10, 11, 12, 13, 14, 15, 16, 17, 18, 19, 20, 25, 30 or more. In some embodiments, a linker is or comprises (Ala-Pro)ₙ (SEQ ID NO: 67), where n represents the number of repeating Ala-Pro units and is 1, 2, 3, 4, 5, 6, 7, 8, 9, 10, 11, 12, 13, 14, 15, 16, 17, 18, 19, 20, 25, 30 or more. In some embodiments, a linker is or comprises (Ala-Pro)ₙ (SEQ ID NO: 68), where n represents the number of repeating Ala-Pro units and is 5, 6, 7, 8, 9, 10, 11, 12, 13, 14, 15, 16, or 17.

In some embodiments, a linker is or comprises (Lys-Pro)ₙ (SEQ ID NO: 69), where n represents the number of repeating Lys-Pro units and is 1, 2, 3, 4, 5, 6, 7, 8, 9, 10, 11, 12, 13, 14, 15, 16, 17, 18, 19, 20, 25, 30 or more. In some embodiments, a linker is or comprises (Glu-Pro)ₙ (SEQ ID NO: 70), where n represents the number of repeating Glu-Pro units and is 1, 2, 3, 4, 5, 6, 7, 8, 9, 10, 11, 12, 13, 14, 15, 16, 17, 18, 19, 20, 25, 30 or more. In some embodiments, a linker is or comprises (Ala-Pro)₇ (SEQ ID NO: 71).

In some embodiments, a linker is or comprises GAPGGGGGAAAAAGGGGGGAP (GAG linker, SEQ ID NO: 72). In some embodiments, a linker is or comprises GAPGGGGGAAAAAGGGGGGAPGGGGGAAAAAGGGGGGAP (GAG2 linker, SEQ ID NO: 73). In some embodiments, a linker is or comprises GAPGGGGGAAAAAGGGGGGAPGGGGGAAAAAGGGGGGAPGGGGGAAAAAGGGGG GAP (GAG3 linker, SEQ ID NO: 74).

In some embodiments, a linker is or comprises VSDP (SEQ ID NO: 75).

In some embodiments, a linker is or comprises AAAA (SEQ ID NO: 76).

In some embodiments, a linker is or comprises GGGG (SEQ ID NO: 77).

Suitable linkers or spacers also include those having an amino acid sequence at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more homologous or identical to the above exemplary linkers. In some embodiments, the linker comprises a polypeptide comprising an amino acid sequence having at least 80%, at least 85%, at least 90%, at least 95%, at least 96%, at least 97%, at least 98%, at least 99%, or 100% identity to the sequence of GGGGSSS (SEQ ID NO: 48).

Additional linkers suitable for use with some embodiments may be found in U.S. Patent Publication No. 2012/0232021, filed on Mar. 2, 2012, and [Chen, 2013] the disclosures of which is hereby incorporated by reference in their entireties.

### Tags

In some embodiments, a polypeptide described herein (e.g., in some embodiments, a composition comprising a rhizavidin polypeptide) may comprise one or more tags. In some embodiments, a fusion protein comprising a rhizavidin polypeptide (including, e.g., a variant rhizavidin polypeptide) comprises one or more tags. A tag may be present at the N-terminal or C-terminal of a polypeptide. For instance, tags may be added to a polypeptide (e.g., via additions or modifications on the encoding DNA sequence) to facilitate purification, detection, solubility, or confer other desirable characteristics on the polypeptide. In some embodiments, a tag may be a peptide, oligopeptide, or polypeptide that may be used in affinity purification. In some embodiments, a tag is, comprises, or is derived from one or more of poly-histidine (His), Glutathione S-transferase (GST), tandem affinity purification (TAP), FLAG, myc, human influenza hemagglutinin (HA), maltose binding protein (MBP), vesicular Stomatitis viral glycoprotein (VSV-G), thioredoxin, V5, avidin, streptavidin, biotin carboxyl carrier protein (BCCP), calmodulin, Nus, S tags, lipoprotein D, and galactosidase. In some embodiments, a His tag is or comprises an amino acid sequence of Hₙ, wherein n is an integer between 2 and 10 (SEQ ID NO: 78). Exemplary His tags include HHHHHH (SEQ ID NO: 79) and MSYYHHHHHH (SEQ ID NO: 80). In other embodiments, a polypeptide described herein is free of tags such as protein purification tags, and is purified by a method not relying on affinity for a purification tag. In some embodiments, a fusion protein described herein comprises (i) a polypeptide of amino acid sequence of any of SEQ ID NOs: 16-43 or fusion protein of amino acid sequence of any of SEQ ID NOs: 95-162, and (ii) a tag having no more than 1, 2, 3, 4, 5, 10, or 20 amino acids on one or both termini of the polypeptide or fusion protein of (i).

In some embodiments, a polypeptide described herein may contain a membrane translocating sequence (MTS), for example, in some embodiments, to facilitate introduction of the fusion protein into a mammalian cell and subsequent stimulation of the cell-mediated immune response. Exemplary membrane translocating sequences include, but are not limited to the hydrophobic region in the signal sequence of Kaposi fibroblast growth factor, the MTS of a synuclein, the third helix of the Antennapedia homeodomain, SN50, integrin 3 h-region, HIV Tat, pAntp, PR-39, abaecin, apidaecin, Bac5, Bac7, P. berghei CS protein, and those MTSs described in U.S. Pat. Nos. 6,248,558; 6,432,680; and 6,248,558.

### Signal Peptides

In some embodiments, a polypeptide described herein (e.g., in some embodiments, a composition comprising a rhizavidin polypeptide comprises a signal peptide. In some embodiments, a fusion protein comprising a rhizavidin polypeptide (including, e.g., a variant rhizavidin polypeptide) comprises a signal peptide. Signal peptides, which may also be called leader peptides, function in translocation of polypeptides through a cellular membrane (e.g., the plasma membrane, endoplasmic reticulum, etc.). In some embodiments, a polypeptide comprises a signal peptide at or near its N-terminus. In some embodiments, the signal sequence may or may not be cleaved off (e.g., by a signal peptidase) after translocation through a membrane.

In some embodiments, a polypeptide comprises a signal sequence and a rhizavidin polypeptide (including, e.g., a variant rhizavidin polypeptide). The signal sequence can be directly linked (e.g., by a peptide bond) or indirectly linked (e.g., by a linker) to the rhizavidin polypeptide. In some embodiments, the signal sequence can be linked to the rhizavidin polypeptide by a peptide linker disclosed herein.

In some embodiments, a signal peptide may be a bacterial signal peptide, a viral signal peptide, or eukaryotic signal peptide. In some embodiments, a eukaryotic signal peptide is a mammalian signal peptide (e.g., a signal peptide from, or derived from, a human protein).

In some embodiments, a signal peptide may be a bacterial signal peptide. Bacterial signal peptides may be used to promote translocation of a polypeptide into periplasmic space of bacteria. In some embodiments, the signal peptide is cleaved off from the polypeptide after translocation into the periplasmic space of a bacterium (e.g., E. coli). Signal peptides that direct secretion of proteins from bacterial cells are well known in the art, for example as disclosed in International application WO 2005/071088.

Any signal peptide known to one of ordinary skill in the art can be used. In some embodiments, a signal peptide is or comprises the amino acid sequence: MKKIWLALAGLVLAFSASA (SEQ ID NO: 81), MAPFEPLASGILLLLWLIAPSRA (SEQ ID NO: 82), MKKVAAFVALSLLMAGC (SEQ ID NO: 83), MKKIMLVITLILVSPIAQQTEAKD (SEQ ID NO: 84), MKKKIISAILMSTVILSAAAPLSGVYADT (SEQ ID NO: 85), MKKRKVLIPLMALSTILVSSTGNLEVIQAEV (SEQ ID NO: 86), MNMKKATIAATAGIAVTAFAAPTIASAST (SEQ ID NO: 87), MQKTRKERILEALQEEKKNKKSKKFKTGATIAGVTAIATSITVPGIEVIVSADE (SEQ ID NO: 88), MKKLKMASCALVAGLMFSGLTPNAFAED (SEQ ID NO: 89), MAKKFNYKLPSMVALTLVGSAVTAHQVQAAE (SEQ ID NO: 90), MTDKKSENQTEKTETKENKGMTRREMLKLSAVAGTGIAVGATGLGTILNVVDQVDKA LT (SEQ ID NO: 91), MAYDSRFDEWVQKLKEESFQNNTFDRRKFIQGAGKIAGLGLGLTIAQSVGAFG (SEQ ID NO: 92) or a derivative or functional portion thereof. In some embodiments, a signal peptide is or comprises the amino acid sequence of a human Ig heavy chain V-III region VH26 signal peptide or a derivative or functional portion thereof. In some embodiments, a signal peptide is or comprises the amino acid sequence MEFGLSWLFLVAILKGVQC (SEQ ID NO: 93) or a derivative or functional portion thereof. In some embodiments, a signal peptide is or comprises the amino acid sequence of human IgG2 heavy chain signal peptide or a derivative or functional portion thereof. In some embodiments, a signal peptide is or comprises the amino acid sequence MGWSCIILFLVATATGVHS (SEQ ID NO: 94) or a derivative or functional portion thereof.

Other examples of signal peptides can be found at Signal Peptide Website, a signal peptide database, which is found at www.signalpeptide.de.

In some embodiments, a polypeptide described herein lacks a signal peptide.

### Lipidated Rhizavidin Polypeptides

In some embodiments, a rhizavidin polypeptide provided herein (including, e.g., a variant rhizavidin polypeptide described herein) is a lipidated rhizavidin polypeptide, e.g., in some embodiments as further described in WO 2012/155053, the contents of which are herein incorporated by reference in their entirety for the purposes described herein. As used herein, the term “lipidated rhizavidin polypeptide” refers to a rhizavidin polypeptide that is covalently conjugated with a lipid. The lipid moieties could be a diacyl or triacyl lipid.

### Fusion Proteins

The present disclosure encompasses fusion proteins comprising a member of an affinity molecule pair and one or more polypeptides. In some embodiments, an affinity molecule pair is or comprises a biotin/biotin-binding molecule pair. In some such embodiments, a member of an affinity molecule pair is a rhizavidin polypeptide (including, e.g., a variant rhizavidin polypeptide described herein). In some embodiments, such fusion proteins comprises a rhizavidin polypeptide (including, e.g., a variant rhizavidin polypeptide described herein) and one or more polypeptides. In some embodiments, the one or more polypeptides are antigenic polypeptides.

In some embodiments, a fusion protein described herein comprises one antigenic polypeptide described herein. In some embodiments, a fusion protein comprises two antigenic polypeptides described herein. In some embodiments, a fusion protein comprises three antigenic polypeptides described herein. In some embodiments, a fusion protein comprises four antigenic polypeptides described herein. In some embodiments, a fusion protein comprises five antigenic polypeptides described herein. In some embodiments, a fusion protein comprises 6-10 antigenic polypeptides described herein. In some embodiments, a fusion protein comprises 11-15 antigenic polypeptides described herein. In some embodiments, a fusion protein comprises 15-20 antigenic polypeptides described herein.

In some embodiments, a fusion protein has carrier properties. In some embodiments, a fusion protein has antigenic properties. In some embodiments, a fusion protein has carrier properties and antigenic properties.

In some embodiments, a fusion protein comprises at least one antigenic polypeptide of, or derived from, a bacteria, a fungus, a parasite, a virus, or a cancer or tumor. In some embodiments, a fusion protein comprises an antigenic polypeptide that is or comprises a coronavirus polypeptide antigen (e.g., in some embodiments, a SARS-CoV-2 polypeptide antigen (e.g., in some embodiments, Spike(S) RBD)). In some embodiments, a fusion protein comprises an antigenic polypeptide that is or comprises a S. pneumoniae polypeptide antigen (e.g., in some embodiments, a pneumolysin polypeptide antigen, a SP0435 polypeptide antigen, a SP0785 polypeptide antigen, or a SP1500 polypeptide antigen).

In some embodiments, a fusion protein comprises at least one antigenic polypeptide having an amino acid sequence that is least 80%, at least 85%, at least 90%, at least 95%, at least 96%, at least 97%, at least 98%, at least 99% identical to any of SEQ ID NOs: 16-43, or antigenic fragments thereof. In some embodiments, a fusion protein comprises at least one antigenic polypeptide having the amino acid sequence of any of SEQ ID NOs: 16-43. In some embodiments, a fusion protein comprises one antigenic polypeptide having an amino acid sequence that is least 80%, at least 85%, at least 90%, at least 95%, at least 96%, at least 97%, at least 98%, at least 99% identical to any of SEQ ID NOs: 16-43, or an antigenic fragment thereof. In some embodiments, a fusion protein comprises one antigenic polypeptide having the amino acid sequence of any of SEQ ID NOs: 16-43, or an antigenic fragment thereof. In some embodiments, a fusion protein as described herein further comprises a rhizavidin polypeptide, for example, in some embodiments, a rhizavidin polypeptide having an amino acid sequence that is least 80%, at least 85%, at least 90%, at least 95%, at least 96%, at least 97%, at least 98%, at least 99% identical to any of SEQ ID NOs: 1-15, or biotin-binding fragments thereof. In some embodiments, a fusion protein as described herein further comprises a rhizavidin polypeptide, for example, in some embodiments, a rhizavidin polypeptide having the amino acid sequence of any of SEQ ID NOs: 1-15, or biotin-binding fragments thereof.

In some embodiments, a fusion protein comprises at least two antigenic polypeptides, at least one (including, e.g., at least two) of which has an amino acid sequence that is least 80%, at least 85%, at least 90%, at least 95%, at least 96%, at least 97%, at least 98%, at least 99% identical to any of SEQ ID NOs: 16-43, or antigenic fragments thereof. In some embodiments, a fusion protein comprises at least two antigenic polypeptides, at least one (including, e.g., at least two) of which has the amino acid sequence of any of SEQ ID NOs: 16-43, or antigenic fragments thereof. In some embodiments, a fusion protein as described herein further comprises a rhizavidin polypeptide, for example, in some embodiments, a rhizavidin polypeptide having an amino acid sequence that is least 80%, at least 85%, at least 90%, at least 95%, at least 96%, at least 97%, at least 98%, at least 99% identical to any of SEQ ID NOs: 1-15, or biotin-binding fragments thereof. In some embodiments, a fusion protein as described herein further comprises a rhizavidin polypeptide, for example, in some embodiments, a rhizavidin polypeptide having the amino acid sequence of any of SEQ ID NOs: 1-15, or biotin-binding fragments thereof.

In some embodiments, a fusion protein comprises an S-RBD polypeptide as described herein. For example, in some embodiments, a fusion protein comprises a polypeptide comprising an amino acid sequence having at least 80%, at least 85%, at least 90%, at least 95%, at least 96%, at least 97%, at least 98%, at least 99%, at least 99.5%, or 100% identity to the sequence of any one of SEQ ID NOs: 16-33, or an antigenic fragment thereof. In some embodiments, an S-RBD polypeptide described herein may be truncated from its N-terminal portion and/or C-terminal portion. In some embodiments, a fusion protein as described herein further comprises a rhizavidin polypeptide, for example, in some embodiments, a rhizavidin polypeptide having the amino acid sequence of any of SEQ ID NOs: 1-15, or biotin-binding fragments thereof.

In some embodiments, a fusion protein comprises a pneumolysin polypeptide as described herein. For example, in some embodiments, a fusion protein comprises a polypeptide comprising an amino acid sequence having at least 80%, at least 85%, at least 90%, at least 95%, at least 96%, at least 97%, at least 98%, at least 99%, at least 99.5%, or 100% identity to the sequence of any one of SEQ ID NOs: 34-37, or an antigenic fragment thereof. In some embodiments, a pneumolysin polypeptide described herein may be truncated from its N-terminal portion and/or C-terminal portion. In some embodiments, a fusion protein as described herein further comprises a rhizavidin polypeptide, for example, in some embodiments, a rhizavidin polypeptide having the amino acid sequence of any of SEQ ID NOs: 1-15, or biotin-binding fragments thereof.

In some embodiments, a fusion protein comprises an SP0435 polypeptide as described herein. For example, in some embodiments, a fusion protein comprises a polypeptide comprising an amino acid sequence having at least 80%, at least 85%, at least 90%, at least 95%, at least 96%, at least 97%, at least 98%, at least 99%, at least 99.5%, or 100% identity to the sequence of SEQ ID NO: 38 or SEQ ID NO: 39, or an antigenic fragment thereof. In some embodiments, an SP0435 polypeptide described herein may be truncated from its N-terminal portion and/or C-terminal portion. In some embodiments, a fusion protein as described herein further comprises a rhizavidin polypeptide, for example, in some embodiments, a rhizavidin polypeptide having the amino acid sequence of any of SEQ ID NOs: 1-15, or biotin-binding fragments thereof.

In some embodiments, a fusion protein comprises an SP0785 polypeptide as described herein. For example, in some embodiments, a fusion protein comprises a polypeptide comprising an amino acid sequence having at least 80%, at least 85%, at least 90%, at least 95%, at least 96%, at least 97%, at least 98%, at least 99%, at least 99.5%, or 100% identity to the sequence of SEQ ID NO: 40 or SEQ ID NO: 41, or an antigenic fragment thereof. In some embodiments, an SP0785 polypeptide described herein may be truncated from its N-terminal portion and/or C-terminal portion. In some embodiments, a fusion protein as described herein further comprises a rhizavidin polypeptide, for example, in some embodiments, a rhizavidin polypeptide having the amino acid sequence of any of SEQ ID NOs: 1-15, or biotin-binding fragments thereof.

In some embodiments, a fusion protein comprises an SP1500 polypeptide as described herein. For example, in some embodiments, a fusion protein comprises a polypeptide comprising an amino acid sequence having at least 80%, at least 85%, at least 90%, at least 95%, at least 96%, at least 97%, at least 98%, at least 99%, at least 99.5%, or 100% identity to the sequence of SEQ ID NO: 42 or SEQ ID NO: 43, or an antigenic fragment thereof. In some embodiments, an SP1500 polypeptide described herein may be truncated from its N-terminal portion and/or C-terminal portion. In some embodiments, a fusion protein as described herein further comprises a rhizavidin polypeptide, for example, in some embodiments, a rhizavidin polypeptide having the amino acid sequence of any of SEQ ID NOs: 1-15, or biotin-binding fragments thereof.

In some embodiments, a fusion protein comprises one or more polypeptides homologous to antigenic polypeptides described herein (e.g., an antigenic polypeptide isolated from different serotypes, strains, or species). Individual serotypes, strains, or species can contain numerous mutations relative to each other, and some of these can result in different protein sequences between the different serotypes, strains, or species. In some embodiments, an antigenic polypeptide has at least 80%, at least 85%, at least 90%, at least 95%, at least 96%, at least 97%, at least 98%, at least 99%, or at least 99.5% identity to a polypeptide of any of SEQ ID NOs: 16-43, or antigenic fragments thereof. Serotype, strain, or species variation may be used to design such variants of the antigenic polypeptides described herein.

In some embodiments, a fusion protein described herein comprises one or more fragments of one or more polypeptides described herein, e.g., a biotin-binding fragment of a rhizavidin polypeptide or antigenic fragments of an antigenic polypeptide. In some embodiments, a fusion protein described herein comprises a truncated version of a polypeptide described herein, which truncated version is close in size to a polypeptide having the amino acid sequences of any of SEQ ID NOs: 16-43. For example, in some embodiments, such a truncated polypeptide may lack at most one, two, three, four, five, ten, or twenty amino acids from one or both termini from its parent polypeptide. In some embodiments, the same number of residues is removed from the N-terminus and the C-terminus, while in other embodiments, a different number of residues is removed from the N-terminus compared to the C-terminus. In some embodiments, a truncated polypeptide has an amino acid sequence that is least 80%, at least 85%, at least 90%, at least 95%, at least 96%, at least 97%, at least 98%, at least 99% identical to any of SEQ ID NOs: 16-43 lacking 1-5, 1-10, or 1-20 amino acid residues from the N-terminus, C-terminus, or both. In some embodiments, a truncated polypeptide has the amino acid sequence of any of SEQ ID NOs: 16-43 lacking 1-5, 1-10, or 1-20 amino acid residues from the N-terminus, C-terminus, or both. In some embodiments, a truncated polypeptide has an amino acid sequence that is least 80%, at least 85%, at least 90%, at least 95%, at least 96%, at least 97%, at least 98%, at least 99% identical to any of SEQ ID NOs: 16-43 lacking 1-10 acid residues from the N-terminus, C-terminus, or both. In some embodiments, a truncated polypeptide has the amino acid sequence of any of SEQ ID NOs: 16-43 lacking 1-10 amino acid residues from the N-terminus, C-terminus, or both. For instance, a truncated polypeptide may lack 10 amino acid residues at both the N-terminus and C-terminus of any one of SEQ ID NOs: 16-43, resulting in a protein lacking 20 amino acid residues. Internal deletions, e.g., of 1-10, 11-20, 21-30, or 31-40 amino acids, are also contemplated.

In some embodiments, a fusion protein comprises an N-terminal polypeptide and a C-terminal polypeptide. In some embodiments, one or both of the N-terminal polypeptide and the C-terminal polypeptide is an antigenic polypeptide described herein, or an antigenic fragment or variant thereof. In some embodiments, one or both of the N-terminal polypeptide and the C-terminal polypeptide is a biotin-binding moiety (e.g., in some embodiments, a rhizavidin polypeptide disclosed herein). In some embodiments, one of the N-terminal polypeptide or the C-terminal polypeptide is a biotin-binding moiety (e.g., a rhizavidin polypeptide disclosed herein), and the other terminal polypeptide is an antigenic polypeptide described herein. In some embodiments, one of the N-terminal polypeptide or the C-terminal polypeptide is a rhizavidin polypeptide comprising an amino acid sequence that is at least 80%, at least 85%, at least 90%, at least 95%, at least 96%, at least 97%, at least 98%, or at least 99% identical to any one of SEQ ID NOs: 1-15), and the other terminal polypeptide is an antigenic polypeptide described herein.

In some embodiments, an N-terminal polypeptide and a C-terminal polypeptide present in a fusion protein are directly bound to each other. In some embodiments, an N-terminal polypeptide and a C-terminal polypeptide present in a fusion protein are linked via a linker peptide (e.g., a linker disclosed herein). The length and/or amino acids of a linker, when present, can be adjusted to obtain a more flexible, semi-rigid, or rigid linker. In some embodiments, a linker can be a GS-enriched linker. In some embodiments, a linker can be an A-enriched linker. A linker can generally be from 1-40, such as 3-10 or 10-30 and specifically 3, 4, 5, 6, 7, 8, 9, 10, 11, 12, 13, 14, 15, 16, 17, 18, 19 or 20 amino acids in length. Exemplary flexible peptide linkers are shown as SEQ ID NOs: 48-52 and 55-57.

In some embodiments, a fusion protein comprises one or more linkers described herein. In some embodiments, a fusion protein comprises at least one linker. In some embodiments, a fusion protein comprises at least two linkers. In some embodiments, a linker comprises a polypeptide comprising an amino acid sequence of GGGGSSS (SEQ ID NO: 48). In some embodiments, a linker comprises a polypeptide comprising an amino acid sequence of AAA. In some embodiments, a linker comprises a polypeptide comprising an amino acid sequence of AAAA (SEQ ID NO: 76). In some embodiments, a linker comprises a polypeptide comprising an amino acid sequence of GGGGSGGGGSGGGGS (SEQ ID NO: 51). In some embodiments, a fusion protein comprises a first linker having the amino acid sequence of GGGGSSS (SEQ ID NO: 48) and a second linker having the amino acid sequence of AAA. In some embodiments, a linker sequence may be a residual amino acid sequence from a restriction site. For example, in some embodiments, a fusion protein comprises an amino acid sequence AAA residual from a Not I restriction site. In some embodiments, a fusion protein comprises a linker of GGGGSSS (SEQ ID NO: 48) and an amino acid sequence AAA residual from a Not I restriction site. In some embodiments, the one or more linkers are selected from GGGGSSS (SEQ ID NO: 48) and AAAA (SEQ ID NO: 76). In some embodiments, a fusion protein comprises a linker of GGGGSSS (SEQ ID NO: 48) and AAAA (SEQ ID NO: 76). In some embodiments, a fusion protein comprises a linker described herein (e.g., a GS-enriched linker or an A-enriched linker) between a rhizavidin polypeptide comprising an amino acid sequence that is at least 80%, at least 85%, at least 90%, at least 95%, at least 96%, at least 97%, at least 98%, at least 99%, at least 99.5%, or 100% identical to any one of SEQ ID NOs: 1-15), and a polypeptide of interest (e.g., in some embodiments, an antigenic polypeptide described herein).

In some embodiments, a fusion protein described herein comprises a biotin-binding moiety (e.g., a rhizavidin polypeptide described herein). In some embodiments, a fusion protein comprises a biotin-binding moiety (e.g., a rhizavidin polypeptide described herein), and one or more polypeptide antigens. In some embodiments, a fusion protein comprises a biotin-binding moiety (e.g., a rhizavidin polypeptide) and two or more polypeptide antigens. In some embodiments, the biotin-binding moiety of a fusion protein comprises a rhizavidin polypeptide or a biotin-binding fragment thereof, which is in some embodiments as described in WO 2012/155053, the contents of which are herein incorporated by reference in their entirety for the purposes described herein. In some embodiments, the biotin-binding moiety of a fusion protein is or comprises a rhizavidin variant described herein or a biotin-binding fragment thereof.

In some embodiments, a fusion protein comprises a biotin-binding moiety (e.g., a rhizavidin polypeptide) that is or comprises a polypeptide having an amino acid sequence that is at least 80%, at least 85%, at least 90%, at least 95%, at least 96%, at least 97%, at least 98%, at least 99%, at least 99.5%, or 100% identity to the sequence of SEQ ID NO: 1, or biotin-binding fragment thereof. In some embodiments, a fusion protein comprises a biotin-binding moiety (e.g., a rhizavidin polypeptide) that is or comprises a polypeptide having an amino acid sequence at least 80%, 85%, 90%, 95%, 96%, 97%, 98%, 99%, or 100% identity to the sequence of SEQ ID NO: 2 or SEQ ID NO: 3, or biotin-binding fragment thereof.

In some embodiments, a fusion protein comprises a biotin-binding moiety (e.g., a rhizavidin polypeptide) that is or comprises a polypeptide having an amino acid sequence at least 80%, at least 85%, at least 90%, at least 95%, at least 96%, at least 97%, at least 98%, at least 99%, at least 99.5%, or 100% identity to the sequence of SEQ ID NO: 4, or biotin-binding fragment thereof. In some embodiments, a fusion protein comprises a biotin-binding moiety (e.g., a rhizavidin polypeptide) that is or comprises a polypeptide having an amino acid sequence at least 80%, 85%, 90%, 95%, 96%, 97%, 98%, 99%, or 100% identity to the sequence of SEQ ID NO: 5 or SEQ ID NO: 6, or biotin-binding fragment thereof.

In some embodiments, a fusion protein comprises a biotin-binding moiety (e.g., a rhizavidin polypeptide) that is or comprises a polypeptide having an amino acid sequence at least 80%, at least 85%, at least 90%, at least 95%, at least 96%, at least 97%, at least 98%, at least 99%, at least 99.5%, or 100% identity to the sequence of SEQ ID NO: 7, or biotin-binding fragment thereof. In some embodiments, a fusion protein comprises a biotin-binding moiety (e.g., a rhizavidin polypeptide) that is or comprises a polypeptide having an amino acid sequence at least 80%, 85%, 90%, 95%, 96%, 97%, 98%, 99%, or 100% identity to the sequence of SEQ ID NO: 8 or SEQ ID NO: 9, or biotin-binding fragment thereof.

In some embodiments, a fusion protein comprises a biotin-binding moiety (e.g., a rhizavidin polypeptide) that is or comprises a polypeptide having an amino acid sequence at least 80%, at least 85%, at least 90%, at least 95%, at least 96%, at least 97%, at least 98%, at least 99%, at least 99.5%, or 100% identity to the sequence of SEQ ID NO: 10, or biotin-binding fragment thereof. In some embodiments, a fusion protein comprises a biotin-binding moiety (e.g., a rhizavidin polypeptide) that is or comprises a polypeptide having an amino acid sequence at least 80%, 85%, 90%, 95%, 96%, 97%, 98%, 99%, or 100% identity to the sequence of SEQ ID NO: 11 or SEQ ID NO: 12, or biotin-binding fragment thereof.

In some embodiments, a fusion protein comprises a biotin-binding moiety (e.g., a rhizavidin polypeptide) that is or comprises a polypeptide having an amino acid sequence at least 80%, at least 85%, at least 90%, at least 95%, at least 96%, at least 97%, at least 98%, at least 99%, at least 99.5%, or 100% identity to the sequence of SEQ ID NO: 13, or biotin-binding fragment thereof. In some embodiments, a fusion protein comprises a biotin-binding moiety (e.g., a rhizavidin polypeptide) that is or comprises a polypeptide having an amino acid sequence at least 80%, 85%, 90%, 95%, 96%, 97%, 98%, 99%, or 100% identity to the sequence of SEQ ID NO: 14 or SEQ ID NO: 15, or biotin-binding fragment thereof.

In some embodiments, a fusion protein described herein comprises each of: (a) a biotin-binding moiety (e.g., a rhizavidin polypeptide) that is or comprises (i) a polypeptide having at least 80%, at least 85%, at least 90%, at least 95%, at least 96%, at least 97%, at least 98%, at least 99%, at least 99.5%, or 100% identity to the sequence of any one of SEQ ID NOs: 1-3, or biotin-binding fragment thereof, or (ii) a polypeptide having at least 80%, at least 85%, at least 90%, at least 95%, at least 96%, at least 97%, at least 98%, at least 99%, at least 99.5%, or 100% identity to the sequence of any one of SEQ ID NOs: 4-15, wherein the polypeptide retains one or more, or all, of the mutations present in the sequence of the one of SEQ ID NOs: 4-15 relative to a wild-type rhizavidin polypeptide (e.g., as set forth in any one of SEQ ID NOs: 1-3), or biotin-binding fragment thereof; and (b) a polypeptide antigen from a coronavirus. In some embodiments, such a polypeptide antigen from a coronavirus is or comprises a polypeptide antigen from SARS-CoV-2. In some embodiments, such a polypeptide antigen from a coronavirus is or comprises an amino acid sequence having at least 80%, at least 85%, at least 90%, at least 95%, at least 96%, at least 97%, at least 98%, at least 99%, at least 99.5%, or 100% identity to the sequence of any one of SEQ ID NOs: 16-33, or an antigenic fragment thereof. In some embodiments, the fusion protein further comprises one or more linkers. In some embodiments, such one or more linkers are independently selected from the group consisting of: AAA and the amino acid sequence of any one of SEQ ID NOs: 48-77. In some embodiments, such a linker is or comprises the amino acid sequence of GGGGSSS (SEQ ID NO: 48).

In some embodiments, a fusion protein described herein comprises each of: (a) a biotin-binding moiety (e.g., a rhizavidin polypeptide) that is or comprises (i) a polypeptide having at least 80%, at least 85%, at least 90%, at least 95%, at least 96%, at least 97%, at least 98%, at least 99%, at least 99.5%, or 100% identity to the sequence of any one of SEQ ID NOs: 1-3, or biotin-binding fragment thereof, or (ii) a polypeptide having at least 80%, at least 85%, at least 90%, at least 95%, at least 96%, at least 97%, at least 98%, at least 99%, at least 99.5%, or 100% identity to the sequence of any one of SEQ ID NOs: 4-15, wherein the polypeptide retains one or more, or all, of the mutations present in the sequence of the one of SEQ ID NOs: 4-15 relative to a wild-type rhizavidin polypeptide (e.g., as set forth in any one of SEQ ID NOs: 1-3), or biotin-binding fragment thereof; (b) a polypeptide antigen from a bacteria. In some embodiments, such a polypeptide antigen from a bacteria is or comprises a polypeptide antigen from S. pneumoniae. In some embodiments, such a polypeptide antigen from a bacteria is or comprises an amino acid sequence having at least 80%, at least 85%, at least 90%, at least 95%, at least 96%, at least 97%, at least 98%, at least 99%, at least 99.5%, or 100% identity to the sequence of any one of SEQ ID NOs: 34-37, or an antigenic fragment thereof, and (c) a polypeptide antigen from a bacteria. In some embodiments, such a polypeptide antigen from a bacteria is or comprises a polypeptide antigen from S. pneumoniae. In some embodiments, such a polypeptide antigen from a bacteria is or comprises an amino acid sequence having at least 80%, at least 85%, at least 90%, at least 95%, at least 96%, at least 97%, at least 98%, at least 99%, at least 99.5%, or 100% identity to the sequence of SEQ ID NO: 38 or SEQ ID NO: 39, or an antigenic fragment thereof. In some embodiments, the fusion protein further comprises one or more linkers. In some embodiments, such one or more linkers are independently selected from the group consisting of: AAA and the amino acid sequence of any one of SEQ ID NOs: 48-77. In some embodiments, such one or more linkers are GGGGSSS (SEQ ID NO: 48) and AAA.

In some embodiments, a fusion protein described herein comprises each of: (a) a biotin-binding moiety (e.g., a rhizavidin polypeptide) that is or comprises (i) a polypeptide having at least 80%, at least 85%, at least 90%, at least 95%, at least 96%, at least 97%, at least 98%, at least 99%, at least 99.5%, or 100% identity to the sequence of any one of SEQ ID NOs: 1-3, or biotin-binding fragment thereof, or (ii) a polypeptide having at least 80%, at least 85%, at least 90%, at least 95%, at least 96%, at least 97%, at least 98%, at least 99%, at least 99.5%, or 100% identity to the sequence of any one of SEQ ID NOs: 4-15, wherein the polypeptide retains one or more, or all, of the mutations present in the sequence of the one of SEQ ID NOs: 4-15 relative to a wild-type rhizavidin polypeptide (e.g., as set forth in any one of SEQ ID NOs: 1-3), or biotin-binding fragment thereof; (b) a polypeptide antigen from a bacteria. In some embodiments, such a polypeptide antigen from a bacteria is or comprises a polypeptide antigen from S. pneumoniae. In some embodiments, such a polypeptide antigen from a bacteria is or comprises an amino acid sequence having at least 80%, at least 85%, at least 90%, at least 95%, at least 96%, at least 97%, at least 98%, at least 99%, at least 99.5%, or 100% identity to the sequence of SEQ ID NO: 42 or SEQ ID NO: 43, or an antigenic fragment thereof, and (c) a polypeptide antigen from a bacteria. In some embodiments, such a polypeptide antigen from a bacteria is or comprises a polypeptide antigen from S. pneumoniae. In some embodiments, such a polypeptide antigen from a bacteria is or comprises an amino acid sequence having at least 80%, at least 85%, at least 90%, at least 95%, at least 96%, at least 97%, at least 98%, at least 99%, at least 99.5%, or 100% identity to the sequence of SEQ ID NO: 40 or SEQ ID NO: 41, or an antigenic fragment thereof. In some embodiments, the fusion protein further comprises one or more linkers. In some embodiments, such one or more linkers are independently selected from the group consisting of: AAA and the amino acid sequence of any one of SEQ ID NOs: 48-77. In some embodiments, such one or more linkers are GGGGSSS (SEQ ID NO: 48) and AAA.

In some embodiments, the present disclosure provides fusion proteins with at least 80%, at least 85%, at least 90%, at least 95%, at least 96%, at least 97%, at least 98%, at least 99%, at least 99.5%, or 100% sequence identity to a fusion protein having an amino acid sequence of any one of SEQ ID NOs: 95-162.

In some embodiments, a fusion protein described herein includes a variant or fragment of a polypeptide having an amino acid sequence of SEQ ID NOs: 1-15 and 16-43. In some embodiments, a fusion protein described herein includes a polypeptide encoded by a variant or fragment of a gene having a nucleic acid sequence of SEQ ID NOs: 44-47. In some embodiments, a fragment included in a fusion protein described herein is close in size to a full-length polypeptide or a polypeptide having an amino acid sequence of SEQ ID NOs: 1-15 and 16-43. For example, they may lack at most one, two, three, four, five, ten, twenty, or thirty amino acids from one or both termini. In some embodiments, the fragment is 25-50 amino acids in length, or 50-100, or 100-150, or 150-200, or 200-250, or 250-300, or 300-350 amino acids in length. In some embodiments, the fragments result from processing, or partial processing, of signal sequences by an expression host, e.g. E. coli, an insect cell line (e.g., the baculovirus expression system), a yeast (e.g., S. cerevisiae or S. pombe) cell line, or a mammalian (e.g., human or Chinese Hamster Ovary) cell line. The fragments described above or sub-fragments thereof (e.g., fragments of 8-50, 8-30, or 8-20 amino acid residues) preferably have one of the biological activities described below, such as increasing the amount of IL-17 released by at least 30%, at least 40%, at least 50%, at least 60%, at least 70%, at least 80%, at least 90%, at least 100%, at least 150%, at least 200%, at or more (e.g., either as an absolute measure or relative to a control protein).

In some embodiments, fusion proteins described herein can be useful in immunogenic complexes described herein.

### Immunogenic Complexes

The present disclosure encompasses immunogenic complexes that include one or more polypeptides and one or more polymers.

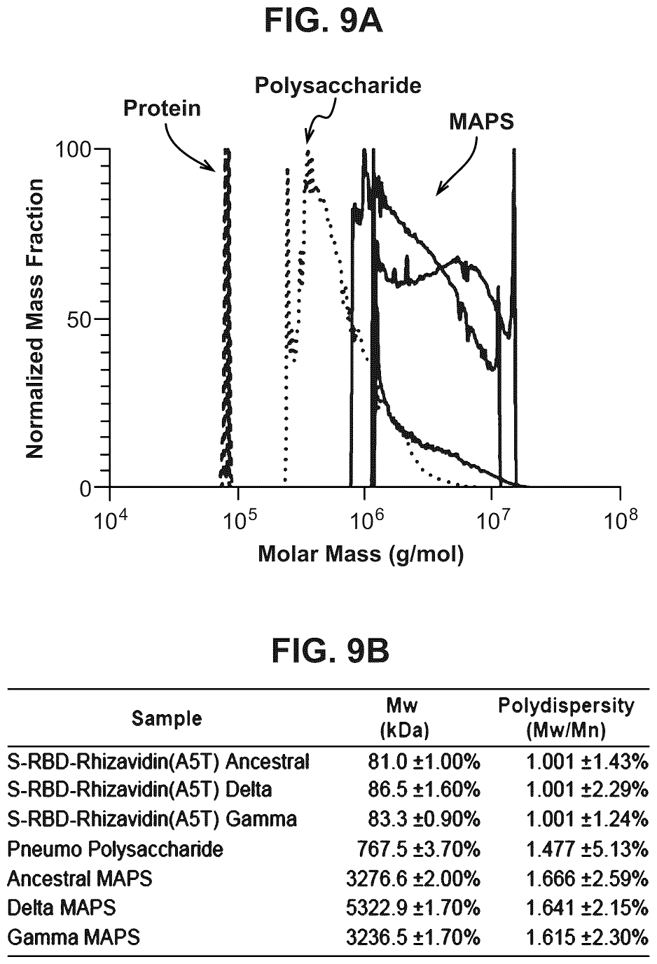

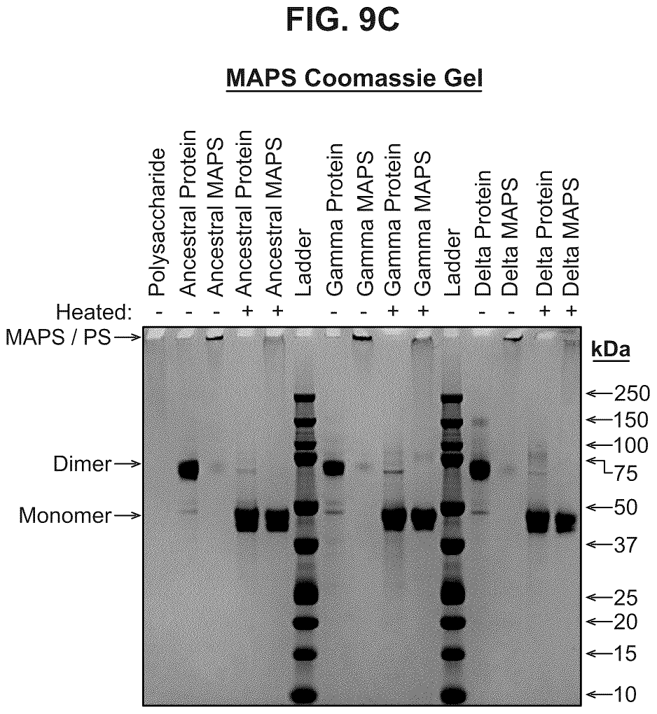

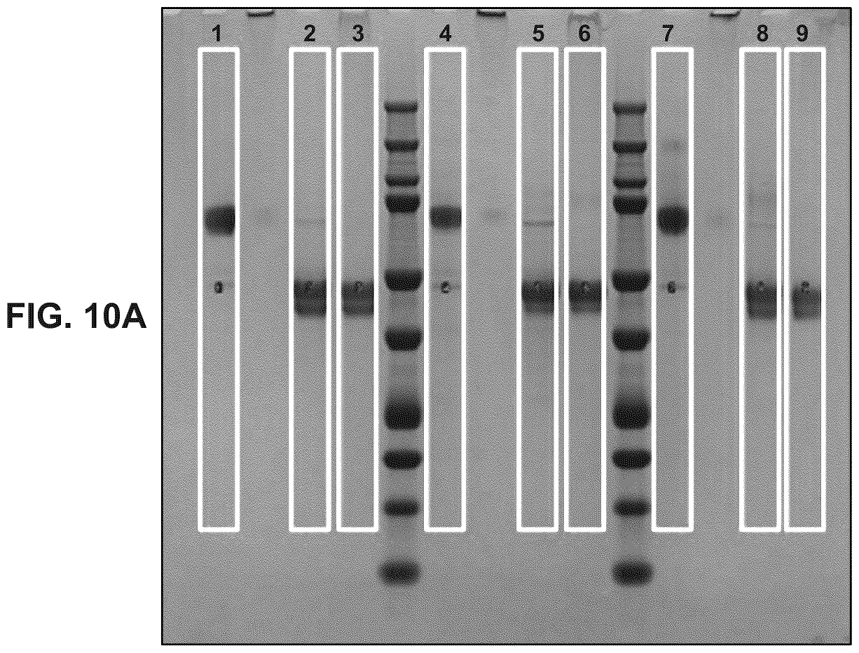

In some embodiments, immunogenic complexes are, or are based on, Multiple Antigen Presenting System (MAPS) complexes. Certain aspects of the MAPS platform have been previously described in WO2012/155007 and WO2020/056202, the contents of which are herein incorporated by reference in their entirety, and are shown schematically in FIG. 9. See also, Zhang et al., 2013.

In some embodiments, an immunogenic complex includes one or more polypeptides (e.g., but not limited to, antigenic polypeptides) described herein non-covalently complexed with one or more polymers (e.g., but not limited to, polysaccharides) described herein. In some embodiments, one or more polypeptides (e.g., but not limited to, antigenic polypeptides) are complexed via affinity interaction with one or more polymers (e.g., but not limited to, polysaccharides). In some embodiments, immunogenic complexes of the disclosure include one or more polypeptides (e.g., but not limited to, antigenic polypeptides) non-covalently complexed with one or more polymers (e.g., but not limited to antigenic polysaccharides) using one or more affinity molecule pairs each comprising a first affinity molecule and a second affinity molecule complementary to the first affinity molecule (“complementary affinity molecule”). Upon association of the first affinity molecule and the complementary affinity molecule, the one or more polypeptides (e.g., antigenic polypeptides) are non-covalently complexed to the one or more polymers (e.g., antigenic polysaccharides).

In some embodiments, an immunogenic complex includes (i) one or more polymers (e.g., antigenic polysaccharides) associated with (e.g., in some embodiments, by chemical conjugation) a first affinity molecule described herein, and (ii) a fusion protein that comprises a complementary affinity molecule described herein and one or more polypeptides (e.g., antigenic polypeptides). In some embodiments, an immunogenic complex includes (i) one or more polymers (e.g., antigenic polysaccharides) associated with (e.g., in some embodiments, by chemical conjugation) a plurality of a first affinity molecule described herein, and (ii) a fusion protein that comprises a complementary affinity molecule described herein and one or more polypeptides (e.g., antigenic polypeptides).

In some embodiments, one or more polypeptides (e.g., antigenic polypeptides) are complexed via affinity interaction with one polymer (e.g., an antigenic polysaccharide). In some embodiments, immunogenic complexes of the disclosure include one or more polypeptides (e.g., antigenic polypeptides) non-covalently complexed with one polymer (e.g., an antigenic polysaccharide) using one affinity molecule/complementary affinity molecule pair. In some embodiments, immunogenic complexes of the disclosure include one or more polypeptides (e.g., antigenic polypeptides) non-covalently complexed with one polymer (e.g., an antigenic polysaccharide) using one or more affinity molecule/complementary affinity molecule pairs. In some embodiments, each of the affinity molecule/complementary affinity molecule pairs is the same, e.g., biotin/biotin-binding moiety pairs. In some embodiments, an immunogenic complex includes (i) one polymer (e.g., an antigenic polysaccharide) associated with (e.g., in some embodiments, by chemical conjugation) a first affinity molecule described herein, and (ii) a fusion protein that comprises a complementary affinity molecule described herein and one or more polypeptides (e.g., antigenic polypeptides). In some embodiments, an immunogenic complex includes (i) one polymer (e.g., an antigenic polysaccharide) associated with (e.g., in some embodiments, by chemical conjugation) a plurality of first affinity molecule described herein, and (ii) a fusion protein that comprises a complementary affinity molecule described herein and one or more polypeptides (e.g., antigenic polypeptides). Upon association of the first affinity molecule and the complementary affinity molecule, the one or more polypeptides (e.g., antigenic polypeptides) are non-covalently complexed to the one polymer (e.g., an antigenic polysaccharide).

In some embodiments, an immunogenic complex of the present disclosure is an immunogenic complex as described in WO 2018/237221, WO 2020/056202, PCT/US2022/043156, or PCT/US2022/042964, wherein at least one of the biotin-binding moieties is a variant rhizavidin polypeptide disclosed herein. The contents of each of the aforementioned references are incorporated by reference in their entirety for their purposes described herein.

### Affinity Molecules Pairs

As described herein, immunogenic complexes of the present disclosure include complementary affinity molecule pairs comprising an affinity molecule (i.e., a first affinity molecule) and a complementary affinity molecule (i.e., a second affinity molecule that is complementary to the first affinity molecule). In some embodiments, the affinity molecule/complementary affinity molecule pair is selected from one or more of biotin/biotin-binding moiety, antibody/antigen, enzyme/substrate, receptor/ligand, metal/metal-binding protein, carbohydrate/carbohydrate binding protein, lipid/lipid-binding protein, and His tag/His tag-binding molecule. In some embodiments, a first affinity molecule is associated with a polymer (e.g., but not limited to a polysaccharide). In some embodiments a second affinity molecule is associated with a polypeptide of interest (e.g., but not limited to a polypeptide antigen).

In some embodiments, the first affinity molecule is biotin (or a derivative or fragment thereof), and the complementary affinity molecule is a biotin-binding moiety, e.g., a biotin-binding protein or polypeptide, or a biotin-binding domain or biotin-binding fragment thereof. In some embodiments, the biotin-binding moiety is a biotin-binding moiety that can form a dimer. In some embodiments, the biotin-binding moiety is a biotin-binding moiety that can form a tetramer. In some embodiments, the biotin-binding moiety is or comprises rhizavidin, avidin, streptavidin, bradavidin, tamavidin, lentiavidin, zebavidin, NeutrAvidin, CaptAvidin™, or a biotin-binding domain or biotin-binding fragment thereof, or a combination thereof. In some embodiments, the biotin-binding moiety is a rhizavidin polypeptide disclosed herein, or a biotin-binding domain or biotin-binding fragment thereof. In some embodiments, the biotin-binding moiety is or comprises a polypeptide of any of SEQ ID NOs: 1-15, or a biotin-binding domain or biotin-binding fragment thereof. In some embodiments, a biotin-binding moiety is or comprises a variant rhizavidin as described herein.

In some embodiments, a fusion protein of an immunogenic complex comprises a biotin-binding moiety (e.g., a rhizavidin polypeptide), and one or more polypeptides (e.g., polypeptide antigens). In some embodiments, a fusion protein of an immunogenic complex comprises a biotin-binding moiety (e.g., a rhizavidin polypeptide) and two or more polypeptides (e.g., polypeptide antigens).

### Polymers

In some embodiments, an immunogenic complex described herein (e.g., a MAPS complex) comprises a polymer. In some embodiments, a component of MAPS is or comprises a polymer. The polymer may be antigenic or non-antigenic. The polymer can be made of a wide variety on substances. In some embodiments, the polymer is a synthetic polymer. In some embodiments, the polymer is a naturally occurring polymer. In some embodiments, the polymer is a polysaccharide. In some embodiments, the polysaccharide is an antigenic polysaccharide (e.g., an antigenic polysaccharide described herein). In some embodiments, the polysaccharide is or is derived from bacterial cells, eukaryotic cells (e.g., fungi, insect, or plant cells). In some embodiments, the polymer is or is derived from mammalian cells (e.g., virus-infected cells or cancer cells). In general, such polymers are well known in the art and are encompassed for use in the methods and compositions as disclosed herein.

In some embodiments, a polymer is a chimeric polymer comprising more than one type of polymer. For example, a polymer of an immunogenic complex as disclosed herein can comprise a first portion that is or is derived from a first polymer, and a second portion that is or is derived from a second polymer. There is no limit to the amount of different types of polymers that can be used in a single immunogenic complex. In some embodiments, a polymer is a single chain polymer. In some embodiments, a polymer is a branched polymer comprising a chain polymer and one or more branch polymers. The chain polymer can be or be derived from a first polymer. The branch polymers can be or be derived from at least 1, at least 2, at least 3 or more different polymers.

In some embodiments, a polymer is a polysaccharide. In some embodiments, the polysaccharide comprises at least 10, at least 20, at least 30, at least 40, at least 50, at least 75, at least 100, at least 150, at least 200, at least 250, at least 300, at least 350, at least 400, at least 450, or at least 500 carbohydrate repeating units. In some embodiments, the polysaccharide has a molecular mass of less than 500 kDa or greater than 500 kDa. In some embodiments, the polysaccharide has a molecular mass of less than 70 kDa.

In some embodiments, a polymer is a large molecular weight polymer, e.g., a polymer can be of an average molecular weight of between about 425 to about 500 kDa, for example, at least 300 kDa, at least 350 kDa, at least 400 kDa, at least 425 kDa, at least 450 kDa, at least 500 kDa, or greater than 500 kDa, but typically less than 500 kDa.

In some embodiments, a polymer is a small molecular weight polymer, e.g., a polymer can be of an average molecular weight of between about 60 kDa to about 90 kDa, for example, at least 50 kDa, at least 60 kDa, at least 70 kDa, at least 80 kDa, at least 90 kDa, at least 100 kDa, or greater than 100 kDa, but generally less than about 120 kDa.

In some embodiments, a polymer (e.g., a polysaccharide) is harvested and/or purified from a natural source; and in other embodiments, the polymer is synthetic. Methods to produce synthetic polymers, including synthetic polysaccharides, are known to persons of ordinary skill and are encompassed in the compositions and methods as disclosed herein.

In some embodiments, a polymer that can be used in an immunogenic complex (e.g., a MAPS complex) described herein includes: dextran, polyethylene glycol-based polymers, poly(ortho ester) polymers, polyacryl carriers, PLGA, polyethylenimine (PEI), polyamidoamine (PAMAM) dendrimers, β-amino ester polymers, polyphophoester (PPE), liposomes, polymerosomes, nucleic acids, polyphosphorothioated oligonucleotides, chitosan, silk, polymeric micelles, protein polymers, virus particles, virus-like particles (VLPs), or other microparticles. See, e.g., El-Sayed et al., Smart Polymer Carriers for Enhanced Intracellular Delivery of Therapeutic Molecules, 5 Exp. Op. Biol. Therapy, 23 (2005). Biocompatible polymers developed for nucleic acid delivery may be adapted for use as a polymer herein. See, e.g., Biocompatible Pol. Nucl. Acid. Deliv. (Domb et al., eds., John Wiley & Sons, Inc. Hoboken, NJ, 2011).

For example, VLPs resemble viruses, but are non-infectious because they do not contain any viral genetic materials. The expression, including recombinant expression, of viral structural proteins, such as envelope or capsid components, can result in the self-assembly of VLPs. VLPs have been produced from components of a wide variety of virus families including Parvoviridae (e.g., adeno-associated virus), Retroviridae (e.g., HIV), and Flaviviridae (e.g., Hepatitis B or C viruses). VLPs can be produced in a variety of cell culture systems, including mammalian cell lines, insect cell lines, yeast, and plant cells. Recombinant VLPs are particular advantageous because the viral component can be fused to recombinant antigens as described herein.

### Antigenic Polysaccharides

In some embodiments, a polymer used in an immunogenic complex is or comprises an antigenic polysaccharide. In some embodiments, an antigenic polysaccharide is derived from an organism selected from the group consisting of: bacteria, archaea, viruses, or eukaryotic cells like fungi, insect, plant, or chimeras thereof. In some embodiments, an antigenic polysaccharide is a polysaccharide that is found present in or on surfaces of mammalian cells, e.g., in some embodiments cancerous cells. In some embodiments, an antigenic polysaccharide is derived from a pathogenic bacterium or virus. In some embodiments, an antigenic polysaccharide is or is derived from a glycoprotein. In specific embodiments, the antigenic polysaccharide is a pneumococcal capsular polysaccharide, a pneumococcal cell-wall polysaccharide, a meningococcal polysaccharide, a Haemophilus influenze type b polysaccharide, a Streptococcus agalactiae polysaccharide, a Salmonella typhi Vi polysaccharide, a Klebsiella polysaccharide, a Pseudomonas polysaccharide, a Escherichia coli polysaccharide, or a Staphylococcus aureus polysaccharide. In some embodiments, an antigenic polysaccharide is derived from a tumor.

In some embodiments, an antigenic polysaccharide is, or is derived from Gram-negative bacteria and/or Gram-positive bacteria. In some embodiments, an antigenic polysaccharide is, or is derived from one or more glycoproteins. In some embodiments, one or more such glycoproteins are, or are derived from one or more viruses. In some embodiments, an antigenic polysaccharide is, or is derived from S. pneumoniae. In some embodiments, antigenic polysaccharides included in an immunogenic composition described herein are, or are derived from one or more pathogens. In some embodiments, one or more antigenic polysaccharides are, or are derived from 1, 2, 3, 4, 5, 6, 7, 8, 9, 10, 11, 12, 13, 14, 15, 16, 17, 18, 19, 20, 21, 22, 23, 24, or 25 serotypes or strains (variants) of a pathogen. In some embodiments, one or more antigenic polysaccharides are, or are derived from more than 25 serotypes or strains (variants) of a pathogen, e.g., 26, 27, 28, 29, 30, 35, 40, 45, or 50 serotypes or strains. In some embodiments, one or more antigenic polysaccharides are, or are derived from more than 60, 70, 80, 90, or 100 serotypes or strains (variants) of a pathogen.

In some embodiments, an antigenic polysaccharide is a branched polysaccharide, or alternatively, can be a straight chain polysaccharide.

In some embodiments, an antigenic polysaccharide is a Vi antigen (Salmonella typhi capsular polysaccharide), pneumococcal capsular polysaccharides, pneumococcal cell wall polysaccharide, Hib (Haemophilus influenza type B) capsular polysaccharide, meningococcal capsular polysaccharides, the polysaccharide of Bacillus anthracis (the causative agent of anthrax), and other bacterial capsular or cell wall polysaccharides, or any combinations thereof.

In some embodiments, an antigenic polysaccharide consists of or comprises a sugar moiety. For example, in some embodiments, a polysaccharide is a Vi polysaccharide of Salmonella typhi. The Vi capsular polysaccharide has been developed against bacterial enteric infections, such as typhoid fever. Robbins et al., 150 J. Infect. Dis. 436 (1984); Levine et al., 7 Baillieres Clin. Gastroenterol. 501 (1993). Vi is a polymer of α-1→4-galacturonic acid with an N acetyl at position C-2 and variable O-acetylation at C-3. The virulence of S. typhi correlates with the expression of this molecule. Sharma et al., 101 PNAS 17492 (2004). The Vi polysaccharide vaccine of Salmonella typhi has several advantages: side effects are infrequent and mild, a single dose yields consistent immunogenicity and efficacy. Vi polysaccharide may be reliably standardized by physicochemical methods verified for other polysaccharide vaccines, Vi is stable at room temperature and it may be administered simultaneously with other vaccines without affecting immunogenicity and tolerability. Azze et al., 21 Vaccine 2758 (2003).

In some embodiments, an antigenic polysaccharide can be derived from Neisseria meningitidis, e.g., capsular polysaccharides from at least one, two, three or four of the serogroups A, C, W, W135, or Y. In some embodiments, the polysaccharide comprises Type 5, Type 8, or any of the polysaccharides or oligosaccharides of Staphylococcus aureus.

In some embodiments, an antigenic polysaccharide can be derived from Klebsiella pneumoniae, e.g., lipopolysaccharide (LPS)-derived polysaccharides or capsular polysaccharides. In some embodiments, LPS-derived polysaccharides are O polysaccharides (OPS). In some embodiments, LPS-derived polysaccharides are core O polysaccharides (COPS). In some embodiments, the polysaccharide is from, or derived from, an OPS from Klebsiella pneumoniae serotypes O1, O2, O2ac, O3, O4, O5, O7, O8, or O12. In some embodiments, the polysaccharide is from, or derived from, a CPS from Klebsiella pneumoniae K1, K2, K10, K16, or K19.

In some embodiments, an antigenic polysaccharide is from, or derived from, Pseudomonas aeruginosa, e.g., OPS, LPS, or exopolysaccharides. In some embodiments, an antigenic polysaccharide is from, or derived from, an OPS from a Pseudomonas aeruginosa serotype selected from O1, O2, O3, O4, O5, O6, O7, O8, O9, O10, O11, O12, O13, O14, O15, O16, O17, O18, O19, and O20. In some embodiments, an antigenic polysaccharide is, or is derived from, a capsular or capsular-like polysaccharide from Pseudomonas aeruginosa alginate, PsL, or Pel. In some embodiments, an antigenic polysaccharide is, or is derived from, an exopolysaccharide from Pseudomonas aeruginosa PsL.

In some embodiments, an antigenic polysaccharide is from, or derived from, a S. pneumoniae polysaccharides. In some embodiments, an antigenic polysaccharide present in an immunogenic complex described herein includes one S. pneumoniae polysaccharide. Capsular polysaccharides are used to distinguish serotypes of S. pneumoniae. There are at least 97 distinct serotypes of S. pneumoniae polysaccharides, each having a different chemical structure. FIG. 13 depicts exemplary structures and chemical information for certain S. pneumoniae capsular polysaccharides. All structures are from European Pharmacopoeia 9.0. Serotype designations as used herein are designations according to Danish nomenclature (Kauffmann et al, Intl. Bull. Bact. Nomenclature and Taxonomy 10:31-41 (1960); Geno et al, Clin Microbiol Rev 28(3):871-899 (2015)).

In some embodiments, an antigenic polysaccharide present in an immunogenic complex includes one or more S. pneumoniae capsular polysaccharides from, or derived from, one or more S. pneumoniae serotypes selected from 1, 2, 3, 4, 5, 6A, 6B, 6C, 6D, 6E, 6F, 6G, 6H, 7A, 7B, 7C, 7F, 8, 9A, 9L, 9N, 9V, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F, and 48.

In some embodiments, an antigenic polysaccharide present in an immunogenic complex includes one or more S. pneumoniae capsular polysaccharides from, or derived from, one or more S. pneumoniae serotypes selected from 1, 9N, and 19A.

In some embodiments, an antigenic polysaccharide present in an immunogenic complex includes one S. pneumoniae capsular polysaccharide from, or derived from, one S. pneumoniae serotype. In some embodiments, an antigenic polysaccharide present in an immunogenic complex includes one S. pneumoniae capsular polysaccharide from, or derived from, one S. pneumoniae serotype selected from 1, 9N, and 19A.

In some embodiments, an antigenic polysaccharide present in an immunogenic complex described herein includes one or more polysaccharide from a tumor.

### Exemplary Methods of Isolating and Purifying Polysaccharides

In some embodiments, the disclosure provides methods of purifying one or more polysaccharides described herein from one or more cellular components of bacteria. In some embodiments, methods comprise purifying capsular polysaccharides from one or more cellular components of bacteria.

In some embodiments, the bacteria are Gram-negative. In some embodiments, the bacteria are Gram-positive. In some embodiments, the bacteria are S. pneumoniae.

In some embodiments, the cellular components include protein. In some embodiments, the cellular proteins include nucleic acid. In some embodiments, the cellular components include lipids. In some embodiments, the cellular components include polysaccharides. In some embodiments, the cellular components are part of a lysate.

In some embodiments, the polysaccharide purification processes incorporate a series of ethanol precipitations, washes of crude polysaccharide preparations with ethanol, diethyl ether, and/or acetone, and drying under vacuum to furnish purified products. In some embodiments, a phenol extraction step is incorporated for polysaccharide purifications. In some embodiments, the purification process employs a CTAB (cetyltrimethyl ammonium bromide) precipitation step in addition to using ethanol and phenol precipitation steps.

### Exemplary Methods of Biotinylated Polysaccharides

In some embodiments, the disclosure provides methods of biotinylating one or more polysaccharides described herein. In some embodiments, the method comprises reacting purified polysaccharides with 1-cyano-4-dimethylaminopyridinium tetrafluoroborate (CDAP) for activation of hydroxyl groups in the polysaccharides followed by the addition of amine PEG biotin under conditions that result in covalent linkage of biotin to the polysaccharides. In some embodiments, the desired level of biotinylation is achieved by varying the ratio of CDAP to polysaccharide. In some embodiments, the biotinylated polysaccharides are purified by filtration to remove process residuals such as unreacted biotin, dimethylaminopyridine, acetonitrile, cyanide and unreacted glycine. In some embodiments, the level of polysaccharide biotinylation described herein is optimized to reduce the amount of accessible biotin following MAPS complexation.

### Immunogenic Conjugates

In some embodiments, immunogenic complexes described herein comprising a provided variant rhizavidin polypeptide can induce an immune response against a polypeptide and/or a polysaccharide at a higher level (e.g., by at least 30% or more, including, at least 40%, at least 50%, at least 60%, at least 70%, at least 80%, at least 90%, at least 100%, or more) than that is observed with an immunogenic conjugate comprising the same polypeptide and polysaccharide. As described herein, immunogenic conjugates include (i) one or more polypeptides (e.g., antigenic polypeptides) described herein conjugated to (ii) one or more polymers (e.g., antigenic polysaccharides) described herein. In some embodiments, the one or more polypeptides (e.g., antigenic polypeptides) described herein are covalently associated with the one or more polysaccharides described herein.

In some embodiments, one or more conjugated polysaccharides comprise a capsular polysaccharide of S. pneumoniae. In some embodiments, one or more polypeptides of an immunogenic conjugate comprise an antigenic polypeptide of S. pneumoniae. In some embodiments, one or more polypeptides of an immunogenic conjugate comprise an antigenic polypeptide of SARS-CoV-2. In some embodiments, an antigenic polypeptide of an immunogenic conjugate is or comprises a fusion protein.

### Manufacture of Immunogenic Complexes and Immunogenic Conjugates

The present disclosure includes methods for manufacturing immunogenic complexes described herein. In some embodiments, a method of manufacturing an immunogenic complex comprises complexing at least one biotinylated polymer (e.g., a biotinylated polysaccharide (e.g., a biotinylated polysaccharide described herein)) with at least one biotin-binding fusion protein described herein. In some embodiments, the present disclosure includes methods for manufacturing immunogenic conjugates described herein. In some embodiments, a method of manufacturing an immunogenic conjugate comprises conjugating at least one polymer (e.g., a polysaccharide (e.g., an antigenic polysaccharide)) described herein with at least one polypeptide (e.g., an antigenic polypeptide) described herein.

In some embodiments, the average (e.g., the mean) protein (e.g., antigenic protein) to polymer (e.g., polysaccharide) ratio of a plurality of immunogenic complexes or immunogenic conjugates is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]). In some embodiments, the average protein to polymer (e.g., polysaccharide) ratio of a plurality of immunogenic complexes or immunogenic conjugates is approximately 1:1 (w/w). In some embodiments, the average protein to polymer (e.g., polysaccharide) ratio of a plurality of immunogenic complexes or immunogenic conjugates is approximately 2:1 (w/w). In some embodiments, the average protein to polymer (e.g., polysaccharide) ratio of a plurality of immunogenic complexes or immunogenic conjugates is approximately 3:1 (w/w). In some embodiments, the average protein to polymer (e.g., polysaccharide) ratio of a plurality of immunogenic complexes or immunogenic conjugates is approximately 4:1 (w/w). In some embodiments, the average protein to polymer (e.g., polysaccharide) ratio of a plurality of immunogenic complexes or immunogenic conjugates is approximately 5:1 (w/w). In some embodiments, the average protein to polymer (e.g., polysaccharide) ratio of a plurality of immunogenic complexes or immunogenic conjugates is approximately 6:1 (w/w). In some embodiments, the average protein to polymer (e.g., polysaccharide) ratio of a plurality of immunogenic complexes or immunogenic conjugates is approximately 7:1 (w/w). In some embodiments, the average protein to polymer (e.g., polysaccharide) ratio of a plurality of immunogenic complexes or immunogenic conjugates is approximately 8:1 (w/w). In some embodiments, the average protein to polymer (e.g., polysaccharide) ratio of a plurality of immunogenic complexes or immunogenic conjugates is approximately 9:1 (w/w). In some embodiments, the average protein to polymer (e.g., polysaccharide) ratio of a plurality of immunogenic complexes or immunogenic conjugates is approximately 10:1 (w/w). Immunogenic compositions and vaccines of the present disclosure may comprise mixtures of immunogenic complexes or immunogenic conjugates with different average protein to polymer (e.g., polysaccharide) ratios. In some embodiments, the average ratio of protein to polymer in the plurality of immunogenic complexes is chosen to enhance the polymer immunogenicity potential (carrier or presentation function) and/or to elicit protection against, or to inhibit, a pathogen or a tumor through a protein-specific immune response. Immunogenic compositions (e.g., vaccines) of the present disclosure may comprise mixtures of immunogenic complexes with different average protein to polymer ratios.

In some embodiments, an immunogenic composition (e.g., a vaccine) comprises a plurality of immunogenic complexes comprising a fusion protein described herein and a capsular polysaccharide. In some embodiments, the average ratio of fusion protein to capsular polysaccharide in the plurality of immunogenic complexes is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]). In some embodiments, the average ratio of fusion protein to capsular polysaccharide in the plurality of immunogenic complexes is approximately 1:1 (w/w). In some embodiments, the average ratio of fusion protein to capsular polysaccharide in the plurality of immunogenic complexes is approximately 2:1 (w/w). In some embodiments, the average ratio of fusion protein to capsular polysaccharide in the plurality of immunogenic complexes is approximately 3:1 (w/w). In some embodiments, the average ratio of fusion protein to capsular polysaccharide in the plurality of immunogenic complexes is approximately 4:1 (w/w). In some embodiments, the average ratio of fusion protein to capsular polysaccharide in the plurality of immunogenic complexes is approximately 5:1 (w/w). In some embodiments, the average ratio of fusion protein to capsular polysaccharide in the plurality of immunogenic complexes is approximately 6:1 (w/w). In some embodiments, the average ratio of fusion protein to capsular polysaccharide in the plurality of immunogenic complexes is approximately 7:1 (w/w). In some embodiments, the average ratio of fusion protein to capsular polysaccharide in the plurality of immunogenic complexes is approximately 8:1 (w/w). In some embodiments, the average ratio of fusion protein to capsular polysaccharide in the plurality of immunogenic complexes is approximately 9:1 (w/w). In some embodiments, the average ratio of fusion protein to capsular polysaccharide in the plurality of immunogenic complexes is approximately 10:1 (w/w). In some embodiments, the average ratio of fusion protein to capsular polysaccharide in the plurality of immunogenic complexes is chosen to enhance the polysaccharide immunogenicity potential (carrier or presentation function) and/or to elicit protection against, or to inhibit, pneumococcal colonization by any pneumococcus (independent of polysaccharide serotype) through a protein-specific immune response. Immunogenic compositions (e.g., vaccines) of the present disclosure may comprise mixtures of immunogenic complexes with different average fusion protein to capsular polysaccharide ratios.

### Immunogenic Compositions

Another aspect of the disclosure provides compositions that include one or more immunogenic complexes described herein. For example, an immunogenic composition, e.g., vaccine composition, can include one or more immunogenic complexes described herein. In some embodiments, such compositions can include a plurality of one type of immunogenic complex described herein. For example, a composition can include a population of one type of immunogenic complex, where all of the immunogenic complexes include the same polypeptide (e.g., antigenic polypeptide) and the same polymer (e.g., antigenic polysaccharide). Additionally or alternatively, such compositions can include a plurality of more than one type of immunogenic complex described herein. For example, a composition can include populations of different types of immunogenic complexes. In some embodiments, a composition can include a population of a first type of immunogenic complex and a population of a second type of immunogenic complex, where the first type and the second type of the immunogenic complex have different polypeptide (e.g., antigenic polypeptides) and/or different polymers (e.g., antigenic polysaccharides). In some embodiments, a composition can include a population of a first type of immunogenic complex and a population of a second type of immunogenic complex, where the first type and the second type of the immunogenic complex include the same polypeptide (e.g., antigenic polypeptide) and different polymers (e.g., antigenic polysaccharides (e.g., polysaccharides of different serotypes)).

In some embodiments, an immunogenic composition is a monovalent immunogenic composition. In some embodiments, an immunogenic composition is a polyvalent or multivalent immunogenic composition. In some embodiments, an immunogenic composition is a monovariant immunogenic composition, comprising one or more antigens from one strain or variant of a pathogen. In some embodiments, an immunogenic composition is a multivariant immunogenic composition, comprising one or more antigens from more than one strain or variant of a pathogen. In some embodiments, the valency of an immunogenic composition refers to the number of species of immunogenic complexes present in the immunogenic composition. The valency of an immunogenic described herein is not limiting with respect to the total antigens present in said immunogenic composition, or to the number of pathogen strains for which administration of said immunogenic composition may induce an immune-protective response.

In some embodiments, an immunogenic composition comprises between 1-60 species of immunogenic complexes. In some embodiments, an immunogenic composition comprises between 1-50 species of immunogenic complexes. In some embodiments, an immunogenic composition comprises between 1-45 species of immunogenic complexes. In some embodiments, an immunogenic composition comprises between 1-40 species of immunogenic complexes. In some embodiments, an immunogenic composition comprises between 1-35 species of immunogenic complexes. In some embodiments, an immunogenic composition comprises between 1-34 species of immunogenic complexes. In some embodiments, an immunogenic composition comprises between 1-33 species of immunogenic complexes. In some embodiments, an immunogenic composition comprises between 1-32, 1-31, or 1-30 species of immunogenic complexes. In some embodiments, an immunogenic composition comprises between 1-30 species of immunogenic complexes. In some embodiments, an immunogenic composition comprises between 1-30 species of immunogenic complexes. In some embodiments, an immunogenic composition comprises between 1-24 species of immunogenic complexes. In some embodiments, an immunogenic composition comprises between 1-15 species of immunogenic complexes. In some embodiments, an immunogenic composition comprises between 1-9 species of immunogenic complexes. In some embodiments, an immunogenic composition comprises between 1-5 species of immunogenic complexes. In some embodiments, an immunogenic composition is a polyvalent composition.

In some embodiments, an immunogenic composition comprises at least 10, at least 15, at least 20, at least 21, at least 22, at least 23, at least 24, at least 25, at least 26, at least 27, at least 28, at least 29, at least 30, at least 31, at least 32, at least 33, or at least 34 species of immunogenic complexes. In some embodiments, an immunogenic composition comprises at least 25, at least 26, at least 27, at least 28, at least 29, at least 30, at least 31, at least 32, at least 33, or at least 34 species of immunogenic complexes. In some embodiments, an immunogenic composition comprises at least 30 (including, e.g., at least 31, at least 32, at least 33, at least 34) species of immunogenic complexes described herein. In some embodiments, an immunogenic composition comprises 30-40 species of immunogenic complexes described herein. In some embodiments, an immunogenic composition comprises 30-35 species of immunogenic complexes described herein. In some embodiments, an immunogenic composition comprises 25-50 or 25-40 species of immunogenic complexes described herein. In some embodiments, an immunogenic composition comprises no more than 60 species of immunogenic complexes described herein. In some embodiments, an immunogenic composition comprises no more than 50 species of immunogenic complexes described herein. In some embodiments, an immunogenic composition comprises no more than 45 species of immunogenic complexes described herein. In some embodiments, an immunogenic composition comprises no more than 40 species of immunogenic complexes described herein. In some embodiments, an immunogenic composition comprises no more than 35 species of immunogenic complexes described herein.

In some embodiments, an immunogenic composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 species of immunogenic complexes described herein.

In some embodiments, an immunogenic composition comprises two or more species of immunogenic complexes in amounts such that the weight of polymers (e.g., polysaccharides) in the immunogenic composition from each immunogenic complex is about the same, e.g., present in a w/w ratio of about 1:1. In some embodiments, the weight of polymers (e.g., polysaccharides) in the immunogenic composition contributed by each immunogenic complex is about 0.20 μg, about 0.25 μg, about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 7 μg, about 8 μg, about 9 μg, about 10 μg, about 11 μg, or about 12 μg. In some embodiments, the weight of polymers (e.g., polysaccharides) in the immunogenic composition contributed by each immunogenic complex is more than 12 μg, e.g., 13 μg, 14 μg, 15 μg, 16 μg, 17 μg, 18 μg, 19 μg, 20 μg, 21 μg, 22 μg, 23 μg, 24 μg, 25 μg, or more.

In some embodiments, an immunogenic composition comprises two or more species of immunogenic complexes in amounts such that the weight of polymers (e.g., polysaccharides) in the immunogenic composition contributed by each immunogenic complex is different, e.g., present in a w/w ratio that is not about 1:1. In some embodiments, an immunogenic composition comprises two or more species of immunogenic complexes in amounts such that the weight of polymers (e.g., polysaccharides) in the immunogenic composition contributed by a first immunogenic complex and a second immunogenic complex is 1:2, 1:3, 1:4, 1:5, 1:6, 1:7, 1:8, 1:9, or 1:10. In some embodiments, the immunogenic composition comprises a mixture of immunogenic complexes, such that the weight of polymers (e.g., polysaccharides) in an immunogenic composition contributed by an immunogenic complex ranges from about 0.20 μg to about 6 μg. In some embodiments, the immunogenic composition comprises a mixture of immunogenic complexes, such that the weight of polymers (e.g., polysaccharides) in an immunogenic composition contributed by an immunogenic complex ranges from about 0.20 μg to about 12 μg. In some embodiments, the immunogenic composition comprises a mixture of immunogenic complexes, such that the weight of polymers (e.g., polysaccharides) in the immunogenic composition contributed by each immunogenic complex ranges from about 0.20 μg to about 20 μg. In some embodiments, the immunogenic composition comprises a mixture of immunogenic complexes, such that the weight of polymers (e.g., polysaccharides) in the immunogenic composition contributed by each immunogenic complex ranges from about 0.20 μg to about 40 μg.

In some embodiments, an immunogenic composition comprises two or more species of immunogenic complexes in amounts such that the combined weight of polymers (e.g., polysaccharides) and polypeptides in the immunogenic composition contributed by each immunogenic complex is about the same, e.g., present in a w/w protein: PS ratio of about 1:1. In some embodiments, an immunogenic composition comprises two or more species of immunogenic complexes in amounts such that the combined weight of polymers (e.g., polysaccharides) and polypeptides in the immunogenic composition contributed by each immunogenic complex is present in a w/w protein: PS ratio of about 2:1, about 3:1, about 4:1, about 5:1, about 6:1, about 7:1, about 8:1, about 9:1, or about 10:1.

In some embodiments, the combined weight of polymers (e.g., polysaccharides) and polypeptides in an immunogenic composition contributed by each immunogenic complex is about 0.20 μg, about 0.40 μg, about 1 μg, about 2 μg, about 3 μg, about 4 μg, about 5 μg, about 6 ug, about 7 μg, about 8 μg, about 9 μg, about 10 μg, about 11 μg, about 12 μg, about 14 μg, about 16 μg, about 18 μg, about 20 μg, about 21 μg, about 22 μg, about 23 μg, about 24 μg, about 25μ, about 30μ, about 40μ, about 50μ, about 60μ, about 70μ, about 80μ g, about 90 μg, about 100 μg, or about 110 μg.

In some embodiments, an immunogenic composition comprises two or more species of immunogenic complexes (e.g., in immunogenic compositions) in amounts such that the combined weight of polymers (e.g., polysaccharides) and polypeptides in the immunogenic composition contributed by each immunogenic complex is different, e.g., present in a w/w protein: PS ratio that is not about 1:1, e.g., a protein: PS ratio that is 2:1, 3:1, 4:1. 5:1. 6:1, 7:1, 8:1, 9:1, or 10:1. In some embodiments, the immunogenic composition comprises a mixture of immunogenic complexes, such that the combined weight of polymers (e.g., polysaccharides) and polypeptides in the immunogenic composition contributed by each immunogenic complex ranges from about 0.4 μg to about 110 μg.

In some embodiments, an immunogenic composition described herein comprises an adjuvant.

In some embodiments, an immunogenic composition described herein is a vaccine composition.

In some embodiments, an immunogenic composition is an immunogenic composition described in WO 2020/056202, the contents of which are hereby incorporated by reference in their entirety for purposes described herein. In some embodiments, an immunogenic composition is an immunogenic composition described in International application PCT/US2022/043156, the contents of which are hereby incorporated by reference in their entirety for purposes described herein.

### Pharmaceutical Compositions

In some embodiments, a composition described herein is formulated into a pharmaceutical composition. In some embodiments, a pharmaceutical composition comprises a pharmaceutically acceptable carrier. In some embodiments, a pharmaceutical composition comprises an adjuvant.

Optimal amounts of components for a particular pharmaceutical composition can be ascertained by standard studies involving observation of appropriate immune responses in subjects. Following an initial immunization, subjects can receive one or several booster immunizations adequately spaced in time.

The immunogenic complexes described herein, and/or preparations thereof, may be formulated in a unit dosage form for ease of administration and uniformity of dosage. The specific therapeutically effective dose level for any particular subject or organism may depend upon a variety of factors including the severity or degree of risk of infection; the activity of the specific pharmaceutical composition employed; other characteristics of the specific pharmaceutical composition employed; the age, body weight, general health, sex of the subject, diet of the subject, pharmacokinetic condition of the subject, the time of administration (e.g., with regard to other activities of the subject such as eating, sleeping, receiving other medicines including other pharmaceutical composition doses, etc.), route of administration, rate of excretion of the specific pharmaceutical composition employed; pharmaceutical compositions used in combination or coincidental with the pharmaceutical composition employed; and like factors well known in the medical arts.

Immunogenic complexes for use in accordance with the present disclosure may be formulated into compositions (e.g., pharmaceutical compositions) according to known techniques. In some embodiments where a pharmaceutical composition comprises a vaccine composition, vaccine preparation is generally described in Vaccine Design (Powell and Newman, 1995). For example, an immunologically effective amount of a vaccine composition can be formulated together with one or more organic or inorganic, liquid or solid, pharmaceutically suitable carrier materials.

In general, pharmaceutically acceptable carrier(s) include solvents, dispersion media, and the like, which are compatible with pharmaceutical administration. For example, materials that can serve as pharmaceutically acceptable carriers include, but are not limited to sugars such as lactose, glucose, dextrose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; polyols such as glycerol, propylene glycol, and liquid polyethylene glycol; esters such as ethyl oleate and ethyl laurate; agar; buffering agents such as magnesium hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as preservatives, and antioxidants can also be present in the composition, according to the judgment of the formulator (Martin, 1975).

Pharmaceutical compositions may be formulated by combining one or more of the immunogenic complexes disclosed herein with carriers and/or other optional components by any available means including, for example, conventional mixing, granulating, dissolving, lyophilizing, or similar processes.

Pharmaceutical compositions useful in the provided methods may be lyophilized up until they are about to be used, at which point they are extemporaneously reconstituted with diluent. In some embodiments, pharmaceutical compositions or components thereof are lyophilized in the presence of one or more other components (e.g., adjuvants), and are extemporaneously reconstituted with saline solution. Alternatively, individual components, or sets of components may be separately lyophilized and/or stored (e.g., in a vaccination kit), the components being reconstituted and either mixed prior to use or administered separately to the subject.

Lyophilization can produce a more stable composition (e.g., by preventing or reducing breakdown of polysaccharide antigens). Lyophilizing of pharmaceutical compositions or components thereof is well known in the art. Typically, a liquid pharmaceutical composition or component thereof is freeze dried, often in the presence of an anti-caking agent (such as, for example, sugars such as sucrose or lactose). In some embodiments, the anti-caking agent is present, for example, at an initial concentration of 10-200 mg/ml. Lyophilization typically occurs over a series of steps, for instance a cycle starting at −69° C., gradually adjusting to −24° C. over 3 h, then retaining this temperature for 18 h, then gradually adjusting to −16° C. over 1 h, then retaining this temperature for 6 h, then gradually adjusting to +34° C. over 3 h, and finally retaining this temperature over 9 h.

In some embodiments, a pharmaceutical composition is a liquid. In some embodiments, the liquid is a reconstituted lyophylate. In some embodiments, a pharmaceutical composition has a pH of about 5, about 6, about 7, or about 8. In some embodiments, a pharmaceutical composition has a pH between about 5 and about 7.5. In some embodiments, a pharmaceutical composition has a pH between 5 and 7.5. In some embodiments, a pharmaceutical composition has a pH between about 5.3 and about 6.3. In some embodiments, a pharmaceutical composition has a pH between 5.3 and 6.3. In some embodiments, a pharmaceutical composition has a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5.

Pharmaceutical compositions or components thereof for use in accordance with the present disclosure may be incorporated into liposomes, cochleates, biodegradable polymers such as poly-lactide, poly-glycolide and poly-lactide-co-glycolides, or immune-stimulating complexes (ISCOMs).

In certain situations, it may be desirable to prolong the effect of a pharmaceutical composition for use in accordance with the present disclosure, for example by slowing the absorption of one or more pharmaceutical composition components. Such delay of absorption may be accomplished, for example, by the use of a liquid suspension of crystalline or amorphous material with poor water solubility. The rate of absorption of the product then depends upon its rate of dissolution, which in turn, may depend upon size and form. Alternatively, or additionally, delayed absorption may be accomplished by dissolving or suspending one or more pharmaceutical composition components in an oil vehicle. Injectable depot forms can also be employed to delay absorption. Such depot forms can be prepared by forming microcapsule matrices of one or more pharmaceutical composition components a biodegradable polymers network. Depending upon the ratio of polymer to pharmaceutical composition component, and the nature of the particular polymer(s) employed, the rate of release can be controlled.

Examples of biodegradable polymers that can be employed in accordance with the present disclosure include, for example, poly(orthoesters) and poly(anhydrides). One particular exemplary polymer is polylactide-polyglycolide.

Depot injectable formulations may also be prepared by entrapping the product in liposomes or microemulsions, which are compatible with body tissues.

Polymeric delivery systems can also be employed in non-depot formulations including, for example, oral formulations. For example, biodegradable, biocompatible polymers such as ethylene vinyl acetate, polyanhydrides, polyglycolic acid, collagen, polyorthoesters, and polylactic acid, etc., can be used in oral formulations. Polysaccharide antigens or conjugates may be formulated with such polymers, for example to prepare particles, microparticles, extrudates, solid dispersions, admixtures, or other combinations in order to facilitate preparation of useful formulations (e.g., oral).

Pharmaceutical compositions for use in accordance with the present disclosure include immunogenic compositions, and may additionally include one or more additional active agents (i.e., agents that exert a biological effect—not inert ingredients). For example, it is common in pharmaceutical composition preparation to include one or more adjuvants. It will be appreciated that such additional agents may be formulated together with one or more other pharmaceutical composition components, or may be maintained separately and combined at or near the time of administration. In some embodiments, such additional components may be administered separately from some or all of the other pharmaceutical composition components, within an appropriate time window for the relevant effect to be achieved.

### Adjuvants

The immunogenic compositions (e.g., vaccine compositions) and pharmaceutical compositions described herein may include an adjuvant. Adjuvants, generally, are agents that enhance the immune response to an antigen. Adjuvants can be broadly separated into two classes, based on their principal mechanisms of action: vaccine delivery systems and immunostimulatory adjuvants (see, e.g., Singh et al., 2003). In most immunogenic composition or pharmaceutical composition formulations, the adjuvant provides a signal to the immune system so that it generates a response to the antigen, and the antigen is required for driving the specificity of the response to the pathogen. Vaccine delivery systems are often particulate formulations, e.g., emulsions, microparticles, immune-stimulating complexes (ISCOMs), nanoparticles, which may be, for example, particles and/or matrices, and liposomes. In contrast, immunostimulatory adjuvants are sometimes from or derived from pathogens and can represent pathogen associated molecular patterns (PAMP), e.g., lipopolysaccharides (LPS), monophosphoryl lipid A (MPL), or CpG-containing DNA, which activate cells of the innate immune system.

Alternatively, adjuvants may be classified as organic and inorganic. Inorganic adjuvants include aluminum-based adjuvants (e.g., alum salts) such as aluminum phosphate, amorphous aluminum hydroxyphosphate sulfate, and aluminum hydroxide, which are commonly used in human immunogenic compositions and pharmaceutical compositions. Organic adjuvants comprise organic molecules including macromolecules. Non-limiting examples of organic adjuvants include cholera toxin/toxoids, other enterotoxins/toxoids or labile toxins/toxoids of Gram-negative bacteria, interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), and tumor necrosis factor (TNF).

Adjuvants may also be classified by the response they induce. In some embodiments, an adjuvant induces generation, proliferation, or activation of T_{H}1 cells or T_{H}2 cells. In other embodiments, an adjuvant induces generation, proliferation, or activation of B cells. In yet other embodiments, an adjuvant induces activation of antigen-presenting cells. These categories are not mutually exclusive; in some cases, an adjuvant activates more than one type of cell.

In certain embodiments, the adjuvant induces the generation, proliferation, or activation of T_{H}17 cells. The adjuvant may promote the CD4+ or CD8+ T cells to secrete IL-17. In some embodiments, an adjuvant that induces the generation, proliferation, or activation of T_{H}17 cells is one that produces at least a 2-fold, and in some cases a 10-fold, experimental sample to control ratio in the following assay. In the assay, an experimenter compares the IL-17 levels secreted by two populations of cells: (1) cells from animals immunized with the adjuvant and a polypeptide known to induce T_{H}17 generation, proliferation, or activation, and (2) cells from animals treated with the adjuvant and an irrelevant (control) polypeptide. An adjuvant that induces the generation, proliferation, or activation of T_{H}17 cells may cause the cells of population (1) to produce more than 2-fold, or more than 10-fold more IL-17 than the cells of population (2). IL-17 may be measured, for example, by ELISA or ELISPOT. Certain toxins, such as cholera toxin and labile toxin (produced by enterotoxigenic E. coli, or ETEC), activate a T_{H}17 response. Thus, in some embodiments, the adjuvant is a toxin or toxoid. Mutant derivates of labile toxin (toxoids) that are active as adjuvants but significantly less toxic can be used as well. Exemplary detoxified mutant derivatives of labile toxin include mutants lacking ADP-ribosyltransferase activity. Particular detoxified mutant derivatives of labile toxin include LTK7 (Douce et al., 1995) and LTK63 (Williams et al., 2004), LT-G192 (Douce et al., 1999), and LTR72 (Giuliani et al., 1998).

In some embodiments, the adjuvant comprises a VLP (virus-like particle). One such adjuvant platform, Alphavirus replicons, induces the activation of T_{H}17 cells using alphavirus and is produced by Alphavax. In certain embodiments of the Alphavirus replicon system, alphavirus may be engineered to express an antigen of interest, a cytokine of interest (for example, IL-17 or a cytokine that stimulates IL-17 production), or both, and may be produced in a helper cell line. More detailed information may be found in U.S. Pat. Nos. 5,643,576 and 6,783,939. In some embodiments, an immunogenic composition (e.g., vaccine) formulation is administered to a subject in combination with a nucleic acid encoding a cytokine.

Certain classes of adjuvants activate toll-like receptors (TLRs) in order to activate a T_{H}17 response. TLRs are well known proteins that may be found on leukocyte membranes, and recognize foreign antigens (including microbial antigens). Administering a known TLR ligand together with an antigen of interest (for instance, as a fusion protein) can promote the development of an immune response specific to the antigen of interest. One exemplary adjuvant that activates TLRs comprises Monophosphoryl Lipid A (MPL). Traditionally, MPL has been produced as a detoxified lipopolysaccharide (LPS) endotoxin obtained from Gram-negative bacteria, such as S. minnesota. In particular, sequential acid and base hydrolysis of LPS produces an immunoactive lipid A fraction (which is MPL), and lacks the saccharide groups and all but one of the phosphates present in LPS. A number of synthetic TLR agonists (in particular, TLR-4 agonists) are disclosed in Evans et al., 2003. Like MPL adjuvants, these synthetic compounds activate the innate immune system via TLR. Another type of TLR agonist is a synthetic phospholipid dimer, for example E6020 (Ishizaka et al., 2007). Various TLR agonists (including TLR-4 agonists) have been produced and/or sold by, for example, the Infectious Disease Research Institute (IRDI), Corixa, Esai, Avanti Polar Lipids, Inc., and Sigma Aldrich. Another exemplary adjuvant that activates TLRs comprises a mixture of MPL, Trehalose Dicoynomycolate (TDM), and dioctadecyldimethylammonium bromide (DDA). Another TLR-activating adjuvant is R848 (resiquimod).

In some embodiments, the adjuvant is or comprises a saponin. Typically, the saponin is a triterpene glycoside, such as those isolated from the bark of the Quillaja saponaria tree. A saponin extract from a biological source can be further fractionated (e.g., by chromatography) to isolate the portions of the extract with the best adjuvant activity and with acceptable toxicity. Typical fractions of extract from Quillaja saponaria tree used as adjuvants are known as fractions A and C.

In certain embodiments, combinations of adjuvants are used. Three exemplary combinations of adjuvants are MPL and alum, E6020 and alum, and MPL and an ISCOM.

Adjuvants may be covalently or non-covalently bound to antigens. In some embodiments, the adjuvant may comprise a protein which induces inflammatory responses through activation of antigen-presenting cells (APCs). In some embodiments, one or more of these proteins can be recombinantly fused with an antigen of choice, such that the resultant fusion molecule promotes dendritic cell maturation, activates dendritic cells to produce cytokines and chemokines, and ultimately, enhances presentation of the antigen to T cells and initiation of T cell responses (e.g., see Wu et al., 2005).

In some embodiments, immunogenic complexes described herein are formulated and/or administered in combination with an adjuvant. In some embodiments, such an adjuvant is or comprises an aluminum-based adjuvant (e.g., an aluminum salt); see e.g., Danielsson and Eriksson, “Aluminum adjuvants in vaccines—a way to modulate the immune response”, Semin Cell Dev Biol, 115:3-9 (2021) and HogenEsch et al., “Optimizing the utilization of aluminum adjuvants in vaccines: you might just get what you want”, NPJ Vaccines, 3:51 (2018), the contents of which are incorporated herein by reference in their entirety for purposes described herein. Examples of an aluminum-based adjuvant include, but are not limited to, aluminum phosphate, aluminum hydroxide, potassium aluminum sulfate (alum), aluminum hydroxide phosphate, aluminum hydroxyphosphate sulfate, and combinations thereof. In some embodiments, an aluminum-based adjuvant comprises aluminum phosphate. In some embodiments, an aluminum-based adjuvant is aluminum phosphate.

In some embodiments, the same adjuvant or mixture of adjuvants is present in each dose of an immunogenic composition or pharmaceutical composition. Optionally, however, an adjuvant may be administered with the first dose of an immunogenic composition or pharmaceutical composition and not with subsequent doses (i.e., booster shots). Alternatively, a strong adjuvant may be administered with the first dose of an immunogenic composition or pharmaceutical composition and a weaker adjuvant or lower dose of the strong adjuvant may be administered with subsequent doses. The adjuvant can be administered before the administration of the antigen, concurrent with the administration of the antigen or after the administration of the antigen to a subject (sometimes within 1, 2, 6, or 12 hours, and sometimes within 1, 2, or 5 days). Certain adjuvants are appropriate for human subjects, non-human animals, or both.

Immunogenic compositions and pharmaceutical compositions for use in accordance with the present disclosure may include, or be administered concurrently with, other antimicrobial, antiviral, or anti-inflammatory therapies. For example, such immunogenic compositions and pharmaceutical compositions may include or be administered with one or more agents that kills or retards growth of a pathogen. Such agents include, for example, remdesivir, lopinavir and/or ritonavir (e.g., Kaletra), oseltamivir (Tamiflu), favipiravir, umifenovir, galidesivir, dexamethasone, colchicine, convalescent plasma, monoclonal antibodies (e.g., one or more of bamlanivimab, LY-CoV016, etesevimab, casirivimab, indevimab, sarilumab, tocilizumab), IL-6 inhibitors, kinase inhibitors, interferons, penicillin, vancomycin, erythromycin, azithromycin, and clarithromycin, cefotaxime, ceftriaxone, levoflaxin, gatifloxacin.

Alternatively or additionally, immunogenic compositions and pharmaceutical compositions for use in accordance with the present disclosure may include, or be administered with, one or more other immunogenic compositions, pharmaceutical compositions, or therapies.

### Additional Components and Excipients

In addition to the antigens and the adjuvants described above, an immunogenic composition (e.g., a vaccine composition) or pharmaceutical composition may include one or more additional components.

In certain embodiments, the immunogenic composition or pharmaceutical composition may include one or more stabilizers such as sugars (such as sucrose, glucose, or fructose), phosphate (such as sodium phosphate dibasic, potassium phosphate monobasic, dibasic potassium phosphate, or monosodium phosphate), glutamate (such as monosodium L-glutamate), gelatin (such as processed gelatin, hydrolyzed gelatin, or porcine gelatin), amino acids (such as arginine, asparagine, histidine, L-histidine, alanine, valine, leucine, isoleucine, serine, threonine, lysine, phenylalanine, tyrosine, and the alkyl esters thereof), inosine, or sodium borate.

In certain embodiments, the immunogenic composition or pharmaceutical composition includes one or more buffers such as histidine, glycine, succinate, Tris, or a mixture of sodium bicarbonate and ascorbic acid. In some embodiments, the vaccine formulation may be administered in saline, such as phosphate buffered saline (PBS), or distilled water.

In certain embodiments, the immunogenic composition or pharmaceutical composition includes one or more surfactants, for example, but not limited to, polysorbate 80 (TWEEN 80), polysorbate 20 (TWEEN 20), Polyethylene glycol p-(1,1,3,3-tetramethylbutyl)-phenyl ether (TRITON X-100), and 4-(1,1,3,3-Tetramethylbutyl) phenol polymer with formaldehyde and oxirane (TYLOXAPOL). A surfactant can be ionic or non-ionic.

In certain embodiments, the immunogenic composition or pharmaceutical composition includes one or more salts such as sodium chloride, ammonium chloride, calcium chloride, or potassium chloride.

In certain embodiments, a preservative is included in the immunogenic composition or pharmaceutical composition. In other embodiments, no preservative is used. A preservative is most often used in multi-dose immunogenic composition (e.g., vaccine) or pharmaceutical composition vials, and is less often needed in single-dose immunogenic composition (e.g., vaccine) or pharmaceutical composition vials. In certain embodiments, the preservative is 2-phenoxyethanol, thimerosal, methyl and propyl parabens, benzyl alcohol, and/or sorbic acid.

### Uses of Immunogenic Compositions and Pharmaceutical Compositions

In some embodiments, an immunogenic composition (e.g., vaccine) or pharmaceutical composition described herein that includes one or more antigenic polysaccharides is characterized in that one or more of the opsonization potential, or immune response to one or more antigenic polysaccharides is increased relative to a predetermined level, as measured by ELISA and or by a functional antibody assay. In some embodiments, one or more of the opsonization potential, immune response to the one or more antigenic polysaccharides is increased at least 1-fold, 2-fold, 3-fold, 4-fold, or 5-fold relative to a predetermined level, as measured by ELISA and or by a functional antibody assay. In some embodiments, the predetermined level is a pre-immune level. In some embodiments, one or more polypeptides (e.g., antigenic polypeptides) are carrier proteins for one or more antigenic polysaccharides.

In some embodiments, an immunogenic composition or pharmaceutical composition described herein, upon administration to a subject, induces an immune response against one or more tumors or pathogens in the subject at a level greater than a composition comprising an antigenic polysaccharide alone. In some embodiments, an immunogenic composition (e.g., vaccine) or pharmaceutical composition described herein, upon administration to a subject, induces an immune response against one or more tumors or pathogens in the subject at a level greater than a composition comprising an antigenic polypeptide alone. In some embodiments, an immunogenic composition (e.g., vaccine) or pharmaceutical composition described herein, upon administration to a subject, induces a protective immune response. In some embodiments, an immunogenic composition (e.g., vaccine) or pharmaceutical composition described herein, upon administration to a subject, induces an immune response against one or more strains (or variants, or serotypes) of a pathogen. In some embodiments, a pathogen is a bacterial, fungal, parasitic, and/or viral pathogen. In some embodiments, a viral pathogen is coronavirus (e.g., in some embodiments, SARS-CoV-2). In some embodiments, a bacterial pathogen is S. pneumoniae. In some embodiments, an immunogenic composition (e.g., vaccine) or pharmaceutical composition described herein comprising a tumor antigen polypeptide (e.g., as described herein), upon administration to a subject, induces an immune response against one or more tumors characterized by expression of such a tumor or cancer antigen.

In some embodiments, the immune response is an antibody or B cell response. In some embodiments, the antibody or B cell response is a memory B cell response. In some embodiments, the immune response is a T cell response. In some embodiments, the T cell response is a memory T cell response. In some embodiments, the immune response is an innate immune response. In some embodiments, the immune response is a CD4+ T cell response, including T_{H}1, T_{H}2, or T_{H}17 response, or a CD8+ T cell response, or a CD4+ and CD8+ T cell response, or a CD4−/CD8− T cell response. In some embodiments, the immune response is an antibody or B cell response, and a T cell response. In some embodiments, the immune response is an antibody or B cell response, a T cell response, and an innate immune response. In some embodiments, the immune response is a protective immune response. In some embodiments, the immune response comprises neutralizing antibodies. In some embodiments, the immune response is a memory response.

In some embodiments, an immunogenic composition (e.g., vaccine) or pharmaceutical composition described herein, upon administration to a subject, induces antibody production against one or more tumors or pathogens in the subject at a level greater than a composition comprising an antigenic polysaccharide alone. In some embodiments, an immunogenic composition (e.g., vaccine) or pharmaceutical composition described herein, upon administration to a subject, induces antibody production against one or more tumors or pathogens in the subject at level greater than a composition comprising a polypeptide antigen alone.

In some embodiments, an immunogenic composition (e.g., vaccine) or pharmaceutical composition described herein, upon administration to a subject, induces an immune response against one or more tumors or pathogens in the subject at a level greater than a composition comprising an antigenic polysaccharide alone. In some embodiments, an immunogenic composition (e.g., vaccine) or pharmaceutical composition described herein, upon administration to a subject, induces an immune response against one or more tumors or pathogens in the subject at a level greater than a composition comprising a polypeptide antigen alone. In some embodiments, an immunogenic composition (e.g., vaccine) or pharmaceutical composition described herein, upon administration to a subject, induces a protective immune response.

In some embodiments, immunogenic compositions (e.g., vaccines) and pharmaceutical compositions described herein may be used for therapy. In some embodiments, immunogenic compositions (e.g., vaccines) and pharmaceutical compositions described herein may be used for prophylactic and/or therapeutic treatment of cancer or pathogen infection and/or disease. Accordingly, in some embodiments, the present disclosure provides a method for immunizing a subject suffering from or susceptible to cancer or pathogen infection, comprising administering an immunologically effective amount of any of the immunogenic compositions (e.g., vaccines) or pharmaceutical compositions described herein. In some embodiments, the present disclosure provides a use of an immunologically effective amount of any of the immunogenic compositions (e.g., vaccines) or pharmaceutical compositions described herein in the manufacture of a medicament for administration to a subject to immunize the subject against cancer or pathogen infection. In some embodiments, the present disclosure provides a use of an immunologically effective amount of any of the immunogenic compositions (e.g., vaccines) or pharmaceutical compositions described herein for administration to a subject to immunize the subject against cancer or pathogen infection. The subject receiving the immunization may be a male or a female, and may be an infant, child, adolescent, or adult. In some embodiments, the subject being treated is a human. In other embodiments, the subject is a non-human animal. In other embodiments, such a non-human animal is an agriculture or non-domestic animal. In other embodiments, such a non-human animal is a domestic animal. In some embodiments, an immunogenic composition (e.g., vaccine) or pharmaceutical composition described herein, upon administration to a subject, induces a protective immune response against cancer or pathogen infection and/or disease. In some embodiments, the present disclosure provides an immunogenic composition (e.g., vaccine) or pharmaceutical composition described herein for use in the treatment of cancer or pathogen infection and/or disease. In some embodiments, the present disclosure provides a use of an immunogenic composition (e.g., vaccine) or pharmaceutical composition described herein in the manufacture of a medicament for the treatment of cancer or pathogen infection and/or disease. In some embodiments, the present disclosure provides a method of immunizing a subject by administering to the subject an immunogenic composition (e.g., vaccine) or pharmaceutical composition described herein. In some embodiments, the present disclosure provides an immunogenic composition (e.g., vaccine) or pharmaceutical composition described herein for use in administration to a subject to immunize the subject. In some embodiments, the present disclosure provides a use of an immunogenic composition (e.g., vaccine) or pharmaceutical composition described herein in the manufacture of a medicament for administration to a subject to immunize the subject. The characteristics of methods of treatment or prevention disclosed herein are equally applicable to an immunogenic composition (e.g., vaccine) or pharmaceutical composition for use, or a use of an immunogenic composition (e.g., vaccines) or pharmaceutical composition in the manufacture of a medicament.

In prophylactic embodiments, an immunogenic composition (e.g., vaccine) or pharmaceutical composition described herein is administered to a subject to induce an immune response that can help protect against the establishment of one or more strains (variants) of a pathogen, for example by protecting against asymptomatic infection. In some aspects, the method inhibits infection by a pathogen in an uninfected subject. In another aspect, the method may reduce transmission, replication, and/or pathogen load of one or more strains (variants) of a pathogen in a subject who is already infected.

In prophylactic embodiments, an immunogenic composition (e.g., vaccine) or pharmaceutical composition described herein comprising a tumor antigen polypeptide (e.g., as described herein) is administered to a subject to induce an immune response that can help protect against the establishment of one or more tumors characterized by expression of such a tumor or cancer antigen, for example by protecting against asymptomatic cancer. In some aspects, the method inhibits cancer by a tumor in an unaffected subject. In another aspect, the method may reduce metastasis, replication, and/or tumor load of one or more tumors in a subject who is already affected.

In therapeutic embodiments, the immunogenic composition (e.g., vaccine) or pharmaceutical composition may be administered to a subject suffering from cancer or a pathogen infection, in an amount sufficient to treat the subject. Treating the subject, in this case, refers to reducing cancer or pathogen symptoms and/or tumor or pathogen load and/or sequelae in an subject. In some embodiments, treating the subject refers to reducing the duration of symptoms or sequelae, or reducing the intensity of symptoms or sequelae. In some embodiments, the immunogenic composition (e.g., vaccine) or pharmaceutical composition reduces transmissibility of one or more strains (variants) of a pathogen from the immunized subject to another subject. In some embodiments, the immunogenic composition (e.g., vaccine) or pharmaceutical composition reduces metastasis of one or more tumors in the immunized subject. In certain embodiments, the reductions described above are at least 25%, 30%, 40%, 50%, 60%, 70%, 80%, or 90%, e.g., relative to a control, e.g., a control subject.

In therapeutic embodiments, the immunogenic composition (e.g., vaccine) or pharmaceutical composition is administered to a subject post-tumor development or post-pathogen infection. The immunogenic composition (e.g., vaccine) or pharmaceutical composition may be administered shortly after tumor development or pathogen infection, e.g. before symptoms or sequelae manifest, or may be administered during or after manifestation of symptoms or sequelae.

In some embodiments, the immunogenic compositions (e.g., vaccines) or pharmaceutical compositions described herein confer protective immunity, allowing an immunized subject to exhibit delayed onset of symptoms or sequelae, or reduced severity of symptoms or sequelae, as the result of his or her exposure to the immunogenic composition (e.g., vaccine) or pharmaceutical composition. In certain embodiments, the reduction in severity of symptoms or sequelae is at least 25%, 40%, 50%, 60%, 70%, 80%, or 90%, e.g., relative to a control. In particular embodiments, immunized subjects may display no symptoms or sequelae upon development of a tumor or infection with a pathogen (asymptomatic cancer or infection), or do not develop a tumor or become infected by a pathogen. Protective immunity is typically achieved by one or more of the following mechanisms: mucosal, humoral, or cellular immunity. Mucosal immunity is primarily the result of secretory IgA (sIGA) antibodies on mucosal surfaces of the respiratory, gastrointestinal, and genitourinary tracts. The sIGA antibodies are generated after a series of events mediated by antigen-processing cells, B and T lymphocytes, that result in sIGA production by B lymphocytes on mucosa-lined tissues of the body. Humoral immunity is typically the result of IgG antibodies and IgM antibodies in serum. Cellular immunity can be achieved through cytotoxic T lymphocytes or through delayed-type hypersensitivity that involves macrophages and T lymphocytes, as well as other mechanisms involving T cells without a requirement for antibodies. In particular, cellular immunity may be mediated by T_{H}1 or T_{H}17 cells.

In some embodiments, an immunogenic composition (e.g., vaccine) or pharmaceutical composition described herein, upon administration to a subject, induces an immune response against one or more strains (variants) of a pathogen. In some embodiments, an immunogenic composition (e.g., vaccine) or pharmaceutical composition described herein comprising a tumor antigen polypeptide (e.g., as described herein), upon administration to a subject, induces an immune response against one or more tumors characterized by expression of such a tumor or cancer antigen.

In some embodiments, the immune response is an antibody or B cell response. In some embodiments, the antibody or B cell response is a memory B cell response. In some embodiments, the immune response is a T cell response. In some embodiments, the T cell response is a memory T cell response. In some embodiments, the immune response is an innate immune response. In some embodiments, the immune response is a CD4+ T cell response, including T_{H}1, T_{H}2, or T_{H}17 response, or a CD8+ T cell response, or a CD4+ and CD8+ T cell response, or CD4−/CD8− T cell response. In some embodiments, the immune response is an antibody or B cell response, and a T cell response. In some embodiments, the immune response is an antibody or B cell response, a T cell response, and an innate immune response. In some embodiments, the immune response is a protective immune response. In some embodiments, the immune response comprises neutralizing antibodies. In some embodiments, the immune response is a memory response.

In some embodiments, an immunogenic composition (e.g., vaccine) or pharmaceutical composition described herein, upon administration to a subject, induces an antibody or B cell response against one or more tumors or pathogens in the subject at a level greater than a composition comprising an antigenic polysaccharide alone. In some embodiments, an immunogenic composition (e.g., vaccine) or pharmaceutical composition described herein, upon administration to a subject, induces an antibody or B cell response against one or more tumors or pathogens in the subject at level greater than a composition comprising a polypeptide antigen alone. In some embodiments, the immune response is a protective immune response. In some embodiments, the immune response comprises neutralizing antibodies.

In some embodiments, an immunogenic composition (e.g., vaccine) or pharmaceutical composition described herein, upon administration to a subject, induces a T cell response against one or more tumors or pathogens in the subject at a level greater than a composition comprising an antigenic polysaccharide alone. In some embodiments, an immunogenic composition (e.g., vaccine) or pharmaceutical composition described herein, upon administration to a subject, induces a T cell response against one or more tumors or pathogens in the subject at level greater than a composition comprising a polypeptide antigen alone. In some embodiments, the immune response is a protective immune response. In some embodiments, the immune response comprises neutralizing antibodies.

In some embodiments, upon administration to a subject, an immunogenic composition (e.g., vaccine) or pharmaceutical composition described herein treats or prevents infection by one or more strains (variants) of a pathogen. In some embodiments, upon administration to a subject, an immunogenic composition (e.g., vaccine) or pharmaceutical composition described herein treats or prevents disease due to infection by one or more strains (variants) of a pathogen. In some embodiments, upon administration to a subject, an immunogenic composition (e.g., vaccine) or pharmaceutical composition described herein treats or prevents one or more symptoms due to infection by one or more strains (variants) of a pathogen. In some embodiments, the one or more symptoms include pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), and/or multi-inflammatory syndrome of children or adults (MIS-C, MIS-A).

In some embodiments, upon administration to a subject, an immunogenic composition (e.g., vaccine) or pharmaceutical composition described herein inhibits or reduces the rate of occurrence or recurrence of infection by one or more strains (variants) of a pathogen. In some embodiments, upon administration to a subject, an immunogenic composition (e.g., vaccine) or pharmaceutical composition described herein inhibits or reduces the rate of occurrence or recurrence of disease due to infection by one or more strains (variants) of a pathogen. In some embodiments, upon administration to a subject, an immunogenic composition (e.g., vaccine) or pharmaceutical composition described herein inhibits or reduces the rate of occurrence or recurrence of one or more symptoms due to infection by one or more strains (variants) of a pathogen. In some embodiments, the one or more symptoms include pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), and/or multi-inflammatory syndrome of children or adults (MIS-C, MIS-A).

In some embodiments, upon administration to a subject, an immunogenic composition (e.g., vaccine) or pharmaceutical composition described herein reduces the severity of infection by one or more strains (variants) of a pathogen. In some embodiments, upon administration to a subject, an immunogenic composition (e.g., vaccine) or pharmaceutical composition described herein reduces the severity of disease due to infection by one or more strains (variants) of a pathogen. In some embodiments, upon administration to a subject, an immunogenic composition (e.g., vaccine) or pharmaceutical composition described herein reduces the severity of one or more symptoms due to infection by one or more strains (variants) of a pathogen. In some embodiments, the one or more symptoms include pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), and/or multi-inflammatory syndrome of children or adults (MIS-C, MIS-A).

In some embodiments, an immunogenic composition (e.g., vaccine) or pharmaceutical composition described herein comprising a tumor antigen polypeptide (e.g., as described herein) treats or prevents cancer by one or more tumors characterized by expression of such a tumor or cancer antigen. In some embodiments, upon administration to a subject, an immunogenic composition (e.g., vaccine) or pharmaceutical composition described herein comprising a tumor antigen polypeptide (e.g., as described herein) treats or prevents cancer by one or more tumors characterized by expression of such a tumor or cancer antigen. In some embodiments, upon administration to a subject, an immunogenic composition (e.g., vaccine) or pharmaceutical composition described herein comprising a tumor antigen polypeptide (e.g., as described herein) reduces the severity of disease due to cancer by one or more tumors characterized by expression of such a tumor or cancer antigen. In some embodiments, upon administration to a subject, an immunogenic composition (e.g., vaccine) or pharmaceutical composition described herein comprising a tumor antigen polypeptide (e.g., as described herein) reduces the severity of one or more symptoms due to cancer by one or more tumors characterized by expression of such a tumor or cancer antigen. In some embodiments, a tumor or cancer antigen polypeptide (e.g., as described herein) present in an immunogenic composition (e.g., vaccine) or pharmaceutical composition described herein to be administered to a cancer subject is identified to be present in the subject's tumor or cancer cells.

In some embodiments, upon administration to a subject, an immunogenic composition (e.g., vaccine) or pharmaceutical composition described herein comprising a tumor antigen polypeptide (e.g., as described herein) inhibits or reduces the rate of occurrence or recurrence of cancer by one or more tumors characterized by expression of such a tumor or cancer antigen. In some embodiments, upon administration to a subject, an immunogenic composition (e.g., vaccine) or pharmaceutical composition described herein comprising a tumor antigen polypeptide (e.g., as described herein) inhibits or reduces the rate of occurrence or recurrence of disease due to cancer by one or more tumors characterized by expression of such a tumor or cancer antigen. In some embodiments, upon administration to a subject, an immunogenic composition (e.g., vaccine) or pharmaceutical composition described herein comprising a tumor antigen polypeptide (e.g., as described herein) inhibits or reduces the rate of occurrence or recurrence of one or more symptoms due to cancer by one or more tumors characterized by expression of such a tumor or cancer antigen.

In some embodiments, an immunogenic composition (e.g., vaccine) or pharmaceutical composition described herein is useful to slow down the progression of cancer or growth of a tumor. In some embodiments, In some embodiments, upon administration to a subject, an immunogenic composition (e.g., vaccine) or pharmaceutical composition described herein comprising a tumor antigen polypeptide (e.g., as described herein) reduces the severity of cancer by one or more tumors characterized by expression of such a tumor or cancer antigen. In some embodiments, upon administration to a subject, an immunogenic composition (e.g., vaccine) or pharmaceutical composition described herein comprising a tumor antigen polypeptide (e.g., as described herein) reduces the severity of disease due to cancer by one or more tumors characterized by expression of such a tumor or cancer antigen. In some embodiments, upon administration to a subject, an immunogenic composition (e.g., vaccine) or pharmaceutical composition described herein comprising a tumor antigen polypeptide (e.g., as described herein) reduces the severity of one or more symptoms due to cancer by one or more tumors characterized by expression of such a tumor or cancer antigen.

In some embodiments, upon administration to a subject, an immunogenic composition (e.g., vaccine) or pharmaceutical composition described herein inhibits transmission of one or more strains (variants) of a pathogen from the subject to another subject. In some embodiments, upon administration to a subject, an immunogenic composition (e.g., vaccine) or pharmaceutical composition described herein inhibits asymptomatic infection by one or more strains (variants) of a pathogen in the subject. In some embodiments, upon administration to a subject, an immunogenic composition (e.g., vaccine) or pharmaceutical composition described herein inhibits replication and/or reduces pathogen load of one or more strains (variants) of a pathogen in the subject.

In some embodiments, upon administration to a subject, an immunogenic composition (e.g., vaccine) or pharmaceutical composition described herein comprising a tumor antigen polypeptide (e.g., as described herein) inhibits metastasis of one or more tumors characterized by expression of such a tumor or cancer. In some embodiments, upon administration to a subject, an immunogenic composition (e.g., vaccine) or pharmaceutical composition described herein comprising a tumor antigen polypeptide (e.g., as described herein) inhibits asymptomatic cancer by one or more tumors characterized by expression of such a tumor or cancer antigen in the subject. In some embodiments, upon administration to a subject, an immunogenic composition (e.g., vaccine) or pharmaceutical composition described herein comprising a tumor antigen polypeptide (e.g., as described herein) inhibits replication and/or reduces tumor load of one or more tumors characterized by expression of such a tumor or cancer antigen in the subject.

In some embodiments, an immunogenic composition (e.g., vaccine) or pharmaceutical composition described herein, upon administration to a subject, induces an immune response against one or more tumors or pathogens in the subject at a level greater than a control composition. In some embodiments, an immunogenic composition (e.g., vaccine) or pharmaceutical composition described herein, upon administration to a subject, induces a protective immune response against one or more tumors or pathogens in the subject at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, the subject is a human. In some embodiments, the human is between about 2 weeks of age and about 6 weeks of age. In some embodiments, the human is between about 6 weeks of age and about 6 years of age. In some embodiments, the human is between about 6 years of age and about 18 years of age. In some embodiments, the human is between about 18 years of age and about 50 years of age. In some embodiments, the human is about 50 years of age and about 75 years of age. In some embodiments, the human is about 75 years of age or older.

In some embodiments, an immunogenic composition (e.g., vaccine) or pharmaceutical composition described herein, upon administration to a subject, induces an immune response against one or more strains (variants) of a pathogen at a level greater than a control composition. In some embodiments, an immunogenic composition (e.g., vaccine) or pharmaceutical composition described herein comprising a tumor antigen polypeptide (e.g., as described herein), upon administration to a subject, induces an immune response against one or more tumors characterized by expression of such a tumor or cancer antigen at a level greater than a control composition. In some embodiments, the immune response is an antibody or B cell response. In some embodiments, the immune response is a T cell response. In some embodiments, the immune response is an innate immune response. In some embodiments, the immune response is a CD4+ T cell response, including T_{H}1, T_{H}2, or T_{H}17 response, or a CD8+ T cell response, or a CD4+ and CD8+ T cell response, or CD4−/CD8− T cell response. In some embodiments, the immune response is an antibody or B cell response, and a T cell response. In some embodiments, the immune response is an antibody or B cell response, a T cell response, and an innate immune response. In some embodiments, the immune response is a protective immune response. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, an immunogenic composition (e.g., vaccine) or pharmaceutical composition described herein, upon administration to a subject, induces an antibody or B cell response against one or more tumors or pathogens in the subject at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, an immunogenic composition (e.g., vaccine) or pharmaceutical composition described herein, upon administration to a subject, induces an antibody or B cell response against one or more tumors or pathogens in the subject at level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, an immunogenic composition (e.g., vaccine) or pharmaceutical composition described herein, upon administration to a subject, induces a T cell response against one or more tumors or pathogens in the subject at a level greater than a control composition. In some embodiments, an immunogenic composition (e.g., vaccine) or pharmaceutical composition described herein, upon administration to a subject, induces a T cell response against one or more tumors or pathogens in the subject at level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, upon administration to a subject, an immunogenic composition (e.g., vaccine) or pharmaceutical composition described herein treats or prevents infection by one or more strains (variants) of a pathogen in the subject at a level greater than a control composition. In some embodiments, upon administration to a subject, an immunogenic composition (e.g., vaccine) or pharmaceutical composition described herein treats or prevents disease due to infection by one or more strains (variants) of a pathogen in the subject at a level greater than a control composition. In some embodiments, upon administration to a subject, an immunogenic composition (e.g., vaccine) or pharmaceutical composition described herein treats or prevents one or more symptoms due to infection by one or more strains (variants) of a pathogen in the subject at a level greater than a control composition. In some embodiments, the one or more symptoms include pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), and/or multi-inflammatory syndrome of children or adults (MIS-C, MIS-A). In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, upon administration to a subject, an immunogenic composition (e.g., vaccine) or pharmaceutical composition described herein inhibits or reduces the rate of occurrence or recurrence of infection by one or more strains (variants) of a pathogen in the subject at a level greater than a control composition. In some embodiments, upon administration to a subject, an immunogenic composition (e.g., vaccine) or pharmaceutical composition described herein inhibits or reduces the rate of occurrence or recurrence of disease due to infection by one or more strains (variants) of a pathogen in the subject at a level greater than a control composition. In some embodiments, upon administration to a subject, an immunogenic composition (e.g., vaccine) or pharmaceutical composition described herein inhibits or reduces the rate of occurrence or recurrence of one or more symptoms due to infection by one or more strains (variants) of a pathogen in the subject at a level greater than a control composition. In some embodiments, the one or more symptoms include pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), and/or multi-inflammatory syndrome of children or adults (MIS-C, MIS-A). In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, upon administration to a subject, an immunogenic composition (e.g., vaccine) or pharmaceutical composition described herein reduces the severity of infection by one or more strains (variants) of a pathogen in the subject at a level greater than a control composition. In some embodiments, upon administration to a subject, an immunogenic composition (e.g., vaccine) or pharmaceutical composition described herein reduces the severity of disease due to infection by one or more strains (variants) of a pathogen in the subject at a level greater than a control composition. In some embodiments, upon administration to a subject, an immunogenic composition (e.g., vaccine) or pharmaceutical composition described herein reduces the severity of one or more symptoms due to infection by one or more strains (variants) of a pathogen in the subject at a level greater than a control composition. In some embodiments, the one or more symptoms include pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), and/or multi-inflammatory syndrome of children or adults (MIS-C, MIS-A). In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, upon administration to a subject, an immunogenic composition (e.g., vaccine) or pharmaceutical composition described herein comprising a tumor antigen polypeptide (e.g., as described herein) treats or prevents cancer by one or more tumors characterized by expression of such a tumor or cancer antigen in the subject at a level greater than a control composition. In some embodiments, upon administration to a subject, an immunogenic composition (e.g., vaccine) or pharmaceutical composition described herein comprising a tumor antigen polypeptide (e.g., as described herein) treats or prevents disease due to cancer by one or more tumors characterized by expression of such a tumor or cancer antigen in the subject at a level greater than a control composition. In some embodiments, upon administration to a subject, an immunogenic composition (e.g., vaccine) or pharmaceutical composition described herein comprising a tumor antigen polypeptide (e.g., as described herein) treats or prevents one or more symptoms due to cancer by one or more tumors characterized by expression of such a tumor or cancer antigen in the subject at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, upon administration to a subject, an immunogenic composition (e.g., vaccine) or pharmaceutical composition described herein comprising a tumor antigen polypeptide (e.g., as described herein) inhibits or reduces the rate of occurrence or recurrence of cancer by one or more tumors characterized by expression of such a tumor or cancer antigen in the subject at a level greater than a control composition. In some embodiments, upon administration to a subject, an immunogenic composition (e.g., vaccine) or pharmaceutical composition described herein comprising a tumor antigen polypeptide (e.g., as described herein) inhibits or reduces the rate of occurrence or recurrence of disease due to cancer by one or more tumors characterized by expression of such a tumor or cancer antigen in the subject at a level greater than a control composition. In some embodiments, upon administration to a subject, an immunogenic composition (e.g., vaccine) or pharmaceutical composition described herein comprising a tumor antigen polypeptide (e.g., as described herein) inhibits or reduces the rate of occurrence or recurrence of one or more symptoms due to cancer by one or more tumors characterized by expression of such a tumor or cancer antigen in the subject at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, upon administration to a subject, an immunogenic composition (e.g., vaccine) or pharmaceutical composition described herein comprising a tumor antigen polypeptide (e.g., as described herein) reduces the severity of cancer by one or more tumors characterized by expression of such a tumor or cancer antigen in the subject at a level greater than a control composition. In some embodiments, upon administration to a subject, an immunogenic composition (e.g., vaccine) or pharmaceutical composition described herein comprising a tumor antigen polypeptide (e.g., as described herein) reduces the severity of disease due to cancer by one or more tumors characterized by expression of such a tumor or cancer antigen in the subject at a level greater than a control composition. In some embodiments, upon administration to a subject, an immunogenic composition (e.g., vaccine) or pharmaceutical composition described herein comprising a tumor antigen polypeptide (e.g., as described herein) reduces the severity of one or more symptoms due to cancer by one or more tumors characterized by expression of such a tumor or cancer antigen in the subject at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, upon administration to a subject, an immunogenic composition (e.g., vaccine) or pharmaceutical composition described herein inhibits transmission of one or more strains (variants) of a pathogen from the subject to another subject at a level greater than a control composition. In some embodiments, upon administration to a subject, an immunogenic composition (e.g., vaccine) or pharmaceutical composition described herein inhibits asymptomatic infection by one or more strains (variants) of a pathogen in the subject at a level greater than a control composition. In some embodiments, upon administration to a subject, an immunogenic composition (e.g., vaccine) or pharmaceutical composition described herein inhibits replication and/or reduces pathogen load of one or more strains (variants) of a pathogen in the subject at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, upon administration to a subject, an immunogenic composition (e.g., vaccine) or pharmaceutical composition described herein comprising a tumor antigen polypeptide (e.g., as described herein) inhibits metastasis of one or more tumors characterized by expression of such a tumor or cancer antigen at a level greater than a control composition. In some embodiments, upon administration to a subject, an immunogenic composition (e.g., vaccine) or pharmaceutical composition described herein comprising a tumor antigen polypeptide (e.g., as described herein) inhibits asymptomatic cancer by one or more tumors characterized by expression of such a tumor or cancer antigen in the subject at a level greater than a control composition. In some embodiments, upon administration to a subject, an immunogenic composition (e.g., vaccine) or pharmaceutical composition described herein comprising a tumor antigen polypeptide (e.g., as described herein) inhibits replication and/or reduces tumor load of one or more tumors characterized by expression of such a tumor or cancer antigen in the subject at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, an immunogenic composition (e.g., vaccine) or pharmaceutical composition described herein is administered to a subject between about 6 weeks and about 5 years (e.g., prior to the 6^{th} birthday) for active immunization for the prevention of disease caused by one or more strains (variants) of a pathogen. In some embodiments, an immunogenic composition (e.g., vaccine) or pharmaceutical composition described herein comprising a tumor antigen polypeptide (e.g., as described herein) is administered to a subject between about 6 weeks and about 5 years (e.g., prior to the 6^{th} birthday) for active immunization for the prevention of disease caused by one or more tumors characterized by expression of such a tumor or cancer antigen.

In some embodiments, an immunogenic composition (e.g., vaccine) or pharmaceutical composition described herein is administered to a subject between about 6 years and about 17 years (e.g., prior to the 18^{th} birthday) for active immunization for the prevention of disease caused by one or more strains (variants) of a pathogen. In some embodiments, an immunogenic composition (e.g., vaccine) or pharmaceutical composition described herein comprising a tumor antigen polypeptide (e.g., as described herein) is administered to a subject between about 6 years to about 17 years (e.g., prior to the 18^{th} birthday) for active immunization for the prevention of disease caused by one or more tumors characterized by expression of such a tumor or cancer antigen.

In some embodiments, an immunogenic composition (e.g., vaccine) or pharmaceutical composition described herein is administered to a subject 18 years or older for active immunization for the prevention of disease caused by one or more strains (variants) of a pathogen. In some embodiments, an immunogenic composition (e.g., vaccine) or pharmaceutical composition described herein comprising a tumor antigen polypeptide (e.g., as described herein) is administered to a subject 18 years or older for active immunization for the prevention of disease caused by one or more tumors characterized by expression of such a tumor or cancer antigen.

### Antibody Compositions

Some embodiments provide for an antibody composition comprising antibodies raised in a mammal immunized with an immunogenic complex or immunogenic composition of the present disclosure. In some embodiments, an antibody comprises at least one antibody selected from the group consisting of mAbs and anti-idiotype antibodies. In some embodiments, an antibody composition comprises neutralizing antibodies. In some embodiments, an antibody composition comprises an isolated gamma globulin fraction. In some embodiments, an antibody composition comprises polyclonal antibodies. In some embodiments, the antibody composition is administered to a subject. In some embodiments, the antibody composition administered to a subject confers passive immunization.

### Methods of Administration

Any effective route of administration of an immunogenic composition (e.g., vaccine) or pharmaceutical composition described herein may be utilized such as, for example, oral, nasal, enteral, parenteral, intramuscular or intravenous, subcutaneous, transdermal, intradermal, rectal, vaginal, topical, ocular, pulmonary, or by contact application. In some embodiments, immunogenic compositions (e.g., vaccines) or pharmaceutical compositions may be injected (e.g., via intramuscular, intraperitoneal, intradermal and/or subcutaneous routes); or delivered via the mucosa (e.g., to the oral/alimentary, respiratory, and/or genitourinary tracts). In some embodiments, it may be desirable to administer different doses of an immunogenic composition (e.g., vaccine) or pharmaceutical composition by different routes; in some embodiments, it may be desirable to administer different components of one dose via different routes. In some embodiments, an immunogenic composition (e.g., vaccine) or pharmaceutical composition disclosed herein is administered intramuscularly. In some embodiments, an immunogenic composition (e.g., vaccine) or pharmaceutical composition disclosed herein is administered subcutaneously.

In some embodiments, immunogenic compositions (e.g., vaccines) or pharmaceutical compositions are administered intradermally. Conventional technique of intradermal injection, the “Mantoux procedure”, comprises steps of cleaning the skin, and then stretching with one hand, and with the bevel of a narrow gauge needle (26-31 gauge) facing upwards the needle is inserted at an angle of between 10-15°. Once the bevel of the needle is inserted, the barrel of the needle is lowered and further advanced while providing a slight pressure to elevate it under the skin. The liquid is then injected very slowly thereby forming a bleb or bump on the skin surface, followed by slow withdrawal of the needle.

Devices that are specifically designed to administer liquid agents into or across the skin have been described, for example the devices described in WO 99/34850 and EP 1092444, also the jet injection devices described for example in WO 01/13977; U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO 97/37705 and WO 97/13537. Other methods of intradermal administration of the vaccine preparations may include conventional syringes and needles, or devices designed for ballistic delivery of solid vaccines (WO 99/27961), or transdermal patches (WO 97/48440; WO 98/28037); or applied to the surface of the skin (transdermal or transcutaneous delivery WO 98/20734; WO 98/28037).

As described above, immunogenic compositions (e.g., vaccines) or pharmaceutical compositions may be administered as a single dose or as multiple doses. It will be appreciated that an administration is a single “dose” so long as all relevant components are administered to a subject within a window of time; it is not necessary that every component be present in a single composition. For example, administration of two different immunogenic compositions (e.g., vaccines) or pharmaceutical compositions, within a period of less than 24 h, is considered a single dose. To give but one example, immunogenic compositions (e.g., vaccines) or pharmaceutical compositions having different antigenic components may be administered in separate compositions, but as part of a single dose. As noted above, such separate compositions may be administered via different routes or via the same route. Alternatively or additionally, in embodiments wherein an immunogenic composition (e.g., vaccine) or pharmaceutical composition comprises a combination of immunogenic compositions and additional types of active agents, immunogenic compositions may be administered via one route, and an additional active agent may be administered by the same route or by a different route.

Immunogenic compositions (e.g., vaccines) and pharmaceutical compositions are administered in such amounts and for such time as is necessary to achieve a desired result. In certain embodiments of the present disclosure, an immunogenic composition (e.g., vaccine) or pharmaceutical composition comprises an immunologically effective amount of at least immunogenic composition. The exact amount required to achieve an immunologically effective amount may vary, depending on the immunogenic composition, and from subject to subject, depending on the species, age, and general condition of the subject, the stage of the disease, the particular pharmaceutical mixture, its mode of administration, and the like.

The amount of polypeptide(s) (e.g., antigenic polypeptide(s)), polymer(s) (e.g., antigenic polysaccharide(s)), or conjugate(s) in each immunogenic composition (e.g., vaccine) or pharmaceutical composition dose is selected to allow the composition, when administered as described herein, to induce an appropriate immune-protective response without significant, adverse side effects.

In some embodiments, administration of an immunogenic composition (e.g., vaccine) or pharmaceutical composition described herein may involve the delivery of a single dose. In some embodiments, administration may involve an initial dose followed by one or several additional immunization doses, adequately spaced. An immunization schedule or regimen is a program for the administration of one or more specified doses of one or more specified immunogenic compositions (e.g., vaccines) or pharmaceutical compositions, by one or more specified routes of administration, at one or more specified ages of a subject.

Immunization schedules of the present disclosure are provided to induce an immune response (e.g., an immunoprotective response) in a subject sufficient to reduce at least one measure selected from the group consisting of incidence, prevalence, frequency, and/or severity of at least one infection, disease, or disorder, and/or at least one surrogate marker of the infection, disease, or disorder, in a population and/or subpopulation of the subject(s). A supplemental immunization schedule is one which has this effect relative to the standard schedule which it supplements. A supplemental schedule may call for additional administrations and/or supra-immunogenic doses of the immunogenic compositions (e.g., vaccines) or pharmaceutical compositions disclosed herein, found in the standard schedule, or for the administration of immunogenic compositions (e.g., vaccines) or pharmaceutical compositions not part of the standard schedule. A full immunization schedule of the present disclosure may comprise both a standard schedule and a supplemental schedule. Exemplary sample immunization schedules are provided for illustrative purposes. Detailed descriptions of methods to assess immunogenic response discussed herein allow one to develop alterations to the sample immunization schedules without undue experimentation.

In some embodiments of the present disclosure, a first administration of an immunogenic composition (e.g., vaccine) or pharmaceutical composition occurs when a subject is more than about 2 weeks old, more than about 5 weeks old, more than about 1 year old, more than about 2 years old, more than about 15 years old, or more than about 18 years old.

In some embodiments, a first administration of an immunogenic composition (e.g., vaccine) or pharmaceutical composition occurs when a subject is about two months old. In some embodiments, a second administration of an immunogenic composition (e.g., vaccine) or pharmaceutical composition occurs when a subject is about four months old. In some embodiments, a third administration of an immunogenic composition (e.g., vaccine) or pharmaceutical composition occurs when a subject is about six months old. In some embodiments, a fourth administration of an immunogenic composition (e.g., vaccine) or pharmaceutical composition occurs when a subject is between about twelve months old and about fifteen months old.

In some embodiments of the present disclosure, a first administration of an immunogenic composition (e.g., vaccine) or pharmaceutical composition occurs when a subject is more than about 18 years old, more than about 50 years old, more than about 55 years old, more than about 60 years old, more than about 65 years old, or more than about 70 years old.

In some embodiments of the disclosure, a single administration of an immunogenic composition (e.g., vaccine) or pharmaceutical composition is employed. It is possible that the purposes of the present disclosure can be served with a single administration, especially when one or more utilized polypeptide(s) (e.g., antigenic polypeptide(s)), polymers (e.g., polysaccharide(s)), and/or immunogenic complex(es) or combinations thereof is/are strong, and in such a situation a single dose schedule is sufficient to induce a lasting immune-protective response.

In certain embodiments, it is desirable to administer two or more doses of an immunogenic composition (e.g., vaccine) or pharmaceutical composition, for greater immune-protective efficacy and coverage. Thus, in some embodiments, a number of doses is at least two, at least three or more doses. There is no set maximum number of doses, however it is good clinical practice not to immunize more often than necessary to achieve the desired effect.

Without being bound by theory, a first dose of an immunogenic composition (e.g., vaccine) or pharmaceutical composition administered according to the disclosure may be considered a “priming” dose. In certain embodiments, more than one dose is included in an immunization schedule. In such a scenario, a subsequent dose may be considered a “boosting” dose.

A priming dose may be administered to a naïve subject (a subject who has never previously received an immunogenic composition (e.g., vaccine) or pharmaceutical composition). In some embodiments, a priming dose may be administered to a subject who has previously received an immunogenic composition (e.g., vaccine) or pharmaceutical composition at least five or more years previous to administration of an initial immunogenic composition (e.g., vaccine) or pharmaceutical composition dose according to the present disclosure. In other embodiments, a priming dose may be administered to a subject who has previously received an immunogenic composition (e.g., vaccine) or pharmaceutical composition at least twenty or more years previous to administration of a priming immunogenic composition (e.g., vaccine) or pharmaceutical composition according to the disclosure.

When an immunization schedule calls for two or more separate doses, the interval between doses is considered. The interval between two successive doses may be the same throughout an immunization schedule, or it may change as the subject ages. In immunization schedules of the present disclosure, once a first immunogenic composition (e.g., vaccine) or pharmaceutical composition dose has been administered, there is a first interval before administration of a subsequent dose. A first interval is generally at least about 2 weeks, 1 month, 6 weeks, 2 months, 3 months, 6 months, 9 months, 12 months, or longer. Where more than one subsequent dose(s) are administered, second (or higher) intervals may be provided between such subsequent doses. In some embodiments, all intervals between subsequent doses are of the same length; in other embodiments, second intervals may vary in length. In some embodiments, the interval between subsequent doses may be at least about 12 months, at least about 15 months, at least about 18 months, at least about 21 months or at least about 2 years. In certain embodiments, the interval between doses may be up to 3 years, up to about 4 years, or up to about 5 years or 10 years or more. In certain embodiments, intervals between subsequent doses may decrease as the subject ages.

It will be appreciated by those skilled in the art that a variety of possible combinations and sub-combinations of the various conditions of timing of the first administration, shortest interval, largest interval and total number of administrations (in absolute terms, or within a stated period) exist, and all of these combinations and sub-combinations should be considered to be within the inventor's contemplation though not explicitly enumerated here.

### Exemplary Assays for Determining Immune Response

In some embodiments, a method of assessing the immunogenicity of an immunogenic composition described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by opsonophagocytic killing (OPK, OPA), plaque reduction neutralization test (PRNT), serum bactericidal killing (SBA), agglutination, motility, cytotoxicity, or adherence; and in vivo assays in animal models of disease. In some embodiments, where the disease is a viral disease, parameters of in vivo assays include viral clearance, reduction of mortality, and passive and active protection following challenge with one or more strains (variants) of a virus that are the targets of the immunogenic composition. In some embodiments, where the disease is a bacterial disease, parameters of in vivo assays include bacterial clearance from mucosal surfaces or bloodstream, reduction or prevention of bacteremia, meningitis, sepsis, or otitis media, reduction or prevention of colonization of the nasopharynx, inhibition or reduction of asymptomatic infection, reduction of mortality, and passive and active protection following challenge with one or more strains (variants) of bacteria that are the targets of the immunogenic composition. In some embodiments, where the disease is a cancer or tumor, parameters of in vivo assays include reduction in tumor size and/or number, reduction or prevention of tumor metastasis, inhibition or reduction of asymptomatic cancer, reduction in mortality, and passive and active protection following challenge with one or more tumors characterized by expression of one or more tumor or cancer antigens that are the targets of the immunogenic composition. In some embodiments, the immune response is compared to a control composition. In some embodiments, a control composition may comprise a polymer (e.g., an antigenic polysaccharide) present in the immunogenic composition and not comprise a polypeptide (e.g., an antigenic polypeptide) present in the immunogenic composition. In some embodiments, a control composition may comprise a polypeptide (e.g., an antigenic polypeptide) present in the immunogenic composition and not comprise a polymer (e.g., an antigenic polysaccharide) present in the immunogenic composition. In some embodiments, a control composition may comprise an adjuvant present in the immunogenic composition, and not comprise a polymer (e.g., an antigenic polysaccharide) and/or a polypeptide (e.g., an antigenic polypeptide) present in the immunogenic composition.

In some embodiments, a method of assessing the potency of an immunogenic composition described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by OPK (OPA), plaque reduction neutralization test (PRNT), serum bactericidal killing (SBA), internalization, activity neutralization, agglutination, motility, cytotoxicity, or adherence; and in vivo assays in animal models of disease. In some embodiments, where the disease is a viral disease, parameters of in vivo assays include viral clearance, reduction of mortality, and passive and active protection following challenge with one or more strains (variants) of a virus that are the targets of the immunogenic composition. In some embodiments, where the disease is a bacterial disease, parameters of in vivo assays include bacterial clearance or reduction from mucosal surfaces or bloodstream, reduction or prevention of bacteremia, meningitis, sepsis, or otitis media, reduction or prevention of colonization of the nasopharynx, inhibition or reduction of asymptomatic infection, reduction of mortality, and passive and active protection following challenge with one or more strains (variants) of bacteria that are the targets of the immunogenic composition. In some embodiments, where the disease is a cancer or tumor, parameters of in vivo assays include reduction in tumor size and/or number, reduction or prevention of tumor metastasis, inhibition or reduction of asymptomatic cancer, reduction in mortality, and passive and active protection following challenge with one or more tumors characterized by expression of one or more tumor or cancer antigens that are the targets of the immunogenic composition. In some embodiments, the immune response is compared to a control composition. In some embodiments, a control composition may comprise a polymer (e.g., an antigenic polysaccharide) present in the immunogenic composition and not comprise a polypeptide (e.g., an antigenic polypeptide) present in the immunogenic composition. In some embodiments, a control composition may comprise a polypeptide (e.g., an antigenic polypeptide) present in the immunogenic composition and not comprise a polymer (e.g., an antigenic polysaccharide) present in the immunogenic composition. In some embodiments, a control composition may comprise an adjuvant present in the immunogenic composition, and not comprise a polymer (e.g., an antigenic polysaccharide) and/or a polypeptide (e.g., an antigenic polypeptide) present in the immunogenic composition.

In some embodiments, a method of assessing the immunogenicity of an vaccine composition described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by opsonophagocytic killing (OPK, OPA), plaque reduction neutralization test (PRNT), serum bactericidal killing (SBA), agglutination, motility, cytotoxicity, or adherence; and in vivo assays in animal models of disease. In some embodiments, where the disease is a viral disease, parameters of in vivo assays include viral clearance, reduction of mortality, and passive and active protection following challenge with one or more strains (variants) of a virus that are the targets of the vaccine composition. In some embodiments, where the disease is a bacterial disease, parameters of in vivo assays include bacterial clearance from mucosal surfaces or bloodstream, reduction or prevention of bacteremia, meningitis, sepsis, or otitis media, reduction or prevention of colonization of the nasopharynx, inhibition or reduction of asymptomatic infection, reduction of mortality, and passive and active protection following challenge with one or more strains (variants) of bacteria that are the targets of the vaccine composition. In some embodiments, where the disease is a cancer or tumor, parameters of in vivo assays include reduction in tumor size and/or number, reduction or prevention of tumor metastasis, inhibition or reduction of asymptomatic cancer, reduction in mortality, and passive and active protection following challenge with one or more tumors characterized by expression of one or more tumor or cancer antigens that are the targets of the immunogenic composition. In some embodiments, the immune response is compared to a control composition. In some embodiments, a control composition may comprise a polymer (e.g., an antigenic polysaccharide) present in the vaccine composition and not comprise a polypeptide (e.g., an antigenic polypeptide) present in the vaccine composition. In some embodiments, a control composition may comprise a polypeptide (e.g., an antigenic polypeptide) present in the vaccine composition and not comprise a polymer (e.g., an antigenic polysaccharide) present in the vaccine composition. In some embodiments, a control composition may comprise an adjuvant present in the vaccine composition, and not comprise a polymer (e.g., an antigenic polysaccharide) and/or a polypeptide (e.g., an antigenic polypeptide) present in the vaccine composition.

In some embodiments, a method of assessing the potency of an vaccine composition described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by OPK (OPA), plaque reduction neutralization test (PRNT), serum bactericidal killing (SBA), internalization, activity neutralization, agglutination, motility, cytotoxicity, or adherence; and in vivo assays in animal models of disease. In some embodiments, where the disease is a viral disease, parameters of in vivo assays include viral clearance, reduction of mortality, and passive and active protection following challenge with one or more strains (variants) of a virus that are the targets of the vaccine composition. In some embodiments, where the disease is a bacterial disease, parameters of in vivo assays include bacterial clearance or reduction from mucosal surfaces or bloodstream, reduction or prevention of bacteremia, meningitis, sepsis, or otitis media, reduction or prevention of colonization of the nasopharynx, inhibition or reduction of asymptomatic infection, reduction of mortality, and passive and active protection following challenge with one or more strains (variants) of bacteria that are the targets of the vaccine composition. In some embodiments, where the disease is a cancer or tumor, parameters of in vivo assays include reduction in tumor size and/or number, reduction or prevention of tumor metastasis, inhibition or reduction of asymptomatic cancer, reduction in mortality, and passive and active protection following challenge with one or more tumors characterized by expression of one or more tumor or cancer antigens that are the targets of the immunogenic composition. In some embodiments, the immune response is compared to a control composition. In some embodiments, a control composition may comprise a polymer (e.g., an antigenic polysaccharide) present in the vaccine composition and not comprise a polypeptide (e.g., an antigenic polypeptide) present in the vaccine composition. In some embodiments, a control composition may comprise a polypeptide (e.g., an antigenic polypeptide) present in the vaccine composition and not comprise a polymer (e.g., an antigenic polysaccharide) present in the vaccine composition. In some embodiments, a control composition may comprise an adjuvant present in the vaccine composition, and not comprise a polymer (e.g., an antigenic polysaccharide) and/or a polypeptide (e.g., an antigenic polypeptide) present in the vaccine composition.

In some embodiments, a method of assessing the immunogenicity of an pharmaceutical composition described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by opsonophagocytic killing (OPK, OPA), plaque reduction neutralization test (PRNT), serum bactericidal killing (SBA), agglutination, motility, cytotoxicity, or adherence; and in vivo assays in animal models of disease. In some embodiments, where the disease is a viral disease, parameters of in vivo assays include viral clearance, reduction of mortality, and passive and active protection following challenge with one or more strains (variants) of a virus that are the targets of the pharmaceutical composition. In some embodiments, where the disease is a bacterial disease, parameters of in vivo assays include bacterial clearance from mucosal surfaces or bloodstream, reduction or prevention of bacteremia, meningitis, sepsis, or otitis media, reduction or prevention of colonization of the nasopharynx, inhibition or reduction of asymptomatic infection, reduction of mortality, and passive and active protection following challenge with one or more strains (variants) of bacteria that are the targets of the pharmaceutical composition. In some embodiments, where the disease is a cancer or tumor, parameters of in vivo assays include reduction in tumor size and/or number, reduction or prevention of tumor metastasis, inhibition or reduction of asymptomatic cancer, reduction in mortality, and passive and active protection following challenge with one or more tumors characterized by expression of one or more tumor or cancer antigens that are the targets of the immunogenic composition. In some embodiments, the immune response is compared to a control composition. In some embodiments, a control composition may comprise a polymer (e.g., an antigenic polysaccharide) present in the pharmaceutical composition and not comprise a polypeptide (e.g., an antigenic polypeptide) present in the pharmaceutical composition. In some embodiments, a control composition may comprise a polypeptide (e.g., an antigenic polypeptide) present in the pharmaceutical composition and not comprise a polymer (e.g., an antigenic polysaccharide) present in the pharmaceutical composition. In some embodiments, a control composition may comprise an adjuvant present in the pharmaceutical composition, and not comprise a polymer (e.g., an antigenic polysaccharide) and/or a polypeptide (e.g., an antigenic polypeptide) present in the pharmaceutical composition.

In some embodiments, a method of assessing the potency of an pharmaceutical composition described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by OPK (OPA), plaque reduction neutralization test (PRNT), serum bactericidal killing (SBA), internalization, activity neutralization, agglutination, motility, cytotoxicity, or adherence; and in vivo assays in animal models of disease. In some embodiments, where the disease is a viral disease, parameters of in vivo assays include viral clearance, reduction of mortality, and passive and active protection following challenge with one or more strains (variants) of a virus that are the targets of the pharmaceutical composition. In some embodiments, where the disease is a bacterial disease, parameters of in vivo assays include bacterial clearance or reduction from mucosal surfaces or bloodstream, reduction or prevention of bacteremia, meningitis, sepsis, or otitis media, reduction or prevention of colonization of the nasopharynx, inhibition or reduction of asymptomatic infection, reduction of mortality, and passive and active protection following challenge with one or more strains (variants) of bacteria that are the targets of the pharmaceutical composition. In some embodiments, where the disease is a cancer or tumor, parameters of in vivo assays include reduction in tumor size and/or number, reduction or prevention of tumor metastasis, inhibition or reduction of asymptomatic cancer, reduction in mortality, and passive and active protection following challenge with one or more tumors characterized by expression of one or more tumor or cancer antigens that are the targets of the immunogenic composition. In some embodiments, the immune response is compared to a control composition. In some embodiments, a control composition may comprise a polymer (e.g., an antigenic polysaccharide) present in the pharmaceutical composition and not comprise a polypeptide (e.g., an antigenic polypeptide) present in the pharmaceutical composition. In some embodiments, a control composition may comprise a polypeptide (e.g., an antigenic polypeptide) present in the pharmaceutical composition and not comprise a polymer (e.g., an antigenic polysaccharide) present in the pharmaceutical composition. In some embodiments, a control composition may comprise an adjuvant present in the pharmaceutical composition, and not comprise a polymer (e.g., an antigenic polysaccharide) and/or a polypeptide (e.g., an antigenic polypeptide) present in the pharmaceutical composition.

In some embodiments, a method of assessing the immunogenicity and/or potency of an immunogenic complex comprises evaluating an immune response to immunogenic or vaccine compositions comprising one or more immunogenic complexes. In some embodiments, the method of assessing the immunogenicity and/or potency of an immunogenic complex described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by OPK, plaque reduction neutralization test (PRNT), serum bactericidal killing (SBA), agglutination, motility, cytotoxicity, or adherence; and in vivo assays in animal models of disease. In some embodiments, where the disease is a viral disease, parameters of in vivo assays include viral clearance, reduction of mortality, and passive and active protection following challenge with one or more strains (variants) of a virus that are the targets of the immunogenic composition. In some embodiments, where the disease is a bacterial disease, parameters of in vivo assays include bacterial clearance or reduction from mucosal surfaces or bloodstream, reduction or prevention of bacteremia, meningitis, sepsis, or otitis media, reduction or prevention of colonization of the nasopharynx, inhibition or reduction of asymptomatic infection, reduction of mortality, and passive and active protection following challenge with one or more strains (variants) of bacteria that are the targets of the immunogenic composition. In some embodiments, where the disease is a cancer or tumor, parameters of in vivo assays include reduction in tumor size and/or number, reduction or prevention of tumor metastasis, inhibition or reduction of asymptomatic cancer, reduction in mortality, and passive and active protection following challenge with one or more tumors characterized by expression of one or more tumor or cancer antigens that are the targets of the immunogenic composition.

Generally speaking, it may be desirable to assess humoral responses, cellular responses, and/or interactions between the two. Where humoral responses are being assessed, antibody titers and/or types (e.g., total IgG, IgG1, IgG2, IgM, IgA, etc.) to specific pathogen polysaccharides or polypeptides (either serotype-specific or conserved across two or more serotypes) may be determined, for example before and/or after administration of an initial or a boosting dose of vaccine (and/or as compared with antibody levels in the absence of antigenic stimulation). Cellular responses may be assessed by monitoring reactions such as delayed type hypersensitivity responses, etc. to the carrier protein. Cellular responses can also be measured directly by evaluating the response of peripheral blood mononuclear cells (PBMCs) monocytes to stimulation with the antigens of interest. Precursor and memory B cell populations may be assessed in enzyme linked immunospot (ELISpot) assays directed against specific pathogen polysaccharides or polypeptides.

Any of a variety of assays may be employed to detect levels and/or activity of antibodies in subject sera. Suitable assays include, for example, ligand binding assays, such as radioimmunoassay (RIAs), ELISAs, and multiplex assays (Luminex, Bioplex, MSD); functional assays, such as opsonophagocytic assays or internalization assays; and in vivo assays in animal models of disease. In some embodiments, where the disease is a viral disease, parameters of in vivo assays include viral clearance, reduction of mortality, and passive and active protection following challenge with one or more strains (variants) of a virus that are the targets of the immunogenic composition. In some embodiments, where the disease is a bacterial disease, parameters of in vivo assays include bacterial clearance or reduction from mucosal surfaces or bloodstream, reduction or prevention of bacteremia, meningitis, sepsis, or otitis media, reduction or prevention of colonization of the nasopharynx, inhibition or reduction of asymptomatic infection, reduction of mortality, and passive and active protection following challenge with one or more strains (variants) of bacteria that are the targets of the immunogenic composition. In some embodiments, where the disease is a cancer or tumor, parameters of in vivo assays include reduction in tumor size and/or number, reduction or prevention of tumor metastasis, inhibition or reduction of asymptomatic cancer, reduction in mortality, and passive and active protection following challenge with one or more tumors characterized by expression of one or more tumor or cancer antigens that are the targets of the immunogenic composition.

The RIA method detects specific antibodies through incubation of sera with radio-labeled polysaccharides or polypeptides in suspension (e.g., Schiffiman et al, 1980). The antigen-antibody complexes are then precipitated with ammonium sulfate and the radiolabeled pellets assayed for counts per minute (cpm).

In the ELISA detection method, specific antibodies from the sera of vaccinated subjects are quantitated by incubation with polysaccharides or polypeptides (either serotype-specific or conserved across two or more serotypes) which have been adsorbed to a solid support (e.g., Koskela and Leinonen, 1981; Kojima et al., 1990; Concepcion and Frasch, 2001). The bound antibody is detected using enzyme-conjugated secondary detection antibodies. The ELISA also allows isotyping and subclassing of the immune response (i.e., IgM vs. IgG or IgG1 vs. IgG2) by using isotype- or subclass-specific secondary antibodies and can be adapted to evaluate the avidity of the antibodies (Anttila et al, 1998; Romero-Steiner et al, 2005). Multiplex assays (e.g., Luminex) facilitate simultaneous detection of antibodies to multiple antigens. Capsular polysaccharide(s) or polypeptides are conjugated to spectrally distinct microspheres that are mixed and incubated with serum. The antibodies bound to the polysaccharides or polypeptides on the coated microspheres are detected using a secondary antibody (e.g., R-Phycoerythrin-conjugated goat anti-human IgG).

An approach for assessing functional antibody in serum is an opsonophagocytic assay (OPA) or a concentrated opsonophagocytic assay (COPA), which quantitates only the antibodies that can opsonize the bacteria, leading to ingestion and killing of the bacteria. The standard assay utilizes a human phagocytic effector cell, a source of complement, bacteria, and diluted sera. The assay readout is the serum endpoint titer at which there is ≥50% killing compared to bacteria incubated with complement and human cells alone (Romero-Steiner et al., 1997). This killing OPA can also be multiplexed by utilizing target strains of pathogen that carry different antibiotic resistance markers (Kim et al., 2003). Another type of multiplex opsonic assay is a nonkilling assay in which the uptake by phagocytic effector cells of fluorescent stained encapsulated pathogen or fluorescent microspheres conjugated with antigenic polysaccharides or polypeptides from a target pathogen in the presence of diluted sera plus a complement source is evaluated by flow cytometry (Martinez et al., 1999). Opsonic activity of serum antibody plus complement can also be evaluated by measuring the oxidative response of phagocytic human effector cells to ingested pathogen (Munro et al., 1985; Ojo-Amaize et al., 1995).

Certain in vivo model systems can be used to evaluate the protection afforded by serum antibodies induced by vaccines of the present disclosure. In such passive protection systems, mice or rats are challenged with the pathogen plus diluted sera, and the endpoint titer of the sera which provides protection against pneumonia, bacteremia, colonization of organs or tissues, or mortality is determined (Stack et al., 1998; Saeland et al., 2000).

In some embodiments, efficacy of immunization may be determined by assaying one or more cytokine levels by stimulating T cells from a subject after immunization. The one or more cytokine levels may be compared to the one or more cytokine levels in the same subject before immunization. Increased levels of the one or more cytokine, such as a 1.5 fold, 2-fold, 5-fold, 10-fold, 20-fold, 50-fold or 100-fold or more increase over pre-immunization cytokine levels, would indicate an increased response to the immunogenic composition (e.g., vaccine) or pharmaceutical composition. In some embodiments, the one or more cytokines are selected from GM-CSP; IL-1α; IL-1β; IL-2; IL-3; IL-4; IL-5; IL-6; IL-7; IL-8; IL-10; IL-12; IL-17A, IL-17F or other members of the IL-17 family; IL-22; IL-23; IFN-α; IFN-β; IFN-γ; MIP-1α; MIP-1β; TGF-β; TNFα, or TNF-β. In a non-limiting example, efficacy of immunization may be determined by assaying IL-17 levels (particularly IL-17A) by stimulating T cells from a subject after immunization. The IL-17 levels may be compared to IL-17 levels in the same subject before immunization. Increased IL-17 (e.g., IL-17A) levels, such as a 1.5 fold, 2-fold, 5-fold, 10-fold, 20-fold, 50-fold or 100-fold or more increase, would indicate an increased response to the immunogenic composition (e.g., vaccine) or pharmaceutical composition.

In some embodiments, one may assay neutrophils in the presence of T cells or antibodies from the patient for pathogen (e.g., viral or bacterial) or tumor killing. Increased pathogen (e.g., viral or bacterial) or tumor killing, such as a 1.5 fold, 2-fold, 5-fold, 10-fold, 20-fold, 50-fold or 100-fold or more increase, would indicate an increased response to the immunogenic composition (e.g., vaccine) or pharmaceutical composition. For example, one may measure T_{H}17 cell activation, where increased T_{H}17 cell activation, such as a 1.5 fold, 2-fold, 5-fold, 10-fold, 20-fold, 50-fold or 100-fold or more increase, correlates with an increased response to the immunogenic composition (e.g., vaccine) or pharmaceutical composition. In another non-limiting example, one may measure T_{H}1 cell activation, where increased T_{H}1 cell activation, such as a 1.5 fold, 2-fold, 5-fold, 10-fold, 20-fold, 50-fold or 100-fold or more increase, correlates with an increased response to the immunogenic composition (e.g., vaccine) or pharmaceutical composition. One may also measure levels of an antibody specific to the vaccine, where increased levels of the specific antibody, such as a 1.5 fold, 2-fold, 5-fold, 10-fold, 20-fold, 50-fold or 100-fold or more increase, are correlated with increased immunogenic composition (e.g., vaccine) or pharmaceutical composition efficacy. In certain embodiments, two or more of these assays are used. For example, one may measure IL-17 levels and the levels of immunogenic composition (e.g., vaccine) or pharmaceutical composition-specific antibody. Alternatively, one may follow epidemiological markers such as incidence of, severity of, or duration of pathogen (e.g., viral or bacterial) infection or tumor development in vaccinated individuals compared to unvaccinated individuals. Immunogenic composition (e.g., vaccine) or pharmaceutical composition efficacy may also be assayed in various model systems such as the mouse challenge model. For instance, BALB/c or C57BL/6 strains of mice may be used. After administering the test immunogenic composition (e.g., vaccine) or pharmaceutical composition to a subject (as a single dose or multiple doses), the experimenter administers a challenge dose of pathogen or tumor. In some cases, a challenge dose administered intranasally or intratracheally is sufficient to cause pathogen infection and/or a high rate of lethality in unvaccinated animals. In some cases, a challenge dose administered intranasally is sufficient to cause pathogen colonization (especially nasal colonization) in an unvaccinated animal, and in some cases a challenge dose administered via aspiration is sufficient to cause sepsis and a high rate of lethality in unvaccinated animals. In some cases, a challenge dose administered via intraperitoneal injection is sufficient to cause sepsis and a high rate of lethality in unvaccinated animals. In some cases, a challenge dose administered via intravenous injection is sufficient to cause sepsis and a high rate of lethality in unvaccinated animals. In some cases, a challenge dose administered via intraperitoneal injection is sufficient to cause tumor development and a high rate of lethality in unvaccinated animals. In some cases, a challenge dose administered via intravenous injection is sufficient to cause tumor development and a high rate of lethality in unvaccinated animals. One can then measure the reduction in infection, the reduction in colonization, reduction in tumor development, or the reduction in lethality in vaccinated animals.

Certain in vivo model systems can be used to evaluate the protection afforded by serum antibodies induced by immunogenic compositions (e.g., vaccines) or pharmaceutical compositions of the present disclosure. In such passive protection systems, mice or rats are challenged with the pathogen or the tumor plus diluted sera, and the endpoint titer of the sera which provides protection against bacteremia, colonization of organs or tissues, tumor development, or mortality is determined (see, e.g., Stack et al. 1998; Saeland et al. 2000).

### Nucleic Acids Encoding Provided Polypeptides (e.g., Variant Rhizavidin Polypeptides)

The present disclosure, among other things, provides nucleic acids encoding polypeptides (e.g., a variant rhizavidin polypeptide) described herein. Such nucleic acids may be present in a vector. Such nucleic acids may be present in the genome of a cell, e.g. a mammalian cell for production of a polypeptide provided herein (e.g., a variant rhizavidin polypeptide).

Nucleic acids encoding polypeptides provided herein (e.g., a variant rhizavidin polypeptide) may be modified to include codons that are optimized for expression in a particular cell type or organism. Codon optimized sequences are synthetic sequences, and preferably encode an identical polypeptide (or biologically active fragment of a full-length polypeptide which has substantially the same activity as the full-length polypeptide) encoded by a non-codon optimized reference polynucleotide. In some embodiments, a coding region of a nucleic acid encoding a polypeptide (e.g., a variant rhizavidin polypeptide) described herein, in whole or in part, may include an altered sequence to optimize codon usage for a particular cell type (e.g., a eukaryotic or prokaryotic cell). For example, a coding sequence described herein may be optimized for expression in bacterial cells. Alternatively, the coding sequence may be optimized for expression in a mammalian cell (e.g., a CHO cell), or for expression in a yeast or insect cell. Such a sequence may be described as a codon-optimized sequence.

Nucleic acid constructs of the present disclosure may be inserted into an expression vector or viral vector by methods known to the art, and nucleic acids may be operably linked to an expression control sequence. A vector comprising any nucleic acids or fragments thereof described herein is further provided by the present disclosure. Any nucleic acids or fragments thereof described herein can be cloned into any suitable vector and can be used to transform or transfect any suitable host. Selection of vectors and methods to construct them are commonly known to persons of ordinary skill in the art (see, e.g., “Recombinant DNA Part D,” Methods in Enzymology, Vol. 153, Wu and Grossman, eds., Academic Press (1987)).

Conventionally used techniques including, for example, electrophoresis, calcium phosphate precipitation, DEAE-dextran transfection, or lipofection, may be used to introduce a foreign nucleic acid (e.g., DNA or RNA) into a prokaryotic or eukaryotic host cell. Desirably, a vector may include regulatory sequences, such as transcription and/or translation initiation and/or termination codons, which are specific to the type of host (e.g., bacterium, fungus, plant, or animal) into which a vector is to be introduced, as appropriate and taking into consideration whether a vector is DNA or RNA. In some embodiments, a vector comprises regulatory sequences that are specific to a genus of a host cell. In some embodiments, a vector comprises regulatory sequences that are specific to a species of a host.

In addition to a replication system and an inserted nucleic acid, a nucleic acid construct can include one or more marker genes, which allow for selection of transformed or transfected hosts. Exemplary marker genes include, e.g., biocide resistance (e.g., resistance to antibiotics or heavy metals) or complementation in an auxotrophic host to provide prototrophy.

An expression vector can comprise a native or nonnative promoter operably linked to an isolated or purified nucleic acid as described above. Selection of promoters, e.g., strong, weak, inducible, tissue-specific, and/or developmental-specific, is within the skill of one in the art. Similarly, combining a nucleic acid as described above with a promoter is also within the skill of one in the art.

Suitable vectors include those designed for propagation and expansion and/or for expression. For example, a cloning vector may be selected from the pUC series, the pBluescript series (Stratagene, LaJolla, Calif.), the pET series (Novagen, Madison, Wis.), the pGEX series (Pharmacia Biotech, Uppsala, Sweden), the pcDNA3 series (Invitrogen) or the pEX series (Clontech, Palo Alto, Calif.). Bacteriophage vectors, such as AGT10, AGT11, AZapII (Stratagene), NEMBL4, and λNM1149, may be used. Examples of plant expression vectors that can be used include pBI110, pBI101.2, pBI101.3, pBI121, or pBIN19 (Clontech). Examples of animal expression vectors that can be used include pEUK-C1, pMAM, or pMAMneo (Clontech). The TOPO cloning system (Invitrogen, Carlsbad, CA) also can be used in accordance with the manufacturer's recommendations.

Additional sequences can be added to such cloning and/or expression sequences to optimize their function in cloning and/or expression, to aid in isolation of a nucleic acid encoding a polypeptide (e.g., a variant rhizavidin polypeptide) described herein, or to improve introduction of a nucleic acid into a cell. Use of cloning vectors, expression vectors, adapters, and linkers is well known in the art (see, e.g., Sambrook et al., Molecular Cloning, a Laboratory Manual, 2d edition, Cold Spring Harbor Press, Cold Spring Harbor, N.Y. (1989); and Ausubel et al., Current Protocols in Molecular Biology, Greene Publishing Associates and John Wiley & Sons, New York, N.Y. (1994), each of which is hereby incorporated by reference in its entirety).

In some embodiments, nucleic acids and vectors of the present disclosure are isolated and/or purified. The present disclosure also provides a composition comprising an isolated or purified nucleic acid, optionally in the form of a vector. Isolated nucleic acids and vectors may be prepared using standard techniques known in the art including, for example, alkali/SDS treatment, CsCl binding, column chromatography, agarose gel electrophoresis, and/or other techniques well known in the art. The composition can comprise other components as described further herein.

Any method known to one skilled in the art for the insertion of nucleic acids into a vector may be used to construct expression vectors encoding polypeptides (e.g., a variant rhizavidin polypeptide) described herein under control of transcriptional and/or translational control signals. These methods may include in vitro recombinant DNA and synthetic techniques and in vivo recombination (see, e.g., Ausubel, supra; or Sambrook, supra).

### Kits

The present disclosure also provides for kits for producing an immunogenic complex as disclosed herein which is useful for an investigator to tailor an immunogenic complex with their preferred polymer (e.g., an antigenic polysaccharide) and polypeptide (e.g., an antigenic polypeptide), e.g., for research purposes to assess the effect of an antigen, or a combination of antigens on immune response. Such kits can be prepared from readily available materials and reagents. For example, such kits can comprise any one or more of the following materials: a container comprising a polymer (e.g., an antigenic polysaccharide) cross-linked with a plurality of first affinity molecules; a container comprising a complementary affinity molecule which associates with the first affinity molecule, wherein the complementary affinity molecule associates with a polypeptide (e.g., an antigenic polypeptide or a carrier protein); a container comprising an antigenic polypeptide; a container comprising a carrier protein; a container comprising a variant rhizavidin variant polypeptide; a container comprising a fusion protein (e.g., a fusion protein comprising a variant rhizavidin polypeptide); a container comprising an antigenic polypeptide associated with a complementary affinity molecule; a container comprising a fusion protein associated with a complementary affinity molecule.

In another embodiment, the kit comprises a container comprising a polymer (e.g., a polysaccharide); a container comprising a plurality of first affinity molecules; and a container comprising a cross-linking reagent for cross-linking the first affinity molecules to the polysaccharide, for example, but not limited to, CDAP (1-cyano-4-dimethylaminopyridinium tetrafluoroborate), and EDC (1-Ethyl-3-[3-dimethylaminopropyl]carbodiimide hydrochloride).

In another embodiment, the kit comprises a container comprising a polypeptide (e.g., an antigenic polypeptide or carrier protein), and a container comprising a complementary affinity molecule, which associates with a first affinity molecule. In some embodiments, the kit further comprises a means to attach the complementary affinity molecule to the polypeptide (e.g., the antigenic polypeptide or carrier protein), where the means can be by a cross-linking reagent or by some intermediary protein.

In some embodiments, the kit can comprise at least one co-stimulation factor, which can be added to the polymer (e.g., a polysaccharide) or another polymer. In some embodiments, the kit comprises a cross-linking reagent, for example, but not limited to, CDAP (1-cyano-4-dimethylaminopyridinium tetrafluoroborate); EDC (1-Ethyl-3-[3-dimethylaminopropyl]carbodiimide hydrochloride); sodium cyanoborohydride; cyanogen bromide; and ammonium bicarbonate/iodoacetic acid, for linking the co-factor to the polymer (e.g., a polysaccharide) or to another polymer.

A variety of kits and components can be prepared for use in the methods described herein, depending upon the intended use of the kit, the particular target polymer (e.g., antigenic polysaccharide) and polypeptide (e.g., antigenic polypeptide) and the needs of the user. In some embodiments, a kit further includes informational material. The informational material can be descriptive, instructional, marketing, or other material that relates to the methods described herein and/or the use of compositions (e.g., composition comprising a variant rhizavidin polypeptide) described herein. The informational material of a kit is not limited in its form. In some instances, the informational material can include information about production of a variant rhizavidin polypeptide, amino acid of a variant rhizavidin polypeptide, nucleic acid encoding a variant rhizavidin polypeptide, molecular weight of a variant rhizavidin polypeptide, concentration, date of expiration, batch or production site information, and so forth.

In some cases, the informational material, e.g., instructions, is provided in printed matter, e.g., a printed text, drawing, and/or photograph, e.g., a label or printed sheet. The informational material can also be provided in other formats, such as Braille, computer readable material, video recording, or audio recording. In other instances, the informational material of the kit is contact information, e.g., a physical address, email address, website, or telephone number, where a user of the kit can obtain substantive information about an antibody therein and/or their use in the methods described herein. The informational material can also be provided in any combination of formats.

In addition to the components described above, a kit can include other ingredients, such as a solvent or buffer, a stabilizer, or a preservative. A kit can also include other agents, e.g., a second or third agent. The components can be provided in any form, e.g., liquid, dried or lyophilized form. The components can be substantially pure (although they can be combined together or delivered separate from one another) and/or sterile. When the components are provided in a liquid solution, the liquid solution can be an aqueous solution, such as a sterile aqueous solution. When the components are provided as a dried form, reconstitution generally is by the addition of a suitable solvent. The solvent, e.g., sterile water or buffer, can optionally be provided in the kit.

### EXEMPLARY EMBODIMENTS

Exemplary embodiments as described below are also within the scope of the present disclosure.
1. A variant rhizavidin polypeptide, comprising a mutation at one or more N-linked glycosylation sites of a wild-type rhizavidin polypeptide.
2. The variant rhizavidin polypeptide of embodiment 1, wherein the variant rhizavidin polypeptide comprises a mutation at each of four or more N-linked glycosylation sites of a wild-type rhizavidin polypeptide.
3. The variant rhizavidin polypeptide of embodiment 1 or 2, wherein the variant rhizavidin polypeptide comprises a mutation at each of five or more N-linked glycosylation sites of a wild-type rhizavidin polypeptide.
4. The variant rhizavidin polypeptide any one of embodiments 1-3, wherein at least one of the N-linked glycosylation sites comprises a consensus sequence of NXaaS or NXaaT, wherein Xaa is any amino acid residue except proline (P).
5. The variant rhizavidin polypeptide of any one of embodiments 1-4, wherein each of the N-linked glycosylation sites comprises a consensus sequence of NXaaS or NXaaT, wherein Xaa is any amino acid residue except proline (P).
6. The variant rhizavidin polypeptide of any one of embodiments 1-5, wherein the wild-type rhizavidin polypeptide comprises the amino acid sequence as set forth below:
FDASNFKDFSSIASASSSWQNQSGSTMIIQVDSFGNVSGQYVNRAQGTGCQNSPYPLTGR VNGTFIAFSVGWNNSTENCNSATGWTGYAQVNGNNTEIVTSWNLAYEGGSGPAIEQGQ DTFQYVPTTENKSLL (SEQ ID NO: 2), wherein the amino acid sequence of SEQ ID NO: 2 corresponds to amino acid residues 45-179 of the wild-type rhizavidin polypeptide.
7. The variant rhizavidin polypeptide of any one of embodiments 1-6, wherein the wild-type rhizavidin polypeptide comprises the amino acid sequence as set forth below:
MIITSLYATFGTIADGRRTSGGKTMIRTNAVAALVFAVATSALAFDASNFKDFSSIASASS SWQNQSGSTMIIQVDSFGNVSGQYVNRAQGTGCQNSPYPLTGRVNGTFIAFSVGWNNS TENCNSATGWTGYAQVNGNNTEIVTSWNLAYEGGSGPAIEQGQDTFQYVPTTENKSLL KD (SEQ ID NO: 1), wherein the amino acid sequence of SEQ ID NO: 1 corresponds to amino acid residues 1-179 of the wild-type rhizavidin polypeptide.
8. The variant rhizavidin polypeptide of any one of embodiments 1-7, wherein at least one of the N-linked glycosylation sites does not comprise amino acid residues 65-67 or 173-175 of a wild-type rhizavidin polypeptide.
9. The variant rhizavidin polypeptide of any one of embodiments 1-8, wherein at least one of the N-linked glycosylation sites comprises amino acid residues selected from the group consisting of amino acid residues 80-82, 106-108, 117-119, 118-120, and 138-140 of the wild-type rhizavidin polypeptide.
10. The variant rhizavidin polypeptide of any one of embodiments 1-9, wherein each of the N-linked glycosylation sites comprises amino acid residues selected from the group consisting of amino acid residues 80-82, 106-108, 117-119, 118-120, and 138-140 of the wild-type rhizavidin polypeptide.
11. The variant rhizavidin polypeptide of embodiment 10, wherein the one or more N-linked glycosylation sites are each of amino acid residues 80-82, 106-108, 117-119, 118-120, and 138-140 of the wild-type rhizavidin polypeptide.
12. The variant rhizavidin polypeptide of any one of embodiments 1, 2, and 4-11, wherein each of the N-linked glycosylation sites comprises amino acid residues selected from the group consisting of amino acid residues 80-82, 106-108, 118-120, and 138-140 of the wild-type rhizavidin polypeptide.
13. The variant rhizavidin polypeptide of embodiment 12, wherein the one or more N-linked glycosylation sites are each of amino acid residues 80-82, 106-108, 118-120, and 138-140 of the wild-type rhizavidin polypeptide.
14. The variant rhizavidin polypeptide of any one of embodiments 1-13, wherein at least one of the mutations is at a position selected from the group consisting of amino acid residues 80, 106, 108, 118, 119, and 138 of the wild-type rhizavidin polypeptide.
15. The variant rhizavidin polypeptide of any one of embodiments 1-14, wherein each of the mutations is at a position independently selected from the group consisting of amino acid residues 80, 106, 108, 118, 119, and 138 of the wild-type rhizavidin polypeptide.
16. The variant rhizavidin polypeptide of any one of embodiments 1-15, wherein each of the mutations is at a position independently selected from the group consisting of amino acid residues 80, 108, 118, 119, and 138 of the wild-type rhizavidin polypeptide.
17. The variant rhizavidin polypeptide of embodiment 16, comprising mutations at each of amino acid residues 80, 108, 118, 119, and 138 of the wild-type rhizavidin polypeptide.
18. The variant rhizavidin polypeptide of any one of embodiments 1-15, wherein each of the mutations is at a position independently selected from the group consisting of amino acid residues 80, 106, 118, 119, and 138 of the wild-type rhizavidin polypeptide.
19. The variant rhizavidin polypeptide of embodiment 18, comprising mutations at each of amino acid residues 80, 106, 118, 119, and 138 of the wild-type rhizavidin polypeptide.
20. The variant rhizavidin polypeptide of any one of embodiments 1, 2, and 4-15, wherein each of the mutations is at a position independently selected from the group consisting of amino acid residues 80, 106, 118, and 138 of the wild-type rhizavidin polypeptide.
21. The variant rhizavidin polypeptide of embodiment 20, comprising mutations at each of amino acid residues 80, 106, 118, and 138 of the wild-type rhizavidin polypeptide.
22. The variant rhizavidin polypeptide of any one of embodiments 1-21, wherein at least one of the mutations is selected from the group consisting of: (i) residue 80 N to A/Q, (ii) residue 106 N to A/Q, (iii) residue 108 T to A/Q, (iv) residue 118 N to A/Q, (v) residue 119 S to A/Q, and (vi) residue 138 N to A/Q.
23. The variant rhizavidin polypeptide of any one of embodiments 1-22, wherein each of the mutations is independently selected from the group consisting of: (i) residue 80 N to A/Q, (ii) residue 106 N to A/Q, (iii) residue 108 T to A/Q, (iv) residue 118 N to A/Q, (v) residue 119 S to A/Q, and (vi) residue 138 N to A/Q.
24. The variant rhizavidin polypeptide of any one of embodiments 1-23, wherein each of the mutations is independently selected from the group consisting of: (i) residue 80 N to A, (ii) residue 108 T to A, (iii) residue 118 N to A, (iv) residue 119 S to A, and (v) residue 138 N to A.
25. The variant rhizavidin polypeptide of embodiment 24, comprising mutations at each of: (i) residue 80 N to A, (ii) residue 108 T to A, (iii) residue 118 N to A, (iv) residue 119 S to A, and (v) residue 138 N to A.
26. The variant rhizavidin polypeptide of any one of embodiments 1-23, wherein each of the mutations is independently selected from the group consisting of: (i) residue 80 N to A, (ii) residue 106 N to A, (iii) residue 118 N to A, (iv) residue 119 S to A, and (v) residue 138 N to A.
27. The variant rhizavidin polypeptide of embodiment 26, comprising mutations at each of: (i) residue 80 N to A, (ii) residue 106 N to A, (iii) residue 118 N to A, (iv) residue 119 S to A, and (v) residue 138 N to A.
28. The variant rhizavidin polypeptide of any one of embodiments 1, 2, and 4-23, wherein each of the mutations is independently selected from the group consisting of: (i) residue 80 N to A, (ii) residue 106 N to A, (iii) residue 118 N to A, and (iv) residue 138 N to A.
29. The variant rhizavidin polypeptide of embodiment 28, comprising mutations at each of: (i) residue 80 N to A, (ii) residue 106 N to A, (iii) residue 118 N to A, and (iv) residue 138 N to A.
30. The variant rhizavidin polypeptide of any one of embodiments 1, 2, and 4-23, wherein each of the mutations is independently selected from the group consisting of: (i) residue 80 N to Q, (ii) residue 106 N to Q, (iii) residue 118 N to Q, and (iv) residue 138 N to Q.
31. The variant rhizavidin polypeptide of embodiment 30, comprising mutations at each of: (i) residue 80 N to Q, (ii) residue 106 N to Q, (iii) residue 118 N to Q, and (iv) residue 138 N to
32. The variant rhizavidin polypeptide of any one of embodiments 1-31, wherein the variant rhizavidin polypeptide does not comprise amino acid residues 1-44 of wild-type rhizavidin.
33. The variant rhizavidin polypeptide of any one of embodiments 1-32, wherein the variant rhizavidin polypeptide is or comprises an amino acid sequence having at least 80%, at least 85%, at least 90%, at least 95%, at least 96%, at least 97%, at least 98%, or at least 99% identity to the amino acid sequence of any one of SEQ ID NOs: 4-15.
34. The variant rhizavidin polypeptide of any one of embodiments 1-33, wherein the variant rhizavidin polypeptide is or comprises an amino acid sequence that is identical to the amino acid sequence of any one of SEQ ID NOs: 4-15.
35. The variant rhizavidin polypeptide of any one of embodiments 1-34, wherein the variant rhizavidin polypeptide is characterized in that it binds to biotin or a derivative or mimic molecule thereof, at an affinity that is at least 50%, at least 60%, at least 70%, at least 80%, or at least 90% of a wild-type rhizavidin polypeptide.
36. The variant rhizavidin polypeptide of any one of embodiments 1-35, wherein the variant rhizavidin polypeptide is characterized in that it binds to a biotin derivative, lipoic acid, HABA (hydroxyazobenzene-benzoic acid), dimethyl-HABA, or an amine-PEG3-biotin ((+)-biotinylation-3-6,9-trixaundecanediamine), at an affinity that is at least 50%, at least 60%, at least 70%, at least 80%, or at least 90% of a wild-type rhizavidin polypeptide.
37. The variant rhizavidin polypeptide of any one of embodiments 1-36, wherein the variant rhizavidin polypeptide is characterized in that it is substantially non-glycosylated when expressed in a mammalian cell.
38. The variant rhizavidin polypeptide of any one of embodiments 1-37, wherein the variant rhizavidin polypeptide is characterized in that its degree of glycosylation when expressed in a mammalian cell is reduced by at least 70%, at least 80%, or at least 90% as compared to a wild-type rhizavidin polypeptide.
39. The variant rhizavidin polypeptide of any one of embodiments 1-38, wherein the variant rhizavidin polypeptide is characterized in that when a population of the variant rhizavidin polypeptide is expressed in mammalian cells the population of variant rhizavidin polypeptides have an average monomeric molecular weight between 17 kDa and 19 kDa.
40. The variant rhizavidin polypeptide of any one of embodiments 1-39, wherein the variant rhizavidin polypeptide is characterized in that when a population of the variant rhizavidin polypeptide is expressed in mammalian cells the population of variant rhizavidin polypeptides have an average dimeric molecular weight between 26 kDa and 28 kDa.
41. A fusion protein comprising the variant rhizavidin polypeptide of any one of embodiments 1-40 and at least one additional polypeptide.
42. The fusion protein of embodiment 41, wherein the at least one additional polypeptide is or comprises one or more polypeptide antigens.
43. The fusion protein of embodiments 42, wherein at least one of the polypeptide antigens is or comprises an antigen selected from the group consisting of: bacterial polypeptide antigens, fungal polypeptide antigens, parasitic polypeptide antigens, viral polypeptide antigens, and mammalian polypeptide antigens (e.g., tumor antigens).
44. The fusion protein of embodiment 42 or 43, wherein at least one of the polypeptide antigens is or comprises an antigen selected from the group consisting of: streptococcal antigens (e.g., S. pneumoniae, group A, group B, and viridans antigens), tuberculosis antigens, tetanus antigens, anthrax antigens, pertussis antigens, staphylococcal antigens (e.g., S. aureus), Haemophilus antigens, Enterobacter, antigens, Acinetobacter antigens, Citrobacter antigens, Serratia antigens, Clostridia antigens, Campylobacter antigens, Vibriocholera antigens, Pseudomonas antigens, meningococcal antigens, Neisseria gonorrhoeae antigens, Chlamydia trachomatis antigens, Klebsiella antigens, Shigella antigens, Salmonella antigens, E. coli antigens, malaria antigens, HIV antigens, HPV antigens, influenza (e.g., seasonal or epidemic) antigens, coronavirus antigens (e.g., SARS-CoV-2 antigens), herpes (e.g., HSV) antigens, tumor antigens, and combinations thereof.
45. The fusion protein of any one of embodiments 42-44, wherein at least one of the polypeptide antigens is or comprises a pneumococcal polypeptide antigen.
46. The fusion protein of embodiment 45, wherein at least one polypeptide antigen is or comprises a polypeptide antigen selected from the group consisting of: a pneumolysin polypeptide antigen, a SP1500 polypeptide antigen, a SP0785 polypeptide antigen, and a SP0435 polypeptide antigen.
47. The fusion protein of embodiment 45 or embodiment 46, wherein the fusion protein comprises: (i) an SP1500 polypeptide antigen or fragment thereof, and (ii) an SP0785 polypeptide antigen or fragment thereof.
48. The fusion protein of embodiment 45 or embodiment 46, wherein the fusion protein comprises: (i) a pneumolysin polypeptide antigen or fragment thereof, and (ii) an SP0435 polypeptide antigen or fragment thereof.
49. The fusion protein of any one of embodiments 45-58, wherein at least one of the polypeptide antigens has an amino acid sequence that is or comprises the amino acid sequence of any one of SEQ ID NOs: 619-628.
50. The fusion protein of any one of embodiments 41-49, further comprising a peptide linker positioned between the variant rhizavidin polypeptide and the at least one additional polypeptide.
51. The fusion protein of any one of embodiments 41-50, further comprising a bacterial signal sequence at the N-terminus.
52. The fusion protein of any one of embodiments 41-51, further comprising a purification tag at the C-terminus, for example, wherein the purification tag is histidine tag, a c-myc tag, a Halo tag, a FLAG tag, or a combination thereof.
53. The fusion protein of any one of embodiments 42-52, wherein the fusion protein comprises an N terminal polypeptide and a C terminal polypeptide and the variant rhizavidin polypeptide is the C terminal polypeptide and the one or more polypeptide antigen is the N terminal polypeptide.
54. The fusion protein of any one of embodiments 41-53, wherein the fusion protein has an amino acid sequence that is or comprises the amino acid sequence of any one of SEQ ID NOs: 801-868.
55. A nucleic acid comprising a nucleotide sequence encoding the variant rhizavidin polypeptide of any one of embodiments 1-40 or the fusion protein of any one of embodiment 41-54.
56. The nucleic acid of embodiment 55, further comprising one or more regulatory sequences that directs expression of the variant rhizavidin polypeptide or the fusion protein.
57. The nucleic acid of embodiment 55 or 56, wherein the one or more regulatory sequences comprise one or more of the following: a promoter, an intron, an enhancer, a polyadenylation signal, a terminator, a silencer, a TATA box, or a Woodchuck Hepatitis Virus (WHP) Posttranscriptional Regulatory Element (WPRE).
58. A vector comprising the nucleic acid of any one of embodiments 55-57.
59. A method of producing the variant rhizavidin polypeptide of any one of embodiments 1-40 or the fusion protein of any one of embodiment 41-54, the method comprising using the nucleic acid of any one of embodiments 55-57 or the vector of embodiment 58 to express the variant rhizavidin polypeptide or the fusion protein in a host cell.
60. The method of embodiment 59, wherein the host cell is a bacterial cell, a yeast cell, an insect cell, or a mammalian cell.
61. A composition comprising the variant rhizavidin polypeptide of any one of embodiments 1-40 or the fusion protein of any one of embodiment 41-54.
62. The composition of embodiment 61, further comprising a biotinylated polymer comprising biotin and a polymer, wherein the biotinylated polymer is non-covalently associated with the variant rhizavidin polypeptide.
63. The composition of embodiment 62, wherein the polymer is or comprises a polysaccharide antigen.
64. The composition of embodiment 63, wherein the polysaccharide antigen is or comprises a polysaccharide selected from the group consisting of: a bacterial polysaccharide antigen, a fungal polysaccharide antigen, a parasitic polysaccharide antigen, a viral polysaccharide antigen, a mammalian polysaccharide antigen (e.g., a tumor polysaccharide antigen).
65. The composition of embodiment 63 or embodiment 64, wherein the polysaccharide antigen is or comprises a polysaccharide selected from the group consisting of: Salmonella polysaccharide, pneumococcal polysaccharides, Haemophili polysaccharides, meningococcal polysaccharides, staphylococcal polysaccharides, Bacillus anthracis polysaccharide, Streptococcus polysaccharide, Pseudomonas polysaccharide, Klebisella polysaccharide, Cryptococcus polysaccharide, other bacterial capsular or cell wall polysaccharides, viral polysaccharides (e.g., viral glycoproteins), or combinations thereof.
66. A pharmaceutical composition comprising the composition of any one of embodiments 61-65, and a pharmaceutically acceptable carrier.
67. The pharmaceutical composition of embodiment 66, further comprising one or more adjuvants.
68. The pharmaceutical composition of embodiment 67, wherein the one or more adjuvants are or comprise a co-stimulation factor.
69. The pharmaceutical composition of embodiment 67 or 68, wherein the one or more adjuvants are selected from the group consisting of: aluminum phosphate, aluminum hydroxide, and phosphate aluminum hydroxide.
70. The pharmaceutical composition of any one of embodiments 67-69, wherein the one or more adjuvants are or comprise aluminum phosphate.
71. The pharmaceutical composition of any one of embodiments 67-70, wherein the pharmaceutical composition is formulated for injection.
72. The pharmaceutical composition of any one of embodiments 67-71, wherein upon administration to a subject the pharmaceutical composition induces an immune response.
73. The composition of any one of embodiments 61-65 or the pharmaceutical composition of any one of embodiments 67-72, for use in administration to a subject to immunize the subject.
74. A use of the composition of any one of embodiments 61-65 or the pharmaceutical composition of any one of embodiments 67-72, in the manufacture of a medicament for administration to a subject to immunize the subject.
75. A method of immunizing a subject, comprising administering to the subject a composition of any one of embodiments 61-65 or the pharmaceutical composition of any one of embodiments 66-72.
76 The composition or pharmaceutical composition for use, use, or method of any one of embodiments 73-75, wherein the subject is a human.
77. The composition or pharmaceutical composition for use, use, or method of any one of embodiments 73-75, wherein the subject is an agricultural or non-domestic animal.
78. The composition or pharmaceutical composition for use, use, or method of any one of embodiments 73-75, wherein the subject is a domestic animal.
79. The composition or pharmaceutical composition for use, use, or method of any one of embodiments 73-78, wherein administration is by subcutaneous, intranasal, intradermal, or intramuscular injection.
80. The composition or pharmaceutical composition for use, use, or method of any one of embodiments 73-79, wherein the composition or pharmaceutical composition induces an immune response.
81. The composition or pharmaceutical composition for use, use, or method of embodiment 80, wherein the immune response comprises an antibody or B cell response.
82. The composition or pharmaceutical composition for use, use, or method of embodiment 81, wherein the immune response comprises a CD4+ T cell response (e.g., T_{H}1, T_{H}2, or T_{H}17 response), a CD8+ T cell response, a CD4+ and CD8+ T cell response, or a CD4−/CD8− T cell response.
83. The composition or pharmaceutical composition for use, use, or method of embodiment 80, wherein the immune response comprises (i) an antibody or B cell response, and (ii) a T cell response.
84. The composition or pharmaceutical composition for use, use, or method of embodiment 80, wherein the immune response comprises (i) an antibody or B cell response, and (ii) a CD4+ T cell response (e.g., T_{H}1, T_{H}2, or T_{H}17 response), a CD8+ T cell response, a CD4+ and CD8+ T cell response, or a CD4−/CD8− T cell response.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which the present disclosure belongs. Although methods and materials similar or equivalent to those described herein can be used in the practice or testing of the methods and materials of the present disclosure, suitable methods and materials are described herein.

The disclosure is further illustrated by the following examples. The examples are provided for illustrative purposes only. They is not to be construed as limiting the scope or content of the disclosure in any way.

### EXEMPLIFICATION

### Example 1: Design of Engineered Rhizavidin Variants

The present disclosure recognizes that expression of rhizavidin in mammalian cells resulted in glycosylation of the protein in a variable manner, generating multiple protein species with a gradient of molecular weights and low expression levels. The present Example demonstrates design of rhizavidin variants that provide improved expression of such proteins or fusion proteins comprising the same in mammalian expression systems.

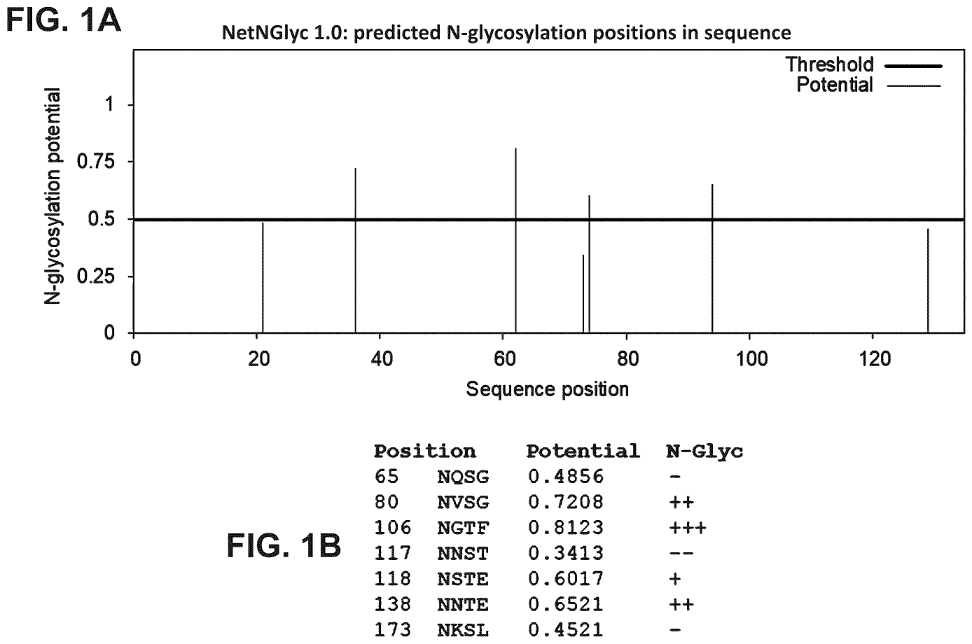

In some embodiments, such rhizavidin variants are engineered to reduce or abolish its glycosylation activity. Potential for N-linked glycosylation to occur at glycosylation motifs (e.g., NXT or NXS) present in rhizavidin was examined using NetNGlyc-1.0 [Gupta et al., 2002]. Four N-linked glycosylation sites having glycosylation positions corresponding to amino acid residues N80, N106, N118, and N138 of a full-length wild-type rhizavidin (e.g., as set forth in SEQ ID NO: 1), were predicted with high confidence (predicted by 9 of 9 neural networks). Three additional motifs, corresponding to amino acid residues N65 to S67, N117 to S119, and N173 to S175, were predicted to not be glycosylated. FIG. 1A shows the N-linked glycosylation potential of glycosylation positions present within rhizavidin [amino acids 45-179]. FIG. 1B shows the glycosylation motifs with glycosylation potential values and N-Glyc predicted glycosylation status.

Predicted N-linked glycosylation sites were then mapped to the structure of rhizavidin (PDB: 3EW2) [Meir et al., 2009] to determine which residue substitutions in the N-linked glycosylation motif (NXT or NXS) would be the least disruptive to folding and biotin binding. Analysis of the rhizavidin amino acid sequence and structure revealed proximity of the predicted glycosylation sites to residues involved in biotin binding. Residues N80, N106, N118, and N138 are located in flexible loops. To see whether substitution of amino acid residues that are located in flexible loops would disrupt folding or biotin binding of rhizavidin, each of the four asparagine residues was substituted with alanine to make an initial engineered rhizavidin A4 variant.

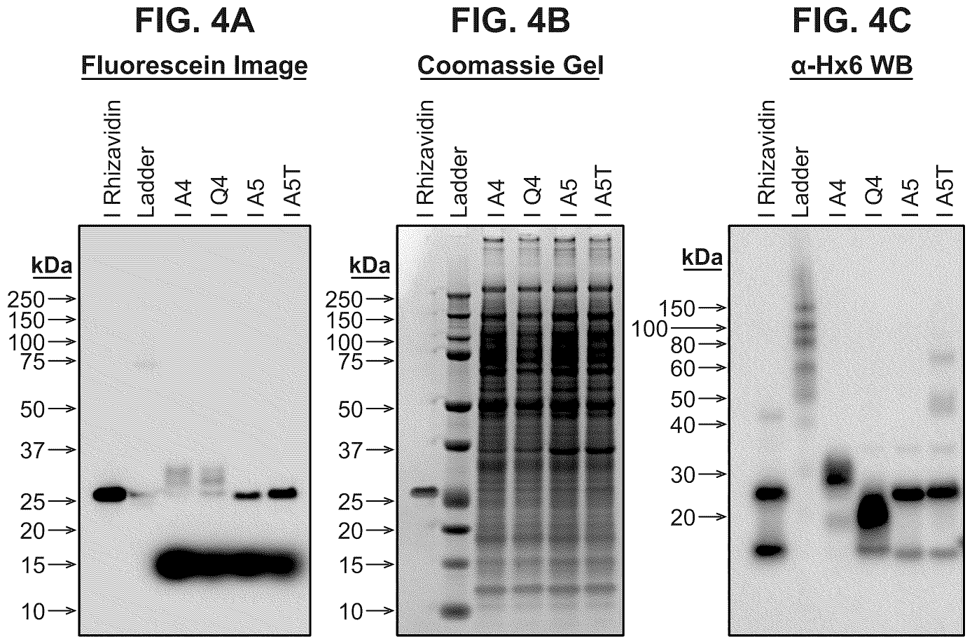

A version with glutamine substitutions rather than alanine, referred to as the rhizavidin Q4 variant, was also made based on side chain similarities between glutamine and asparagine. Biotin capture ELISA indicated that the rhizavidin Q4 variant expressed better than the A4 variant per liter of expression culture (see Example 2, Table 2, below). However, a Western blot surprisingly revealed that the majority of Q4 variant protein with the more conservative substitution was expressed in monomer form, while rhizavidin A4 variant with the less conservative substitution maintained the expected dimeric form of rhizavidin (FIG. 4C). This unexpected result provides an insight that the more conservative glutamine substitution, but not the less conservative alanine substitution, interfered with rhizavidin dimerization and thus generated a monomeric variant of rhizavidin, while the less conservative alanine substitution maintained the dimeric form of rhizavidin. The present disclosure appreciates that both monomeric and dimeric rhizavidin variants may be useful for a variety of applications.

Imaging of rhizavidin variants bound with biotin-4-fluorescein (B4F) (see Example 2, FIG. 4A) revealed multiple bands for rhizavidin A4 and Q4 variants, indicating that the A4 and Q4 variants unexpectedly retained at least one glycosylation site. Rhizavidin A4 variant was further analyzed using NetNGlyc-1.0, revealing a secondary N-linked glycosylation site having a glycosylation position at residue N117. Residue N117 is located next to the biotin-binding residue W116. To disrupt this secondary glycosylation site while minimizing impact on biotin binding of rhizavidin, residue S119 in the N-linked motif was selected for substitution with alanine in lieu of N117. The resulting rhizavidin variant, comprising substitutions N80A, N106A, N118A, S119A, and N138A, is referred to in this Example as rhizavidin A5 variant.

Additionally, it was found that in the folded rhizavidin protein, residue N106 is in close proximity to N80, in a strict beta-turn joining beta-sheet four (4) and beta-sheet five (5). To maintain the beta-pin structure, residue N106 was retained, and residue T108 in the NGT motif was instead substituted with alanine. The resulting construct, comprising substitutions N80A, T108A, N118A, S119A, and N138A, is referred to in this Example as rhizavidin A5T variant.

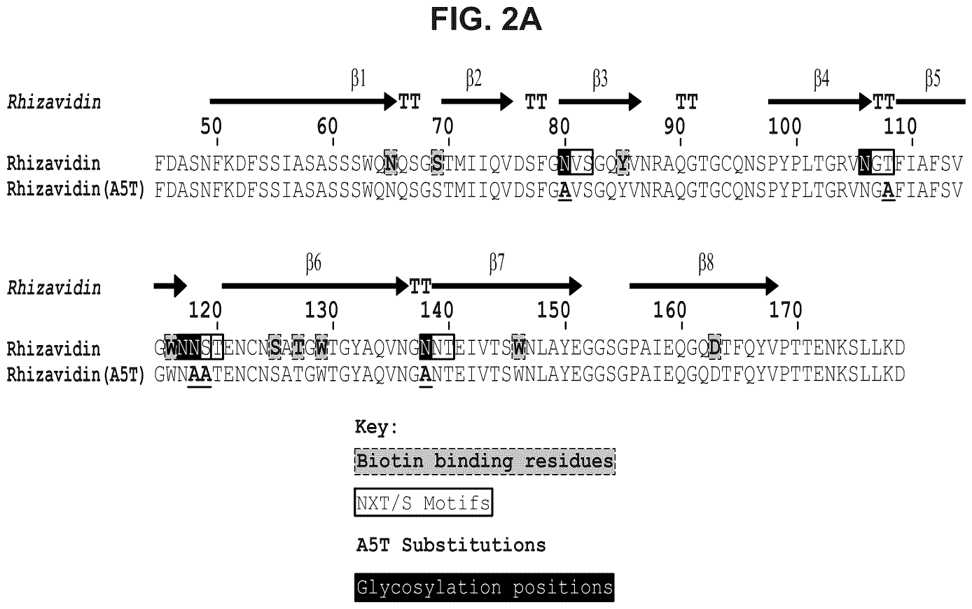

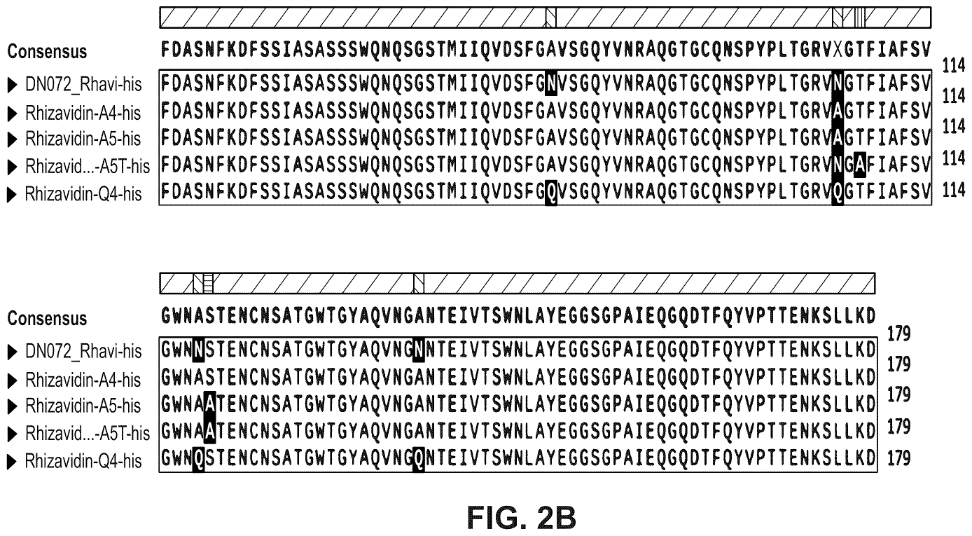

FIG. 2 shows sequence alignment of rhizavidin [amino acids 45-179] and engineered rhizavidin A4, Q5, A5, and A5T variants. Protein sequence alignment of rhizavidin (Rhizobium etli CFN 42) with and without substitutions was generated and analyzed using ClustalW2 [Larkin et al., 2007] and ESPript 3.x [Robert et al., 2014]. FIG. 2A: Sequence alignment of rhizavidin [amino acids 45-179] and rhizavidin A5T variant. The secondary structure of rhizavidin is displayed above the alignment (PDB: 3EW2) [Meir et al., 2009]. Beta-sheets are rendered as arrows and strict beta-turns as TT letters. Predicted glycosylation positions are shown in black boxes, NXT/S motifs are outlined in black, and residues involved in biotin binding are shown in dark gray boxes. A5T substitutions are in underlined black lettering. FIG. 2B: Sequence alignment of rhizavidin [amino acids 45-179] against rhizavidin A4, Q4, A5, and A5T variants. Consensus amino acids are highlighted in gray.

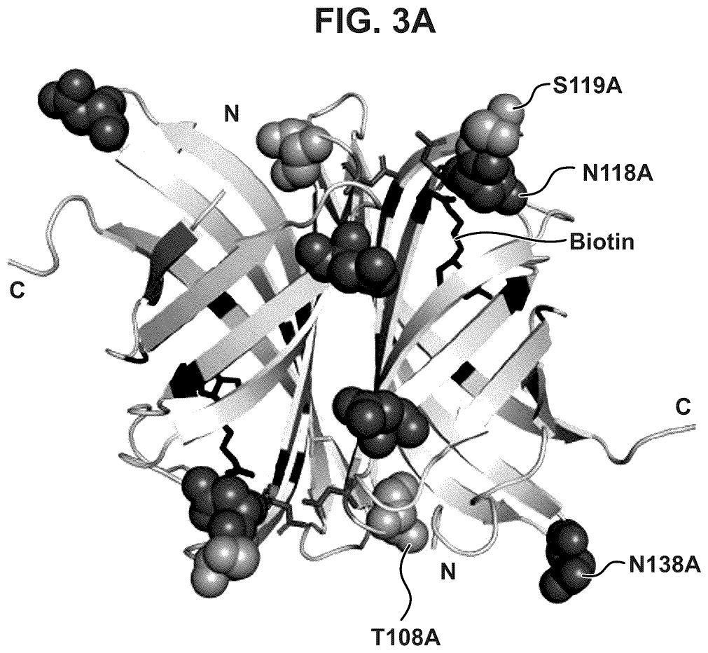

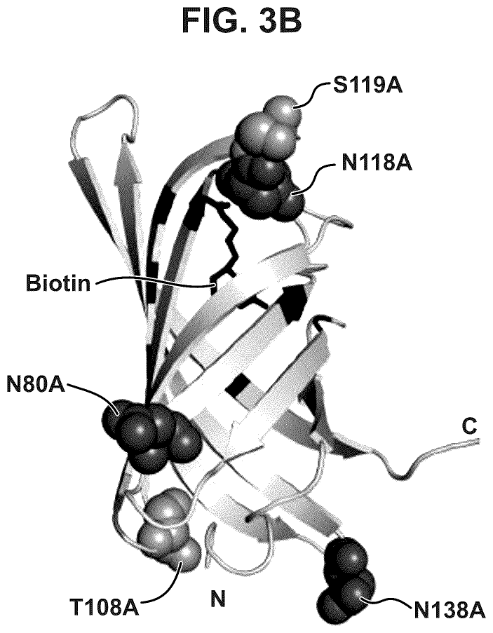

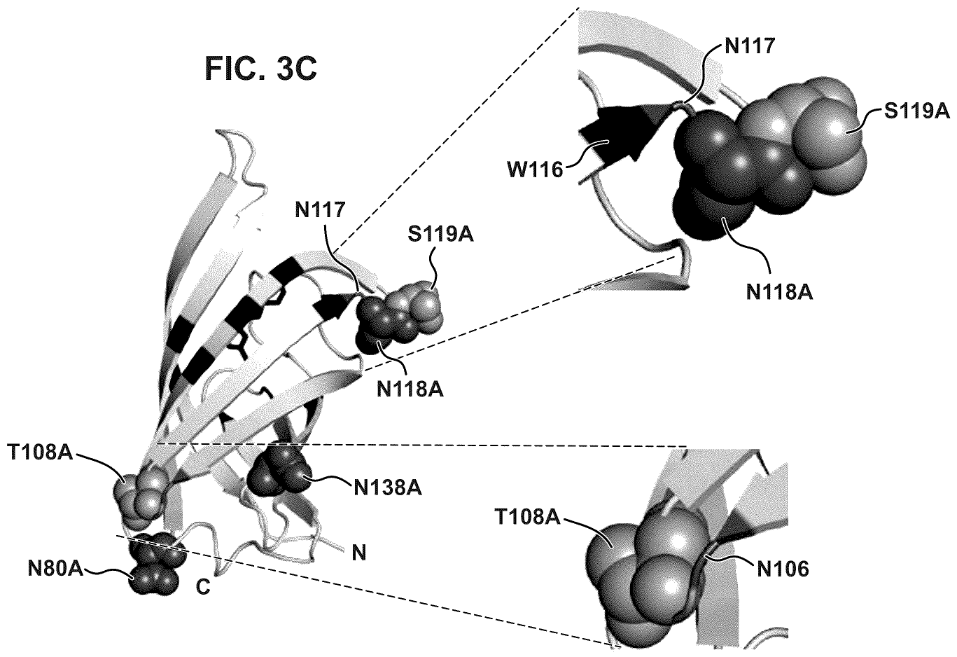

FIG. 3 shows a model of rhizavidin A5T variant dimer and monomer. The model is based on native rhizavidin structure (PBD: 3EW2) [Meir et al., 2009] and created in PyMol (Schrödinger, LLC, New York, NY, USA). Beta-sheets are rendered as flat arrows. Bound biotin molecules are shown as stick structures, residues involved in biotin binding as black boxes, and A5T substitutions in spheres (dark gray for N substitutions, light gray for S/T). The N and C termini are labeled with black N and C, respectively. FIG. 3A: Rhizavidin A5T variant dimer. FIG. 3B: Rhizavidin A5T variant monomer. FIG. 3C: Rotated and expanded rhizavidin A5T variant monomer, displaying amino acid residues 116-119 and 106-108.

### Materials & Methods

Engineering and Cloning: Rhizavidin and the engineered rhizavidin A4, Q4, A5, and AST variants were synthesized with a C-terminal portion comprising a GGGGSSS (SEQ ID NO: 48) linker and 6×His (SEQ ID NO: 79) tag at the C-terminal end, and without the native rhizavidin signal sequence [amino acids 1-44]. A start codon was added, leading to addition of an N-terminal methionine. The synthesized genes were cloned into the bacterial expression vector, pET21b, or the mammalian expression vector, pCI-Neo, with an H6 signal peptide, using XhoI and SalI restriction enzymes. Plasmids were sequence-confirmed. Codons were optimized for each expression system.

### Example 2: Expression and Characterization of Engineered Rhizavidin Variants

The present Example demonstrates expression and characterization of exemplary rhizavidin variants as described herein. Specifically, rhizavidin and engineered rhizavidin A4, Q4, A5, and AST variants were expressed in E. coli and HEK-293 cells to compare expression profiles. Expressed proteins were purified and characterized by biotin capture ELISA, SDS-PAGE, fluorescein imaging to detect biotin-binding activity against biotin-4-fluorescein (B4F), Coomassie staining, and Western blot (FIGS. 4A-4D).

Levels of biotin-binding protein, i.e., dimerized rhizavidin, were evaluated and are summarized in Table 2. The rhizavidin A4, Q4, and A5 variant constructs expressed relatively low levels of biotin-binding protein compared to rhizavidin A5T variant. Overall, the rhizavidin A5T variant construct expressed biotin-binding protein at a 4.6-fold greater level than rhizavidin A5 variant. These results provide an insight that disruption of glycosylation motif involving S119 promotes biotin-binding protein expression level. In addition, the results also provide an insight that disruption of a glycosylation motif present at amino acid residue positions 106 to 108 by mutating T108 in lieu of N106, promotes biotin-binding protein expression level.

TABLE 2
Biotin-binding protein expression levels per liter of expression culture

| | | Amino Acid Substitutions (the amino | |
|---|---|---|---|
| | | acid numbering is made relative to a | |
| | | full-length wild-type rhizavidin set | |
| Protein | Variant | forth in SEQ ID NO: 1) | Expression (mg/L) |
| Rhizavidin- | A4 | N80A, N106A, N118A, N138A | 0.029 mg/L |
| his | Q4 | N80Q, N106Q, N118Q, N138Q | 0.053 mg/L |
| | A5 | N80A, N106A, N118A, S119A, N138A | 0.071 mg/L |
| | A5T | N80A, T108A, N118A, S119A, N138A | 0.327 mg/L |

FIG. 4 compares biotin-binding activity and presence of dimers in mammalian-expressed rhizavidin A4, Q4, A5, and A5T variants. FIG. 4A show that samples of purified mammalian-expressed rhizavidin A4, Q4, A5, and A5T variants and reference bacterially-expressed rhizavidin were combined with biotin-4-fluorescein (B4F) and separated by SDS-PAGE. Fluorescein imaging produced a single ~27 kDa band in reference rhizavidin, and in A5 and A5T variant samples, consistent with elimination of N-linked glycosylation sites in the A5 and A5T variant constructs and presence of rhizavidin dimers able to bind biotin. The broad fuzzy band at the bottom of the gel is unbound B4F. The smeared higher molecular weight bands in A4 and Q1 variant samples are consistent with glycosylation and show minimal biotin-binding activity. Coomassie staining in FIG. 4B showed equivalent amounts of protein in each of the rhizavidin A4, Q4, A5, and AST variant lanes, indicating that the lack of biotin-binding activity in the A4 and Q4 variant samples was not due to differences in loading of the SDS-PAGE gel. FIG. 4C shows that a Western blot with an anti-histidine monoclonal antibody detected a band at ~27 kDa in reference rhizavidin, and A5 and AST variant samples, consistent with the presence of rhizavidin dimers. In FIG. 4C, the lower molecular weight band in reference rhizavidin, and A5 and A5T variant samples is likely monomeric rhizavidin at ~18 kDa. The smeared bands in A4 and Q4 variant samples may represent glycosylated dimers and monomers.

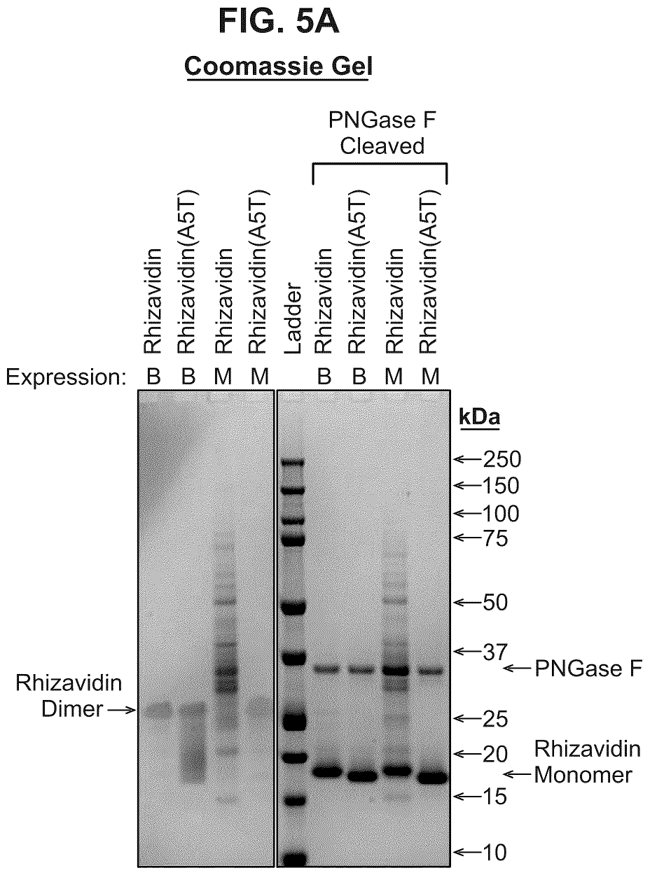

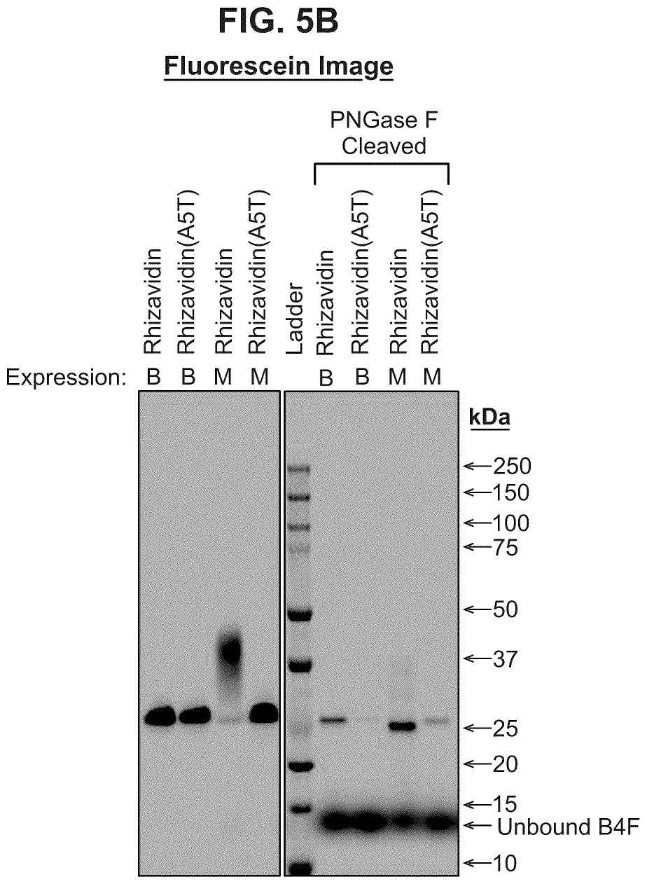

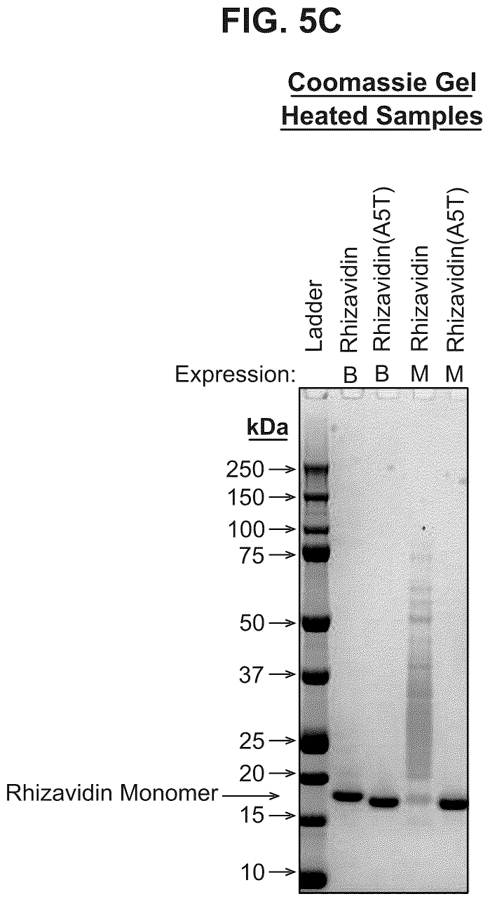

FIG. 5 compares glycosylation, biotin-binding activity, and presence of dimers in bacterially-expressed (B) and mammalian-expressed (M) reference rhizavidin to bacterially- and mammalian-expressed rhizavidin A5T variant. PNGase F, an amidase that cleaves the innermost GlcNAc and asparagine residues in oligosaccharides, was used to cleave all N-linked glycans from purified reference rhizavidin and rhizavidin A5T variant under heat denaturing conditions. The cleavage products were run on SDS-PAGE with biotin-4-fluorescein (B4F); gels were imaged on fluorescein and then stained with Coomassie. Due to denaturing conditions of the PNGase F cleavage reaction, rhizavidin dimers are destabilized and expected to be largely reduced to their monomeric forms at ~18 kDa [Helppolainen et al., 2007]. FIGS. 5A and 5B: Samples of purified bacterially-expressed (B) and mammalian-expressed (M) reference rhizavidin and rhizavidin A5T variant were incubated in the presence or absence of PNGase F, as indicated, then combined with B4F and separated by SDS-PAGE. In the absence of PNGase F cleavage, bacterially-expressed reference rhizavidin, bacterially-expressed rhizavidin A5T variant, and mammalian-expressed rhizavidin A5T variant produced a band at ~27 kDa by Coomassie staining (FIG. 5A, left) and fluorescein imaging (FIG. 5B, left). This shows the presence of rhizavidin dimers capable of binding biotin. The smeared higher molecular weight bands seen with mammalian-expressed reference rhizavidin are consistent with glycosylation and show minimal biotin-binding activity.

Following PNGase F cleavage, both bacterially-expressed (B) and mammalian-expressed (M) reference rhizavidin and rhizavidin A5T variant collapsed to a nearly uniform ~18 kDa band, the expected size of monomer rhizavidin, shown by Coomassie staining (FIG. 5A, right). A band at ~34 kDa corresponds to PNGase F. As denaturing conditions of the PNGase F cleavage reaction were insufficient to denature all dimerized rhizavidin, residual biotin-binding activity was observed in the fluorescein image at ~27 kDa (FIG. 5B, right). The broad fuzzy band at the bottom of the gel is unbound B4F (FIG. 5B, right).

FIG. 5C shows samples of purified bacterially-expressed (B) and mammalian-expressed (M) reference rhizavidin and rhizavidin A5T variant heated at 100° C. for 10 minutes with a reducing agent, separated by SDS-PAGE, and Coomassie stained. Bacterially-expressed reference rhizavidin, bacterially-expressed rhizavidin A5T variant, and mammalian-expressed rhizavidin A5T variant collapsed completely into a band at ~18 kDa, the expected size of rhizavidin monomers. As in FIGS. 5A and 5B, the smeared higher molecular weight bands seen with mammalian-expressed reference rhizavidin are consistent with glycosylation.

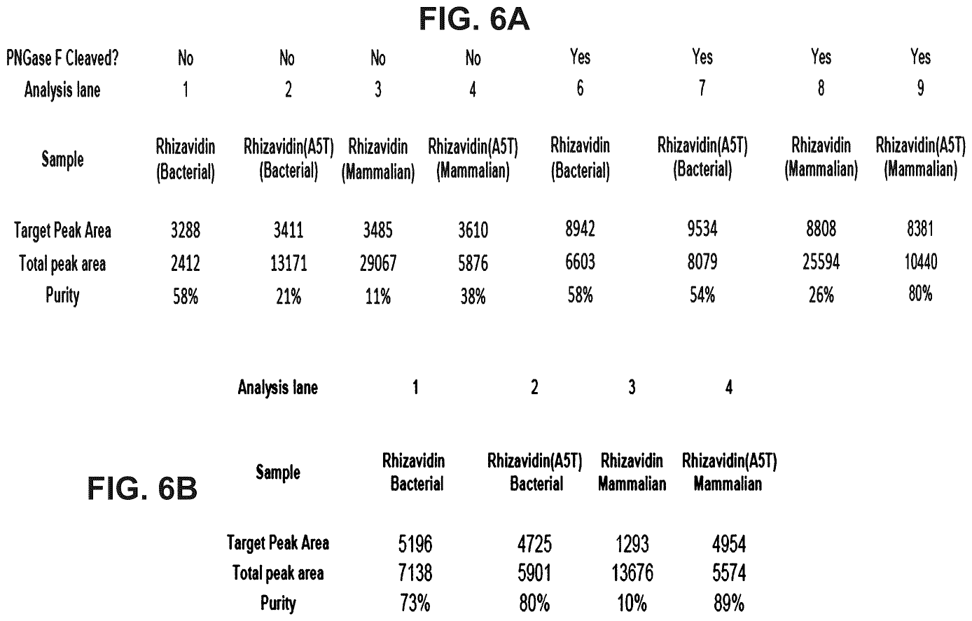

FIG. 6 illustrates protein purity based on densitometry results for select samples of FIGS. 5A-5C. FIG. 6A summarizes densitometry results and purity of PNGase F-cleaved proteins of FIG. 5A. FIG. 6B summarizes densitometry results and purity of heated rhizavidin proteins of FIG. 5C. These results show that when expressed in mammalian cells, rhizavidin A5T variant shows higher purity than reference rhizavidin.

In conclusion, mammalian expression of rhizavidin [amino acids 45-179] resulted in heterogeneous high molecular weight species consistent with glycosylation that did not form dimers and that did not have significant biotin-binding activity. Through stepwise substitution of residues at the predicted N-linked glycosylation sites, rhizavidin A5T variant construct with desirable attributes was engineered. As shown herein, rhizavidin A5T variant can be expressed in mammalian cells at significantly higher levels than reference rhizavidin or rhizavidin A4, Q4, and A5 variants, while retaining dimer-forming ability and biotin-binding activity.

### Materials & Methods

Expression and Purification: All bacterial plasmids were transformed into the E. coli strain SHuffle T7 Express (NEB, Ipswich, MA) and were induced with isopropyl-β-D-1-thiogalactopyranoside (IPTG). All mammalian plasmids were transiently transfected into Expi293F cells (ThermoFisher, Waltham, MA) for expression. Expi293F cells (ThermoFisher Scientific, Waltham, MA, USA) were maintained in Erlenmeyer flasks at 37° C. in an orbital shaker. On the day of transfection, cells were diluted to the appropriate density and mixed with an optimal ratio of DNA, Fectopro transfection reagent (Polyplus, Illkirch-Graffenstaden, France), and Opti-mem media (ThermoFisher Scientific, Waltham, MA, USA). Cells were then incubated for 2 days, at which point valproic acid (Sigma-Aldrich, St. Louis, MO, USA) and glucose (Sigma-Aldrich, St. Louis, MO, USA) were added to the flasks. Cells were then incubated for 3 more days under the same conditions until harvest via centrifugation. His-tagged protein present in the supernatant was purified via nickel affinity chromatography using complete His-Tag Purification Columns (Roche, Basel, Switzerland). In some instances, the eluents of the Ni-NTA column underwent additional anion exchange chromatography using a HiTrap Q HP column (Cytiva, Marlborough, MA, USA) and/or were further purified by size exclusion chromatography (SEC) on a Superdex 200 column (Cytiva, Marlborough, MA, USA). Peak fractions containing the proteins were collected, evaluated by SDS-PAGE, and flash-frozen in liquid nitrogen and stored at −80° C. until use. Concentration was determined using a bicinchoninic acid (BCA) protein assay kit (Bio-Rad, Hercules, CA, USA).

SDS PAGE and Western Blot Analysis: Equal SDS sample buffer and were loaded either unheated or heated for 10 minutes at 100° C. on 4-12% Bis-Tris SDS-PAGE in triplicate. To evaluate biotin binding, the samples were imaged with the fluorescein setting and then stained with ReadyBlue Protein Gel Stain (Sigma, RSB-1L). For Western blot analysis, samples were transferred to a PVDF membrane using standard settings on an iBlot2 Gel Transfer Device. The membrane was then incubated with anti-6×His tag antibody (Abcam, ab18184, diluted 1:5000) followed by peroxidase secondary antibody (Jackson ImmunoResearch, 115-035-071, diluted 1:1000) in an iBind Western device. Chemiluminescence was captured by adding Radiance Q (Azure, AC2101) imaging the membranes with a chemiluminescent setting. All imaging was done using a ChemiDoc MP Imaging System.

Enzymatic Cleavage and Analysis of Rhizavidin Variants: Rhizavidin and rhizavidin A5T variant proteins were cleaved following the PNGase F protocol (Cat #P0704s, New England Biolabs, Ipswich, MA, USA) for denaturing reaction conditions. The denaturing protocol used a 10-minute incubation at 100° C. combined with glycoprotein denaturing buffer to initially denature the protein and make the glycans more accessible. This was followed with an hour-long incubation with PNGase F at 37° C. 10 μg of protein was used in each incubation reaction for a final concentration of 4 μg/μl. Following the cleavage reaction with PNGase F, the cleaved samples were prepared alongside non-cleaved samples to run on 4-12% Bis-Tris SDS-PAGE with 50 ng biotin-4-fluorescein. 2.4 μg of each protein was loaded into wells, except for mammalian-expressed reference rhizavidin, which had 3 μg of total protein due to low purity. Gels were analyzed via fluorescein imaging, followed by staining with Instantblue Coomassie stain (Abcam, Cambridge, United Kingdom).

### Example 3: Characterization of Biotin-Binding Affinity of Rhizavidin A5T Variant

The present Example demonstrates biotin-binding affinity of exemplary rhizavidin variants described herein and the impact of using different expression systems to express such rhizavidin variants on their biotin-binding affinity. For example, to evaluate the impact of the rhizavidin A5T variant substitutions and different expression systems on affinity of rhizavidin to biotin, equilibrium constants (Kd) were measured and compared for each of bacterially-expressed reference rhizavidin, bacterially-expressed rhizavidin A5T variant, and mammalian-expressed rhizavidin A5T variant through microscale thermophoresis (MST) [Jerabek-Willemsen et al., 2014]. Purified proteins were mixed with a Biotin, Flamma 648, a 927.2 Da molecule consisting of a molecule of biotin covalently attached to Flamma 648 dye, and evaluated on MST. A Kd could not be determined for mammalian-expressed reference rhizavidin because of protein heterogeneity (e.g., see FIG. 5A).

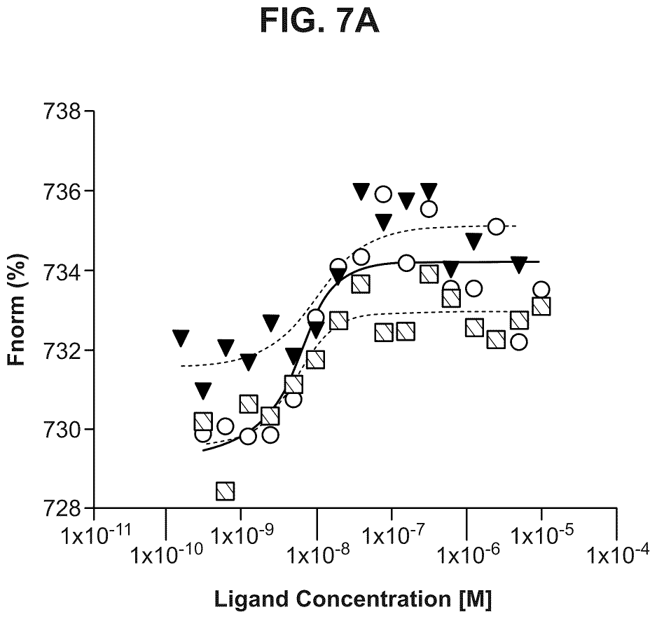

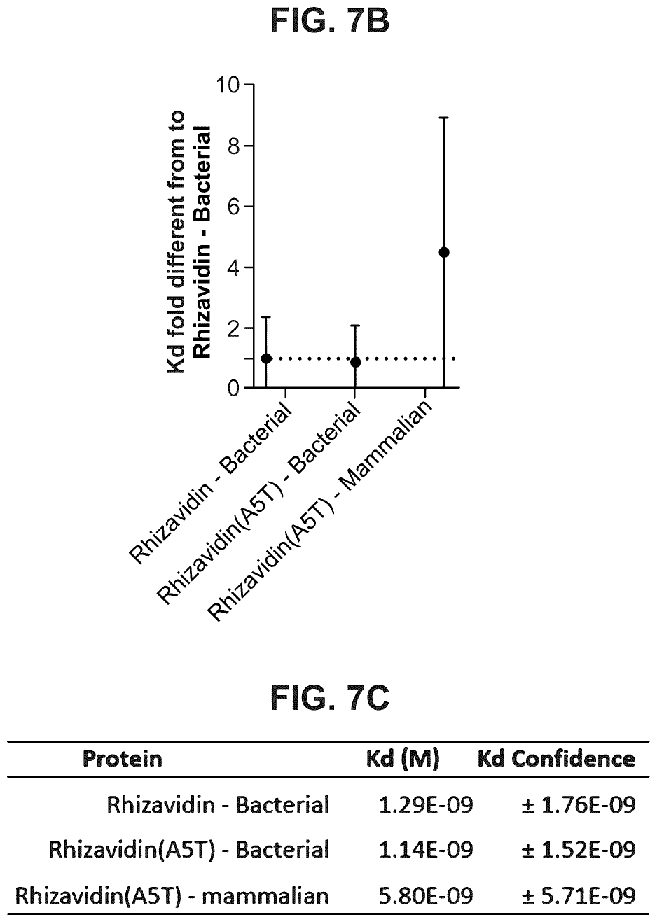

FIG. 7 shows MST biotin-binding kinetics of rhizavidin A5T variant compared to reference rhizavidin, obtained by maintaining constant Biotin, Flamma 648 concentration while titrating protein. FIG. 7A: Binding curves with dose response. Bacterially-expressed reference rhizavidin is shown as dots, bacterially-expressed rhizavidin A5T variant as squares, and mammalian-expressed rhizavidin A5T variant as triangles. FIG. 7B: Normalized Kd and confidence intervals calculated in MO.Control software were normalized by dividing them by Kd for bacterially-expressed reference rhizavidin. Affinity between the rhizavidin proteins and Biotin, Flamma 648 was in the single digit nanomolar range with overlapping confidence intervals. FIG. 7C: Calculated equilibrium constants of rhizavidin proteins to biotin, Flamma 648 with confidence intervals.

These MST biotin-binding assay results show that rhizavidin A5T variant expressed by a mammalian expression system is characterized by a higher biotin-binding affinity, for example, at least about 2-5 fold higher, as compared to that of a bacterially-expressed reference rhizavidin or a bacterially-expressed rhizavidin A5T variant.

### Materials & Methods

Expression and Purification: As described in Example 2 above.

MST Comparison of Biotin-Binding: Purified proteins were examined on a Monolith NT.115Pico (Nanotemper, Cambridge, MA, USA). Biotin, Flamma 648 (Cat #RFP0611, BioActs Co., Incheon, Korea) was resuspended in deionized water to 3.6 M and diluted to 18 nM with 1×DPBS-T (Boston Bioproducts, Milford, MA, USA). Protein samples were also diluted in 1×DPBS-T to starting concentrations of approximately 20 μM and serially diluted with 1×DPBS-T. Samples were then mixed with an equal volume of 18 nM Biotin, Flamma 648 and loaded in Monolith NT.115 capillaries. Using MO.Control 2.0 software, MST detector was set to Pico RED, LED power was set to 5%, and MST strength was set to high. Data was analyzed in MO.Affinity Analysis 3.0 software.

### Example 4: Use of Exemplary Engineered Rhizavidin Variants in Fusion Proteins and MAPS Complexes

The Multiple Antigen Presenting System (MAPS) utilizes the biotin-binding function of rhizavidin to couple proteins to polysaccharides via non-covalent interaction [Zhang et al., 2013]. In the present Example, exemplary rhizavidin variants as described herein were genetically fused to polypeptides of interest (e.g., antigenic polypeptides), allowing their non-covalent attachment to biotinylated polymers of interest (e.g., polysaccharides) and generating a macromolecular complex. The MAPS platform has clear applications for bacterial vaccines and has been shown to produce protective immune responses against bacterial pathogens such as Streptococcus pneumoniae and Staphylococcus aureus, generating robust T-cell and B-cell responses [Zhang et al., 2013; Zhang et al., 2018]. In addition, Applicant recently demonstrated the versatility of the MAPS platform by coupling viral protein antigens (e.g., particularly from SARS-CoV-2) to a bacterial polysaccharide, which displayed protective efficacy in a pre-clinical SARS-CoV-2 non-human primates challenge model [Cieslewicz et al., 2022].

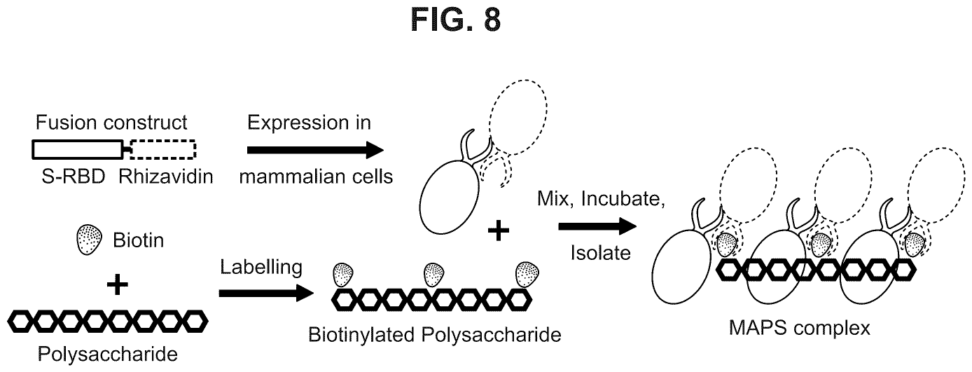

Specifically, to test whether the AST substitutions described in Example 2 and expression of rhizavidin A5T variant in mammalian cells impacted the protein's ability to form MAPS complexes, recombinant fusion proteins comprising rhizavidin A5T and the receptor binding domain of SARS-CoV-2 spike protein (S-RBD) (amino acids 324-521) were generated. Three versions of recombinant fusion proteins were made, each containing a different S-RBD sequence from the original SARS-CoV-2 viral strain (D614G; referred to as ancestral), or two variant strains (delta and gamma). Then, the S-RBD-rhizavidin A5T fusion proteins were mixed with biotinylated pneumococcal capsular polysaccharide (from serotype 1) as shown in FIG. 8, which is a schematic of MAPS technology. After assembly and purification, MAPS complexes, alongside purified fusion proteins and polysaccharide alone, were analyzed by HPLC-SEC-MALS to define their molar mass. Samples were further analyzed via SDS-PAGE and stained with Coomassie.

FIG. 9 shows normalized mass fraction distribution determined by HPLC-SEC-MALS of mammalian-expressed ancestral and variant S-RBD-rhizavidin A5T fusion proteins, MAPS complexes comprising ancestral and variant S-RBD-rhizavidin A5T fusion proteins, and pneumococcal capsular polysaccharide, together with correlation by SDS-PAGE. FIG. 9A: Peaks for fusion proteins (Proteins), polysaccharide, and MAPS complexes (MAPS) are labeled. The molecular weight distribution curve for all three fusion proteins displayed a single peak corresponding to their calculated molecular weight (39.5 kDa). The polysaccharide profile shows a highly polydisperse molecular weight distribution, similarly observed for the three MAPS complexes. The profile of MAPS complexes is shifted to the right due to the association of polysaccharide and fusion protein; different MAPS complexes showed increased average molecular weight. FIG. 9B: Molecular weights and polydispersity of each sample determined by HPLC-SEC-MALS in tabular form. FIG. 9C: Coomassie stained SDS-PAGE loaded with 3 μg of pneumococcal capsular polysaccharide (PS), 1.5 μg of the indicated mammalian-expressed S-RBD-rhizavidin A5T fusion proteins, 1.5 μg of the indicated MAPS complexes (by protein) combined with 1× reducing Laemmli buffer. Samples to the left of each set were not heated; samples to the right of each set were heated for 10 minutes at 100° C. Bands at ~80 kDa corresponding to S-RBD-rhizavidin A5T fusion protein dimers are noted. Polysaccharide samples and unheated MAPS complexes were retained in the well due to their size; however, after heating, ~40 kDa bands corresponding to S-RBD-rhizavidin A5T fusion protein monomers were observed as well.

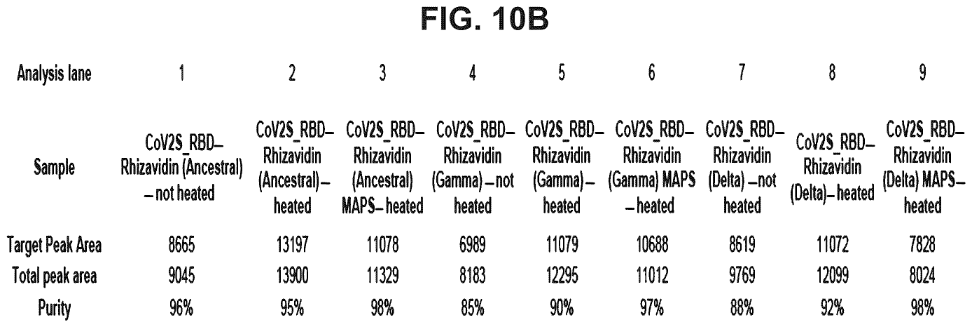

FIG. 10 illustrates purity based on densitometry results for fusion proteins and MAPS complexes of FIG. 9B. FIG. 10A: Raw Coomassie stained SDS-PAGE. FIG. 10B: Summary of densitometry results and purity.

These results show that mammalian-expressed fusion proteins comprising a rhizavidin A5T variant and S-RBD of ancestral and variant SARS-CoV-2 formed MAPS complexes. Thus, these results indicate that mammalian-expressed fusion proteins comprising a rhizavidin variant as described herein and a polypeptide of interest retain the biotin-binding activity of such rhizavidin variants, allowing its non-covalent interaction with a biotinylated polymer (e.g., a biotinylated polysaccharide).

### Materials & Methods

S-RBD-rhizavidin A5T Constructs: The ancestral S-RBD-rhizavidin A5T fusion protein was synthesized with S-RBD sequences (amino acids 331-524) fused to the rhizavidin A5T fragment with an C-terminal GS-His-tag, as generally described [Gorman et al., 2021]. The synthesized insert was then cloned into the pcDNA3.4 vector with a human IgGH signal peptide. The gamma (K417N, E484K, N501Y) and delta (L452R, E484Q) variants of S-RBD were made by site-directed mutagenesis from the ancestral construct.

Generation and Analysis of MAPS Complexes: MAPS complexes comprising biotinylated polysaccharide PS1 and each of the three variants of SARS-CoV-2 S-RBD fused to rhizavidin A5T were assembled and isolated via SEC as previously described [Cieslewicz et al., 2022]. The concentration of polysaccharide in MAPS complexes was measured using a uronic acid assay as previously described [Cieslewicz et al., 2022], while concentration of protein was measured using a bicinchoninic acid (BCA) protein assay kit (Pierce, Waltham, MA, USA). MAPS complexes were analyzed via SDS-PAGE and HPLC-SEC-MALS as previously described [Cieslewicz et al., 2022]. Briefly, 1.5 μg of samples combined with reducing Laemmli buffer and loaded or heated to 100° C. for ten minutes and loaded on a 4-12% Bis-Tris Protein gel (Invitrogen, Waltham, MA, USA) for 50 minutes at 180 volts. Polydispersity was determined by a ratio of the weight average molecular weight and number average molecular weight.

### Example 5: Immunogenicity of MAPS Vaccines Comprising Fusion Proteins that Include Exemplary Rhizavidin Variants Described Herein

The present Example demonstrates the immunogenicity of MAPS vaccines using exemplary rhizavidin variants described herein in lieu of reference rhizavidin without such amino acid substitutions. To test immunogenicity, female C57Bl/6 mice (n=10/group) were immunized twice, three weeks apart, with a 20 μg dose of MAPS complexes comprising mammalian-expressed ancestral, delta, or gamma S-RBD-rhizavidin A5T fusion proteins described in Example 4, or an equal mixture of the three S-RBD-rhizavidin A5T fusion proteins totaling 20 μg (multivariant). The MAPS complexes were formulated as vaccines with an adjuvant (e.g., alum). IgG levels were measured using ELISA coating with the S-RBD from each strain.

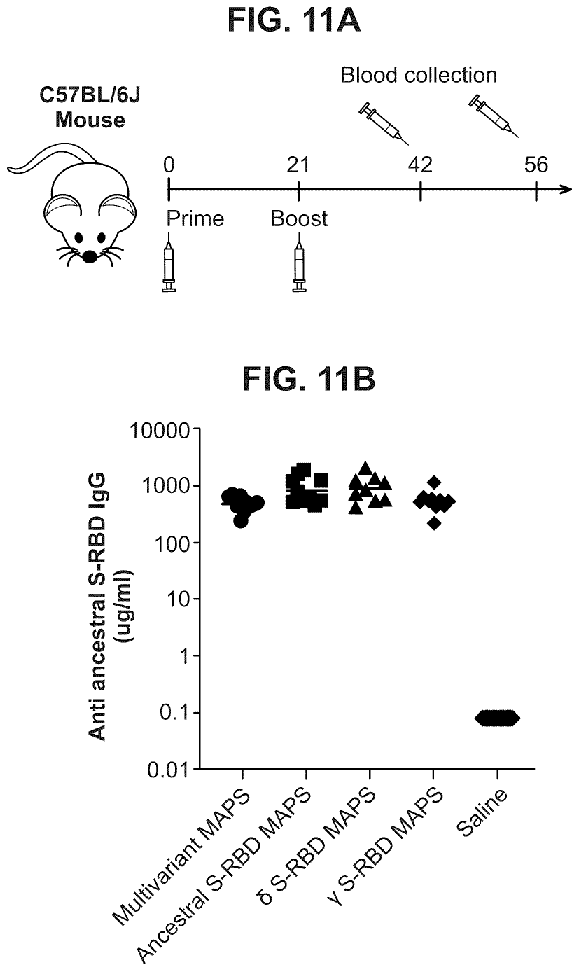

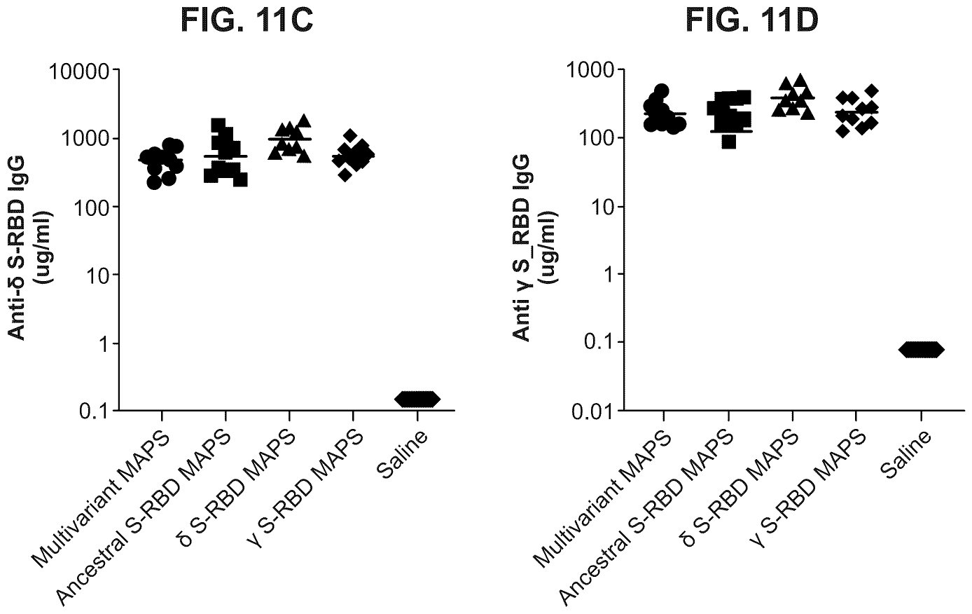

FIG. 11 shows immunogenicity results for exemplary S-RBD monovariant and multivariant MAPS vaccines. Anti-S-RBD IgG titers of mice that were immunized with any of the MAPS vaccines increased significantly compared to the saline control group. Immunization with any of the three S-RBD MAPS vaccines produced similar anti-S-RBD IgG titers. There was no significant difference between IgG titers against any of the strains of SARS-CoV2 S-RBD when animals were immunized with the multivariant MAPS vaccine compared to monovariant S-RBD MAPS vaccines. FIG. 11A: Schematic of the study, FIG. 11B: IgG levels directed to ancestral S-RBD. FIG. 11C: IgG levels directed to delta S-RBD. FIG. 11D: IgG levels directed to gamma S-RBD.

These results show that MAPS vaccines comprising mammalian-expressed fusion proteins comprising rhizavidin A5T can elicit immunogenicity to S-RBD. Thus, these results indicate that mammalian-expressed rhizavidin variants as described herein are useful to generate MAPS complexes for inducing immune responses.

### Materials & Methods

Animal Studies: Female C57Bl/6 mice (n=10/group, 50 total) received two subcutaneous immunizations of 20 μg of SARS-CoV-2 MAPS vaccine (by protein mass) comprising the S-RBD fragment from the ancestral (D614G), delta, gamma, or all three SARS-CoV-2 strains (6.7 μg each, 20 μg total). Control animals received saline injections. Sera were obtained before each immunization and at a terminal bleed on Day 56 for measurement of specific IgG responses. Observations for morbidity, mortality, clinical signs, body temperature and food and water consumption were conducted for all animals on a regular basis.

SARS-CoV-2 S-RBD IgG ELISA. Zollinger sandwich ELISA was performed to quantify serum IgG levels. To examine mouse-anti-S-RBD IgG, MaxiSorp 96-well plates (Nunc, Rochester, NY, USA) were coated with 100 μl of 4 μg/ml of his-tagged SARS-CoV-2 S-RBD (Acrobio systems, Newark, DE, USA) and incubated overnight at 4° C. Similarly, AffiniPure Goat anti-mouse F(ab′)2 (Jackson laboratory, Bar Harbor, ME, USA) was coated for standards. Plates were washed with 0.05% DPBST (405TS Microplate washer, Bio Tek, Winooski, VT, USA) and blocked with 1% BSA-DPBS (Millipore sigma, Burlington, MA, USA) for 1 hour at room temperature. After washing, 100 μl of diluted serum from immunized mice/purified mouse IgG (MP Biomedicals LLC, CA, USA) was added to the wells and incubated for 1 hour at room temperature. After 1 hour, plates were washed and 100 μl of 1:50,000 goat anti-mouse IgG (Fc)-HRP (Jackson laboratory, PA) was added to the wells. After 1 hour incubation at room temperature, plates were washed, 100 μl of SureBlue TMB (VWR, Radnor, PA, USA) was added and developed for 10 minutes. The reaction was stopped by adding 100 μl of 1N HCl. Absorbance was measured at 450 nm using SoftMax Pro 7.0 software on a SpectraMax i3x plate reader (Spectramax, Sunnyvale, CA, USA).

### Example 6: Rhizavidin AST can Form a Biotin-Binding Fusion Protein with the Mammalian Cancer Antigen TRP2

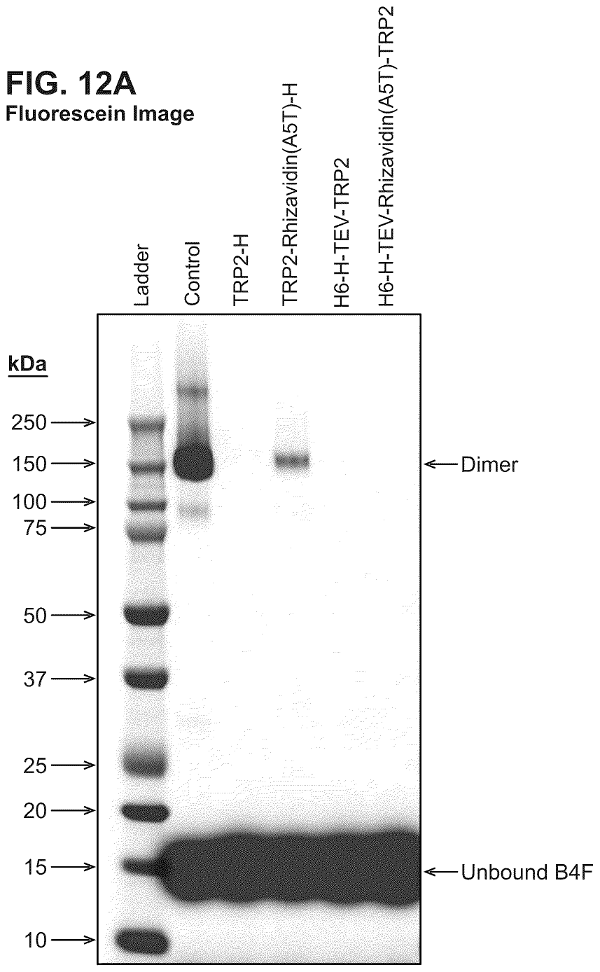

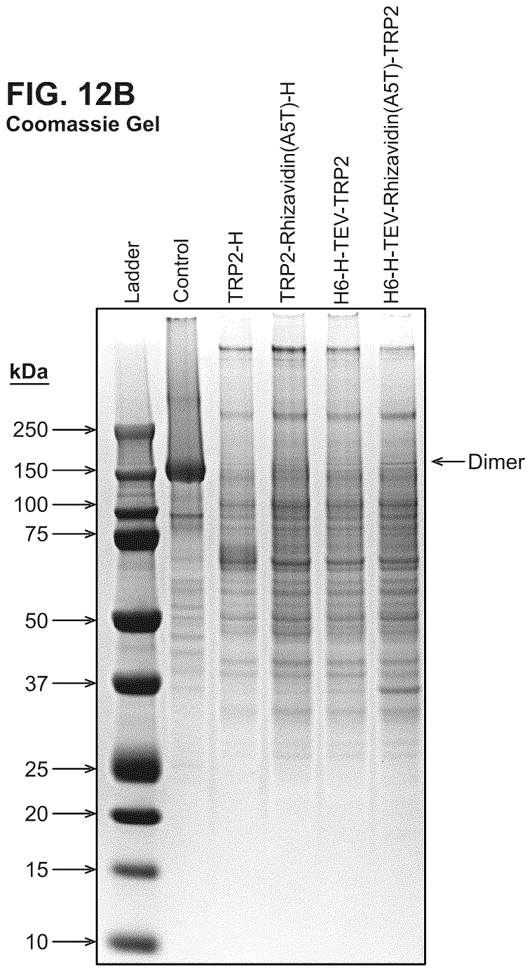

A series of constructs were made to assess whether rhizavidin A5T could form a fusion protein with the cancer antigen TRP2 which could retain biotin binding function following expression in a eukaryotic expression system. The constructs included TRP2 with a His-tag at the C-terminus, TRP2-Rhizavidin (A5T) with a Histag at the C-terminus, H6 leader sequence-His-tag-TEV-TRP2 and H6-His-tag-TEV-Rhizavidin (A5T)-TRP2. The constructs were expressed in Expi293F cells and purified as described in paragraph [0418] and were analyzed by SDS-PAGE and to assess biotin binding as described in paragraph [0419]. The results shown in FIG. 12 demonstrate that the TRP2-Rhizavidin (A5T)-H construct retained the ability to bind biotin. This construct is a further fusion protein that is capable to forming MAPS complexes with biotinylated molecules. Such MAPS complexes are interesting candidates for a vaccine for eliciting immune responses against cancer.

Sequence Listing

SEQ ID NO: 1, rhizavidin protein, full-length [aa 1-179]:
MIITSLYATFGTIADGRRTSGGKTMIRTNAVAALVFAVATSALAFDASNFKDFSSIASASS
SWQNQSGSTMIIQVDSFGNVSGQYVNRAQGTGCQNSPYPLTGRVNGTFIAFSVGWNNS
TENCNSATGWTGYAQVNGNNTEIVTSWNLAYEGGSGPAIEQGQDTFQYVPTTENKSLL
KD
SEQ ID NO: 2, truncated rhizavidin protein [aa 45-179], denoted Rhavi:
FDASNFKDFSSIASASSSWQNQSGSTMIIQVDSFGNVSGQYVNRAQGTGCQNSPYPLTGR
VNGTFIAFSVGWNNSTENCNSATGWTGYAQVNGNNTEIVTSWNLAYEGGSGPAIEQGQ
DTFQYVPTTENKSLLKD
SEQ ID NO: 3, truncated rhizavidin protein [aa 45-179], denoted Rhavi,
includes leading methionine:
MFDASNFKDFSSIASASSSWQNQSGSTMIIQVDSFGNVSGQYVNRAQGTGCQNSPYPLT
GRVNGTFIAFSVGWNNSTENCNSATGWTGYAQVNGNNTEIVTSWNLAYEGGSGPAIEQ
GQDTFQYVPTTENKSLLKD
SEQ ID NO: 4, rhizavidin protein, full-length [aa 1-179] with mutations N80A,
N106A, N118A, and N138A, denoted A4 variant:
MIITSLYATFGTIADGRRTSGGKTMIRTNAVAALVFAVATSALAFDASNFKDFSSIASASS
SWQNQSGSTMIIQVDSFGAVSGQYVNRAQGTGCQNSPYPLTGRVAGTFIAFSVGWNAS
TENCNSATGWTGYAQVNGANTEIVTSWNLAYEGGSGPAIEQGQDTFQYVPTTENKSLL
KD
SEQ ID NO: 5, truncated rhizavidin protein [aa 45-179] with mutations N80A,
N106A, N118A, and N138A, denoted A4 variant:
FDASNFKDFSSIASASSSWQNQSGSTMIIQVDSFGAVSGQYVNRAQGTGCQNSPYPLTGR
VAGTFIAFSVGWNASTENCNSATGWTGYAQVNGANTEIVTSWNLAYEGGSGPAIEQGQ
DTFQYVPTTENKSLLKD
SEQ ID NO: 6, truncated rhizavidin protein [aa 45-179] with mutations N80A,
N106A, N118A, and N138A, denoted A4 variant includes leading methionine:
MFDASNFKDFSSIASASSSWQNQSGSTMIIQVDSFGAVSGQYVNRAQGTGCQNSPYPLT
GRVAGTFIAFSVGWNASTENCNSATGWTGYAQVNGANTEIVTSWNLAYEGGSGPAIEQ
GQDTFQYVPTTENKSLLK
SEQ ID NO: 7, rhizavidin protein, full-length [aa 1-179] with mutations N80Q,
N106Q, N118Q, and N138Q, denoted Q4 variant:
MIITSLYATFGTIADGRRTSGGKTMIRTNAVAALVFAVATSALAFDASNFKDFSSIASASS
SWQNQSGSTMIIQVDSFGQVSGQYVNRAQGTGCQNSPYPLTGRVQGTFIAFSVGWNQS
TENCNSATGWTGYAQVNGQNTEIVTSWNLAYEGGSGPAIEQGQDTFQYVPTTENKSLL
KD
SEQ ID NO: 8, truncated rhizavidin protein [aa 45-179] with mutations N80Q,
N106Q, N118Q, and N138Q, denoted Q4 variant:
FDASNFKDFSSIASASSSWQNQSGSTMIIQVDSFGQVSGQYVNRAQGTGCQNSPYPLTG
RVQGTFIAFSVGWNQSTENCNSATGWTGYAQVNGQNTEIVTSWNLAYEGGSGPAIEQG
QDTFQYVPTTENKSLLKD
SEQ ID NO: 9, truncated rhizavidin protein [aa 45-179] with mutations N80Q,
N106Q, N118Q, and N138Q, denoted Q4 variant includes leading methionine:
MFDASNFKDFSSIASASSSWQNQSGSTMIIQVDSFGQVSGQYVNRAQGTGCQNSPYPLT
GRVQGTFIAFSVGWNQSTENCNSATGWTGYAQVNGQNTEIVTSWNLAYEGGSGPAIEQ
GQDTFQYVPTTENKSLLK
SEQ ID NO: 10, rhizavidin protein, full-length [aa 1-179] with mutations
N80A, N106A, N118A, S119A, and N138A, denoted A5 variant:
MIITSLYATFGTIADGRRTSGGKTMIRTNAVAALVFAVATSALAFDASNFKDFSSIASASS
SWQNQSGSTMIIQVDSFGAVSGQYVNRAQGTGCQNSPYPLTGRVAGTFIAFSVGWNAA
TENCNSATGWTGYAQVNGANTEIVTSWNLAYEGGSGPAIEQGQDTFQYVPTTENKSLL
KD
SEQ ID NO: 11, truncated rhizavidin protein [aa 45-179] with mutations
N80A, N106A, N118A, S119A, and N138A, denoted A5 variant:
FDASNFKDFSSIASASSSWQNQSGSTMIIQVDSFGAVSGQYVNRAQGTGCQNSPYPLTGR
VAGTFIAFSVGWNAATENCNSATGWTGYAQVNGANTEIVTSWNLAYEGGSGPAIEQGQ
DTFQYVPTTENKSLLKD
SEQ ID NO: 12, truncated rhizavidin protein [aa 45-179] with mutations
N80A, N106A, N118A, S119A, and N138A, denoted A5 variant includes leading methionine:
MFDASNFKDFSSIASASSSWQNQSGSTMIIQVDSFGAVSGQYVNRAQGTGCQNSPYPLT
GRVAGTFIAFSVGWNAATENCNSATGWTGYAQVNGANTEIVTSWNLAYEGGSGPAIEQ
GQDTFQYVPTTENKSLLKD
SEQ ID NO: 13, rhizavidin protein, full-length [aa 1-179] with mutations
N80A, T108A, N118A, S119A, and N138A, denoted A5T variant:
MIITSLYATFGTIADGRRTSGGKTMIRTNAVAALVFAVATSALAFDASNFKDFSSIASASS
SWQNQSGSTMIIQVDSFGAVSGQYVNRAQGTGCQNSPYPLTGRVNGAFIAFSVGWNAA
TENCNSATGWTGYAQVNGANTEIVTSWNLAYEGGSGPAIEQGQDTFQYVPTTENKSLL
KD
SEQ ID NO: 14, truncated rhizavidin protein [aa 45-179] with mutations
N80A, T108A, N118A, S119A, and N138A, denoted A5T variant:
FDASNFKDFSSIASASSSWQNQSGSTMIIQVDSFGAVSGQYVNRAQGTGCQNSPYPLTGR
VNGAFIAFSVGWNAATENCNSATGWTGYAQVNGANTEIVTSWNLAYEGGSGPAIEQG
QDTFQYVPTTENKSLLKD
SEQ ID NO: 15, truncated rhizavidin protein [aa 45-179] with mutations
N80A, T108A, N118A, S119A, and N138A, denoted A5T variant includes leading methionine:
MFDASNFKDFSSIASASSSWQNQSGSTMIIQVDSFGAVSGQYVNRAQGTGCQNSPYPLT
GRVNGAFIAFSVGWNAATENCNSATGWTGYAQVNGANTEIVTSWNLAYEGGSGPAIEQ
GQDTFQYVPTTENKSLLKD
SEQ ID NO: 16
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSET
KCTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGST
PCNGVEGFNCYFPLQSYGFQPTNGVGYQPYRVVVLSFELLHAPATVCGPKKSTNLVKN
KCVNFNFNGLTGTGVLTESNKKFLPFQQFGRDIADTTDAVRDPQTLEILDITPCSFGGVS
VITPGTNTSNQVAVLYQDVNCTEVPVAIHADQLTPTWRVYSTGSNVFQTRAGCLIGAEH
VNNSYECDIPIGAGICASYQTQTNSPRRARSVASQSIIAYTMSLGAENSVAYSNNSIAIPT
NFTISVTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNKVTLADAGFIKQY
GDCLGDIAARDLICAQKFNGLTVLPPLLTDEMIAQYTSALLAGTITSGWTFGAGAALQIP
FAMQMAYRFNGIGVTQNVLYENQKLIANQFNSAIGKIQDSLSSTASALGKLQDVVNQN
AQALNTLVKQLSSNFGAISSVLNDILSRLDKVEAEVQIDRLITGRLQSLQTYVTQQLIRAA
EIRASANLAATKMSECVLGQSKRVDFCGKGYHLMSFPQSAPHGVVFLHVTYVPAQEKN
FTTAPAICHDGKAHFPREGVFVSNGTHWFVTQRNFYEPQIITTDNTFVSGNCDVVIGIVN
NTVYDPLQPELDSFKEELDKYFKNHTSPDVDLGDISGINASVVNIQKEIDRLNEVAKNLN
ESLIDLQELGKYEQYIKWPWYIWLGFIAGLIAIVMVTIMLCCMTSCCSCLKGCCSCGSCC
KFDEDDSEPVLKGVKLHYT
SEQ ID NO: 17, surface glycoprotein RBD [Ancestral Wuhan seafood market
pneumonia virus]
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTNGVGYQPY
RVVVLSFELLHAPATV
SEQ ID NO: 18, Receptor Binding Motif (RBM) in surface glycoprotein RBD
[Ancestral Wuhan seafood market pneumonia virus]
NSNNLDSKVGGNYNYLYRLFRKSNLKPFERDISTEIYQAGSTPCNGVEGFNCYFPLQSY
GFQPTNGVGYQPY
SEQ ID NO: 19, SARS-CoV-2 Spike glycoprotein RBD (Ancestral Wuhan D614G)
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTNGVGYQPY
RVVVLSFELLHAPATVCGP
SEQ ID NO: 20, SARS-CoV-2 Spike glycoprotein RBD (Alpha, B.1.1.7, UK)
(N501Y)
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTYGVGYQPY
RVVVLSFELLHAPATVCGP
SEQ ID NO: 21, SARS-CoV-2 Spike glycoprotein RBD (Beta, B.1.351, South
Africa)(K417N, E484K, N501Y)
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYLYRLFRKSNLKPFERDISTEIYQAGSTPCNGVKGFNCYFPLQSYGFQPTYGVGYQP
YRVVVLSFELLHAPATVCGP
SEQ ID NO: 22, SARS-CoV-2 Spike glycoprotein RBD (Delta, B.1.617.2,
India)(L452R, T478K)
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSKPCNGVEGFNCYFPLQSYGFQPTNGVGYQP
YRVVVLSFELLHAPATVCGP
SEQ ID NO: 23, SARS-CoV-2 Spike glycoprotein RBD (Delta plus, AY.1,
India)(K417N, L452R, T478K)
NITNLCPFGEVFNATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSKPCNGVEGFNCYFPLQSYGFQPTNGVGYQP
YRVVVLSFELLHAPATVCGP
SEQ ID NO: 24, SARS-CoV-2 Spike glycoprotein RBD (Gamma, P.1,
Japan/Brazil) (K417T, E484K, N501Y)
NITNLCPFGEVENATRFASVYAWNRKRISNCVADYSVLYNSASFSTFKCYGVSPTKLND
LCFTNVYADSFVIRGDEVRQIAPGQTGTIADYNYKLPDDFTGCVIAWNSNNLDSKVGGN
YNYLYRLFRKSNLKPFERDISTEIYQAGSTPCNGVKGFNCYFPLQSYGFQPTYGVGYQP
YRVVVLSFELLHAPATVCGP
SEQ ID NO: 25, SARS-CoV-2 Spike glycoprotein RBD (Epsilon, B.1.427,
B.1.429, California) (L452R)
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVEGFNCYFPLQSYGFQPTNGVGYQPY
RVVVLSFELLHAPATVCGP
SEQ ID NO: 26, SARS-CoV-2 Spike glycoprotein RBD (Eta, B.1.525,
UK/Nigeria) (E484K)
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVKGFNCYFPLQSYGFQPTNGVGYQP
YRVVVLSFELLHAPATVCGP
SEQ ID NO: 27, SARS-CoV-2 Spike glycoprotein RBD (Iota, B.1.526,
US/NY)(L452R, E484K)
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVKGFNCYFPLQSYGFQPTNGVGYQP
YRVVVLSFELLHAPATVCGP
SEQ ID NO: 28, SARS-CoV-2 Spike glycoprotein RBD (Kappa, B.1.617.1,
India) (L452R, E484Q)
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVQGFNCYFPLQSYGFQPTNGVGYQP
YRVVVLSFELLHAPATVCGP
SEQ ID NO: 29, SARS-CoV-2 Spike glycoprotein RBD (Omicron, B.1.1.529,
Multiple Countries) (K417N, S477N, E484A, Q493K, G496S, N501Y, Y505H)
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYLYRLFRKSNLKPFERDISTEIYQAGNTPCNGVAGFNCYFPLKSYSFQPTYGVGHQP
YRVVVLSFELLHAPATVCGP
SEQ ID NO: 30, SARS-CoV-2 Spike glycoprotein RBD potential VOC #1
(K417N, L452R, E484Q, N501Y)
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSTPCNGVQGFNCYFPLQSYGFQPTYGVGYQP
YRVVVLSFELLHAPATVCGP
SEQ ID NO: 31, SARS-CoV-2 Spike glycoprotein RBD potential VOC #2
(K417N, L452R, E484K, N501Y)
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSTPCNGVKGFNCYFPLQSYGFQPTYGVGYQP
YRVVVLSFELLHAPATVCGP
SEQ ID NO: 32, SARS-CoV-2 Spike glycoprotein RBD potential VOC #3
(L452R, E484Q, N501Y)
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVQGFNCYFPLQSYGFQPTYGVGYQP
YRVVVLSFELLHAPATVCGP
SEQ ID NO: 33, SARS-CoV-2 Spike glycoprotein RBD potential VOC #4
(L452R, E484K, N501Y)
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVKGFNCYFPLQSYGFQPTYGVGYQP
YRVVVLSFELLHAPATVCGP
SEQ ID NO: 34, Streptococcus pneumoniae Pneumolysin protein:
MANKAVNDFILAMNYDKKKLLTHQGESIENRFIKEGNQLPDEFVVIERKKRSLSTNTSDI
SVTATNDSRLYPGALLVVDETLLENNPTLLAVDRAPMTYSIDLPGLASSDSFLQVEDPSN
SSVRGAVNDLLAKWHQDYGQVNNVPARMQYEKITAHSMEQLKVKFGSDFEKTGNSLD
IDFNSVHSGEKQIQIVNFKQIYYTVSVDAVKNPGDVFQDTVTVEDLKQRGISAERPLVYI
SSVAYGRQVYLKLETTSKSDEVEAAFEALIKGVKVAPQTEWKQILDNTEVKAVILGGDP
SSGARVVTGKVDMVEDLIQEGSRFTADHPGLPISYTTSFLRDNVVATFQNSTDYVETKV
TAYRNGDLLLDHSGAYVAQYYITWDELSYDHQGKEVLTPKAWDRNGQDLTAHFTTSIP
LKGNVRNLSVKIRECTGLAWEWWRTVYEKTDLPLVRKRTISIWGTTLYPQVEDKVEND
SEQ ID NO: 35, Streptococcus pneumoniae Pneumolysin protein with
mutations D385N, C428G, and W433F, denoted PdT:
MANKAVNDFILAMNYDKKKLLTHQGESIENRFIKEGNQLPDEFVVIERKKRSLSTNTSDI
SVTATNDSRLYPGALLVVDETLLENNPTLLAVDRAPMTYSIDLPGLASSDSFLQVEDPSN
SSVRGAVNDLLAKWHQDYGQVNNVPARMQYEKITAHSMEQLKVKFGSDFEKTGNSLD
IDFNSVHSGEKQIQIVNFKQIYYTVSVDAVKNPGDVFQDTVTVEDLKQRGISAERPLVYI
SSVAYGRQVYLKLETTSKSDEVEAAFEALIKGVKVAPQTEWKQILDNTEVKAVILGGDP
SSGARVVTGKVDMVEDLIQEGSRFTADHPGLPISYTTSFLRDNVVATFQNSTDYVETKV
TAYRNGDLLLDHSGAYVAQYYITWDELSYNHQGKEVLTPKAWDRNGQDLTAHFTTSIP
LKGNVRNLSVKIREGTGLAFEWWRTVYEKTDLPLVRKRTISIWGTTLYPQVEDKVEND
SEQ ID NO: 36, Streptococcus pneumoniae Pneumolysin protein with
mutations G294P, D385N, C428G, and W433F, denoted PdT(G294P):
MANKAVNDFILAMNYDKKKLLTHQGESIENRFIKEGNQLPDEFVVIERKKRSLSTNTSDI
SVTATNDSRLYPGALLVVDETLLENNPTLLAVDRAPMTYSIDLPGLASSDSFLQVEDPSN
SSVRGAVNDLLAKWHQDYGQVNNVPARMQYEKITAHSMEQLKVKFGSDFEKTGNSLD
IDFNSVHSGEKQIQIVNFKQIYYTVSVDAVKNPGDVFQDTVTVEDLKQRGISAERPLVYI
SSVAYGRQVYLKLETTSKSDEVEAAFEALIKGVKVAPQTEWKQILDNTEVKAVILGPDP
SSGARVVTGKVDMVEDLIQEGSRFTADHPGLPISYTTSFLRDNVVATFQNSTDYVETKV
TAYRNGDLLLDHSGAYVAQYYITWDELSYNHQGKEVLTPKAWDRNGQDLTAHFTTSIP
LKGNVRNLSVKIREGTGLAFEWWRTVYEKTDLPLVRKRTISIWGTTLYPQVEDKVEND
SEQ ID NO: 37, Streptococcus pneumoniae Pneumolysin PdT(G294P) [aa 2-470]
protein:
ANKAVNDFILAMNYDKKKLLTHQGESIENRFIKEGNQLPDEFVVIERKKRSLSTNTSDIS
VTATNDSRLYPGALLVVDETLLENNPTLLAVDRAPMTYSIDLPGLASSDSFLQVEDPSNS
SVRGAVNDLLAKWHQDYGQVNNVPARMQYEKITAHSMEQLKVKFGSDFEKTGNSLDI
DFNSVHSGEKQIQIVNFKQIYYTVSVDAVKNPGDVFQDTVTVEDLKQRGISAERPLVYIS
SVAYGRQVYLKLETTSKSDEVEAAFEALIKGVKVAPQTEWKQILDNTEVKAVILGPDPS
SGARVVTGKVDMVEDLIQEGSRFTADHPGLPISYTTSFLRDNVVATFQNSTDYVETKVT
AYRNGDLLLDHSGAYVAQYYITWDELSYNHQGKEVLTPKAWDRNGQDLTAHFTTSIPL
KGNVRNLSVKIREGTGLAFEWWRTVYEKTDLPLVRKRTISIWGTTLYPQVEDKVEND
SEQ ID NO: 38, Streptococcus pneumoniae SP0435 protein, full length, TIGR4
strain (GenBank: ABJ54475.1):
MIEASKLKAGMTFETADGKLIRVLEASHHKPGKGNTIMRMKLRDVRTGSTEDTSYRPEE
KFEQAIIETVPAQYLYKMDDTAYFMNTETYDQYEIPVVNVENELLYILENSDVKIQFYGT
EVIGVTVPTTVELTVAETQPSIKGATVTGSGKPATMETGLVVNVPDFIEAGQKLVINTAE
GTYVSRA
SEQ ID NO: 39, Streptococcus pneumoniae SP0435 [aa 62-185] protein,
TIGR4 strain:
EQAIIETVPAQYLYKMDDTAYFMNTETYDQYEIPVVNVENELLYILENSDVKIQFYGTEV
IGVTVPTTVELTVAETQPSIKGATVTGSGKPATMETGLVVNVPDFIEAGQKLVINTAEGT
YVSR
SEQ ID NO: 40, SP0785 protein, full-length [aa 1-399], TIGR4 strain: (Note:
One T394A mismatch with SP0785 NCBI Sequences ABJ54007.1 and YP816180)
MKKKNGKAKKWQLYAAIGAASVVVLGAGGILLFRQPSQTALKDEPTHLVVAKEGSVA
SSVLLSGTVTAKNEQYVYFDASKGDLDEILVSVGDKVSEGQALVKYSSSEAQAAYDSA
SRAVARADRHINELNQARNEAASAPAPQLPAPVGGEDATVQSPTPVAGNSVASIDAQLG
DARDARADAAAQLSKAQSQLDATTVLSTLEGTVVEVNSNVSKSPTGASQVMVHIVSNE
NLQVKGELSEYNLANLSVGQEVSFTSKVYPDKKWTGKLSYISDYPKNNGEAASPAAGN
NTGSKYPYTIDVTGEVGDLKQGFSVNIEVKSKTKAILVPVSSLVMDDSKNYVWIVDEQQ
KAKKVEVSLGNADAENQEITSGLINGAKVISNPTSSLEEGKEVKADEATN
SEQ ID NO: 41, SP0785 protein lacking signal sequence [aa 33-399]: (Note:
One T394A mismatch with SP0785 NCBI Sequences ABJ54007.1 and YP816180)
FRQPSQTALKDEPTHLVVAKEGSVASSVLLSGTVTAKNEQYVYFDASKGDLDEILVSVG
DKVSEGQALVKYSSSEAQAAYDSASRAVARADRHINELNQARNEAASAPAPQLPAPVG
GEDATVQSPTPVAGNSVASIDAQLGDARDARADAAAQLSKAQSQLDATTVLSTLEGTV
VEVNSNVSKSPTGASQVMVHIVSNENLQVKGELSEYNLANLSVGQEVSFTSKVYPDKK
WTGKLSYISDYPKNNGEAASPAAGNNTGSKYPYTIDVTGEVGDLKQGFSVNIEVKSKTK
AILVPVSSLVMDDSKNYVWIVDEQQKAKKVEVSLGNADAENQEITSGLTNGAKVISNPT
SSLEEGKEVKADEATN
SEQ ID NO: 42, SP1500 protein, full-length [aa 1-278], TIGR4 strain:
MKKWMLVLVSLMTALFLVACGKNSSETSGDNWSKYQSNKSITIGFDSTFVPMGFAQKD
GSYAGFDIDLATAVFEKYGITVNWQPIDWDLKEAELTKGTIDLIWNGYSATDERREKVA
FSNSYMKNEQVLVTKKSSGITTAKDMTGKTLGAQAGSSGYADFEANPEILKNIVANKEA
NQYQTFNEALIDLKNDRIDGLLIDRVYANYYLEAEGVLNDYNVFTVGLETEAFAVGAR
KEDTNLVKKINEAFSSLYKDGKFQEISQKWFGEDVATKEVKEGQ
SEQ ID NO: 43, SP1500 [aa 27-278]:
TSGDNWSKYQSNKSITIGFDSTFVPMGFAQKDGSYAGFDIDLATAVFEKYGITVNWQPI
DWDLKEAELTKGTIDLIWNGYSATDERREKVAFSNSYMKNEQVLVTKKSSGITTAKDM
TGKTLGAQAGSSGYADFEANPEILKNIVANKEANQYQTFNEALIDLKNDRIDGLLIDRVY
ANYYLEAEGVLNDYNVFTVGLETEAFAVGARKEDTNLVKKINEAFSSLYKDGKFQEISQ
KWFGEDVATKEVKEGQ
SEQ ID NO: 44, Ply gene encoding Ply protein, full-length [aa 1-470]
ATGGCAAATAAAGCAGTAAATGACTTTATACTAGCTATGAATTACGATAAAAAGAA
ACTCTTGACCCATCAGGGAGAAAGTATTGAAAATCGTTTCATCAAAGAGGGTAATC
AGCTACCCGATGAGTTTGTTGTTATCGAAAGAAAGAAGCGGAGCTTGTCGACAAAT
ACAAGTGATATTTCTGTAACAGCTACCAACGACAGTCGCCTCTATCCTGGAGCACTT
CTCGTAGTGGATGAGACCTTGTTAGAGAATAATCCCACTCTTCTTGCGGTCGATCGT
GCTCCGATGACTTATAGTATTGATTTGCCTGGTTTGGCAAGTAGCGATAGCTTTCTCC
AAGTGGAAGACCCCAGCAATTCAAGTGTTCGCGGAGCGGTAAACGATTTGTTGGCT
AAGTGGCATCAAGATTATGGTCAGGTCAATAATGTCCCAGCTAGAATGCAGCATGA
AAAAATCACGGCTCACAGCATGGAACAACTCAAGGTCAAGTTTGGTTCTGACTTTGA
AAAGATAGGGAATTCTCTTGATATTGATTTTAACTCTGTCCATTCAGGCGAAAAGCA
GATTCAGATTGTTAATTTTAAGCAGATTTATTATACAGTCAGCGTAGATGCTGTTAA
AAATCCAGGAGATGTGTTTCAAGATACTGTAACGGTAGAGGATTTAAGGCAGAGAG
GAATTTCTGCAGAGCGTCCTTTGGTCTATATTTCGAGTGTTGCTTATGGGCGCCAAGT
CTATCTCAAGTTGGAAACCACGAGTAAGAGTGATGAAGTAGAGGCTGCTTTTGAATC
TTTGATAAAAGGAGTAGCTCCTCAGACAGAGTGGAAGCAGATTTTGGACAATACAG
AAGTGAAGGCGGTTATTTTAGGGGGCGACCCAAGTTCGGGTGCCCGAGTTGTAACA
GGCAAGGTGGATATGGTAGAGGACTTGATTCAAGAAGGCAGTCGCTTTACAGCCGA
TCATCCAGGCTTGCCGATTTCCTATACAACTTCTTTTTTACGTGACAATGTAGTTGCG
ACCTTTCAAAACAGTACAGACTATGTTGAGACTAAGGTTACAGCTTACAGAAACGG
AGATTTACTGCTGGATCATAGTGGTGCCTATGTTGCTCAATATTATATTACTTGGGAT
GAATTATCCTATGATCATCAAGGCAAGGAAGTCTTGACTCCTAAGGCTTGGGACAGA
AATGGGCAGGATTTGACGGCTCACTTTACCACTAGTATTCCTTTAAAAGGGAATGTT
CGCAATCTCTCTGTCAAAATTAGAGAGTGTACCGGGCTTGCCTGGGAATGGTGGCGT
ACGGTTTATGAAAAAACCGATTTGCCACTAGTGCGTAAGCGGACGATTTCTATTTGG
GGAACAACTCTCTATCCTCAGGTAGAGGATAAGGTAGAAAATGATTAG
SEQ ID NO: 45, SP0435 gene encoding SP0435 protein, full-length [aa 1-186],
TIGR4 strain:
ATGATTGAAGCAAGTAAATTAAAAGCTGGTATGACCTTTGAAACAGCTGACGGCAA
ATTGATTCGCGTTTTGGAAGCTAGTCACCACAAACCAGGTAAAGGAAACACGATCA
TGCGTATGAAATTGCGTGATGTCCGTACTGGTTCTACATTTGACACAAGCTACCGTC
CAGAGGAAAAATTTGAACAAGCTATTATCGAGACTGTCCCAGCTCAATACTTGTACA
AAATGGATGACACAGCATACTTCATGAATACAGAAACTTATGACCAATACGAAATC
CCTGTAGTCAATGTTGAAAACGAATTGCTTTACATCCTTGAAAACTCTGATGTGAAA
ATCCAATTCTACGGAACTGAAGTGATCGGTGTCACCGTTCCTACTACTGTTGAGTTG
ACAGTTGCTGAAACTCAACCATCTATCAAAGGTGCTACTGTTACAGGTTCTGGTAAA
CCAGCAACGATGGAAACTGGACTTGTCGTAAACGTTCCAGACTTCATCGAAGCAGG
ACAAAAACTCGTTATCAACACTGCAGAAGGAACTTACGTTTCTCGTGCC
SEQ ID NO: 46, SP0785 gene encoding SP0785 protein, full-length [aa 1-399],
TIGR4 strain:
ATGAAGAAAAAGAATGGTAAAGCTAAAAAGTGGCAACTGTATGCAGCAATCGGTGC
TGCGAGTGTAGTTGTATTGGGTGCTGGGGGGATTTTACTCTTTAGACAACCTTCTCA
GACTGCTCTAAAAGATGAGCCTACTCATCTTGTTGTTGCCAAGGAAGGAAGCGTGGC
CTCCTCTGTTTTATTGTCAGGGACAGTAACAGCAAAAAATGAACAATATGTTTATTT
TGATGCTAGTAAGGGTGATTTAGATGAAATCCTTGTTTCTGTGGGCGATAAGGTCAG
CGAAGGGCAGGCTTTAGTCAAGTACAGTAGTTCAGAAGCGCAGGCGGCCTATGATT
CAGCTAGTCGAGCAGTAGCTAGGGCAGATCGTCATATCAATGAACTCAATCAAGCA
CGAAATGAAGCCGCTTCAGCTCCGGCTCCACAGTTACCAGCGCCAGTAGGAGGAGA
AGATGCAACGGTGCAAAGCCCAACTCCAGTGGCTGGAAATTCTGTTGCTTCTATTGA
CGCTCAATTGGGTGATGCCCGTGATGCGCGTGCAGATGCTGCGGCGCAATTAAGCA
AGGCTCAAAGTCAATTGGATGCAACAACTGTTCTCAGTACCCTAGAGGGAACTGTG
GTCGAAGTCAATAGCAATGTTTCTAAATCTCCAACAGGGGCGAGTCAAGTTATGGTT
CATATTGTCAGCAATGAAAATTTACAAGTCAAGGGAGAATTGTCTGAGTACAATCTA
GCCAACCTTTCTGTAGGTCAAGAAGTAAGCTTTACTTCTAAAGTGTATCCTGATAAA
AAATGGACTGGGAAATTAAGCTATATTTCTGACTATCCTAAAAACAATGGTGAAGC
AGCTAGTCCAGCAGCCGGGAATAATACAGGTTCTAAATACCCTTATACTATTGATGT
GACAGGCGAGGTTGGTGATTTGAAACAAGGTTTTTCTGTCAACATTGAGGTTAAAAG
CAAAACTAAGGCTATTCTTGTTCCTGTTAGCAGTCTAGTAATGGATGATAGTAAAAA
TTATGTCTGGATTGTGGATGAACAACAAAAGGCTAAAAAAGTTGAGGTTTCATTGGG
AAATGCTGACGCAGAAAATCAAGAAATCACTTCTGGTTTAACGAACGGTGCTAAGG
TCATCAGTAATCCAACATCTTCCTTGGAAGAAGGAAAAGAGGTGAAGGCTGATGAA
GCAACTAAT
SEQ ID NO: 47, SP1500 gene encoding SP1500 protein, full-length [aa 1-278],
TIGR4 strain:
ATGAAAAAATGGATGCTTGTATTAGTCAGTCTGATGACTGCTTTGTTCTTAGTAGCTT
GTGGGAAAAATTCTAGCGAAACTAGTGGAGATAATTGGTCAAAGTACCAGTCTAAC
AAGTCTATTACTATTGGATTTGATAGTACTTTTGTTCCAATGGGATTTGCTCAGAAAG
ATGGTTCTTATGCAGGATTTGATATTGATTTAGCTACAGCTGTTTTTGAAAAATACGG
AATCACGGTAAATTGGCAACCGATTGATTGGGATTTGAAAGAAGCTGAATTGACAA
AAGGAACGATTGATCTGATTTGGAATGGCTATTCCGCTACAGACGAACGCCGTGAA
AAGGTGGCTTTCAGTAACTCATATATGAAGAATGAGCAGGTATTGGTTACGAAGAA
ATCATCTGGTATCACGACTGCAAAGGATATGACTGGAAAGACATTAGGAGCTCAAG
CTGGTTCATCTGGTTATGCGGACTTTGAAGCAAATCCAGAAATTTTGAAGAATATTG
TCGCTAATAAGGAAGCGAATCAATACCAAACCTTTAATGAAGCCTTGATTGATTTGA
AAAACGATCGAATTGATGGTCTATTGATTGACCGTGTCTATGCAAACTATTATTTAG
AAGCAGAAGGTGTTTTAAACGATTATAATGTCTTTACAGTTGGACTAGAAACAGAA
GCTTTTGCGGTTGGAGCCCGTAAGGAAGATACAAACTTGGTTAAGAAGATAAATGA
AGCTTTTTCTAGTCTTTACAAGGACGGCAAGTTCCAAGAAATCAGCCAAAAATGGTT
TGGAGAAGATGTAGCAACCAAAGAAGTAAAAGAAGGACAG
SEQ ID NO: 48, linker sequence [7 amino acids]:
GGGGSSS
SEQ ID NO: 49, linker sequence [5 amino acid repeats]:
(GGGGS)ₙ
SEQ ID NO: 50, linker sequence [5 amino acids]:
GGGGS
SEQ ID NO: 51, linker sequence [15 amino acids]:
GGGGSGGGGSGGGGS
SEQ ID NO: 52, linker sequence [30 amino acids]:
GGGGSGGGGSGGGGSGGGGSGGGGSGGGGS
SEQ ID NO: 53, linker sequence [18 amino acids]:
KESGSVSSEQLAQFRSLD
SEQ ID NO: 54, linker sequence [14 amino acids]:
EGKSSGSGSESKST
SEQ ID NO: 55, linker sequence:
(Gly)ₙ
SEQ ID NO: 56, linker sequence [6 amino acids]:
GGGGGG
SEQ ID NO: 57, linker sequence [8 amino acids]:
GGGGGGGG
SEQ ID NO: 58, linker sequence [12 amino acids]:
GSAGSAAGSGEF
SEQ ID NO: 59, linker sequence [5 amino acid repeats]:
(EAAAK)ₙ
SEQ ID NO: 60, linker sequence:
EAAAK
SEQ ID NO: 61, linker sequence:
A(EAAAK)ₙA
SEQ ID NO: 62, linker sequence:
A(EAAAK)ₙA, n = 1-5
SEQ ID NO: 63, linker sequence:
A(EAAAK)₄ALEA(EAAAK)₄A
SEQ ID NO: 64, linker sequence:
[A(EAAAK)ₙA]ₘ, n = 1-4, m = 1-2
SEQ ID NO: 65, linker sequence [12 amino acids]:
AEAAAKEAAAKA
SEQ ID NO: 66, linker sequence [2 amino acid repeats]:
(XP)ₙ
SEQ ID NO: 67, linker sequence [2 amino acid repeats]:
(AP)ₙ
SEQ ID NO: 68, linker sequence [2 amino acid repeats]:
(AP)ₙ , n = 1-17
SEQ ID NO: 69, linker sequence [2 amino acid repeats]:
(KP)ₙ
SEQ ID NO: 70, linker sequence [2 amino acid repeats]:
(QP)ₙ
SEQ ID NO: 71, linker sequence [14 amino acids]:
APAPAPAPAPAPAP
SEQ ID NO: 72, GAG linker sequence [21 amino acids]:
GAPGGGGGAAAAAGGGGGGAP
SEQ ID NO: 73, GAG2 linker sequence [39 amino acids]:
GAPGGGGGAAAAAGGGGGGAPGGGGGAAAAAGGGGGGAP
SEQ ID NO: 74, GAG3 linker sequence [57 amino acids]:
GAPGGGGGAAAAAGGGGGGAPGGGGGAAAAAGGGGGGAPGGGGGAAAAAGGGGG
GAP
SEQ ID NO: 75, linker sequence [4 amino acids]:
VSDP
SEQ ID NO: 76, linker sequence [4 amino acids]:
AAAA
SEQ ID NO: 77, linker sequence [4 amino acids]:
GGGG
SEQ ID NO: 78, His tag 1:
(His)ₙ, n = 2-10
SEQ ID NO: 79, His tag 2:
HHHHHH
SEQ ID NO: 80, His tag 3:
MSYYHHHHHH
SEQ ID NO: 81, signal peptide sequence:
MKKIWLALAGLVLAFSASA
SEQ ID NO: 82, signal peptide sequence:
MAPFEPLASGILLLLWLIAPSRA
SEQ ID NO: 83, signal peptide sequence:
MKKVAAFVALSLLMAGC
SEQ ID NO: 84, signal peptide sequence:
MKKIMLVITLILVSPIAQQTEAKD
SEQ ID NO: 85, signal peptide sequence:
MKKKIISAILMSTVILSAAAPLSGVYADT
SEQ ID NO: 86, signal peptide sequence:
MKKRKVLIPLMALSTILVSSTGNLEVIQAEV
SEQ ID NO: 87, signal peptide sequence:
MNMKKATIAATAGIAVTAFAAPTIASAST
SEQ ID NO: 88, signal peptide sequence:
MQKTRKERILEALQEEKKNKKSKKFKTGATIAGVTAIATSITVPGIEVIVSADE
SEQ ID NO: 89, signal peptide sequence:
MKKLKMASCALVAGLMFSGLTPNAFAED
SEQ ID NO: 90, signal peptide sequence:
MAKKFNYKLPSMVALTLVGSAVTAHQVQAAE
SEQ ID NO: 91, signal peptide sequence:
MTDKKSENQTEKTETKENKGMTRREMLKLSAVAGTGIAVGATGLGTILNVVDQVDKA
LT
SEQ ID NO: 92, signal peptide sequence:
MAYDSRFDEWVQKLKEESFQNNTFDRRKFIQGAGKIAGLGLGLTIAQSVGAFG
SEQ ID NO: 93, signal peptide sequence:
MEFGLSWLFLVAILKGVQC
SEQ ID NO: 94, signal peptide sequence:
MGWSCIILFLVATATGVHS
SEQ ID NO: 95, S-RBD (Ancestral Wuhan D614G)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTNGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNASTENCNSAT
GWTGYAQVNGANTEIVTSWNLAYEGGSGPAIEQGQDTFQYVPTTENKSLLKD
SEQ ID NO: 96, S-RBD (Alpha)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTYGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNASTENCNSAT
GWTGYAQVNGANTEIVTSWNLAYEGGSGPAIEQGQDTFQYVPTTENKSLLKD
SEQ ID NO: 97, S-RBD (Beta)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYLYRLFRKSNLKPFERDISTEIYQAGSTPCNGVKGFNCYFPLQSYGFQPTYGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNASTENCNSAT
GWTGYAQVNGANTEIVTSWNLAYEGGSGPAIEQGQDTFQYVPTTENKSLLKD
SEQ ID NO: 98, S-RBD (Delta)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSKPCNGVEGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNASTENCNSA
TGWTGYAQVNGANTEIVTSWNLAYEGGSGPAIEQGQDTFQYVPTTENKSLLKD
SEQ ID NO: 99, S-RBD (Delta Plus)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSKPCNGVEGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNASTENCNSA
TGWTGYAQVNGANTEIVTSWNLAYEGGSGPAIEQGQDTFQYVPTTENKSLLKD
SEQ ID NO: 100, S-RBD (Gamma)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGTIADYNYKLPDDFTGCVIAWNSNNLDSKVGGN
YNYLYRLFRKSNLKPFERDISTEIYQAGSTPCNGVKGFNCYFPLQSYGFQPTYGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNASTENCNSAT
GWTGYAQVNGANTEIVTSWNLAYEGGSGPAIEQGQDTFQYVPTTENKSLLKD
SEQ ID NO: 101, S-RBD (Epsilon)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVEGFNCYFPLQSYGFQPTNGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNASTENCNSAT
GWTGYAQVNGANTEIVTSWNLAYEGGSGPAIEQGQDTFQYVPTTENKSLLKD
SEQ ID NO: 102, S-RBD (Eta)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVKGFNCYFPLQSYGFQPTNGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNASTENCNSAT
GWTGYAQVNGANTEIVTSWNLAYEGGSGPAIEQGQDTFQYVPTTENKSLLKD
SEQ ID NO: 103, S-RBD (Iota)-Rhavi-A4:
NITNLCPFGEVFNATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVKGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNASTENCNSA
TGWTGYAQVNGANTEIVTSWNLAYEGGSGPAIEQGQDTFQYVPTTENKSLLKD
SEQ ID NO: 104, S-RBD (Kappa)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVQGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNASTENCNSA
TGWTGYAQVNGANTEIVTSWNLAYEGGSGPAIEQGQDTFQYVPTTENKSLLKD
SEQ ID NO: 105, S-RBD (Omicron)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYLYRLFRKSNLKPFERDISTEIYQAGNTPCNGVAGFNCYFPLKSYSFQPTYGVGHQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNASTENCNSAT
GWTGYAQVNGANTEIVTSWNLAYEGGSGPAIEQGQDTFQYVPTTENKSLLKD
SEQ ID NO: 106, S-RBD (Potential VOC #1)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSTPCNGVQGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNASTENCNSA
TGWTGYAQVNGANTEIVTSWNLAYEGGSGPAIEQGQDTFQYVPTTENKSLLKD
SEQ ID NO: 107, S-RBD (Potential VOC #2)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSTPCNGVKGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNASTENCNSA
TGWTGYAQVNGANTEIVTSWNLAYEGGSGPAIEQGQDTFQYVPTTENKSLLKD
SEQ ID NO: 108, S-RBD (Potential VOC #3)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVQGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNASTENCNSA
TGWTGYAQVNGANTEIVTSWNLAYEGGSGPAIEQGQDTFQYVPTTENKSLLKD
SEQ ID NO: 109, S-RBD (Potential VOC #4)-Rhavi-A4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVKGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNASTENCNSA
TGWTGYAQVNGANTEIVTSWNLAYEGGSGPAIEQGQDTFQYVPTTENKSLLKD
SEQ ID NO: 110, S-RBD (Ancestral Wuhan D614G)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTNGVGYQPY
RVVVLSFELLHAPATVCGPGGGGSGGGGSGGGGSMFDASNFKDFSSIASASSSWQNQSG
STMIIQVDSFGQVSGQYVNRAQGTGCQNSPYPLTGRVQGTFIAFSVGWNQSTENCNSAT
GWTGYAQVNGQNTEIVTSWNLAYEGGSGPAIEQGQDTFQYVPTTENKSLLKD
SEQ ID NO: 111, S-RBD (Alpha)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTYGVGYQPY
RVVVLSFELLHAPATVCGPGGGGSGGGGSGGGGSMFDASNFKDFSSIASASSSWQNQSG
STMIIQVDSFGQVSGQYVNRAQGTGCQNSPYPLTGRVQGTFIAFSVGWNQSTENCNSAT
GWTGYAQVNGQNTEIVTSWNLAYEGGSGPAIEQGQDTFQYVPTTENKSLLKD
SEQ ID NO: 112, S-RBD (Beta)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYLYRLFRKSNLKPFERDISTEIYQAGSTPCNGVKGFNCYFPLQSYGFQPTYGVGYQPY
RVVVLSFELLHAPATVCGPGGGGSGGGGSGGGGSMFDASNFKDFSSIASASSSWQNQSG
STMIIQVDSFGQVSGQYVNRAQGTGCQNSPYPLTGRVQGTFIAFSVGWNQSTENCNSAT
GWTGYAQVNGQNTEIVTSWNLAYEGGSGPAIEQGQDTFQYVPTTENKSLLKD
SEQ ID NO: 113, S-RBD (Delta)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSKPCNGVEGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGQVSGQYVNRAQGTGCQNSPYPLTGRVQGTFIAFSVGWNQSTENCNSA
TGWTGYAQVNGQNTEIVTSWNLAYEGGSGPAIEQGQDTFQYVPTTENKSLLKD
SEQ ID NO: 114, S-RBD (Delta Plus)-Rhavi-Q4:
NITNLCPFGEVFNATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSKPCNGVEGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGQVSGQYVNRAQGTGCQNSPYPLTGRVQGTFIAFSVGWNQSTENCNSA
TGWTGYAQVNGQNTEIVTSWNLAYEGGSGPAIEQGQDTFQYVPTTENKSLLKD
SEQ ID NO: 115, S-RBD (Gamma)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGTIADYNYKLPDDFTGCVIAWNSNNLDSKVGGN
YNYLYRLFRKSNLKPFERDISTEIYQAGSTPCNGVKGFNCYFPLQSYGFQPTYGVGYQPY
RVVVLSFELLHAPATVCGPGGGGSGGGGSGGGGSMFDASNFKDFSSIASASSSWQNQSG
STMIIQVDSFGQVSGQYVNRAQGTGCQNSPYPLTGRVQGTFIAFSVGWNQSTENCNSAT
GWTGYAQVNGQNTEIVTSWNLAYEGGSGPAIEQGQDTFQYVPTTENKSLLKD
SEQ ID NO: 116, S-RBD (Epsilon)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVEGFNCYFPLQSYGFQPTNGVGYQPY
RVVVLSFELLHAPATVCGPGGGGSGGGGSGGGGSMFDASNFKDFSSIASASSSWQNQSG
STMIIQVDSFGQVSGQYVNRAQGTGCQNSPYPLTGRVQGTFIAFSVGWNQSTENCNSAT
GWTGYAQVNGQNTEIVTSWNLAYEGGSGPAIEQGQDTFQYVPTTENKSLLKD
SEQ ID NO: 117, S-RBD (Eta)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVKGFNCYFPLQSYGFQPTNGVGYQPY
RVVVLSFELLHAPATVCGPGGGGSGGGGSGGGGSMFDASNFKDFSSIASASSSWQNQSG
STMIIQVDSFGQVSGQYVNRAQGTGCQNSPYPLTGRVQGTFIAFSVGWNQSTENCNSAT
GWTGYAQVNGQNTEIVTSWNLAYEGGSGPAIEQGQDTFQYVPTTENKSLLKD
SEQ ID NO: 118, S-RBD (Iota)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVKGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGQVSGQYVNRAQGTGCQNSPYPLTGRVQGTFIAFSVGWNQSTENCNSA
TGWTGYAQVNGQNTEIVTSWNLAYEGGSGPAIEQGQDTFQYVPTTENKSLLKD
SEQ ID NO: 119, S-RBD (Kappa)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVQGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGQVSGQYVNRAQGTGCQNSPYPLTGRVQGTFIAFSVGWNQSTENCNSA
TGWTGYAQVNGQNTEIVTSWNLAYEGGSGPAIEQGQDTFQYVPTTENKSLLKD
SEQ ID NO: 120, S-RBD (Omicron)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYLYRLFRKSNLKPFERDISTEIYQAGNTPCNGVAGFNCYFPLKSYSFQPTYGVGHQPY
RVVVLSFELLHAPATVCGPGGGGSGGGGSGGGGSMFDASNFKDFSSIASASSSWQNQSG
STMIIQVDSFGQVSGQYVNRAQGTGCQNSPYPLTGRVQGTFIAFSVGWNQSTENCNSAT
GWTGYAQVNGQNTEIVTSWNLAYEGGSGPAIEQGQDTFQYVPTTENKSLLKD
SEQ ID NO: 121, S-RBD (Potential VOC #1)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSTPCNGVQGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGQVSGQYVNRAQGTGCQNSPYPLTGRVQGTFIAFSVGWNQSTENCNSA
TGWTGYAQVNGQNTEIVTSWNLAYEGGSGPAIEQGQDTFQYVPTTENKSLLKD
SEQ ID NO: 122, S-RBD (Potential VOC #2)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSTPCNGVKGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGQVSGQYVNRAQGTGCQNSPYPLTGRVQGTFIAFSVGWNQSTENCNSA
TGWTGYAQVNGQNTEIVTSWNLAYEGGSGPAIEQGQDTFQYVPTTENKSLLKD
SEQ ID NO: 123, S-RBD (Potential VOC #3)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVQGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGQVSGQYVNRAQGTGCQNSPYPLTGRVQGTFIAFSVGWNQSTENCNSA
TGWTGYAQVNGQNTEIVTSWNLAYEGGSGPAIEQGQDTFQYVPTTENKSLLKD
SEQ ID NO: 124, S-RBD (Potential VOC #4)-Rhavi-Q4:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVKGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGQVSGQYVNRAQGTGCQNSPYPLTGRVQGTFIAFSVGWNQSTENCNSA
TGWTGYAQVNGQNTEIVTSWNLAYEGGSGPAIEQGQDTFQYVPTTENKSLLKD
SEQ ID NO: 125, S-RBD (Ancestral Wuhan D614G)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTNGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNAATENCNSAT
GWTGYAQVNGANTEIVTSWNLAYEGGSGPAIEQGQDTFQYVPTTENKSLLKD
SEQ ID NO: 126, S-RBD (Alpha)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTYGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNAATENCNSAT
GWTGYAQVNGANTEIVTSWNLAYEGGSGPAIEQGQDTFQYVPTTENKSLLKD
SEQ ID NO: 127, S-RBD (Beta)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYLYRLFRKSNLKPFERDISTEIYQAGSTPCNGVKGFNCYFPLQSYGFQPTYGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNAATENCNSAT
GWTGYAQVNGANTEIVTSWNLAYEGGSGPAIEQGQDTFQYVPTTENKSLLKD
SEQ ID NO: 128, S-RBD (Delta)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSKPCNGVEGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNAATENCNS
ATGWTGYAQVNGANTEIVTSWNLAYEGGSGPAIEQGQDTFQYVPTTENKSLLKD
SEQ ID NO: 129, S-RBD (Delta Plus)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSKPCNGVEGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNAATENCNS
ATGWTGYAQVNGANTEIVTSWNLAYEGGSGPAIEQGQDTFQYVPTTENKSLLKD
SEQ ID NO: 130, S-RBD (Gamma)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGTIADYNYKLPDDFTGCVIAWNSNNLDSKVGGN
YNYLYRLFRKSNLKPFERDISTEIYQAGSTPCNGVKGFNCYFPLQSYGFQPTYGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNAATENCNSAT
GWTGYAQVNGANTEIVTSWNLAYEGGSGPAIEQGQDTFQYVPTTENKSLLKD
SEQ ID NO: 131, S-RBD (Epsilon)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVEGFNCYFPLQSYGFQPTNGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNAATENCNSAT
GWTGYAQVNGANTEIVTSWNLAYEGGSGPAIEQGQDTFQYVPTTENKSLLKD
SEQ ID NO: 132, S-RBD (Eta)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVKGFNCYFPLQSYGFQPTNGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNAATENCNSAT
GWTGYAQVNGANTEIVTSWNLAYEGGSGPAIEQGQDTFQYVPTTENKSLLKD
SEQ ID NO: 133, S-RBD (Iota)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVKGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNAATENCNS
ATGWTGYAQVNGANTEIVTSWNLAYEGGSGPAIEQGQDTFQYVPTTENKSLLKD
SEQ ID NO: 134, S-RBD (Kappa)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVQGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNAATENCNS
ATGWTGYAQVNGANTEIVTSWNLAYEGGSGPAIEQGQDTFQYVPTTENKSLLKD
SEQ ID NO: 135, S-RBD (Omicron)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYLYRLFRKSNLKPFERDISTEIYQAGNTPCNGVAGFNCYFPLKSYSFQPTYGVGHQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVAGTFIAFSVGWNAATENCNSAT
GWTGYAQVNGANTEIVTSWNLAYEGGSGPAIEQGQDTFQYVPTTENKSLLKD
SEQ ID NO: 136, S-RBD (Potential VOC #1)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSTPCNGVQGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNAATENCNS
ATGWTGYAQVNGANTEIVTSWNLAYEGGSGPAIEQGQDTFQYVPTTENKSLLKD
SEQ ID NO: 137, S-RBD (Potential VOC #2)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSTPCNGVKGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNAATENCNS
ATGWTGYAQVNGANTEIVTSWNLAYEGGSGPAIEQGQDTFQYVPTTENKSLLKD
SEQ ID NO: 138, S-RBD (Potential VOC #3)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVQGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNAATENCNS
ATGWTGYAQVNGANTEIVTSWNLAYEGGSGPAIEQGQDTFQYVPTTENKSLLKD
SEQ ID NO: 139, S-RBD (Potential VOC #4)-Rhavi-A5:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVKGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVAGTFIAFSVGWNAATENCNS
ATGWTGYAQVNGANTEIVTSWNLAYEGGSGPAIEQGQDTFQYVPTTENKSLLKD
SEQ ID NO: 140, S-RBD (Ancestral Wuhan D614G)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTNGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVNGAFIAFSVGWNAATENCNSA
TGWTGYAQVNGANTEIVTSWNLAYEGGSGPAIEQGQDTFQYVPTTENKSLLKD
SEQ ID NO: 141, S-RBD (Alpha)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTYGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVNGAFIAFSVGWNAATENCNSA
TGWTGYAQVNGANTEIVTSWNLAYEGGSGPAIEQGQDTFQYVPTTENKSLLKD
SEQ ID NO: 142, S-RBD (Beta)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYLYRLFRKSNLKPFERDISTEIYQAGSTPCNGVKGFNCYFPLQSYGFQPTYGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVNGAFIAFSVGWNAATENCNSA
TGWTGYAQVNGANTEIVTSWNLAYEGGSGPAIEQGQDTFQYVPTTENKSLLKD
SEQ ID NO: 143, S-RBD (Delta)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSKPCNGVEGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVNGAFIAFSVGWNAATENCNS
ATGWTGYAQVNGANTEIVTSWNLAYEGGSGPAIEQGQDTFQYVPTTENKSLLKD
SEQ ID NO: 144, S-RBD (Delta Plus)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSKPCNGVEGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVNGAFIAFSVGWNAATENCNS
ATGWTGYAQVNGANTEIVTSWNLAYEGGSGPAIEQGQDTFQYVPTTENKSLLKD
SEQ ID NO: 145, S-RBD (Gamma)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGTIADYNYKLPDDFTGCVIAWNSNNLDSKVGGN
YNYLYRLFRKSNLKPFERDISTEIYQAGSTPCNGVKGFNCYFPLQSYGFQPTYGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVNGAFIAFSVGWNAATENCNSA
TGWTGYAQVNGANTEIVTSWNLAYEGGSGPAIEQGQDTFQYVPTTENKSLLKD
SEQ ID NO: 146, S-RBD (Epsilon)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVEGFNCYFPLQSYGFQPTNGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVNGAFIAFSVGWNAATENCNSA
TGWTGYAQVNGANTEIVTSWNLAYEGGSGPAIEQGQDTFQYVPTTENKSLLKD
SEQ ID NO: 147, S-RBD (Eta)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVKGFNCYFPLQSYGFQPTNGVGYQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVNGAFIAFSVGWNAATENCNSA
TGWTGYAQVNGANTEIVTSWNLAYEGGSGPAIEQGQDTFQYVPTTENKSLLKD
SEQ ID NO: 148, S-RBD (Iota)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVKGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVNGAFIAFSVGWNAATENCNS
ATGWTGYAQVNGANTEIVTSWNLAYEGGSGPAIEQGQDTFQYVPTTENKSLLKD
SEQ ID NO: 149, S-RBD (Kappa)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVQGFNCYFPLQSYGFQPTNGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVNGAFIAFSVGWNAATENCNS
ATGWTGYAQVNGANTEIVTSWNLAYEGGSGPAIEQGQDTFQYVPTTENKSLLKD
SEQ ID NO: 150, S-RBD (Omicron)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYLYRLFRKSNLKPFERDISTEIYQAGNTPCNGVAGFNCYFPLKSYSFQPTYGVGHQPY
RVVVLSFELLHAPATVCGPGGGGSGGGGSGGGGSMFDASNFKDFSSIASASSSWQNQSG
STMIIQVDSFGAVSGQYVNRAQGTGCQNSPYPLTGRVNGAFIAFSVGWNAATENCNSA
TGWTGYAQVNGANTEIVTSWNLAYEGGSGPAIEQGQDTFQYVPTTENKSLLKD
SEQ ID NO: 151, S-RBD (Potential VOC #1)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSTPCNGVQGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVNGAFIAFSVGWNAATENCNS
ATGWTGYAQVNGANTEIVTSWNLAYEGGSGPAIEQGQDTFQYVPTTENKSLLKD
SEQ ID NO: 152, S-RBD (Potential VOC #2)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYRYRLFRKSNLKPFERDISTEIYQAGSTPCNGVKGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVNGAFIAFSVGWNAATENCNS
ATGWTGYAQVNGANTEIVTSWNLAYEGGSGPAIEQGQDTFQYVPTTENKSLLKD
SEQ ID NO: 153, S-RBD (Potential VOC #3)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVQGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVNGAFIAFSVGWNAATENCNS
ATGWTGYAQVNGANTEIVTSWNLAYEGGSGPAIEQGQDTFQYVPTTENKSLLKD
SEQ ID NO: 154, S-RBD (Potential VOC #4)-Rhavi-A5T:
NITNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVKGFNCYFPLQSYGFQPTYGVGYQP
YRVVVLSFELLHAPATVCGPGGGGSGGGGSGGGGSMFDASNFKDFSSIASASSSWQNQS
GSTMIIQVDSFGAVSGQYVNRAQGTGCQNSPYPLTGRVNGAFIAFSVGWNAATENCNS
ATGWTGYAQVNGANTEIVTSWNLAYEGGSGPAIEQGQDTFQYVPTTENKSLLKD
SEQ ID NO: 155, Rhavi-A4-linker-PdT(G294P)-linker-SP0435 [aa 62-185]
fusion protein, denoted SPP2 (A4):
MFDASNFKDFSSIASASSSWQNQSGSTMIIQVDSFGAVSGQYVNRAQGTGCQNSPYPLT
GRVAGTFIAFSVGWNASTENCNSATGWTGYAQVNGANTEIVTSWNLAYEGGSGPAIEQ
GQDTFQYVPTTENKSLLKDGGGGSSSANKAVNDFILAMNYDKKKLLTHQGESIENRFIK
EGNQLPDEFVVIERKKRSLSTNTSDISVTATNDSRLYPGALLVVDETLLENNPTLLAVDR
APMTYSIDLPGLASSDSFLQVEDPSNSSVRGAVNDLLAKWHQDYGQVNNVPARMQYE
KITAHSMEQLKVKFGSDFEKTGNSLDIDFNSVHSGEKQIQIVNFKQIYYTVSVDAVKNPG
DVFQDTVTVEDLKQRGISAERPLVYISSVAYGRQVYLKLETTSKSDEVEAAFEALIKGVK
VAPQTEWKQILDNTEVKAVILGPDPSSGARVVTGKVDMVEDLIQEGSRFTADHPGLPIS
YTTSFLRDNVVATFQNSTDYVETKVTAYRNGDLLLDHSGAYVAQYYITWDELSYNHQ
GKEVLTPKAWDRNGQDLTAHFTTSIPLKGNVRNLSVKIREGTGLAFEWWRTVYEKTDL
PLVRKRTISIWGTTLYPQVEDKVENDGGGGSSSEQAIIETVPAQYLYKMDDTAYFMNTE
TYDQYEIPVVNVENELLYILENSDVKIQFYGTEVIGVTVPTTVELTVAETQPSIKGATVTG
SGKPATMETGLVVNVPDFIEAGQKLVINTAEGTYVSR
SEQ ID NO: 156, Rhavi-Q4-linker-PdT(G294P)-linker-SP0435 [aa 62-185]
fusion protein, denoted SPP2 (Q4):
MFDASNFKDFSSIASASSSWQNQSGSTMIIQVDSFGQVSGQYVNRAQGTGCQNSPYPLT
GRVQGTFIAFSVGWNQSTENCNSATGWTGYAQVNGQNTEIVTSWNLAYEGGSGPAIEQ
GQDTFQYVPTTENKSLLKDGGGGSSSANKAVNDFILAMNYDKKKLLTHQGESIENRFIK
EGNQLPDEFVVIERKKRSLSTNTSDISVTATNDSRLYPGALLVVDETLLENNPTLLAVDR
APMTYSIDLPGLASSDSFLQVEDPSNSSVRGAVNDLLAKWHQDYGQVNNVPARMQYE
KITAHSMEQLKVKFGSDFEKTGNSLDIDFNSVHSGEKQIQIVNFKQIYYTVSVDAVKNPG
DVFQDTVTVEDLKQRGISAERPLVYISSVAYGRQVYLKLETTSKSDEVEAAFEALIKGVK
VAPQTEWKQILDNTEVKAVILGPDPSSGARVVTGKVDMVEDLIQEGSRFTADHPGLPIS
YTTSFLRDNVVATFQNSTDYVETKVTAYRNGDLLLDHSGAYVAQYYITWDELSYNHQ
GKEVLTPKAWDRNGQDLTAHFTTSIPLKGNVRNLSVKIREGTGLAFEWWRTVYEKTDL
PLVRKRTISIWGTTLYPQVEDKVENDGGGGSSSEQAIIETVPAQYLYKMDDTAYFMNTE
TYDQYEIPVVNVENELLYILENSDVKIQFYGTEVIGVTVPTTVELTVAETQPSIKGATVTG
SGKPATMETGLVVNVPDFIEAGQKLVINTAEGTYVSR
SEQ ID NO: 157, Rhavi-A5-linker-PdT(G294P)-linker-SP0435 [aa 62-185]
fusion protein, denoted SPP2 (A5):
MFDASNFKDFSSIASASSSWQNQSGSTMIIQVDSFGAVSGQYVNRAQGTGCQNSPYPLT
GRVAGTFIAFSVGWNAATENCNSATGWTGYAQVNGANTEIVTSWNLAYEGGSGPAIEQ
GQDTFQYVPTTENKSLLKDGGGGSSSANKAVNDFILAMNYDKKKLLTHQGESIENRFIK
EGNQLPDEFVVIERKKRSLSTNTSDISVTATNDSRLYPGALLVVDETLLENNPTLLAVDR
APMTYSIDLPGLASSDSFLQVEDPSNSSVRGAVNDLLAKWHQDYGQVNNVPARMQYE
KITAHSMEQLKVKFGSDFEKTGNSLDIDFNSVHSGEKQIQIVNFKQIYYTVSVDAVKNPG
DVFQDTVTVEDLKQRGISAERPLVYISSVAYGRQVYLKLETTSKSDEVEAAFEALIKGVK
VAPQTEWKQILDNTEVKAVILGPDPSSGARVVTGKVDMVEDLIQEGSRFTADHPGLPIS
YTTSFLRDNVVATFQNSTDYVETKVTAYRNGDLLLDHSGAYVAQYYITWDELSYNHQ
GKEVLTPKAWDRNGQDLTAHFTTSIPLKGNVRNLSVKIREGTGLAFEWWRTVYEKTDL
PLVRKRTISIWGTTLYPQVEDKVENDGGGGSSSEQAIIETVPAQYLYKMDDTAYFMNTE
TYDQYEIPVVNVENELLYILENSDVKIQFYGTEVIGVTVPTTVELTVAETQPSIKGATVTG
SGKPATMETGLVVNVPDFIEAGQKLVINTAEGTYVSR
SEQ ID NO: 158, Rhavi-A5T-linker-PdT(G294P)-linker-SP0435 [aa 62-185]
fusion protein, denoted SPP2 (A5T):
MFDASNFKDFSSIASASSSWQNQSGSTMIIQVDSFGAVSGQYVNRAQGTGCQNSPYPLT
GRVNGAFIAFSVGWNAATENCNSATGWTGYAQVNGANTEIVTSWNLAYEGGSGPAIEQ
GQDTFQYVPTTENKSLLKDGGGGSSSANKAVNDFILAMNYDKKKLLTHQGESIENRFIK
EGNQLPDEFVVIERKKRSLSTNTSDISVTATNDSRLYPGALLVVDETLLENNPTLLAVDR
APMTYSIDLPGLASSDSFLQVEDPSNSSVRGAVNDLLAKWHQDYGQVNNVPARMQYE
KITAHSMEQLKVKFGSDFEKTGNSLDIDFNSVHSGEKQIQIVNFKQIYYTVSVDAVKNPG
DVFQDTVTVEDLKQRGISAERPLVYISSVAYGRQVYLKLETTSKSDEVEAAFEALIKGVK
VAPQTEWKQILDNTEVKAVILGPDPSSGARVVTGKVDMVEDLIQEGSRFTADHPGLPIS
YTTSFLRDNVVATFQNSTDYVETKVTAYRNGDLLLDHSGAYVAQYYITWDELSYNHQ
GKEVLTPKAWDRNGQDLTAHFTTSIPLKGNVRNLSVKIREGTGLAFEWWRTVYEKTDL
PLVRKRTISIWGTTLYPQVEDKVENDGGGGSSSEQAIIETVPAQYLYKMDDTAYFMNTE
TYDQYEIPVVNVENELLYILENSDVKIQFYGTEVIGVTVPTTVELTVAETQPSIKGATVTG
SGKPATMETGLVVNVPDFIEAGQKLVINTAEGTYVSR
SEQ ID NO: 159, Rhavi-A4-linker-SP1500 [aa 27-278]-linker-SP0785 [aa 33-399]
fusion protein, denoted CP1 (A4):
MFDASNFKDFSSIASASSSWQNQSGSTMIIQVDSFGAVSGQYVNRAQGTGCQNSPYPLT
GRVAGTFIAFSVGWNASTENCNSATGWTGYAQVNGANTEIVTSWNLAYEGGSGPAIEQ
GQDTFQYVPTTENKSLLKDGGGGSSSTSGDNWSKYQSNKSITIGFDSTFVPMGFAQKDG
SYAGFDIDLATAVFEKYGITVNWQPIDWDLKEAELTKGTIDLIWNGYSATDERREKVAF
SNSYMKNEQVLVTKKSSGITTAKDMTGKTLGAQAGSSGYADFEANPEILKNIVANKEA
NQYQTFNEALIDLKNDRIDGLLIDRVYANYYLEAEGVLNDYNVFTVGLETEAFAVGAR
KEDTNLVKKINEAFSSLYKDGKFQEISQKWFGEDVATKEVKEGQAAAFRQPSQTALKD
EPTHLVVAKEGSVASSVLLSGTVTAKNEQYVYFDASKGDLDEILVSVGDKVSEGQALV
KYSSSEAQAAYDSASRAVARADRHINELNQARNEAASAPAPQLPAPVGGEDATVQSPTP
VAGNSVASIDAQLGDARDARADAAAQLSKAQSQLDATTVLSTLEGTVVEVNSNVSKSP
TGASQVMVHIVSNENLQVKGELSEYNLANLSVGQEVSFTSKVYPDKKWTGKLSYISDY
PKNNGEAASPAAGNNTGSKYPYTIDVTGEVGDLKQGFSVNIEVKSKTKAILVPVSSLVM
DDSKNYVWIVDEQQKAKKVEVSLGNADAENQEITSGLINGAKVISNPTSSLEEGKEVK
ADEATN
SEQ ID NO: 160, Rhavi-Q4-linker-SP1500 [aa 27-278]-linker-SP0785 [aa 33-399]
fusion protein, denoted CP1 (Q4):
MFDASNFKDFSSIASASSSWQNQSGSTMIIQVDSFGQVSGQYVNRAQGTGCQNSPYPLT
GRVQGTFIAFSVGWNQSTENCNSATGWTGYAQVNGQNTEIVTSWNLAYEGGSGPAIEQ
GQDTFQYVPTTENKSLLKDGGGGSSSTSGDNWSKYQSNKSITIGFDSTFVPMGFAQKDG
SYAGFDIDLATAVFEKYGITVNWQPIDWDLKEAELTKGTIDLIWNGYSATDERREKVAF
SNSYMKNEQVLVTKKSSGITTAKDMTGKTLGAQAGSSGYADFEANPEILKNIVANKEA
NQYQTFNEALIDLKNDRIDGLLIDRVYANYYLEAEGVLNDYNVFTVGLETEAFAVGAR
KEDTNLVKKINEAFSSLYKDGKFQEISQKWFGEDVATKEVKEGQAAAFRQPSQTALKD
EPTHLVVAKEGSVASSVLLSGTVTAKNEQYVYFDASKGDLDEILVSVGDKVSEGQALV
KYSSSEAQAAYDSASRAVARADRHINELNQARNEAASAPAPQLPAPVGGEDATVQSPTP
VAGNSVASIDAQLGDARDARADAAAQLSKAQSQLDATTVLSTLEGTVVEVNSNVSKSP
TGASQVMVHIVSNENLQVKGELSEYNLANLSVGQEVSFTSKVYPDKKWTGKLSYISDY
PKNNGEAASPAAGNNTGSKYPYTIDVTGEVGDLKQGFSVNIEVKSKTKAILVPVSSLVM
DDSKNYVWIVDEQQKAKKVEVSLGNADAENQEITSGLINGAKVISNPTSSLEEGKEVK
ADEATN
SEQ ID NO: 161, Rhavi-A5-linker-SP1500 [aa 27-278]-linker-SP0785 [aa 33-399]
fusion protein, denoted CP1 (A5):
MFDASNFKDFSSIASASSSWQNQSGSTMIIQVDSFGAVSGQYVNRAQGTGCQNSPYPLT
GRVAGTFIAFSVGWNAATENCNSATGWTGYAQVNGANTEIVTSWNLAYEGGSGPAIEQ
GQDTFQYVPTTENKSLLKDGGGGSSSTSGDNWSKYQSNKSITIGFDSTFVPMGFAQKDG
SYAGFDIDLATAVFEKYGITVNWQPIDWDLKEAELTKGTIDLIWNGYSATDERREKVAF
SNSYMKNEQVLVTKKSSGITTAKDMTGKTLGAQAGSSGYADFEANPEILKNIVANKEA
NQYQTFNEALIDLKNDRIDGLLIDRVYANYYLEAEGVLNDYNVFTVGLETEAFAVGAR
KEDTNLVKKINEAFSSLYKDGKFQEISQKWFGEDVATKEVKEGQAAAFRQPSQTALKD
EPTHLVVAKEGSVASSVLLSGTVTAKNEQYVYFDASKGDLDEILVSVGDKVSEGQALV
KYSSSEAQAAYDSASRAVARADRHINELNQARNEAASAPAPQLPAPVGGEDATVQSPTP
VAGNSVASIDAQLGDARDARADAAAQLSKAQSQLDATTVLSTLEGTVVEVNSNVSKSP
TGASQVMVHIVSNENLQVKGELSEYNLANLSVGQEVSFTSKVYPDKKWTGKLSYISDY
PKNNGEAASPAAGNNTGSKYPYTIDVTGEVGDLKQGFSVNIEVKSKTKAILVPVSSLVM
DDSKNYVWIVDEQQKAKKVEVSLGNADAENQEITSGLINGAKVISNPTSSLEEGKEVK
ADEATN
SEQ ID NO: 162, Rhavi-A5T-linker-SP1500 [aa 27-278]-linker-SP0785 [aa 33-399]
fusion protein, denoted CP1 (A5T):
MFDASNFKDFSSIASASSSWQNQSGSTMIIQVDSFGAVSGQYVNRAQGTGCQNSPYPLTGRVNG
AFIAFSVGWNAATENCNSATGWTGYAQVNGANTEIVTSWNLAYEGGSGPAIEQGQDTFQYVPT
TENKSLLKDGGGGSSSTSGDNWSKYQSNKSITIGFDSTFVPMGFAQKDGSYAGFDIDLATAVFEK
YGITVNWQPIDWDLKEAELTKGTIDLIWNGYSATDERREKVAFSNSYMKNEQVLVTKKSSGITT
AKDMTGKTLGAQAGSSGYADFEANPEILKNIVANKEANQYQTFNEALIDLKNDRIDGLLIDRVY
ANYYLEAEGVLNDYNVFTVGLETEAFAVGARKEDTNLVKKINEAFSSLYKDGKFQEISQKWFGE
DVATKEVKEGQAAAFRQPSQTALKDEPTHLVVAKEGSVASSVLLSGTVTAKNEQYVYFDASKG
DLDEILVSVGDKVSEGQALVKYSSSEAQAAYDSASRAVARADRHINELNQARNEAASAPAPQLP
APVGGEDATVQSPTPVAGNSVASIDAQLGDARDARADAAAQLSKAQSQLDATTVLSTLEGTVV
EVNSNVSKSPTGASQVMVHIVSNENLQVKGELSEYNLANLSVGQEVSFTSKVYPDKKWTGKLS
YISDYPKNNGEAASPAAGNNTGSKYPYTIDVTGEVGDLKQGFSVNIEVKSKTKAILVPVSSLVMD
DSKNYVWIVDEQQKAKKVEVSLGNADAENQEITSGLTNGAKVISNPTSSLEEGKEVKADEATN

### REFERENCES

Anttila M, Eskola J, Ahman H, Käyhty H. Avidity of IgG for Streptococcus pneumoniae type 6B and 23F polysaccharides in infants primed with pneumococcal conjugates and boosted with polysaccharide or conjugate vaccines. J Infect Dis. 177(6):1614-21 (1998).
Ausubel F M, Brent R, Kingston R E, Moore D D, Seidman J G, Smith J A, Struhl K. Current Protocols in Molecular Biology. Wiley, New York (1994).
Avery L B, et al., Establishing in vitro in vivo correlations to screen monoclonal antibodies for physiochemical properties related to favorable human pharmacokinetics. MAbs. 10(2):244-55 (2018).
Azze R F O, Rodriguez J C M, Iniesta M G, Marchena X R F, Alfonso V M R, Padron F T S. Immunogenicity of a new Salmonella Typhi Vi polysaccharide vaccine—vax-TyVi—in Cuban school children and teenagers. Vaccines. 21(21-22):2758-60 (2003).
Berry A M, Alexander J E, Mitchel T J, Andrew P W, Hansman D, Paton J C. Effect of defined point mutations in the pneumolysin gene on the virulence of Streptococcus pneumoniae. Infect Immun. 63(5):1969-74 (1995).
Boerner P, Lafond R, Lu W Z, Brams P, Royston I. Production of antigen-specific human monoclonal antibodies from in vitro-primed human splenocytes. J Immunol. 147(1):86-95 (1991).
Chen X, Zaro J, Shen W C. Fusion protein linkers: property, design and functionality. Adv. Drug Deliv. Rev. 65(10):1357-69 (2013).
Cieslewicz, B.; Makrinos, D.; Burke, H.; Bree, D.; Haridas, R.; Tonkiss, I.; Bartsch, Y.; Alter, G.; Malley, R.; Besin, G. Preclinical Immunogenicity and Efficacy of a Multiple Antigen-Presenting System (MAPS) SARS-CoV-2 Vaccine. Vaccines 10:1069 (2022).
Cole et al., Monoclonal Antibodies and Cancer Therapy, Alan R. Liss (1985).
Concepcion N F, Frasch C E. Pneumococcal type 22f polysaccharide absorption improves the specificity of a pneumococcal-polysaccharide enzyme-linked immunosorbent assay. Clin Diagn Lab Immunol. 8(2):266-72 (2001).
Dao T and Scheinberg D A. Peptide vaccines for myeloid leukaemias. Best Pract Res Clin Haematol. 21(3):391-404 (2008).
Dombs A J and Kumar N. “Biocompatible Polymers for Nucleic Acid Delivery” Biodegradable Polymers in Clinical Use and Clinical Development. Wiley (2011).
Douce et al. Mutants of Escherichia coli heat-labile toxin lacking ADP-ribosyltransferase activity act as non-toxic, mucosal adjuvants. PNAS Vol. 92, pp. 1644-1648 (1995).
Douce et al. Genetically detoxified mutants of heat-labile toxin from Escherichia coli are able to act as oral adjuvants” Infect Immun. 67(9):4400-6 (1999).
El-Sayed M E H, Hoffman A S, Stayton P S. Smart Polymer Carriers for Enhanced Intracellular Delivery of Therapeutic Molecules. Expert. Opin. Biol. Ther. 5(1):23-32 (2005)
Evans J T et al. Enhancement of antigen-specific immunity via the TLR-4 ligands MPL adjuvant and Ribi.529. Expert Rev. Vaccines 2(2):219-29 (2003).
Geno K A, Gilbert G L, Song J Y, Skovsted I C, Klugman K P, Jones C et al. Pneumococcal Capsules and their types: past, present, and future. Clin Microbiol Rev 28(3):871-899 (2015).
Giuliani M M et al. Mucosal adjuvanticity and immunogenicity of LTR72, a novel mutant of Escherichia coli heat-labile enterotoxin with partial knockout of ADP-ribosyltransferase activity. J Exp Med. 187(7):1123-32 (1998).
Gupta R and Brunak S. Prediction of glycosylation across the human proteome and the correlation to protein function. Pac. Symp. Biocompt. 310-22 (2002).
Helppolainen S H, Nurminen K P, Määttä JA, Halling K K, Slotte J P, Huhtala T, et al. Rhizavidin from Rhizobium etli: the first natural dimer in the avidin protein family. Biochem J. 405:397-405 (2007).
Holliger P, Prospero T, Winter G. “Diabodies”: small bivalent and bispecific antibody fragments. Proc Natl Acad Sci USA. 90(14):6444-8 (1993).
Ishizaka S T et al. “E6020: a synthetic Toll-like receptor 4 agonist as a vaccine adjuvant.” Expert Rev. Vaccines. 6(5):773-84 (2007).
Jerabek-Willemsen, M.; André, T.; Wanner, R.; Roth, H. M.; Duhr, S.; Baaske, P.; Breitsprecher, D. MicroScale Thermophoresis: Interaction Analysis and Beyond. J. Mol. Struct. 1077:101-113 (2014).
Jones P T, Dear P H, Foote J, Neuberger M S, Winter G. Replacing the complementarity-determining regions in a human antibody with those from a mouse. Nature. 321(6069):522-5 (1986).
Kaufmann F, Lund E, Eddy B. Proposal for a change in the nomenclature of Diplococcus pneumoniae and a comparison of the Danish and American type designations. Intl Bulletin of Bacterial Nomenclature and Taxonomy 10(1):31-40 (1960).
Kim K H, Yu J, Nahm M H. Efficiency of a pneumococcal opsonophagocytic killing assay improved by multiplexing and by coloring colonies. Clin Diagn Lab Immunol. 10(4):616-21 (2003).
Kojima K, Ishizaka A, Oshika E, Taguchi Y, Tomizawa K, et al. Quantitation of IgG subclass antibodies to pneumococcal capsular polysaccharides by ELISA, using Pneumovax-specific antibodies as a reference. Tohoku J Exp Med. 161(3):209-15 (1990).
Koskela M and Leinonen M. Comparison of ELISA and RIA for measurement of pneumococcal antibodies before and after vaccination with 14-valent pneumococcal capsular polysaccharide vaccine. J Clin Pathol. 34(1):93-8 (1981).
Larkin, M. A.; Blackshields, G.; Brown, N. P.; Chenna, R.; McGettigan, P. A.; McWilliam, H.; Valentin, F.; Wallace, I. M.; Wilm, A.; Lopez, R.; et al. Clustal W and Clustal X Version 2.0. Bioinforma. Oxf. Engl. 23:2947-2948 (2007).
Leung, K., et al., Early empirical assessment of the N501Y mutant strains of SARS-CoV-2 in the United Kingdom, October to November 2020. medRxiv, 2020.12.20.20248581 (2020).
Levine M M and Noriega F. Vaccines to prevent enteric infections, Baillieres Clin. Gastroenterol. 7(2):501-17 (1993).
Liu Y, et al., High-throughput screening for developability during early-stage antibody discovery using self-interaction nanoparticle spectroscopy. MAbs. 6(2):483-92 (2014).
Martin, E W, Ed. Remington's Pharmaceutical Sciences. 15th ed. Easton, PA: Mack Publishing Company, 1975.
Martinez J E, Romero-Steiner S, Pilishvili T, Barnard S, Schinsky J, et al. A flow cytometric opsonophagocytic assay for measurement of functional antibodies elicited after vaccination with the 23-valent pneumococcal polysaccharide vaccine. Clin Diagn Lab Immunol. 6(4):581-6 (1999).
Makowski E K, et al., Highly sensitive detection of antibody non-specific interactions using flow cytometry. Mabs 13(1):1951426 (2021).
Meir A, Helppolainen S H, Podoly E, Nordlund H R, Hytönen VP, Määttä JA, Wilchek M, Bayer E A, Kulomaa M S, Livnah O. Crystal Structure of Rhizavidin: Insights into the Enigmatic High-Affinity Interaction of an Innate Biotin-Binding Protein Dimer. J. Mol. Biol. 386:379-390 (2009).
Meyers E W and Miller W. Optimal alignments in linear space. Comput. Appl. Biosci. 4(1):11-7 (1988).
Mitchell M S. Cancer vaccines, a critical review-Part II. Curr Opin Investig Drugs. 3(1):150-8 (2002).
Munro C S, Stanley P J, Cole P J. Assessment of biological activity of immunoglobulin preparations by using opsonized micro-organisms to stimulate neutrophil chemiluminescence. Clin Exp Immunol. 61(1):183-8 (1985).
Ojo-Amaize E A, Church J A, Barka N E, Agopian M S, Peter J B. A rapid and sensitive chemiluminescence assay for evaluation of functional opsonic activity of Haemophilus influenzae type b-specific antibodies. Clin Diagn Lab Immunol. 2(3):286-90 (1995).
Oloo E O, Yethon J A, Ochs M M, Carpick B, Oomen R. Structure-guided antigen engineering yields pneumolysin mutants suitable for vaccination against pneumococcal disease. J Biol Chem 286(14):12133-40 (2011).
Orlandi R, Gussow D H, Jones P T, Winter G. Cloning immunoglobulin variable domains for expression by the polymerase chain reaction. Proc. Natl. Acad. Sci. USA. 86(1):3833-7 (1989).
Pan American Health Organization, Epidemiological update: occurrence of variants of SARS-CoV-2 in the Americas (2021). Available at iris.paho.org/bitstream/handle/10665.2/53219/EpiUpdate20January2021_eng.pdf.
Poljak R J. Production and structure of diabodies. Structure. 2(12):1121-3 (1994).
Powell M F and Newman M J. Vaccine Design: The Subunit and Adjuvant Approach. Springer. New York, NY (1995).
Queen C, Schneider W P, Selick H E, Payne P W, Landolfi N F, Duncan J F, Avdalovic N M, Levitt M, Junghans R P, Waldmann T A. A humanized antibody that binds to the interleukin 2 receptor. Proc. Natl. Acad. Sci. USA. 86(24):10029-33 (1989).
Raether H. Surface Plasmons on Smooth and Rough Surfaces and on Gratings. Springer (1988).
Rambaut A, Holmes E C, O'Toole A, Hill V, McCrone J T, Ruis C, du Plessis L, Pybus O G. A dynamic nomenclature proposal for SARS-CoV-2 lineages to assist genomic epidemiology. Nat. Microbiol. 5(11); 1403-1407 (2020).
Riechmann L, Clark M, Waldmann H, Winter G. Reshaping human antibodies for therapy. Nature. 332(6162):323-7 (1988).
Robbins J D and Robbins J B. Reexamination of the protective role of the capsular polysaccharide (Vi antigen) of Salmonella typhi. J. Infect. Dis. 150(3):436-49 (1984).
Robert X, Gouet P. Deciphering Key Features in Protein Structures with the New ENDscript Server. Nucleic Acids Res. 42: W320-324 (2014).
Romero-Steiner S, Libutti D, Pais L B, Dykes J, Anderson P, et al. Standardization of an opsonophagocytic assay for the measurement of functional antibody activity against
Streptococcus pneumoniae using differentiated HL-60 cells. Clin Diagn Lab Immunol. 1997 July; 4(4):415-22.
Romero-Steiner S, Holder P F, Gomez de Leon P, Spear W, Hennessy T W, et al. Avidity determinations for Haemophilus influenzae Type b anti-polyribosylribitol phosphate antibodies. Clin Diagn Lab Immunol. 2005 September; 12(9):1029-35.
Roth Z, Yehezkel G, Khalaila I. Identification and Quantification of Protein Glycosylation, Intl. J. Carb. Chem. 2012:640923 (2012).
Saeland E, Vidarsson G, Jonsdottir I. Pneumococcal pneumonia and bacteremia model in mice for the analysis of protective antibodies. Microb Pathog. 29(2):81-91 (2000).
Saif L J. Coronavirus immunogens. Vet. Microbiol. 37(3-4):285-97 (1993).
Sambrook J, Fritsch E R, Maniatis T. Molecular Cloning, a Laboratory Manual. 2^{nd} Ed. Cold Spring Harbor Press. Cold Spring Harbard, NY (1989).
Saunders K O. Conceptual Approaches to Modulating Antibody Effector Functions and Circulation Half-Life. Front. Immunol. 10:1296 (2019).
Schiffman G, Douglas R M, Bonner M J, Robbins M, Austrian R. Method for the radioimmunoassay of anticapsular antibodies and comparison with other techniques. J. Immunol. Methods 33(2):133-44 (1980).
Sharma A and Qadri A. Vi polysaccharide of Salmonella typhi targets the prohibitin family of molecules in intestinal epithelial cells and suppresses early inflammatory responses. Proc. Natl. Acad. Sci. USA. 101(50):17492-7 (2004).
Singh M and Indresh S. Advances in vaccine adjuvants for infectious diseases, Current HIV Research. 1(3):309-320 (2003).
Sjölander S and Urbaniczky C. Integrated fluid handling system for biomolecular interaction analysis. Anal. Chem. 63(20):2338-45 (1991).
Stack A M, Malley R, Thompson C M, Kobzik L, Siber G R, et al. Minimum protective serum concentrations of pneumococcal anti-capsular antibodies in infant rats. J Infect Dis. 1998 April; 177(4):986-90.
Szabo A, Stolz L, Granzow R. Surface plasmon resonance and its use in biomolecular interaction analysis (BIA). Curr. Opin. Struct. Biol. 5(5):699-705 (1995).
Tegally H, et al., Emergence and rapid spread of a new severe acute respiratory syndrome-related coronavirus 2 (SARS-CoV-2) lineage with multiple Spike mutations in South Africa. medRxiv, 2020.12.21.20248640 (2020).
Thomson E C, et al., The circulating SARS-CoV-2 Spike variant N439K maintains fitness while evading antibody-mediated immunity. bioRxiv, 2020.11.04.355842 (2020).
Verhoeyen M, Milstein C, Winter G. Rehaping human antibodies: grating an antilysozyme activity. Science. 239(4847):1534-6 (1988).
Williams et al., Innate imprinting by the modified heat-labile toxin of Escherichia coli (LTK63) provides generic protection against lung infectious disease. The Journal of Immunology. 173:7435-7443 (2004).
Wu Y, Wan T, Zhou X, Wang B, Yang F, Li N, Chen G, Dai S, Liu S, Zhang M, Cao X. HSP70-like protein 1 fusion protein enhances induction of carcinoembryonic antigen-specific CD8+ CTL response by dendritic cell vaccine. Cancer Res. 65(11):4947-54 (2005).
Wu R and Grossman L. “Recombinant DNA Part D” Methods in Enzymology. Vo. 153. Academic Press (1987).
Xu Y, Roach W, Sun T, Jain T, Prinz B, Yu T-Y, Torrey J, Thomas J, Bobrowicz P, Vasquez M, Wittrup K D, Krauland E. Addressing polyspecificity of antibodies selected from an in vitro yeast presentation system: a FACS-based, high-throughput selection and analytical tool. Protein Eng. Des. Sel. 26(10):663-70 (2013).
Zhang F, Lu Y J, Malley R. Multiple antigen-presenting system (MAPS) to induce comprehensive B- and T-cell immunity. Proc Natl Acad Sci USA. 110:13564-9 (2013).
Zhang F, Ledue O, Jun M, Goulart C, Malley R, Lu Y J. Protection against Staphylococcus Aureus Colonization and Infection by B- and T-Cell-Mediated Mechanisms. mBio 9: e01949-18 (2018).

### EQUIVALENTS

Those skilled in the art will recognize, or be able to ascertain using no more than routine experimentation, many equivalents to the specific embodiments described herein. The scope of the present disclosure is not intended to be limited to the above Description, but rather is as set forth in the following claim.

1. A variant rhizavidin polypeptide, comprising a mutation at one or more N-linked glycosylation sites of a wild-type rhizavidin polypeptide, wherein the wild-type rhizavidin polypeptide comprises the amino acid sequence as set forth below:
FDASNFKDFSSIASASSSWQNQSGSTMIIQVDSFGNVSGQYVNRAQGTGCONSPY PLTGRVNGTFIAFSVGWNNSTENCNSATGWTGYAQVNGNNTEIVTSWNLAYEG GSGPAIEQGQDTFQYVPTTENKSLL (SEQ ID NO: 2), wherein the amino acid sequence of SEQ ID NO: 2 corresponds to amino acid residues 45-179 of the wild-type rhizavidin polypeptide.

2. The variant rhizavidin polypeptide of claim 1, wherein the variant rhizavidin polypeptide comprises a mutation at each of four or more N-linked glycosylation sites of the wild-type rhizavidin polypeptide.

3. The variant rhizavidin polypeptide of claim 2, wherein the variant rhizavidin polypeptide comprises a mutation at each of five or more N-linked glycosylation sites of the wild-type rhizavidin polypeptide.

4. The variant rhizavidin polypeptide claim 1, wherein at least one of the N-linked glycosylation sites comprises a consensus sequence of NXaaS or NXaaT, wherein Xaa is any amino acid residue except proline (P).

5. (canceled)

6. (canceled)

7. The variant rhizavidin polypeptide of claim 1, wherein at least one of the N-linked glycosylation sites comprises amino acid residues selected from the group consisting of amino acid residues 80-82, 106-108, 117-119, 118-120, and 138-140 of the wild-type rhizavidin polypeptide.

8. The variant rhizavidin polypeptide of claim 7, wherein at least one of the mutations is at a position selected from the group consisting of amino acid residues 80, 106, 108, 118, 119, and 138 of the wild-type rhizavidin polypeptide.

9. The variant rhizavidin polypeptide of claim 8, wherein at least one of the mutations is selected from the group consisting of:
(i) residue 80 N to A/Q, (ii) residue 106 N to A/Q, (iii) residue 108 T to A/Q, (iv) residue 118 N to A/Q, (v) residue 119 S to A/Q, and (vi) residue 138 N to A/Q.

10. The variant rhizavidin polypeptide of claim 9, comprising mutations at each of:
(a) (i) residue 80 N to A, (ii) residue 108 T to A, (iii) residue 118 N to A, (iv) residue 119 S to A, and (v) residue 138 N to A;
(b) (i) residue 80 N to A, (ii) residue 106 N to A, (iii) residue 118 N to A, (iv) residue 119 S to A, and (v) residue 138 N to A;
(c) (i) residue 80 N to A, (ii) residue 106 N to A, (iii) residue 118 N to A, and (iv) residue 138 N to A; or
(d) (i) residue 80 N to Q, (ii) residue 106 N to Q, (iii) residue 118 N to Q, and (iv) residue 138 N to Q.

11. (canceled)

12. (canceled)

13. (canceled)

14. The variant rhizavidin polypeptide of claim 1, wherein the variant rhizavidin polypeptide does not comprise amino acid residues 1-44 of the wild-type rhizavidin.

15. The variant rhizavidin polypeptide of claim 1, wherein the variant rhizavidin polypeptide comprises an amino acid sequence having at least 80%, at least 85%, at least 90%, at least 95%, at least 96%, at least 97%, at least 98%, or at least 99% identity to the amino acid sequence of any one of SEQ ID NOs: 4-15.

16. The variant rhizavidin polypeptide of claim 1, wherein the variant rhizavidin polypeptide is characterized in that it binds to biotin or a derivative or mimic molecule thereof, at an affinity that is at least 50%, at least 60%, at least 70%, at least 80%, or at least 90% of the wild-type rhizavidin polypeptide.

17. (canceled)

18. The variant rhizavidin polypeptide of claim 16, wherein the variant rhizavidin polypeptide is characterized in that its degree of glycosylation when expressed in a mammalian cell is reduced by at least 70%, at least 80%, or at least 90% as compared to the wild-type rhizavidin polypeptide.

19. The variant rhizavidin polypeptide of claim 1, wherein the variant rhizavidin polypeptide is characterized in that when a population of the variant rhizavidin polypeptide is expressed in mammalian cells the population of variant rhizavidin polypeptides have an average monomeric molecular weight between 17 kDa and 19 kDa.

20. The variant rhizavidin polypeptide of claim 1, wherein the variant rhizavidin polypeptide is characterized in that when a population of the variant rhizavidin polypeptide is expressed in mammalian cells the population of variant rhizavidin polypeptides have an average dimeric molecular weight between 26 kDa and 28 kDa.

21. A fusion protein comprising the variant rhizavidin polypeptide of claim 1 and at least one additional polypeptide.

22. A nucleic acid comprising a nucleotide sequence encoding the fusion protein of claim 21.

23. A vector comprising the nucleic acid of claim 22.

24. A method of producing the fusion protein of claim 21, the method comprising using a nucleic acid comprising a nucleotide sequence encoding the fusion protein to express the variant rhizavidin polypeptide or the fusion protein in a host cell.

25. (canceled)

26. A pharmaceutical composition comprising the fusion protein of claim 21, and a pharmaceutically acceptable carrier.

27. (canceled)

28. (canceled)

29. A method of immunizing a subject, comprising administering to the subject the pharmaceutical composition of claim 26.


SEQUENCE LISTING (162 sequences)

SEQ ID NO: 1 - protein, 179 aa, Rhizobium etli
MIITSLYATFGTIADGRRTSGGKTMIRTNAVAALVFAVATSALAFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 2 - protein, 135 aa, synthetic construct
FDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 3 - protein, 136 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 4 - protein, 179 aa, synthetic construct
MIITSLYATFGTIADGRRTSGGKTMIRTNAVAALVFAVATSALAFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 5 - protein, 135 aa, synthetic construct
FDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 6 - protein, 135 aa, synthetic construct
MFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLK

SEQ ID NO: 7 - protein, 179 aa, synthetic construct
MIITSLYATFGTIADGRRTSGGKTMIRTNAVAALVFAVATSALAFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 8 - protein, 135 aa, synthetic construct
FDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 9 - protein, 135 aa, synthetic construct
MFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLK

SEQ ID NO: 10 - protein, 179 aa, synthetic construct
MIITSLYATFGTIADGRRTSGGKTMIRTNAVAALVFAVATSALAFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 11 - protein, 135 aa, synthetic construct
FDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 12 - protein, 136 aa, synthetic construct
MFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 13 - protein, 179 aa, synthetic construct
MIITSLYATFGTIADGRRTSGGKTMIRTNAVAALVFAVATSALAFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 14 - protein, 135 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 15 - protein, 136 aa, synthetic construct
MFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 16 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - protein, 194 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 18 - protein, 72 aa, SARS-CoV-2
NSNNLDSKVGGNYNYLYRLFRKSNLKPFERDISTEIYQAGSTPCNGVEGFNCYFPLQSYGFQPTNGVGYQPY

SEQ ID NO: 19 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 20 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGP

SEQ ID NO: 21 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGP

SEQ ID NO: 22 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGP

SEQ ID NO: 23 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGP

SEQ ID NO: 24 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGP

SEQ ID NO: 25 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGP

SEQ ID NO: 26 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGP

SEQ ID NO: 27 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTNGVGYQPYRVVVLSFELLHAPATVCGP

SEQ ID NO: 28 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGP

SEQ ID NO: 29 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGNTPCNGVAGFNCYFPLKSYSFQPTYGVGHQPYRVVVLSFELLHAPATVCGP

SEQ ID NO: 30 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGP

SEQ ID NO: 31 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGP

SEQ ID NO: 32 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGP

SEQ ID NO: 33 - protein, 197 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGP

SEQ ID NO: 34 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 35 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIREGTGLAFEWWRTVYEKTDLPLVRKRTISIWGTTLYPQVEDKVEND

SEQ ID NO: 36 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGPDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIREGTGLAFEWWRTVYEKTDLPLVRKRTISIWGTTLYPQVEDKVEND

SEQ ID NO: 37 - protein, 470 aa, Streptococcus pneumoniae
ANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGPDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIREGTGLAFEWWRTVYEKTDLPLVRKRTISIWGTTLYPQVEDKVEND

SEQ ID NO: 38 - protein, 186 aa, Streptococcus pneumoniae
MIEASKLKAGMTFETADGKLIRVLEASHHKPGKGNTIMRMKLRDVRTGSTFDTSYRPEEKFEQAIIETVPAQYLYKMDDTAYFMNTETYDQYEIPVVNVENELLYILENSDVKIQFYGTEVIGVTVPTTVELTVAETQPSIKGATVTGSGKPATMETGLVVNVPDFIEAGQKLVINTAEGTYVSRA

SEQ ID NO: 39 - protein, 124 aa, Streptococcus pneumoniae
EQAIIETVPAQYLYKMDDTAYFMNTETYDQYEIPVVNVENELLYILENSDVKIQFYGTEVIGVTVPTTVELTVAETQPSIKGATVTGSGKPATMETGLVVNVPDFIEAGQKLVINTAEGTYVSR

SEQ ID NO: 40 - protein, 399 aa, Streptococcus pneumoniae
MKKKNGKAKKWQLYAAIGAASVVVLGAGGILLFRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATN

SEQ ID NO: 41 - protein, 367 aa, Streptococcus pneumoniae
FRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATN

SEQ ID NO: 42 - protein, 278 aa, Streptococcus pneumoniae
MKKWMLVLVSLMTALFLVACGKNSSETSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQ

SEQ ID NO: 43 - protein, 252 aa, Streptococcus pneumoniae
TSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQ

SEQ ID NO: 44 - DNA, 1410 bp, synthetic construct
  source 1..1410: mol_type = other DNA
atggcaaataaagcagtaaatgactttatactagctatgaattacgataaaaagaaactcttgacccatcagggagaaagtattgaaaatcgtttcatcaaagagggtaatcagctacccgatgagtttgttgttatcgaaagaaagaagcggagcttgtcgacaaatacaagtgatatttctgtaacagctaccaacgacagtcgcctctatcctggagcacttctcgtagtggatgagaccttgttagagaataatcccactcttcttgcggtcgatcgtgctccgatgacttatagtattgatttgcctggtttggcaagtagcgatagctttctccaagtggaagaccccagcaattcaagtgttcgcggagcggtaaacgatttgttggctaagtggcatcaagattatggtcaggtcaataatgtcccagctagaatgcagcatgaaaaaatcacggctcacagcatggaacaactcaaggtcaagtttggttctgactttgaaaagatagggaattctcttgatattgattttaactctgtccattcaggcgaaaagcagattcagattgttaattttaagcagatttattatacagtcagcgtagatgctgttaaaaatccaggagatgtgtttcaagatactgtaacggtagaggatttaaggcagagaggaatttctgcagagcgtcctttggtctatatttcgagtgttgcttatgggcgccaagtctatctcaagttggaaaccacgagtaagagtgatgaagtagaggctgcttttgaatctttgataaaaggagtagctcctcagacagagtggaagcagattttggacaatacagaagtgaaggcggttattttagggggcgacccaagttcgggtgcccgagttgtaacaggcaaggtggatatggtagaggacttgattcaagaaggcagtcgctttacagccgatcatccaggcttgccgatttcctatacaacttcttttttacgtgacaatgtagttgcgacctttcaaaacagtacagactatgttgagactaaggttacagcttacagaaacggagatttactgctggatcatagtggtgcctatgttgctcaatattatattacttgggatgaattatcctatgatcatcaaggcaaggaagtcttgactcctaaggcttgggacagaaatgggcaggatttgacggctcactttaccactagtattcctttaaaagggaatgttcgcaatctctctgtcaaaattagagagtgtaccgggcttgcctgggaatggtggcgtacggtttatgaaaaaaccgatttgccactagtgcgtaagcggacgatttctatttggggaacaactctctatcctcaggtagaggataaggtagaaaatgattag

SEQ ID NO: 45 - DNA, 558 bp, synthetic construct
  source 1..558: mol_type = other DNA
atgattgaagcaagtaaattaaaagctggtatgacctttgaaacagctgacggcaaattgattcgcgttttggaagctagtcaccacaaaccaggtaaaggaaacacgatcatgcgtatgaaattgcgtgatgtccgtactggttctacatttgacacaagctaccgtccagaggaaaaatttgaacaagctattatcgagactgtcccagctcaatacttgtacaaaatggatgacacagcatacttcatgaatacagaaacttatgaccaatacgaaatccctgtagtcaatgttgaaaacgaattgctttacatccttgaaaactctgatgtgaaaatccaattctacggaactgaagtgatcggtgtcaccgttcctactactgttgagttgacagttgctgaaactcaaccatctatcaaaggtgctactgttacaggttctggtaaaccagcaacgatggaaactggacttgtcgtaaacgttccagacttcatcgaagcaggacaaaaactcgttatcaacactgcagaaggaacttacgtttctcgtgcc

SEQ ID NO: 46 - DNA, 1197 bp, synthetic construct
  source 1..1197: mol_type = other DNA
atgaagaaaaagaatggtaaagctaaaaagtggcaactgtatgcagcaatcggtgctgcgagtgtagttgtattgggtgctggggggattttactctttagacaaccttctcagactgctctaaaagatgagcctactcatcttgttgttgccaaggaaggaagcgtggcctcctctgttttattgtcagggacagtaacagcaaaaaatgaacaatatgtttattttgatgctagtaagggtgatttagatgaaatccttgtttctgtgggcgataaggtcagcgaagggcaggctttagtcaagtacagtagttcagaagcgcaggcggcctatgattcagctagtcgagcagtagctagggcagatcgtcatatcaatgaactcaatcaagcacgaaatgaagccgcttcagctccggctccacagttaccagcgccagtaggaggagaagatgcaacggtgcaaagcccaactccagtggctggaaattctgttgcttctattgacgctcaattgggtgatgcccgtgatgcgcgtgcagatgctgcggcgcaattaagcaaggctcaaagtcaattggatgcaacaactgttctcagtaccctagagggaactgtggtcgaagtcaatagcaatgtttctaaatctccaacaggggcgagtcaagttatggttcatattgtcagcaatgaaaatttacaagtcaagggagaattgtctgagtacaatctagccaacctttctgtaggtcaagaagtaagctttacttctaaagtgtatcctgataaaaaatggactgggaaattaagctatatttctgactatcctaaaaacaatggtgaagcagctagtccagcagccgggaataatacaggttctaaatacccttatactattgatgtgacaggcgaggttggtgatttgaaacaaggtttttctgtcaacattgaggttaaaagcaaaactaaggctattcttgttcctgttagcagtctagtaatggatgatagtaaaaattatgtctggattgtggatgaacaacaaaaggctaaaaaagttgaggtttcattgggaaatgctgacgcagaaaatcaagaaatcacttctggtttaacgaacggtgctaaggtcatcagtaatccaacatcttccttggaagaaggaaaagaggtgaaggctgatgaagcaactaat

SEQ ID NO: 47 - DNA, 834 bp, synthetic construct
  source 1..834: mol_type = other DNA
atgaaaaaatggatgcttgtattagtcagtctgatgactgctttgttcttagtagcttgtgggaaaaattctagcgaaactagtggagataattggtcaaagtaccagtctaacaagtctattactattggatttgatagtacttttgttccaatgggatttgctcagaaagatggttcttatgcaggatttgatattgatttagctacagctgtttttgaaaaatacggaatcacggtaaattggcaaccgattgattgggatttgaaagaagctgaattgacaaaaggaacgattgatctgatttggaatggctattccgctacagacgaacgccgtgaaaaggtggctttcagtaactcatatatgaagaatgagcaggtattggttacgaagaaatcatctggtatcacgactgcaaaggatatgactggaaagacattaggagctcaagctggttcatctggttatgcggactttgaagcaaatccagaaattttgaagaatattgtcgctaataaggaagcgaatcaataccaaacctttaatgaagccttgattgatttgaaaaacgatcgaattgatggtctattgattgaccgtgtctatgcaaactattatttagaagcagaaggtgttttaaacgattataatgtctttacagttggactagaaacagaagcttttgcggttggagcccgtaaggaagatacaaacttggttaagaagataaatgaagctttttctagtctttacaaggacggcaagttccaagaaatcagccaaaaatggtttggagaagatgtagcaaccaaagaagtaaaagaaggacag

SEQ ID NO: 48 - protein, 7 aa, synthetic construct
GGGGSSS

SEQ ID NO: 49 - protein, 5 aa, synthetic construct
GGGGS

SEQ ID NO: 50 - protein, 5 aa, synthetic construct
GGGGS

SEQ ID NO: 51 - protein, 15 aa, synthetic construct
GGGGSGGGGSGGGGS

SEQ ID NO: 52 - protein, 30 aa, synthetic construct
GGGGSGGGGSGGGGSGGGGSGGGGSGGGGS

SEQ ID NO: 53 - protein, 18 aa, synthetic construct
KESGSVSSEQLAQFRSLD

SEQ ID NO: 54 - protein, 14 aa, synthetic construct
EGKSSGSGSESKST

SEQ ID NO: 55 - protein, 5 aa, synthetic construct
GGGGG

SEQ ID NO: 56 - protein, 6 aa, synthetic construct
GGGGGG

SEQ ID NO: 57 - protein, 8 aa, synthetic construct
GGGGGGGG

SEQ ID NO: 58 - protein, 12 aa, synthetic construct
GSAGSAAGSGEF

SEQ ID NO: 59 - protein, 5 aa, synthetic construct
EAAAK

SEQ ID NO: 60 - protein, 5 aa, synthetic construct
EAAAK

SEQ ID NO: 61 - protein, 7 aa, synthetic construct
AEAAAKA

SEQ ID NO: 62 - protein, 12 aa, synthetic construct
AEAAAKEAAAKA

SEQ ID NO: 63 - protein, 46 aa, synthetic construct
AEAAAKEAAAKEAAAKEAAAKALEAEAAAKEAAAKEAAAKEAAAKA

SEQ ID NO: 64 - protein, 12 aa, synthetic construct
AEAAAKEAAAKA

SEQ ID NO: 65 - protein, 12 aa, synthetic construct
AEAAAKEAAAKA

SEQ ID NO: 66 - protein, 8 aa, synthetic construct
XPXPXPXP

SEQ ID NO: 67 - protein, 8 aa, synthetic construct
APAPAPAP

SEQ ID NO: 68 - protein, 32 aa, synthetic construct
APAPAPAPAPAPAPAPAPAPAPAPAPAPAPAP

SEQ ID NO: 69 - protein, 8 aa, synthetic construct
KPKPKPKP

SEQ ID NO: 70 - protein, 8 aa, synthetic construct
QPQPQPQP

SEQ ID NO: 71 - protein, 14 aa, synthetic construct
APAPAPAPAPAPAP

SEQ ID NO: 72 - protein, 21 aa, synthetic construct
GAPGGGGGAAAAAGGGGGGAP

SEQ ID NO: 73 - protein, 39 aa, synthetic construct
GAPGGGGGAAAAAGGGGGGAPGGGGGAAAAAGGGGGGAP

SEQ ID NO: 74 - protein, 57 aa, synthetic construct
GAPGGGGGAAAAAGGGGGGAPGGGGGAAAAAGGGGGGAPGGGGGAAAAAGGGGGGAP

SEQ ID NO: 75 - protein, 4 aa, synthetic construct
VSDP

SEQ ID NO: 76 - protein, 4 aa, synthetic construct
AAAA

SEQ ID NO: 77 - protein, 4 aa, synthetic construct
GGGG

SEQ ID NO: 78 - protein, 4 aa, synthetic construct
HHHH

SEQ ID NO: 79 - protein, 6 aa, synthetic construct
HHHHHH

SEQ ID NO: 80 - protein, 10 aa, synthetic construct
MSYYHHHHHH

SEQ ID NO: 81 - protein, 19 aa, synthetic construct
MKKIWLALAGLVLAFSASA

SEQ ID NO: 82 - protein, 23 aa, synthetic construct
MAPFEPLASGILLLLWLIAPSRA

SEQ ID NO: 83 - protein, 17 aa, synthetic construct
MKKVAAFVALSLLMAGC

SEQ ID NO: 84 - protein, 24 aa, synthetic construct
MKKIMLVITLILVSPIAQQTEAKD

SEQ ID NO: 85 - protein, 29 aa, synthetic construct
MKKKIISAILMSTVILSAAAPLSGVYADT

SEQ ID NO: 86 - protein, 31 aa, synthetic construct
MKKRKVLIPLMALSTILVSSTGNLEVIQAEV

SEQ ID NO: 87 - protein, 29 aa, synthetic construct
MNMKKATIAATAGIAVTAFAAPTIASAST

SEQ ID NO: 88 - protein, 54 aa, synthetic construct
MQKTRKERILEALQEEKKNKKSKKFKTGATIAGVTAIATSITVPGIEVIVSADE

SEQ ID NO: 89 - protein, 28 aa, synthetic construct
MKKLKMASCALVAGLMFSGLTPNAFAED

SEQ ID NO: 90 - protein, 31 aa, synthetic construct
MAKKFNYKLPSMVALTLVGSAVTAHQVQAAE

SEQ ID NO: 91 - protein, 59 aa, synthetic construct
MTDKKSENQTEKTETKENKGMTRREMLKLSAVAGTGIAVGATGLGTILNVVDQVDKALT

SEQ ID NO: 92 - protein, 53 aa, synthetic construct
MAYDSRFDEWVQKLKEESFQNNTFDRRKFIQGAGKIAGLGLGLTIAQSVGAFG

SEQ ID NO: 93 - protein, 19 aa, synthetic construct
MEFGLSWLFLVAILKGVQC

SEQ ID NO: 94 - protein, 19 aa, synthetic construct
MGWSCIILFLVATATGVHS

SEQ ID NO: 95 - protein, 348 aa, unidentified
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 96 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 97 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 98 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 99 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 100 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 101 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 102 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 103 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 104 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 105 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGNTPCNGVAGFNCYFPLKSYSFQPTYGVGHQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 106 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 107 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 108 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 109 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKD

SEQ ID NO: 110 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 111 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 112 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 113 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 114 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 115 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 116 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 117 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 118 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTNGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 119 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 120 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGNTPCNGVAGFNCYFPLKSYSFQPTYGVGHQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 121 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 122 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 123 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 124 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKD

SEQ ID NO: 125 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 126 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 127 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 128 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 129 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 130 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 131 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 132 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 133 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 134 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 135 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGNTPCNGVAGFNCYFPLKSYSFQPTYGVGHQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 136 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 137 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 138 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 139 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKD

SEQ ID NO: 140 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 141 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 142 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 143 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 144 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 145 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 146 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 147 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 148 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 149 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 150 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGNTPCNGVAGFNCYFPLKSYSFQPTYGVGHQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 151 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 152 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 153 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 154 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 155 - protein, 744 aa, synthetic construct
MFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKDGGGGSSSANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGPDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIREGTGLAFEWWRTVYEKTDLPLVRKRTISIWGTTLYPQVEDKVENDGGGGSSSEQAIIETVPAQYLYKMDDTAYFMNTETYDQYEIPVVNVENELLYILENSDVKIQFYGTEVIGVTVPTTVELTVAETQPSIKGATVTGSGKPATMETGLVVNVPDFIEAGQKLVINTAEGTYVSR

SEQ ID NO: 156 - protein, 744 aa, synthetic construct
MFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKDGGGGSSSANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGPDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIREGTGLAFEWWRTVYEKTDLPLVRKRTISIWGTTLYPQVEDKVENDGGGGSSSEQAIIETVPAQYLYKMDDTAYFMNTETYDQYEIPVVNVENELLYILENSDVKIQFYGTEVIGVTVPTTVELTVAETQPSIKGATVTGSGKPATMETGLVVNVPDFIEAGQKLVINTAEGTYVSR

SEQ ID NO: 157 - protein, 744 aa, synthetic construct
MFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKDGGGGSSSANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGPDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIREGTGLAFEWWRTVYEKTDLPLVRKRTISIWGTTLYPQVEDKVENDGGGGSSSEQAIIETVPAQYLYKMDDTAYFMNTETYDQYEIPVVNVENELLYILENSDVKIQFYGTEVIGVTVPTTVELTVAETQPSIKGATVTGSGKPATMETGLVVNVPDFIEAGQKLVINTAEGTYVSR

SEQ ID NO: 158 - protein, 744 aa, synthetic construct
MFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGPDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIREGTGLAFEWWRTVYEKTDLPLVRKRTISIWGTTLYPQVEDKVENDGGGGSSSEQAIIETVPAQYLYKMDDTAYFMNTETYDQYEIPVVNVENELLYILENSDVKIQFYGTEVIGVTVPTTVELTVAETQPSIKGATVTGSGKPATMETGLVVNVPDFIEAGQKLVINTAEGTYVSR

SEQ ID NO: 159 - protein, 765 aa, synthetic construct
MFDASNFKDFSSIASASSSWQNQSGSTMIIQVDSFGAVSGQYVNRAQGTGCQNSPYPLTGRVAGTFIAFSVGWNASTENCNSATGWTGYAQVNGANTEIVTSWNLAYEGGSGPAIEQGQDTFQYVPTTENKSLLKDGGGGSSSTSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQAAAFRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATN

SEQ ID NO: 160 - protein, 765 aa, synthetic construct
MFDASNFKDFSSIASASSSWQNQSGSTMIIQVDSFGQVSGQYVNRAQGTGCQNSPYPLTGRVQGTFIAFSVGWNQSTENCNSATGWTGYAQVNGQNTEIVTSWNLAYEGGSGPAIEQGQDTFQYVPTTENKSLLKDGGGGSSSTSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQAAAFRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATN

SEQ ID NO: 161 - protein, 765 aa, synthetic construct
MFDASNFKDFSSIASASSSWQNQSGSTMIIQVDSFGAVSGQYVNRAQGTGCQNSPYPLTGRVAGTFIAFSVGWNAATENCNSATGWTGYAQVNGANTEIVTSWNLAYEGGSGPAIEQGQDTFQYVPTTENKSLLKDGGGGSSSTSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQAAAFRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATN

SEQ ID NO: 162 - protein, 765 aa, synthetic construct
MFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSTSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQAAAFRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATN